SEQUENCE LISTING (36 sequences)

SEQ ID NO: 1 - protein, 165 aa, Homo sapiens
MAKMNLSSYILILTFSLFSQGILLSASKSIRNLDDDMVFNTFRLGKGFQKEDTAEKSVIAPSLEQYKNDESSFMNEEENKVSKNTGSKHNFLNHGLPLNLAIKPYLALKGSVAFPAENGVQNTESTQEKREIGDEENSAKFPIGRRDFDMLRCMLGRVYRPCWQV

SEQ ID NO: 2 - DNA, 456 bp, Homo sapiens
atggcaaaaatgaatctctcttcctatatattaatactaactttttctttgttttctcaaggtattttactttcagcatccaagtccataagaaatttagatgatgacatggtatttaatacattcaggttggggaaaggctttcagaaggaagacactgcagaaaaatcagttattgctccttccctggaacaatataaaaatgatgagagcagtttcatgaacgaagaggaaaataaagtttcaaagaacacaggctccaaacataatttcttaaatcatggtctgccactgaatctggctataaaaccttatcttgcactaaaaggatctgtagctttcccagctgagaatggagttcagaatactgaatcaacacaagaaaagagagaaattggggatgaagaaaactcagctaaatttcctataggaaggagagattttgacagtgagtag

SEQ ID NO: 3 - protein, 19 aa, Homo sapiens
GSVAFPAENGVQNTESTQE

SEQ ID NO: 4 - protein, 13 aa, Homo sapiens
EIGDEENSAKFPI

SEQ ID NO: 5 - protein, 19 aa, Homo sapiens
DFDMLRCMLGRVYRPCWQV

SEQ ID NO: 6 - protein, 412 aa, Homo sapiens
MPIMGSSVYITVELAIAVLAILGNVLVCWAVWLNSNLQNVTNYFVVSLAAADIAVGVLAIPFAITISTGFCAACHGCLFIACFVLVLTQSSIFSLLAIAIDRYIAIRIPLRYNGLVTGTRAKGIIAICWVLSFAIGLTPMLGWNNCGQPKEGKNHSQGCGEGQVACLFEDVVPMNYMVYFNFFACVLVPLLLMLGVYLRIFLAARRQLKQMESQPLPGERARSTLQKEVHAAKSLAIIVGLFALCWLPLHIINCFTFFCPDCSHAPLWLMYLAIVLSHTNSVVNPFIYAYRIREFRQTFRKIIRSHVLRQQEPFKAAGTSARVLAAHGSDGEQVSLRLNGHPPGVWANGSAPHPERRPNGYALGLVSGGSAQESQGNTGLPDVELLSHELKGVCPEPPGLDDPLAQDGAGVS

SEQ ID NO: 7 - DNA, 2529 bp, Homo sapiens
gaagctctgcctgggcctcagggactgtgacatggagcaggagccgcccccagccaagctgctttcagcacagcgtgggcccccagcaccttggtgcggggtgcggcccctcggaggagggctgtcaggtgaagcctcgtgtgagggggtgcctcaggaaccctgaagctgggctgagccatgatgctgctgccagaacccctgcagagggcctggtttcaggagactcagagtcctctgtgaaaaagcccttggagagcgccccagcagggctgcacttggctcctgtgaggaaggggctcaggggtctgggcccctccgcctgggccgggctgggagccaggcgggcggctgggctgcagcaatggaccgtgagctggcccagcccgcgtccgtgctgagcctgcctgtcgtctgtggccatgcccatcatgggctcctcggtgtacatcacggtggagctggccattgctgtgctggccatcctgggcaatgtgctggtgtgctgggccgtgtggctcaacagcaacctgcagaacgtcaccaactactttgtggtgtcactggcggcggccgacatcgcagtgggtgtgctcgccatcccctttgccatcaccatcagcaccgggttctgcgctgcctgccacggctgcctcttcattgcctgcttcgtcctggtcctcacgcagagctccatcttcagtctcctggccatcgccattgaccgctacattgccatccgcatcccgctccggtacaatggcttggtgaccggcacgagggctaagggcatcattgccatctgctgggtgctgtcgtttgccatcggcctgactcccatgctaggttggaacaactgcggtcagccaaaggagggcaagaaccactcccagggctgcggggagggccaagtggcctgtctctttgaggatgtggtccccatgaactacatggtgtacttcaacttctttgcctgtgtgctggtgcccctgctgctcatgctgggtgtctatttgcggatcttcctggcggcgcgacgacagctgaagcagatggagagccagcctctgccgggggagcgggcacggtccacactgcagaaggaggtccatgctgccaagtcactggccatcattgtggggctctttgccctctgctggctgcccctacacatcatcaactgcttcactttcttctgccccgactgcagccacgcccctctctggctcatgtacctggccatcgtcctctcccacaccaattcggttgtgaatcccttcatctacgcctaccgtatccgcgagttccgccagaccttccgcaagatcattcgcagccacgtcctgaggcagcaagaacctttcaaggcagctggcaccagtgcccgggtcttggcagctcatggcagtgacggagagcaggtcagcctccgtctcaacggccacccgccaggagtgtgggccaacggcagtgctccccaccctgagcggaggcccaatggctatgccctggggctggtgagtggagggagtgcccaagagtcccaggggaacacgggcctcccagacgtggagctccttagccatgagctcaagggagtgtgcccagagccccctggcctagatgaccccctggcccaggatggagcaggagtgtcctgatgattcatggagtttgccccttcctaagggaaggagatctttatctttctggttggcttgaccagtcacgttgggagaagagagagagtgccaggagaccctgagggcagccggttcctactttggactgagagaagggagccccaggctggagcagcatgaggcccagcaagaagggcttgggttctgaggaagcagatgtttcatgctgtgaggccttgcaccaggtgggggccacagcaccagcagcatctttgctgggcagggcccagccctccactgcagaagcatctggaagcaccaccttgtctccacagagcagcttgggcacagcagactggcctggccctgagactggggagtggctccaacagcctcctgccacccacacaccactctccctagactctcctagggttcaggagctgctgggcccagaggtgacatttgacttttttccaggaaaaatgtaagtgtgaggaaaccctttttattttattacctttcactctctggctgctgggtctgccgtcggtcctgctgctaacctggcaccagagcctctgcccggggagcctcaggcagtcctctcctgctgtcacagctgccatccacttctcagtcccagggccatctcttggagtgacaaagctgggatcaaggacagggagttgtaacagagcagtgccagagcatgggcccaggtcccaggggagaggttggggctggcaggccactggcatgtgctgagtagcgcagagctacccagtgagaggccttgtctaactgcctttccttctaaagggaatgtttttttctgagataaaataaaaacgagccacatcgtgttttaagcttgtccaaatga

SEQ ID NO: 8 - protein, 242 aa, Homo sapiens
MPGPWLLLALALTLNLTGVPGGRAQPEAAQQEAVTAAEHPGLDDFLRQVERLLFLRENIQRLQGDQGEHSASQIFQSDWLSKRQHPGKREEEEEEGVEEEEEEEGGAVGPHKRQHPGRREDEASWSVDVTQHKRQHPGRRSPWLAYAVPKRQHPGRRLADPKAQRSWEEEEEEEEREEDLMPEKRQHPGKRALGGPCGPQGAYGQAGLLLGLLDDLSRSQGAEEKRQHPGRRAAWVREPLEE

SEQ ID NO: 9 - DNA, 1560 bp, Homo sapiens
cggcaggcgcccggggtcctcagcgctgcagactcctgacctgccgactgcggatcccgagtccccggatcccggacccatcctgtggagcccactcctggcagacgccgcgatgcccggcccttggttgctgctcgctctggctttgaccctgaacctgaccggtgtccccggcggccgtgctcagccagaggcggcccagcaggaggcagtgacggccgcggagcatccgggcctggatgacttcctgcgccaggtggagcgcctcctcttcctccgggaaaacatccagcggctgcaaggggaccagggtgagcactccgcgtcccagatctttcaatctgactggctctccaaacgtcagcatccaggcaaaagagaggaggaggaggaagagggagttgaagaagaggaagaggaagaagggggggctgtgggaccccacaaacggcagcaccctggccgacgagaagatgaggcttcatggtcagtcgatgtaacccagcacaagcggcagcatcctggccggcgctccccctggcttgcatatgctgtcccgaagcggcagcacccaggcagaaggctggcagatcccaaggctcaaaggagctgggaagaagaggaggaggaggaagagagagaggaagacctgatgcctgaaaaacgccagcatccgggcaagagggccctgggaggcccctgtgggccccagggagcctatggtcaagcgggccttctgctggggctcctggatgacctgagtaggagccagggagctgaggaaaagcggcagcaccctggtcggcgggcagcctgggtcagagagcccctggaggagtgaacccagttttccctgaagtcgagtttgtggtctaaggatgtcttgagccctgtgtgccccaccattcatgacctctgtattctctagttagatccctgaccataagcctgagcccctccctcccagccccatattcacacacatcccagcccctggccttgccctcttcctttaggcatgtgagaaaatcagcctagcagtttaaaccccactttcctccacttagcaccataggcaagggggcagatcccagagcccctctcaccccccccaccacaggcctgctccttccttagccttggctaagatggtccttctgtgtcttgcaaagactccccaagtgggacagggagcccctgggagggcagccagtgagggtggggtgggactgaagcgttgtgtgcaaatccagcttccatcccctccccaacctggcaggattctccatgtgtaaacttcacccccaggacccaggatcttctcctttctgggcatccctttgtgggtgggcagagccctgacccacagctgtgttactgcttggagaagcatatgtaggggcataccctgtggtgttgtgctgtgtctggctgtgggataaatgtgtgtgggaatattgaaacatcgcctaggaattgtggtttgtatataaccctctaagcccctatcccttgtcgatgacagtcatcctaatgataataaaacctgcatccagataa

SEQ ID NO: 10 - protein, 320 aa, Homo sapiens
MGDIPAVGLSSWKASPGKVTEAVKEAIDAGYRHFDCAYFYHNEREVGAGIRCKIKEGAVRREDLFIATKLWCTCHKKSLVETACRKSLKALKLNYLDLYLIHWPMGFKPPHPEWIMSCSELSFCLSHPRVQDLPLDESNMVIPSDTDFLDTWEAMEDLVITGLVKNIGVSNFNHEQLERLLNKPGLRFKPLTNQIECHPYLTQKNLISFCQSRDVSVTAYRPLGGSCEGVDLIDNPVIKRIAKEHGKSPAQILIRFQIQRNVIVIPGSITPSHIKENIQVFDFELTQHDMDNILSLNRNLRLAMFPITKNHKDYPFHIEY

SEQ ID NO: 11 - protein, 263 aa, Homo sapiens
MGDIPAVGLSSWKASPGKVTEAVKEAIDAGYRHFDCAYFYHNEREVGAGIRCKIKEGAVRREDLFIATKLWCTCHKKSLVETACRKSLKALKLNYLDLYLIHWPMGFKPPHPEWIMSCSELSFCLSHPRVQDLPLDESNMVIPSDTDFLDTWEAMEDLVITGLVKNIGVSNFNHEQLERLLNKPGLRFKPLTNQILIRFQIQRNVIVIPGSITPSHIKENIQVFDFELTQHDMDNILSLNRNLRLAMFPITKNHKDYPFHIEY

SEQ ID NO: 12 - protein, 222 aa, Homo sapiens
MGDIPAVGLSSWKASPGKVTEAVKEAIDAGYRHFDCAYFYHNEREVGAGIRCKIKEGAVRREDLFIATKLWCTCHKKSLVETACRKSLKALKLNYLDLYLIHWPMGFKPPHPEWIMSCSELSFCLSHPRVQDLPLDESNMVIPSDTDFLDTWEILIRFQIQRNVIVIPGSITPSHIKENIQVFDFELTQHDMDNILSLNRNLRLAMFPITKNHKDYPFHIEY

SEQ ID NO: 13 - DNA, 1613 bp, Homo sapiens
gtcacaaggcacttccagccagtcgcaacggcgggtcgccagcgccgcagtagctcgcgcggtgcctgtcggtagtcgcgtgcggggcggcggggcggcggggcggccggcggcggccatgggagatatcccagccgtgggcctcagctcctggaaggcttctccagggaaagtgaccgaggcagtgaaagaggccattgacgcagggtaccggcacttcgactgtgcttacttttaccacaatgagagggaggttggagcagggatccgttgcaagatcaaggaaggcgctgtaagacgggaggatctgttcattgccactaagctgtggtgcacctgccataagaagtccttggtggaaacagcatgcagaaagagtctcaaggccttgaagctgaactatttggacctctacctcatacactggcccatgggtttcaagcctcctcatccagaatggatcatgagctgcagtgaactttccttctgcctctcacatcctcgagtgcaggacttgcctctggacgagagcaacatggttattcccagtgacacggacttcctggacacgtgggaggccatggaggacctggtgatcaccgggctggtgaagaacatcggggtgtcaaacttcaaccatgaacagcttgagaggcttttgaataagcctgggttgaggttcaagccactaaccaaccagattgagtgccacccatatcttactcagaagaatctgatcagtttttgccaatccagagatgtgtccgtgactgcttaccgtcctcttggtggctcgtgtgagggggttgacctgatagacaaccctgtgatcaagaggattgcaaaggagcacggcaagtctcctgctcagattttgatccgatttcaaatccagaggaatgtgatagtgatccccggatctatcaccccaagtcacattaaagagaatatccaggtgtttgattttgaattaacacagcacgatatggataacatcctcagcctaaacaggaatctccgactggccatgttccccataactaaaaatcacaaagactatcctttccacatagaatactgaggacgcttccccttccttgtttctgctcagcccagatgcacagacactattggcaatgttgaccctcctctgtcatcacagcgccagggcagctgtgcctgggacaggagccacacagtcagagggggatgtaagagccaccttctctgacaaatctggagaattgagtgtgttctaagtgaaggcaatggggtttctccaagacagcctgtgtggcctctactctgaacaaatacactgatgagtcatcagtgaaatttgccttcacattttaagaaaactttatcttatggagttatttaagccatctacagagctgaggaaacagtgtaatgtgtctctgccccattgcgcagctccacccattgtgccccaggccagcccgcgtcacctacacttccttctgtgccctgccagtgacccccaggttattctaaagcagagtccttcccttcccccagtgagaaggaaaatgggataagtctgggacactgtttcagttcaataaagaggcttttttcttccttaaaaa

SEQ ID NO: 14 - DNA, 1485 bp, Homo sapiens
atgcttccagccattgtcggagtgtcagccgtcacaaggcacttccagccagtcgcaacggcgggtcgccagcgccgcagtagctcgcgcggtgcctgtcggtagtcgcgtgcggggcggcggggcggcggggcggccggcggcggccatgggagatatcccagccgtgggcctcagctcctggaaggcttctccagggaaagtgaccgaggcagtgaaagaggccattgacgcagggtaccggcacttcgactgtgcttacttttaccacaatgagagggaggttggagcagggatccgttgcaagatcaaggaaggcgctgtaagacgggaggatctgttcattgccactaagctgtggtgcacctgccataagaagtccttggtggaaacagcatgcagaaagagtctcaaggccttgaagctgaactatttggacctctacctcatacactggcccatgggtttcaagcctcctcatccagaatggatcatgagctgcagtgaactttccttctgcctctcacatcctcgagtgcaggacttgcctctggacgagagcaacatggttattcccagtgacacggacttcctggacacgtgggaggccatggaggacctggtgatcaccgggctggtgaagaacatcggggtgtcaaacttcaaccatgaacagcttgagaggcttttgaataagcctgggttgaggttcaagccactaaccaaccagattttgatccgatttcaaatccagaggaatgtgatagtgatccccggatctatcaccccaagtcacattaaagagaatatccaggtgtttgattttgaattaacacagcacgatatggataacatcctcagcctaaacaggaatctccgactggccatgttccccataactaaaaatcacaaagactatcctttccacatagaatactgaggacgcttccccttccttgtttctgctcagcccagatgcacagacactattggcaatgttgaccctcctctgtcatcacagcgccagggcagctgtgcctgggacaggagccacacagtcagagggggatgtaagagccaccttctctgacaaatctggagaattgagtgtgttctaagtgaaggcaatggggtttctccaagacagcctgtgtggcctctactctgaacaaatacactgatgagtcatcagtgaaatttgccttcacattttaagaaaactttatcttatggagttatttaagccatctacagagctgaggaaacagtgtaatgtgtctctgccccattgcgcagctccacccattgtgccccaggccagcccgcgtcacctacacttccttctgtgccctgccagtgacccccaggttattctaaagcagagtccttcccttcccccagtgagaaggaaaatgggataagtctgggacactgtttcagttcaataaagaggcttttttcttccttaaaaaaaaaaaaaaaaaa

SEQ ID NO: 15 - DNA, 1362 bp, Homo sapiens
atgcttccagccattgtcggagtgtcagccgtcacaaggcacttccagccagtcgcaacggcgggtcgccagcgccgcagtagctcgcgcggtgcctgtcggtagtcgcgtgcggggcggcggggcggcggggcggccggcggcggccatgggagatatcccagccgtgggcctcagctcctggaaggcttctccagggaaagtgaccgaggcagtgaaagaggccattgacgcagggtaccggcacttcgactgtgcttacttttaccacaatgagagggaggttggagcagggatccgttgcaagatcaaggaaggcgctgtaagacgggaggatctgttcattgccactaagctgtggtgcacctgccataagaagtccttggtggaaacagcatgcagaaagagtctcaaggccttgaagctgaactatttggacctctacctcatacactggcccatgggtttcaagcctcctcatccagaatggatcatgagctgcagtgaactttccttctgcctctcacatcctcgagtgcaggacttgcctctggacgagagcaacatggttattcccagtgacacggacttcctggacacgtgggagattttgatccgatttcaaatccagaggaatgtgatagtgatccccggatctatcaccccaagtcacattaaagagaatatccaggtgtttgattttgaattaacacagcacgatatggataacatcctcagcctaaacaggaatctccgactggccatgttccccataactaaaaatcacaaagactatcctttccacatagaatactgaggacgcttccccttccttgtttctgctcagcccagatgcacagacactattggcaatgttgaccctcctctgtcatcacagcgccagggcagctgtgcctgggacaggagccacacagtcagagggggatgtaagagccaccttctctgacaaatctggagaattgagtgtgttctaagtgaaggcaatggggtttctccaagacagcctgtgtggcctctactctgaacaaatacactgatgagtcatcagtgaaatttgccttcacattttaagaaaactttatcttatggagttatttaagccatctacagagctgaggaaacagtgtaatgtgtctctgccccattgcgcagctccacccattgtgccccaggccagcccgcgtcacctacacttccttctgtgccctgccagtgacccccaggttattctaaagcagagtccttcccttcccccagtgagaaggaaaatgggataagtctgggacactgtttcagttcaataaagaggcttttttcttccttaaaaaaaaaaaaaaaaaa

SEQ ID NO: 16 - protein, 458 aa, Homo sapiens
MEATGTDEVDKLKTKFISAWNNMKYSWVLKTKTYFSRNSPVLLLGKCYHFKYEDEDKTLPAESGCTIEDHVIAGNVEEFRKDFISRIWLTYREEFPQIEGSALTTDCGWGCTLRTGQMLLAQGLILHFLGRAWTWPDALNIENSDSESWTSHTVKKFTASFEASLSGEREFKTPTISLKETIGKYSDDHEMRNEVYHRKIISWFGDSPLALFGLHQLIEYGKKSGKKAGDWYGPAVVAHILRKAVEEARHPDLQGITIYVAQDCTVYNSDVIDKQSASMTSDNADDKAVIILVPVRLGGERTNTDYLEFVKGILSLEYCVGIIGGKPKQSYYFAGFQDDSLIYMDPHYCQSFVDVSIKDFPLETFHCPSPKKMSFRKMDPSCTIGFYCRNVQDFKRASEEITKMLKFSSKEKYPLFTFVNGHSRDYDFTSTTTNEEDLFSEDEKKQLKRFSTEEFVLL

SEQ ID NO: 17 - protein, 418 aa, Homo sapiens
MEATGTDEVDKLKTKFISAWNNMKYSWVLKTKTYFSRNSPVLLLGKCYHFKYEDEDKTLPAESGCTIEDHVIAGNVEEFRKDFISRIWLTYREEFPQIEGSALTTDCGWGCTLRTGQMLLAQGLILHFLGRAWTWPDALNIENSDSESWTSHTVKKFTASFEASLSGEREFKTPTISLKETIGKYSDDHEMRNEVYHRKIISWFGDSPLALFGLHQLIEYGKKSGKKAGDWYGPAVVAHILRKAVEEARHPDLQGITIYVAQDCTVYNSDVIDKQSASMTSDNADDKAVIILVPVRLGGERTNTDYLEFVKGILSLEYCVGIIGGKPKQSYYFAGFQDDSLIYMDPHYCQSFVDVSIKDFPLEMLKFSSKEKYPLFTFVNGHSRDYDFTSTTTNEEDLFSEDEKKQLKRFSTEEFVLL

SEQ ID NO: 18 - protein, 418 aa, Homo sapiens; identical to SEQ ID NO: 17

SEQ ID NO: 19 - DNA, 2655 bp, Homo sapiens
ccgcaatgcatgctggatcttgcagttcctacgccagaggcaagggcgtgatgctgcatggcctgctggtagttgaagtccaagtgctcgagaggaatttatggcagccgtaatgtggtttgtctatcagagagacaatgctcacagacggatactttctcttggttctgcgcttgctgcattggtggattagcggcttggggtggcgaacagtcagtataaaagattaaactctacagaagaatgcaatcaagtgatggcttttcctttagaatttgaatatggaggctacaggaacagatgaagttgacaagctaaaaaccaaatttatatctgcttggaacaacatgaaatatagttgggtgttgaaaacaaagacgtattttagtagaaattctcctgtattattgcttggaaaatgttaccattttaaatatgaagatgaagataaaacgttacctgcagagtcgggatgtacaatagaggatcacgtaattgcaggaaatgtagaagaatttcgtaaagatttcatttctagaatatggctgacctacagggaagaattccctcaaatagaaggctcagctttgacaacagactgtgggtggggctgcacattgagaactggccagatgctcttggctcaaggactcatactacactttcttggtagagcttggacctggcctgatgctttgaatattgaaaattcagactctgaatcatggacttcccacactgtcaaaaaatttactgcatcatttgaagcatcactttcaggggaaagagaattcaaaaccccaacaatttctctgaaggaaacaattgggaaatattctgatgatcatgaaatgcgaaatgaagtttatcataggaaaatcatctcttggtttggtgattcccccttggctctttttggcttacatcaactaatagaatatggaaagaagtctgggaaaaaagcaggagattggtatggaccagctgtggttgctcacattttaagaaaagcagttgaagaagcaaggcatcctgatttacaaggaataactatttatgttgcacaagattgtacagtttacaattctgatgtaattgataaacagagtgcttccatgacttctgataatgcagatgacaaagctgttattattctagttcctgttagacttggtggagaaagaaccaacaccgactacttagaatttgtgaagggtattttaagcctggaatattgtgtgggtattattggtggcaaacctaaacagtcatattactttgctggatttcaagatgacagtttgatttacatggatcctcattactgccaatcttttgtagatgtcagcataaaggatttccctcttgagacattccactgcccttctcccaaaaagatgtcttttcgaaaaatggatcccagctgtacaataggattttactgtcgaaatgttcaggacttcaaacgagcttctgaagaaatcaccaagatgctgaaattttcttctaaggagaaatatcccttatttacttttgtaaatggtcattccagagactatgattttacatctactacaaccaatgaagaagaccttttttcagaggatgaaaagaaacaattaaaaagatttagcacggaagagtttgtcttgctttaaagattagcacatttgtgcttgataagaagaattccattgaaaggggaaaaatgaagagaaacaagtatatctgaaatgtttattttcacaaatatcttaattttatatgttctttaaaaaagaacatttgaaaatataacagttaaagatatttttctaaaagagaaatgatttaatgaatcttgctttctaataaataaattgagtgattctggttgcattcctatttccctaagatctactagtgataattctaccttaactgtaagccttttagtcttcaaagtcttccacctgagcccattgttctcatggaggttttgtgatattaaccctcccccaaagactgggatcaccaaatagtttcaaaattctcagtttgtactgaagaccagaagatcagagaaggaaactttaatgctgtctagcctcctgctattaatgcaatcaaagaatacttttgcatatgtcttgataattaaatagtatttgttaactgtgatatgcatacacttatataagcagaattatgagttaaagtaatacttagcaatatgattttataatggctcctcattatgcttgctgttgaaccttttatgaggagtgaatataaagtattggttttccctcacaaatttaaagattatgttattaatactattataactgcatcaatcaagtcagataaaggcaactataaaatagtagtagtgtttgtttcctatctcaagggcgaaattttatgggaactcaatttattatgcagtttttaagtttaaaataccaagaaagatgtcactagattctcttctatgtgatttttgttttttatataaagcagtgtagtggtgtttagaagctgaggccacctgtaaggcaaatctgccttaagtgtattatgtgttacttaaaggcaaatttgtgatctaaaagtacaagagtgatttttgagctaggattataaaatacataataaagatgtgagaagataaaa

SEQ ID NO: 20 - DNA, 2543 bp, Homo sapiens
ggtggccggggtgctcaaagtacctgtagctgcggcgctgaggtcggaacgtctgcgtgtgtgcgggctggttttgtggcggctgctgctagagctggagcatttgccggtcagtataaaagattaaactctacagaagaatgcaatcaagtgatggcttttcctttagaatttgaatatggaggctacaggaacagatgaagttgacaagctaaaaaccaaatttatatctgcttggaacaacatgaaatatagttgggtgttgaaaacaaagacgtattttagtagaaattctcctgtattattgcttggaaaatgttaccattttaaatatgaagatgaagataaaacgttacctgcagagtcgggatgtacaatagaggatcacgtaattgcaggaaatgtagaagaatttcgtaaagatttcatttctagaatatggctgacctacagggaagaattccctcaaatagaaggctcagctttgacaacagactgtgggtggggctgcacattgagaactggccagatgctcttggctcaaggactcatactacactttcttggtagagcttggacctggcctgatgctttgaatattgaaaattcagactctgaatcatggacttcccacactgtcaaaaaatttactgcatcatttgaagcatcactttcaggggaaagagaattcaaaaccccaacaatttctctgaaggaaacaattgggaaatattctgatgatcatgaaatgcgaaatgaagtttatcataggaaaatcatctcttggtttggtgattcccccttggctctttttggcttacatcaactaatagaatatggaaagaagtctgggaaaaaagcaggagattggtatggaccagctgtggttgctcacattttaagaaaagcagttgaagaagcaaggcatcctgatttacaaggaataactatttatgttgcacaagattgtacagtttacaattctgatgtaattgataaacagagtgcttccatgacttctgataatgcagatgacaaagctgttattattctagttcctgttagacttggtggagaaagaaccaacaccgactacttagaatttgtgaagggtattttaagcctggaatattgtgtgggtattattggtggcaaacctaaacagtcatattactttgctggatttcaagatgacagtttgatttacatggatcctcattactgccaatcttttgtagatgtcagcataaaggatttccctcttgagatgctgaaattttcttctaaggagaaatatcccttatttacttttgtaaatggtcattccagagactatgattttacatctactacaaccaatgaagaagaccttttttcagaggatgaaaagaaacaattaaaaagatttagcacggaagagtttgtcttgctttaaagattagcacatttgtgcttgataagaagaattccattgaaaggggaaaaatgaagagaaacaagtatatctgaaatgtttattttcacaaatatcttaattttatatgttctttaaaaaagaacatttgaaaatataacagttaaagatatttttctaaaagagaaatgatttaatgaatcttgctttctaataaataaattgagtgattctggttgcattcctatttccctaagatctactagtgataattctaccttaactgtaagccttttagtcttcaaagtcttccacctgagcccattgttctcatggaggttttgtgatattaaccctcccccaaagactgggatcaccaaatagtttcaaaattctcagtttgtactgaagaccagaagatcagagaaggaaactttaatgctgtctagcctcctgctattaatgcaatcaaagaatacttttgcatatgtcttgataattaaatagtatttgttaactgtgatatgcatacacttatataagcagaattatgagttaaagtaatacttagcaatatgattttataatggctcctcattatgcttgctgttgaaccttttatgaggagtgaatataaagtattggttttccctcacaaatttaaagattatgttattaatactattataactgcatcaatcaagtcagataaaggcaactataaaatagtagtagtgtttgtttcctatctcaagggcgaaattttatgggaactcaatttattatgcagtttttaagtttaaaataccaagaaagatgtcactagattctcttctatgtgatttttgttttttatataaagcagtgtagtggtgtttagaagctgaggccacctgtaaggcaaatctgccttaagtgtattatgtgttacttaaaggcaaatttgtgatctaaaagtacaagagtgatttttgagctaggattataaaatacataataaagatgtgagaagataaaatgcttttgttttggttttaatgttgggattattttaatcctttcatttgaaaaatcagtgtctcaaatgaattctgttcatttataataaatgcatatattgctctgaaaa

SEQ ID NO: 21 - DNA, 2575 bp, Homo sapiens
aaagtacctgtagctgcggcgctgaggtcggaacgtctgcgtgtgtgcgggctggttttgtggcggctgctgctagagctggagcatttgccgggttggtggctcctgcacatttttacagttctccagtccttctctttcgtcagtataaaagattaaactctacagaagaatgcaatcaagtgatggcttttcctttagaatttgaatatggaggctacaggaacagatgaagttgacaagctaaaaaccaaatttatatctgcttggaacaacatgaaatatagttgggtgttgaaaacaaagacgtattttagtagaaattctcctgtattattgcttggaaaatgttaccattttaaatatgaagatgaagataaaacgttacctgcagagtcgggatgtacaatagaggatcacgtaattgcaggaaatgtagaagaatttcgtaaagatttcatttctagaatatggctgacctacagggaagaattccctcaaatagaaggctcagctttgacaacagactgtgggtggggctgcacattgagaactggccagatgctcttggctcaaggactcatactacactttcttggtagagcttggacctggcctgatgctttgaatattgaaaattcagactctgaatcatggacttcccacactgtcaaaaaatttactgcatcatttgaagcatcactttcaggggaaagagaattcaaaaccccaacaatttctctgaaggaaacaattgggaaatattctgatgatcatgaaatgcgaaatgaagtttatcataggaaaatcatctcttggtttggtgattcccccttggctctttttggcttacatcaactaatagaatatggaaagaagtctgggaaaaaagcaggagattggtatggaccagctgtggttgctcacattttaagaaaagcagttgaagaagcaaggcatcctgatttacaaggaataactatttatgttgcacaagattgtacagtttacaattctgatgtaattgataaacagagtgcttccatgacttctgataatgcagatgacaaagctgttattattctagttcctgttagacttggtggagaaagaaccaacaccgactacttagaatttgtgaagggtattttaagcctggaatattgtgtgggtattattggtggcaaacctaaacagtcatattactttgctggatttcaagatgacagtttgatttacatggatcctcattactgccaatcttttgtagatgtcagcataaaggatttccctcttgagatgctgaaattttcttctaaggagaaatatcccttatttacttttgtaaatggtcattccagagactatgattttacatctactacaaccaatgaagaagaccttttttcagaggatgaaaagaaacaattaaaaagatttagcacggaagagtttgtcttgctttaaagattagcacatttgtgcttgataagaagaattccattgaaaggggaaaaatgaagagaaacaagtatatctgaaatgtttattttcacaaatatcttaattttatatgttctttaaaaaagaacatttgaaaatataacagttaaagatatttttctaaaagagaaatgatttaatgaatcttgctttctaataaataaattgagtgattctggttgcattcctatttccctaagatctactagtgataattctaccttaactgtaagccttttagtcttcaaagtcttccacctgagcccattgttctcatggaggttttgtgatattaaccctcccccaaagactgggatcaccaaatagtttcaaaattctcagtttgtactgaagaccagaagatcagagaaggaaactttaatgctgtctagcctcctgctattaatgcaatcaaagaatacttttgcatatgtcttgataattaaatagtatttgttaactgtgatatgcatacacttatataagcagaattatgagttaaagtaatacttagcaatatgattttataatggctcctcattatgcttgctgttgaaccttttatgaggagtgaatataaagtattggttttccctcacaaatttaaagattatgttattaatactattataactgcatcaatcaagtcagataaaggcaactataaaatagtagtagtgtttgtttcctatctcaagggcgaaattttatgggaactcaatttattatgcagtttttaagtttaaaataccaagaaagatgtcactagattctcttctatgtgatttttgttttttatataaagcagtgtagtggtgtttagaagctgaggccacctgtaaggcaaatctgccttaagtgtattatgtgttacttaaaggcaaatttgtgatctaaaagtacaagagtgatttttgagctaggattataaaatacataataaagatgtgagaagataaaatgcttttgttttggttttaatgttgggattattttaatcctttcatttgaaaaatcagtgtctcaaatgaattctgttcatttataataaatgcatatattgctctgaaaa

SEQ ID NO: 22 - protein, 134 aa, Homo sapiens
MDLVTYDDVHVNFTQEEWALLDPSQKSLYKGVMLETYRNLTAIGYIWEEHTIEDHFQTSRSHGRHERSCTAEQPSEFIQCGKAFAYESHSQMHQIKHTGEKHYDCNQCGKAFKRRSDLQIHKRTHTGEKPYECK

SEQ ID NO: 23 - protein, 134 aa, Homo sapiens; identical to SEQ ID NO: 22

SEQ ID NO: 24 - DNA, 3835 bp, Homo sapiens
ggtcaagcttgcaagctcctttctgtaactcagcagtcatgggagtttctgtgtgatgtgttctgcgtgactctactgggtgttgtgagaggaggttagtcaagctcagaactccaaacatatgtcttcgctggatgctgtgaatttagctcaagcaagctcagaacttcacacatggatttagtcacctatgatgacgtgcatgtgaacttcactcaggaagagtgggctttgctggatccttctcagaagagtctctataaaggtgtgatgctagagacctataggaatctcacagctataggttacatttgggaagaacatacaattgaagaccatttccaaacttctagaagtcatggaaggcatgaaagaagttgtactgcagagcaaccctctgagtttattcaatgtggtaaagcctttgcatatgagagtcatagtcaaatgcatcaaattaaacatactggagagaaacactatgactgtaaccaatgtggtaaagcttttaaaagaaggagtgacctccaaatacataagcgaacacatacaggagagaaaccctatgaatgtaaatgatgtggtaaagcctttgcaagaagcagtcatctccgaatacataagcgaacacatacaggagagaaaccctatgaatgtaaacgatgtggtaaagcctttgcaagaagtgataacctccaacaacataaacgaacacatacaggagagaaaccctatgaatgtaaacaatgtggtaaagccttttcacaaagcagtcatctccgaatacataagcgaatacatacaggagagaaaccctatgaatgtaaccaatgtggaaaagcctttacaagaagtggtgacctccaaaaacataaacgaacacatacaggagagaaaccctataaatgtaaacaatgtggtaaagcctttgcacaaagcagtaatctccgattacataagcaaacacatacaggagagagaccctatcaatgtaactagctttgcaggaagcaggggtctccaatgccataaaagatcacatactggagagacactatatgaatgtaaccaaggtggtaaaccctttgcaggaaggaatgacctttaaagagaaaagaccatacaggagagaaactctatgaaggtatctaatgttataaagcctttgcagcaagcattgatctccaagtacataaatgaacacataccagagaggaaccttttaaatgaaaccaatgtggtaaaactttttcgaaatccaatagtctttacacacataaaagcacatatagttgagagaaatgctttgattttaaccaatgtggttaaacttttttcaaagggcagtcatcaacaaaatcataaaagaacacatataggagagagatttttgaatgttagcaatggtgcttttacacctttttcatgaagcagtggcctccaaatatataaaggaacacatacaagagagaaaccctatgaatattggcaatgttttaaagcacttctacagcacaatcatcaaatccataaagaacatgaggtggagagaaatcctctgaacaacactaattagggaaagcaattgtacaaaaaagtcatctcccaacacaaaagtatacacactggatagaaaccctatgaatataggcagtgtggtaaatattttttaagtcacagtagttcctaagtaaataaaaggagacacagttgagagaaaccctatgaaagttatcaaatgaacaaagccttgaaagttaaaatcttcaaatgcataaaagaagaaatagaggtgagaaaccctatgtttgcaagccatgtggtattgctttcatttgtcagtcatattcaaagacacaaaagaaaacataatgtagacaaaatgttgagtgtatttaatatggtgaagatttttgaatatttctagagtcttcacaatcaaacaatccatactgcaaagaaatttagaaatataatatggtgaaaccttttcagatcccaaatcttcatcattatgaaaaagaaacctatgaatgcattcaatatggggaagcctttaaacatttctaattcttcatcatcatgaaaataatcatacaaaggagatcaaggggatacaaggcatgattcttattgtcttgcttgagttacagtcctgacatcctttggtgatgaatatcagtgtggaaagtgtgagctggattaaactctttcctccccaactgcttcttcgtcatgtttatgcaggaatagaaaccctgactaagacaaattggtatgaggattggggtattccagtgacaacctgaccatgttttgggaggactaaaaggactttggaactttgggctagaagatccattctgtgttaagaactctgtgggatgttgtgtaggagcttggaagataatgttgagaacagtggagaagatgaaggcctcacttttgaaatttcacaggaaaattaaagactcttttgaggtccattgctgttttggattgtgaagattctgtggttctgtgtagctagggctgaagaatcagctgtgatgaacaagataccagaacttctagagtgaaaactttgcattactgggactattgatgctggttagctggagctaagaaattagcagtgattaagaagagaccagcatcattgaggtggcatcttctgggaagtgttttctgagagcacagaggcagtgttctagagatagccaaggttgtaccttgtgctgtgcctggacttggtaatgtgttagagttacccacatggtactggttttgaaggcctgaagggataatgtagagcagctgatgccctgccctgtgagaggccatggaaggccattggtgaaggtgcaaactcaggtgaaattgatgtcccagtactgaagagatcatgcagtgttttggagttgctaggtccatgaatgaccaagaacagcagcaacagtaaagtacaggcatctggagcctagaggacaagctgtgtgctacaaaggacagggctggagaagtgacccaagcccttggaggagcccaataaatggtgattttgaacccagacattggacagttggagtttaatttttgcttttgattgtgactgtacactaatatttttccctcttgaagggagaaaatattttagtggattccacagttaagagacttttagttgtaaaaggctttggattttaaaggatattggatattttaaaaggattgaacttttactatgtaaagactgtgggactttcaaagtttcttagaacttagggatgaataagaaactaagggttaaggcttaattgtgatgtgcttgtgtgtcaagttgacaaggggtcaattgtactggctagttttctgtcaacttgacacaagctggagttatcacagagaaaggagcttcattgggggaaatgtctccatgagatccatctgtaagacattttctcaatttgtgatcaagtggaaaggccccttgtgggtgggaccatctctctgggctggtagtcttgggttctataagagagcagtctgagaaagccaggtgaagcaaaccagtaaagaaagatccctccatggcctctgcatcagctcctgcttcttgacctgcttgagttccagtcctgacttccttggtgatgaacagcaagcagtatgaaagtataaactgaacaaaccctttcctccccaacttgcttcttggtcatgttgtttgtgcacgaatagaaaccctgaataatacaaggggtataaggtgtggattgtaaaaaaagagactagacaataaagaaatgaagaaataaaattattttaaaaaaaa

SEQ ID NO: 25 - DNA, 3835 bp, Homo sapiens; identical to SEQ ID NO: 24

SEQ ID NO: 26 - protein, 131 aa, Homo sapiens
MNLPSTKVSWAAVTLLLLLLLLPPALLSSGAAAQPLPDCCRQKTCSCRLYELLHGAGNHAAGILTLGKRRSGPPGLQGRLQRLLQASGNHAAGILTMGRRAGAEPAPRPCLGRRCSAPAAASVAPGGQSGI

SEQ ID NO: 27 - DNA, 577 bp, Homo sapiens
cacaattgacagcctcaaggttcctggctttttgaaccaccacagacatctcctttcccggctaccccaccctgagcgccagacaccatgaaccttccttccacaaaggtctcctgggccgccgtgacgctactgctgctgctgctgctgctgccgcccgcgctgttgtcgtccggggcggctgcacagcccctgcccgactgctgtcgtcaaaagacttgctcttgccgcctctacgagctgctgcacggcgcgggcaatcacgcggccggcatcctcacgctgggcaagcggaggtccgggcccccgggcctccagggtcggctgcagcgcctcctgcaggccagcggcaaccacgccgcgggcatcctgaccatgggccgccgcgcaggcgcagagccagcgccgcgcccctgcctcgggcgccgctgttccgccccggccgccgcctccgtcgcgcccggaggacagtccgggatctgagtcgttcttcgggccctgtcctggcccaggcctctgccctctgcccacccagcgtcagcccccagaaaaaaggcaataaagacgagtctccatt

SEQ ID NO: 28 - protein, 147 aa, Homo sapiens
MASHRLLLLCLAGLVFVSEAGPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 29 - DNA, 938 bp, Homo sapiens
gttgactaagtcaataatcagaatcagcaggtttgcagtcagattggcagggataagcagcctagctcaggagaagtgagtataaaagccccaggctgggagcagccatcacagaagtccactcattcttggcaggatggcttctcatcgtctgctcctcctctgccttgctggactggtatttgtgtctgaggctggccctacgggcaccggtgaatccaagtgtcctctgatggtcaaagttctagatgctgtccgaggcagtcctgccatcaatgtggccgtgcatgtgttcagaaaggctgctgatgacacctgggagccatttgcctctgggaaaaccagtgagtctggagagctgcatgggctcacaactgaggaggaatttgtagaagggatatacaaagtggaaatagacaccaaatcttactggaaggcacttggcatctccccattccatgagcatgcagaggtggtattcacagccaacgactccggcccccgccgctacaccattgccgccctgctgagcccctactcctattccaccacggctgtcgtcaccaatcccaaggaatgagggacttctcctccagtggacctgaaggacgagggatgggatttcatgtaaccaagagtattccatttttactaaagcagtgttttcacctcatatgctatgttagaagtccaggcagagacaataaaacattcctgtgaaaggcacttttcattccactttaacttgattttttaaattcccttattgtcccttccaaaaaaaagagaatcaaaattttacaaagaatcaaaggaattctagaaagtatctgggcagaacgctaggagagatccaaatttccattgtcttgcaagcaaagcacgtattaaatatgatctgcagccattaaaaagacacattctgtaaaaaaaaaaaa

SEQ ID NO: 30 - protein, 16 aa, Artificial Sequence
  Synthetic peptide
RADARADARADARADA

SEQ ID NO: 31 - protein, 16 aa, Artificial Sequence
  Synthetic peptide
AEAEAKAKAEAEAKAK

SEQ ID NO: 32 - protein, 12 aa, Artificial Sequence
  Synthetic peptide
KLDLKLDLKLDL

SEQ ID NO: 33 - DNA, 18 bp, Artificial Sequence
  Synthetic probe
gaggaattcccagtaagt

SEQ ID NO: 34 - DNA, 18 bp, Artificial Sequence
  Synthetic probe
gagattgagcaataacag

SEQ ID NO: 35 - DNA, 18 bp, Artificial Sequence
  Synthetic probe
gtagttccgaccataaac

SEQ ID NO: 36 - DNA, 18 bp, Artificial Sequence
  Synthetic probe
ggtgactctagataacct

(19) United States
(12) Patent Application Publication
(10) Pub. No.: US 2021/0140982 A1
(43) Pub. Date: May 13, 2021

(54) IDENTIFICATION OF SPATIAL BIOMARKERS OF BRAIN DISORDERS AND METHODS OF USING THE SAME

(71) Applicant: 10x Genomics, Inc., Pleasanton, CA (US)

(72) Inventor: Cedric Uytingco, Pleasanton, CA (US)

(21) Appl. No.: 17/073,242

(22) Filed: Oct. 16, 2020

Related U.S. Application Data
(60) Provisional application No. 62/939,517, filed on Nov. 22, 2019.
(60) Provisional application No. 62/923,330, filed on Oct. 18, 2019.
(60) Provisional application No. 62/964,063, filed on Jan. 21, 2020.

(51) Int. Cl. G01N 33/68; A61K 45/06; G01N 33/53

(52) U.S. Cl. CPC G01N 33/6896; G01N 2458/10; G01N 33/5308; A61K 45/06

(57) ABSTRACT

Provided herein are methods of detecting biomarkers and/or candidate biomarkers for brain disorders and uses of the same.

This application claims priority and benefit from U.S. Provisional Patent Application 62/923,330, filed Oct. 18, 2019, U.S. Provisional Patent Application 62/939,517 filed Nov. 22, 2019, and U.S. Provisional Patent Application 62/964,063 filed Jan. 21, 2020, the contents and disclosures of which are incorporated herein by reference in their entireties.

### REFERENCE TO SEQUENCE LISTING

This instant application contains a Sequence Listing which has been submitted electronically in ASCII format and is hereby incorporated by reference in its entirety. Said ASCII copy, created on Sep. 8, 2020, is named Sequence Listing.txt and is 66.2 kilobytes in size.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, and signaling and cross-talk with other cells in the tissue.

Spatial heterogeneity has been previously studied using techniques that only provide data for a small handful of analytes in the contact of an intact tissue or a portion of a tissue, or provide a lot of analyte data for single cells, but fail to provide information regarding the position of the single cell in a parent biological sample (e.g., tissue sample).

There are an estimated 5.5 million people in the U.S. that have been diagnosed with Alzheimer's disease. There are also a number of other brain disorders that affect millions of other persons in the U.S. Alzheimer's disease is a neurodegenerative disease becoming worse over time. Alzheimer's studies have suggested initial changes in the brain, such as damage to neurons, may occur 20 years or more before symptoms arise. There are only a small handful of useful biomarkers and drug targets for Alzheimer's disease and these other brain disorders (such as glioblastoma).

### SUMMARY

Featured herein is a method of assessing expression levels in a subject. In some instances, the method includes obtaining a biological sample from the subject; and determining an expression level of one or more analytes (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44) selected from the group consisting of pro-melanin concentrating hormone (PMCH), aldo-keto reductase family 1, member E1 (Akr1e1), autophagy related 4C cysteine peptidase (Atg4c), Gm14296, hypocretin neuropeptide precursor (Hcrt), transthyretin (Ttr), adenosine A2a receptor (Adora2a), thyrotropin releasing hormone preproprotein (Trh), prion protein (Prnp), protein kinase C theta (Prkck), complement C1q B chain (C1qb), glutamate-ammonia ligase (Glul), pituitary tumor-transforming gene 1 (Pttg1), aurora kinase A interacting protein 1 (Aurkaip1), cocaine- and amphetamine-regulated transcript protein (Cartpt), complement component 4B (C4b), mitotic spindle organizing protein 1 (Mzt1), tyrosine-protein phosphatase non-receptor type 3 (Ptpn3), phytanoyl-CoA 2-hydroxylase interacting protein (Phyhip), small nucleolar RNA host gene 11 (Snhg11), RAD23 homolog B (Rad23b), netrin G1 (Ntng1), serine/arginine-rich splicing factor 5 (Srsf5), tyrosine-protein phosphatase non-receptor type 4 (Ptpn4), 5′-nucleotidase domain containing 3 (Nt5dc3), insulin induced gene 1 (Insig1), oxytocin (Oxt), delta-aminolevulinate dehydratase (Alad), nudix hydrolase 19 (Nudt19), Gm10076 ribosomal protein L41 pseudogene (Gm10076), cyclase associated actin cytoskeleton regulatory protein 1 (Cap1), regulator of cell cycle (Rgcc), ubiquitin A-52 residue ribosomal protein fusion product 1 (Uba52), protein phosphatase 1 regulatory inhibitor subunit 1B (Ppp1r1b), phosphodiesterase 10A (Pde10a), Ubiquitin Conjugating Enzyme E2 M (Ube2m), hemoglobin alpha, adult chain 1 (Hba-a1), glutathione S-transferase pi gene (Gstp1), Mesencephalic Astrocyte Derived Neurotrophic Factor (Manf), G Protein-Coupled Receptor 88 (Gpr88), Sin3A-associated protein (Sap301), alkB homolog 6 (Alkbh6), Small nucleolar RNA host gene 6 (Snhg6), Arginine Vasopressin (Avp), Profilin-1 (Pfn1), tachykinin, precursor 1 (Tac1), byproducts, precursors and degradation products thereof, in the biological sample obtained from the subject.

In some instances, the method further includes serially obtaining a biological sample from the subject at a plurality of time points. In some instances, the method also includes determining the expression levels in the serially obtained biological samples from the subject.

Also featured herein is a method of diagnosing a subject as having Alzheimer's disease or having an increased likelihood of developing Alzheimer's disease. In some instances, the method includes (a) determining an expression level of one or more analytes selected from the group consisting of PMCH, Akr1e1, Atg4c, Gm14296, Hcrt, Ttr, Adora2a, and Trh, and byproducts, precursors and degradation products thereof, in a biological sample from the subject; and (b) identifying the subject having (1) an elevated expression level of the one or more analytes PMCH, Akr1e1, Atg4c, Gm14296, Hcrt, and Ttr, and byproducts, precursors and degradation products thereof, of step (a), or (2) about the same or a decreased expression level of the one or more analytes Adora2a, Trh, byproducts, precursors and degradation products thereof, of step (a) as having Alzheimer's disease or having an increased likelihood of developing Alzheimer's disease.

Also featured herein is a method of diagnosing a subject as having Alzheimer's disease or having an increased likelihood of developing Alzheimer's disease. In some instances, the method includes (a) determining an expression level of one or more analytes (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44) selected from the group consisting of PMCH, Akr1e1, Atg4c, Gm14296, Hcrt, Ttr, Adora2a, Trh, Prnp, Prkck, C1qb, Glul, Pttg1, Aurkaip1, Cartpt, C4b, Mzt1, Ptpn3, Phyhip, Snhg11, Rad23b, Ntng1, Srsf5, Ptpn4, Nt5dc3, Insig1, Oxt, Alad, Nudt19, Gm10076, Cap1, Rgcc, Uba52, Ppp1r1b, Pde10a, Ube2m, Hba-a1, Gstp1, Manf, Gpr88, Sap301, Alkbh6, Snhg6, Avp, Pfn1, Tac1, byproducts, precursors and degradation products thereof, in a biological sample from the subject; and (b) identifying the subject having (1) an elevated expression level of the one or more analytes PMCH, Akr1e1, Atg4c, Gm14296, Hcrt, Ttr, Prnp, Prkck, C1qb, Glul, Pttg1, Aurkaip1, Cartpt, C4b, Mzt1, Ptpn3, Phyhip, Snhg11, Rad23b, Ntng1, Srsf5, Ptpn4, Nt5dc3, Insig1, and byproducts, precursors and degradation products thereof, of step (a), or (2) about the same or a decreased expression level of the one or more analytes Adora2a, Trh, Oxt, Alad, Nudt19, Gm10076, Cap1, Rgcc, Uba52, Ppp1r1b, Pde10a, Ube2m, Hba-a1, Gstp1, Manf, Gpr88, Sap301, Alkbh6, Snhg6, Avp, Pfn1, Tac1, byproducts, precursors and degradation products thereof, of step (a) as having Alzheimer's disease or having an increased likelihood of developing Alzheimer's disease.

In some instances, the method further includes obtaining the biological sample from the subject.

Also featured herein is a method of monitoring progression of Alzheimer's disease in a subject over time. In some instances, the method includes (a) determining a first expression level of one or more (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44) analytes selected from the group consisting of PMCH, Akr1e1, Atg4c, Gm14296, Hcrt, Ttr, Adora2a, Trh, Prnp, Prkck, C1qb, Glul, Pttg1, Aurkaip1, Cartpt, C4b, Mzt1, Ptpn3, Phyhip, Snhg11, Rad23b, Ntng1, Srsf5, Ptpn4, Nt5dc3, Insig1, Oxt, Alad, Nudt19, Gm10076, Cap1, Rgcc, Uba52, Ppp1r1b, Pde10a, Ube2m, Hba-a1, Gstp1, Manf, Gpr88, Sap301, Alkbh6, Snhg6, Avp, Pfn1, Tac1, byproducts, precursors and degradation products thereof, in a first biological sample obtained from the subject at a first time point; (b) determining a second expression level of the one or more analytes of step (a); and (c) identifying the subject as having (1) an increased second expression level of the one or more analytes of the PMCH, Akr1e1, Atg4c, Gm14296, Hcrt, Ttr, Prnp, Prkck, C1qb, Glul, Pttg1, Aurkaip1, Cartpt, C4b, Mzt1, Ptpn3, Phyhip, Snhg11, Rad23b, Ntng1, Srsf5, Ptpn4, Nt5dc3, Insig1, and byproducts, precursors and degradation products thereof, of step (a), or (2) about the same or a decreased second expression level of the one or more of the Adora2a, Trh, Oxt, Alad, Nudt19, Gm10076, Cap1, Rgcc, Uba52, Ppp1r1b, Pde10a, Ube2m, Hba-a1, Gstp1, Manf, Gpr88, Sap301, Alkbh6, Snhg6, Avp, Pfn1, Tac1, byproducts, precursors and degradation products thereof, of step (a), as having progressing Alzheimer's disease; or (3) about the same or a decreased second level of the PMCH, Akr1e1, Atg4c, Gm14296, Hcrt, Ttr, Prnp, Prkck, C1qb, Glul, Pttg1, Aurkaip1, Cartpt, C4b, Mzt1, Ptpn3, Phyhip, Snhg11, Rad23b, Ntng1, Srsf5, Ptpn4, Nt5dc3, Insig1, or a byproduct or precursor or degradation product thereof, of step (a), or (4) an increased second expression level of the Adora2a, Trh, Oxt, Alad, Nudt19, Gm10076, Cap1, Rgcc, Uba52, Ppp1r1b, Pde10a, Ube2m, Hba-a1, Gstp1, Manf, Gpr88, Sap301, Alkbh6, Snhg6, Avp, Pfn1, Tac1, byproducts, precursors and degradation products thereof, of step (a) as having static or regressing Alzheimer's disease. In some instances, the method further includes administering a treatment for Alzheimer's disease to the subject.

Also featured herein is a method of determining efficacy of treatment of a treatment for Alzheimer's disease in a subject. In some instances, the methods include (a) determining a first expression level of one or more (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44) analytes selected from the group consisting of PMCH, Akr1e1, Atg4c, Gm14296, Hcrt, Ttr, Adora2a, Trh, Prnp, Prkck, C1qb, Glul, Pttg1, Aurkaip1, Cartpt, C4b, Mzt1, Ptpn3, Phyhip, Snhg11, Rad23b, Ntng1, Srsf5, Ptpn4, Nt5dc3, Insig1, Oxt, Alad, Nudt19, Gm10076, Cap1, Rgcc, Uba52, Ppp1r1b, Pde10a, Ube2m, Hba-a1, Gstp1, Manf, Gpr88, Sap301, Alkbh6, Snhg6, Avp, Pfn1, Tac1, byproducts, precursors and degradation products thereof, in a first biological sample obtained from a subject at a first time point; (b) determining a second expression level of the one or more analytes in a second biological sample obtained from the subject at a second time point, wherein the subject is administered one or more doses of a therapeutic treatment between the first and second time points; and (c) identifying (1a) the therapeutic treatment as being effective in the subject having about the same or a decreased second expression level of the PMCH, Akr1e1, Atg4c, Gm14296, Hcrt, Ttr, Adora2a, Trh, Prnp, Prkck, C1qb, Glul, Pttg1, Aurkaip1, Cartpt, C4b, Mzt1, Ptpn3, Phyhip, Snhg11, Rad23b, Ntng1, Srsf5, Ptpn4, Nt5dc3, Insig1, Oxt, Alad, Nudt19, Gm10076, Cap1, Rgcc, Uba52, Ppp1r1b, Pde10a, Ube2m, Hba-a1, Gstp1, Manf, Gpr88, Sap301, Alkbh6, Snhg6, Avp, Pfn1, Tac1, byproducts, precursors and degradation products thereof, of step (a), or (1b) the therapeutic treatment as being effective in a subject having an increased second expression level of the one or more or Adora2a, Trh, and byproducts, precursors and degradation products thereof, of step (a); or (2a) the therapeutic treatment as not being effective in a subject having an increased second expression level of the one or more of PMCH, Akr1e1, Atg4c, Gm14296, Hcrt, Ttr, Prnp, Prkck, C1qb, Glul, Pttg1, Aurkaip1, Cartpt, C4b, Mzt1, Ptpn3, Phyhip, Snhg11, Rad23b, Ntng1, Srsf5, Ptpn4, Nt5dc3, and Insig1, and byproducts, precursors and degradation products thereof, of step (a), or (2b) the therapeutic treatment as not being effective in a subject having about the same or a decreased second expression level of the one or more of Adora2a, Trh, Oxt, Alad, Nudt19, Gm10076, Cap1, Rgcc, Uba52, Ppp1r1b, Pde10a, Ube2m, Hba-a1, Gstp1, Manf, Gpr88, Sap301, Alkbh6, Snhg6, Avp, Pfn1, Tac1, byproducts, precursors and degradation products thereof, of step (a).

Also featured herein is a method of identifying a patient subpopulation for which a therapeutic treatment is effective for Alzheimer's disease. In some instances, the method includes (a) determining (1) a first expression level of one or more (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44) analytes selected from the group consisting of PMCH, Akr1e1, Atg4c, Gm14296, Hcrt, Ttr, Adora2a, Trh, Prnp, Prkck, C1qb, Glul, Pttg1, Aurkaip1, Cartpt, C4b, Mzt1, Ptpn3, Phyhip, Snhg11, Rad23b, Ntng1, Srsf5, Ptpn4, Nt5dc3, Insig1, Oxt, Alad, Nudt19, Gm10076, Cap1, Rgcc, Uba52, Ppp1r1b, Pde10a, Ube2m, Hba-a1, Gstp1, Manf, Gpr88, Sap301, Alkbh6, Snhg6, Avp, Pfn1, Tac1, byproducts, precursors and degradation products thereof, in first biological samples obtained from a patient subpopulation at a first time point; and (2) a second expression level of the one or more analytes of step (a)(1) in second biological samples obtained from the patient population at a second time point, wherein the patient subpopulation is administered one or more doses of a therapeutic treatment for Alzheimer's Disease between the first and second time points; (b) determining a correlation between efficacy of the therapeutic treatment and the second level in the biological samples from the patient subpopulation as compared a biological sample obtained from an untreated patient, wherein (1) a lower second expression level of the one or more analytes PMCH, Akr1e1, Atg4c, Gm14296, Hcrt, Ttr, Prnp, Prkck, C1qb, Glul, Pttg1, Aurkaip1, Cartpt, C4b, Mzt1, Ptpn3, Phyhip, Snhg11, Rad23b, Ntng1, Srsf5, Ptpn4, Nt5dc3, and Insig1, and byproducts, precursors and degradation products thereof, or (2) about the same or an elevated second expression level of the one or more analytes Adora2a, Trh, Oxt, Alad, Nudt19, Gm10076, Cap1, Rgcc, Uba52, Ppp1r1b, Pde10a, Ube2m, Hba-a1, Gstp1, Manf, Gpr88, Sap301, Alkbh6, Snhg6, Avp, Pfn1, Tac1, byproducts, precursors and degradation products thereof, in the biological samples from the patient subpopulation as compared to the biological sample from the untreated patient is indicative that the therapeutic treatment is effective for Alzheimer's disease in the patient subpopulation.

In some instances, the methods disclosed herein further include obtaining the first and second biological samples from the subject.

In some instances, the levels of at least two of the analytes are determined. In some instances, the levels of at least three of the analytes are determined. In some instances, the levels of at least three of the analytes are determined. In some instances, the at least four of the analytes are pro-melanin concentrating hormone (PMCH), hypocretin neuropeptide precursor (Hcrt), oxytocin (Oxt), and phytanoyl-CoA 2-hydroxylase interacting protein (Phyhip), and byproducts, precursors and degradation products thereof. In some instances, the levels of at least four, at least five, at least six, at least seven, at least eight, at least nine, at least 10, or more of the analytes are determined. In some instances, the levels of each (i.e., all) of the analytes are determined.

In some instances, the methods further include administering a treatment or prophylaxis of Alzheimer's disease to the subject, adjusting a dosage of a treatment or prophylaxis of Alzheimer's disease for the subject, or adjusting a treatment or prophylaxis of Alzheimer's disease for the subject.

In some instances, the treatment or prophylaxis includes (a) an antagonist of one or more of the analytes selected from the group consisting of PMCH, Akr1e1, Atg4c, Gm14296, Hcrt, Ttr, Prnp, Prkck, C1qb, Glul, Pttg1, Aurkaip1, Cartpt, C4b, Mzt1, Ptpn3, Phyhip, Snhg11, Rad23b, Ntng1, Srsf5, Ptpn4, Nt5dc3, and Insig1, and byproducts, precursors and degradation products thereof; or (b) an agonist of one or more of the analytes selected from the group consisting of Adora2a, Trh, Oxt, Alad, Nudt19, Gm10076, Cap1, Rgcc, Uba52, Ppp1r1b, Pde10a, Ube2m, Hba-a1, Gstp1, Manf, Gpr88, Sap301, Alkbh6, Snhg6, Avp, Pfn1, Tac1, and byproducts, precursors and degradation products thereof.

In some instances, the treatment or prophylaxis includes administering one or more agents selected from the group consisting of a cholinesterase inhibitor, an N-methyl-D-aspartate (NMDA) inhibitor, an antipsychotic, a tricyclic antidepressant, a benzodiazepine, insulin, and tacrine hydrochloride. In some instances, the cholinesterase inhibitor is galantamine, rivastigmine, or donepezil; the NMDA inhibitor is memantine; the antipsychotic agent is aripiprazole, risperidone, olanzapine, quetiapine, or haloperidol; the benzodiazepine is lorazepam, oxazepam or temazepam; and the tricyclic antidepressant is nortriptyline.

Also featured herein is a kit that includes (a) an antibody that binds specifically to one or more analytes selected from the group consisting of PMCH, Akr1e1, Atg4c, Gm14296, Hcrt, Ttr, Adora2a, Trh, Prnp, Prkck, C1qb, Glul, Pttg1, Aurkaip1, Cartpt, C4b, Mzt1, Ptpn3, Phyhip, Snhg11, Rad23b, Ntng1, Srsf5, Ptpn4, Nt5dc3, Insig1, Oxt, Alad, Nudt19, Gm10076, Cap1, Rgcc, Uba52, Ppp1r1b, Pde10a, Ube2m, Hba-a1, Gstp1, Manf, Gpr88, Sap301, Alkbh6, Snhg6, Avp, Pfn1, Tac1, byproducts, precursors and degradation products thereof and (b) instructions for performing the method of any one of the preceding claims.

Also featured herein is a method of assessing expression levels of certain analytes in a subject. In some instances the method includes (a) obtaining a biological sample from the subject; and (b) determining an expression level of one or more (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, or 86) analytes selected from the group consisting of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, SLN, SRPX2, METTL7B, POSTN, NNMT, TIMP4, SERPINA3, KLHDC8A, NES, F2R, XIST, COL1A2, COL4A1, CA12, ANXA2, WWTR1, COL4A1, LAMB2, SPARC, FN1, TNFRSF1A, HLA-DRA, ALDH1L1, FLNA, NAMPT, VEGFA, C3, HLA-A, GRN, HLA-B, TPP1, HLA-B, HLA-DRA, LAMB2, NAMPT, byproducts, precursors, and degradation products thereof in the biological sample obtained from the subject.

In some instances, the methods further include serially obtaining a biological sample from the subject at a plurality of time points and determining the expression levels in the serially obtained biological samples from the subject.

Also featured herein is a method of diagnosing a subject as having glioblastoma, or having an increased likelihood of developing glioblastoma. In some instances, the method includes (a) determining an expression level of one or more (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, or 86) analytes selected from the group consisting of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, and byproducts, precursors, degradation and degradation products thereof, in a biological sample from a subject; and (b) identifying a subject having an elevated expression level of the one or more analytes of step (a), in the biological sample as compared to a reference level, as having glioblastoma, or having an increased likelihood of developing glioblastoma.

Also featured herein is a method of diagnosing a subject as having glioblastoma, or having an increased likelihood of developing glioblastoma. In some instances, the method includes (a) determining an expression level of one or more (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, or 86) analytes selected from the group consisting of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, SLN, SRPX2, METTL7B, POSTN, NNMT, TIMP4, SERPINA3, KLHDC8A, NES, F2R, XIST, COL1A2, COL4A1, CA12, ANXA2, WWTR1, COL4A1, LAMB2, SPARC, FN1, TNFRSF1A, HLA-DRA, ALDH1L1, FLNA, NAMPT, VEGFA, C3, HLA-A, GRN, HLA-B, TPP1, HLA-B, HLA-DRA, LAMB2, and NAMPT and byproducts, precursors, degradation and degradation products thereof, in a biological sample from a subject; and (b) identifying a subject having an elevated expression level of the one or more analytes of step (a), in the biological sample as compared to a reference level, as having glioblastoma, or having an increased likelihood of developing glioblastoma.

Also featured herein is a method of diagnosing a subject as having glioblastoma, or having an increased likelihood of developing glioblastoma. In some instances, the method includes (a) determining an expression level of one or more (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, or 86) analytes selected from the group consisting of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIFSA, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, byproducts, precursor and degradation products thereof, in a biological sample from a subject; and (b) identifying a subject having a decreased expression level of the one or more analytes of step (a), in the biological sample as compared to a reference level, as having glioblastoma, or having an increased likelihood of developing glioblastoma.

Also featured herein is a method of monitoring progression of glioblastoma in a subject over time. In some instances, the method includes (a) determining a first expression level of (1) one or more (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, or 86) analytes selected from the group consisting of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, SLN, SRPX2, METTL7B, POSTN, NNMT, TIMP4, SERPINA3, KLHDC8A, NES, F2R, XIST, COL1A2, COL4A1, CA12, ANXA2, WWTR1, COL4A1, LAMB2, SPARC, FN1, TNFRSF1A, HLA-DRA, ALDH1L1, FLNA, NAMPT, VEGFA, C3, HLA-A, GRN, HLA-B, TPP1, HLA-B, HLA-DRA, LAMB2, and NAMPT, and byproducts, precursors, and degradation products thereof; or (2) one or more analytes selected from the group consisting of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIFSA, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, and MT-ND5, byproducts, precursors, and degradation products thereof, in a first biological sample obtained from a subject at a first time point; (b) determining a second expression level of the one or more analytes from step (a)(1) or from step (a)(2) in a second biological sample obtained from the subject at a second time point; and (c) identifying (1) a subject having an increased second expression level of the one or more analytes from step (a)(1) or a decreased second expression level of the one or more analytes from step (a)(2), as compared to the first expression level, as having progressing glioblastoma, or (2) a subject having about the same or a decreased second level of the one or more analytes of step (a)(1) as compared to the first expression level, or about the same or an increased second expression level of the one or more analytes of step (a)(2), as having static or regressing glioblastoma.

Also featured herein is a method of determining efficacy of treatment of a treatment for glioblastoma in a subject. In some instances, the method includes (a) determining a first expression level of (1) one or more (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, or 86) analytes selected from the group consisting of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, SLN, SRPX2, METTL7B, POSTN, NNMT, TIMP4, SERPINA3, KLHDC8A, NES, F2R, XIST, COL1A2, COL4A1, CA12, ANXA2, WWTR1, COL4A1, LAMB2, SPARC, FN1, TNFRSF1A, HLA-DRA, ALDH1L1, FLNA, NAMPT, VEGFA, C3, HLA-A, GRN, HLA-B, TPP1, HLA-B, HLA-DRA, LAMB2, NAMPT, byproducts, precursors, and degradation products thereof; or (2) one or more analytes selected from the group consisting of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIFSA, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, byproducts, precursors, and degradation products thereof in a first biological sample obtained from a subject at a first time point; (b) determining a second expression level of the one or more analytes of step (a)(1) or step (a)(2), in a second biological sample obtained from the subject at a second time point, wherein the subject is administered one or more doses of a therapeutic treatment between the first and second time points; and (c) identifying (i) the therapeutic treatment as being effective in a subject having about the same or a decreased second expression level of the one or more analytes of step (a)(1), or an increased second expression level of the one or more analytes of step (a)(2), as compared to the first expression level, or (ii) the therapeutic treatment as not being effective in a subject having an increased second expression level of the one or more analytes of step (a)(1), or about the same or a decreased second level of the one or more analytes of step (a)(2), as compared to the first level.

Also featured herein is a kit that includes an antibody that binds specifically to one or more (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, or 86) analytes selected from the group consisting of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIFSA, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, SLN, SRPX2, METTL7B, POSTN, NNMT, TIMP4, SERPINA3, KLHDC8A, NES, F2R, XIST, COL1A2, COL4A1, CA12, ANXA2, WWTR1, COL4A1, LAMB2, SPARC, FN1, TNFRSF1A, HLA-DRA, ALDH1L1, FLNA, NAMPT, VEGFA, C3, HLA-A, GRN, HLA-B, TPP1, HLA-B, HLA-DRA, LAMB2, NAMPT and byproducts, precursors, and degradation products thereof; and instructions for performing any of the methods disclosed herein.

Also featured herein is a method of identifying a patient subpopulation for which a therapeutic treatment is effective for glioblastoma. In some instances, the method includes (a) determining (1) first expression level of (i) one or more (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, or 86) analytes selected from the group consisting of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, SLN, SRPX2, METTL7B, POSTN, NNMT, TIMP4, SERPINA3, KLHDC8A, NES, F2R, XIST, COL1A2, COL4A1, CA12, ANXA2, WWTR1, COL4A1, LAMB2, SPARC, FN1, TNFRSF1A, HLA-DRA, ALDH1L1, FLNA, NAMPT, VEGFA, C3, HLA-A, GRN, HLA-B, TPP1, HLA-B, HLA-DRA, LAMB2, NAMPT, byproducts, precursors, and degradation products thereof; or (ii) one or more analytes selected from the group consisting of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIFSA, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, byproducts, precursors, and degradation products thereof in first biological samples obtained from a patient subpopulation at a first time point and (2) a second expression level of the one or more analytes of step (a)(1)(i) or step (a)(1)(ii), in second biological samples obtained from the patient population at a second time point, wherein the patient subpopulation is administered one or more doses of a therapeutic treatment for glioblastoma between the first and second time points; and (b) determining a correlation between efficacy of the therapeutic treatment and the second expression level in biological samples from the patient subpopulation as compared to the level in a biological sample obtained from an untreated patient, wherein a lower second expression level of the one or more analytes of step (a)(1)(i) or about the same or an elevated second level of the one or more analytes of step (a)(1)(ii) in the biological samples from the patient subpopulation as compared to the level in the biological sample from the untreated patient is indicative that the therapeutic treatment is effective for glioblastoma in the patient subpopulation.

In some instances, the methods disclosed herein further include obtaining the first biological sample and the second biological sample from the subject.

In some instances, the expression levels of at least two analytes are determined. In some instances, the expression levels of at least three analytes are determined. In some instances, the expression levels of at least four analytes are determined. In some instances, the at least four of the analytes are selected from a group consisting of ADM, CD44, FN1, HLA-A, HLA-B, HLA-DRA, LAMB2, NAMPT, NES, SPARC, SPP1, and VEGFA and byproducts, precursors and degradation products thereof. In some instances, the levels of at least four, at least five, at least six, at least seven, at least eight, at least nine, at least 10, or more of the analytes are determined. In some instances, the expression levels of each (i.e., all) of the analytes are determined.

In some instances, the methods further include administering a therapeutic treatment for glioblastoma to the subject, adjusting a dosage of a treatment for glioblastoma for the subject, or adjusting a treatment for glioblastoma for the subject. In some instances, therapeutic treatment is (a) an antagonist of one or more (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, or 86) analytes selected from the group consisting of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, SLN, SRPX2, METTL7B, POSTN, NNMT, TIMP4, SERPINA3, KLHDC8A, NES, F2R, XIST, COL1A2, COL4A1, CA12, ANXA2, WWTR1, COL4A1, LAMB2, SPARC, FN1, TNFRSF1A, HLA-DRA, ALDH1L1, FLNA, NAMPT, VEGFA, C3, HLA-A, GRN, HLA-B, TPP1, HLA-B, HLA-DRA, LAMB2, NAMPT, byproducts, precursors, and degradation products thereof; or (b) an agonist of one or more analytes selected from the group consisting of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, byproducts, precursors, and degradation products thereof.

In some instances, the methods disclosed herein further include administering a treatment or glioblastoma to the subject, wherein the treatment includes surgery, chemotherapeutic agents, growth inhibitory agents, cytotoxic agents, agents used in radiation therapy, anti-angiogenesis agents, cancer immunotherapeutic agents, apoptotic agents, anti-tubulin agents, or a combination thereof.

In some instances, the methods disclosed herein further include determining step(s) that includes (a) contacting the biological sample with an substrate that includes a plurality of attached capture probes, wherein a capture probe of the plurality includes (i) the spatial barcode and (ii) a capture domain that binds specifically to a sequence present in the analyte; (b) extending a 3′ end of the capture probe using the analyte that is specifically bound to the capture domain as a template to generate an extended capture probe; (c) amplifying the extended capture probe to produce the nucleic acid; (d) determining (i) all or a portion of the sequence of the spatial barcode or the complement thereof, and (ii) all or a portion of the sequence of the analyte from the biological sample; and (e) using the determined sequences of (i) and (ii) in step (d) to identify the location of the analyte in the biological sample.

In some instances, the biological sample or first and second biological samples include cerebrospinal fluid, whole blood, plasma, and/or serum. In some instances, the biological sample or first and second biological samples include a tissue section. In some instances, the biological sample includes serial tissue sections. In some instances, the biological sample includes a tissue structure. In some instances, the expression level is a level of protein or a byproduct or precursor or degradation product thereof. In some instances, the expression level is a level of mRNA or a fragment thereof. In some instances, the biological sample is a cell culture sample.

Also featured herein is a kit that includes a substrate with one or more capture probes, wherein each capture probe includes a spatial barcode and a capture domain, wherein each capture probe binds to a biological analyte from a biological sample; and (b) reagents to detect the biological analyte, wherein the biological analyte is a analyte selected from a group consisting of PMCH, Akr1e1, Atg4c, Gm14296, Hcrt, Ttr, Adora2a, Trh, Prnp, Prkck, C1qb, Glul, Pttg1, Aurkaip1, Cartpt, C4b, Mzt1, Ptpn3, Phyhip, Snhg11, Rad23b, Ntng1, Srsf5, Ptpn4, Nt5dc3, Insig1, Oxt, Alad, Nudt19, Gm10076, Cap1, Rgcc, Uba52, Ppp1r1b, Pde10a, Ube2m, Hba-a1, Gstp1, Manf, Gpr88, Sap301, Alkbh6, Snhg6, Avp, Pfn1, Tac1, byproducts, precursors and degradation products thereof.

Also featured herein is a kit that includes (a) a substrate with one or more capture probes, wherein each capture probe includes a spatial barcode and a capture domain, wherein each capture probe binds to a biological analyte from a biological sample; and (b) reagents to detect the biological analyte, wherein the biological analyte is a biomarker selected from a group consisting of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, SLN, SRPX2, METTL7B, POSTN, NNMT, TIMP4, SERPINA3, KLHDC8A, NES, F2R, XIST, COL1A2, COL4A1, CA12, ANXA2, WWTR1, COL4A1, LAMB2, SPARC, FN1, TNFRSF1A, HLA-DRA, ALDH1L1, FLNA, NAMPT, VEGFA, C3, HLA-A, GRN, HLA-B, TPP1, HLA-B, HLA-DRA, LAMB2, and NAMPT.

In some instances, any one of the kits further includes one or more reagents selected from a group consisting of one or more antibodies, one or more antigen-binding antibody fragments, labeled hybridization probes, primers, or any combination thereof, wherein the one or more reagents enable visualizing one or more features of the biological sample. In some instances, the biological sample is a tissue sample. In some instances, the biological sample is a cell culture sample.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1A shows spatial clustering of the hippocampus and neighboring areas using the spatial gene expression technology (inset—H&E reference).

FIG. 1B shows spatial clustering of the hippocampus and neighboring areas using the spatial gene expression technology. Top genes that are more highly expressed in the hippocampus (than any other cluster) are shown expanded and annotated to the right.

FIG. 1C shows a t-SNE plot of FIG. 1A.

FIG. 2 shows restriction of gene expression in specific hippocampal areas.

FIG. 3 shows spatial clustering and gene expression anteriorly within the olfactory bulb.

FIG. 4 shows spatial clustering and gene expression posteriorly within the cerebellum.

FIG. 5A shows spatially-resolved clustering of the anterior and posterior regions of the mouse brain split across two capture areas.

FIG. 5B shows glutamate ionotropic receptor AMPA type subunit 1 (Gria1) expression across multiple regions of the brain.

FIG. 6A is a plot showing that serial sections between different capture areas demonstrate strong correlation with the examination of 10,000 randomly selected genes.

FIG. 6B is a plot showing that the obtained spatial sequencing data correlate with single-nuclear 3′ RNA sequencing from consecutive sections collected from the same brain sample.

FIG. 7A shows spatially-resolved clustering in a rat olfactory bulb with a demonstration of Gabra1 expression corresponding to the external plexiform layer (inset).

FIG. 7B shows spatially-resolved clustering of cerebellar tissue from a female human with a demonstration of Neurod1 expression corresponding to the granule cell layer (inset).

FIG. 8A are plots illustrating differential gene expression between wt/wt and tg/wt mice.

FIG. 8B illustrates neurogranin (Nrgn) expression in wt/wt and tg/wt mice.

FIG. 8C illustrates pro-melanin concentrating hormone (Pmch) in wt/wt and tg/wt mice.

FIG. 9A is a schematic diagram showing an example sample handling apparatus that can be used to implement various steps and methods described herein.

FIG. 9B is a schematic diagram showing an example imaging apparatus that can be used to obtain images of biological samples, analytes, and arrays of features.

FIG. 9C is a schematic diagram of an example of a control unit of the apparatus of FIGS. 1A and 1B.

FIG. 10 shows plots illustrating differential gene expression between wt/wt and tg/wt mice.

FIG. 11 illustrates Adora2a expression in wt/wt and tg/wt mice.

FIG. 12 illustrates Trh expression in wt/wt and tg/wt mice.

FIG. 13 illustrates Akr1e1 expression in wt/wt and tg/wt mice.

FIG. 14 illustrates Atg4c expression in wt/wt and tg/wt mice.

FIG. 15 illustrates Gm14296 expression in wt/wt and tg/wt mice.

FIG. 16 illustrates Hcrt expression in wt/wt and tg/wt mice.

FIG. 17 illustrates Ttr expression in wt/wt and tg/wt mice.

FIG. 18A shows a histological section of a human cerebral cortex (unspecified) sample.

FIG. 18B shows a tissue plot with spots colored by unsupervised clustering.

FIG. 19A shows a histological section of a human cerebral cortex (temporal) sample.

FIG. 19B shows a tissue plot with spots colored by unsupervised clustering.

FIG. 20A shows a histological section of a human spinal cord sample.

FIG. 20B shows a tissue plot with spots colored by unsupervised clustering.

FIG. 21A shows a histological section of a human cerebellum sample.

FIG. 21B shows a tissue plot with spots colored by unsupervised clustering.

FIG. 22A is a t-SNE plot of spots colored by unsupervised clustering.

FIG. 22B is a UMAP plot of spots colored by unsupervised clustering.

FIG. 23 is a scatter plot showing the differential expression of genes.

FIG. 24A is a t-SNE plot of spots colored by unsupervised clustering.

FIG. 24B is a UMAP plot of spots colored by unsupervised clustering.

FIG. 25 is a scatter plot showing the differential expression of genes.

FIG. 26A is a t-SNE plot of spots colored by unsupervised clustering.

FIG. 26B is a UMAP plot of spots colored by unsupervised clustering.

FIG. 27A is a table illustrating differential gene expression in glioblastoma and healthy brain samples.

FIG. 27B is a scatter plot illustrating differential gene expression in glioblastoma and healthy brain samples.

FIG. 28A is a table illustrating differential gene expression in glioblastoma and healthy brain samples.

FIG. 28B is a scatter plot illustrating differential gene expression in glioblastoma and healthy brain samples.

FIG. 29A is a table illustrating differential gene expression in glioblastoma and healthy brain samples.

FIG. 29B is a table illustrating differential gene expression in glioblastoma and healthy brain samples.

FIG. 30 shows tissue plots from glioblastoma samples with spots colored by unsupervised clustering.

### DETAILED DESCRIPTION

### I. Introduction

Spatial analysis methodologies can provide a vast amount of analyte level and/or expression data for a variety of multiple analytes within a sample at high spatial resolution, e.g., while retaining the native spatial context. Spatial analysis methods can include, e.g., the use of a capture probe including a spatial barcode (e.g., a nucleic acid sequence that provides information as to the position of the capture probe within a cell or a tissue sample (e.g., mammalian cell or a mammalian tissue sample) and a capture domain that is capable of binding to an analyte (e.g., a protein and/or nucleic acid) produced by and/or present in a cell.

Non-limiting aspects of spatial analysis methodologies are described in WO 2011/127099, WO 2014/210233, WO 2014/210225, WO 2016/162309, WO 2018/091676, WO 2012/140224, WO 2014/060483, WO 2020/176788 U.S. Pat. Nos. 10,002,316, 9,727,810, U.S. Patent Application Publication No. 2020/0277663, U.S. Patent Application Publication No. 2017/0016053, Rodrigues et al., Science 363(6434):1463-1467, 2019; WO 2018/045186, Lee et al., Nat. Protoc. 10(3):442-458, 2015; WO 2016/007839, WO 2018/045181, WO 2014/163886, Trejo et al., PLoS ONE 14(2):e0212031, 2019, U.S. Patent Application Publication No. 2018/0245142, Chen et al., Science 348(6233):aaa6090, 2015, Gao et al., BMC Biol. 15:50, 2017, WO 2017/144338, WO 2018/107054, WO 2017/222453, WO 2019/068880, WO 2011/094669, U.S. Pat. Nos. 7,709,198, 8,604,182, 8,951,726, 9,783,841, 10,041,949, WO 2016/057552, WO 2017/147483, WO 2018/022809, WO 2016/166128, WO 2017/027367, WO 2017/027368, WO 2018/136856, WO 2019/075091, U.S. Pat. No. 10,059,990, WO 2018/057999, WO 2015/161173, and Gupta et al., Nature Biotechnol. 36:1197-1202, 2018, each of which is incorporated by reference in its entirety, and can be used herein in any combination. Further non-limiting aspects of spatial analysis methodologies are described herein.

Some general terminology that may be used in this disclosure can be found in Section (I)(b) of WO 2020/176788 and/or U.S. Patent Application Publication No. 2020/0277663, each of which is incorporated by reference in its entirety. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663, each of which is incorporated by reference in its entirety. In some embodiments, an analyte is a biomarker disclosed herein. Each analyte can be referred to in all capital letters, first letter capital, or all small letters (e.g., PMCH, Pmch, or pmch). Each of these alternative forms of identifying an analyte can be used interchangeably to indicate a nucleic acid (e.g., mRNA, cDNA, etc) or a non-nucleic acid analyte (e.g., protein).

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). In some embodiments biological sample can be a cell culture sample. In some embodiments, a biological sample can be nervous tissue, blood, serum, plasma, cerebrospinal fluid, or bone marrow aspirate. Biological samples are also described in Section (I)(d) of WO 2020/176788 and/or U.S. Patent Application Publication No. 2020/0277663, each of which is incorporated by reference in its entirety.

In addition, spatial analysis methods can be performed on various types of samples, including tissues (e.g., tissue slices) or single cells (e.g., cultured cells). Exemplary methods and compositions relating to tissue or single-cell spatial analysis is found at least in WO 2020/176788 and/or U.S. Patent Application Publication No. 2020/0277663, each of which is incorporated by reference in its entirety. In some instances, one biological sample can be used for tissue and single cell analysis. For example, multiple serial slices (e.g., 10 μm in thickness) of a tissue can be cut. A first slice can be placed on an array and analyte capture as described herein can be performed. In some instances, a second slice of tissue can further undergo cellular dissociation, creating a sample with isolated cells that can be analyted using spatial analysis methods. Briefly, in some instances, a tissue is minced into small pieces and treated with lysis buffer to homogenize the sample. The homogenous resultant can be filtered and centrifuged to collect a pellet of nuclei. The nuclei can be resuspended and used for single cell analysis methods described herein. Data captured from the second slice (i.e., the single nuclei data) could then be combined with the data from the first slice (i.e., the whole tissue data) to gain a higher cell type understanding and potentially deconvolve the cell type identity within each spot on the array. Additional methods of single cell isolation is found in Hu et al., Mol Cell. 2017 Dec. 7; 68(5):1006-1015.e7; Habib et al., Science, 2016 Aug. 26; 353(6302):925-8; Habib et al., Nat Methods, 2017 October; 14(10):955-958; Lake et al., Science, 2016 Jun. 24; 352(6293):1586-90; and Lacar et al., Nat Commun, 2016 Apr. 19; 7:11022; each of which is incorporated by reference in its entirety.

In another embodiment, two different samples are collected, whereby one sample is analyzed with intact tissue and a second tissue undergoes cell dissociation. Results from each biological sample can be compared to gain a higher cell type understanding and potentially deconvolve the cell type identity within each spot on the array. Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of each analyte within the biological sample. The spatial location of each analyte within the biological sample is determined based on the feature to which each analyte is bound on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain. In some instances, the capture probe can include functional sequences that are useful for subsequent processing, such as functional sequence 604, which can include a sequencer specific flow cell attachment sequence, e.g., a P5 or P7 sequence, as well as functional sequence 606, which can include sequencing primer sequences, e.g., a R1 primer binding site, a R2 primer binding site. In some embodiments, sequence 604 is a P7 sequence and sequence 606 is a R2 primer binding site. Additional features of capture probes are described in Section (II)(b) of WO 2020/176788 and/or U.S. Patent Application Publication No. 2020/0277663, each of which is incorporated by reference in its entirety. Generation of capture probes can be achieved by any appropriate method, including those described in Section (II)(d)(ii) of WO 2020/176788 and/or U.S. Patent Application Publication No. 2020/0277663, each of which is incorporated by reference in its entirety.

In some embodiments, more than one analyte type from a biological sample can be detected (e.g., simultaneously or sequentially) using any appropriate multiplexing technique, such as those described in Section (IV) of WO 2020/176788 and/or U.S. Patent Application Publication No. 2020/0277663, each of which is incorporated by reference in its entirety.

In some embodiments, detection of one or more analytes (e.g., protein analytes) can be performed using one or more analyte capture agents. As used herein, an “analyte capture agent” refers to an agent that interacts with an analyte (e.g., an analyte in a sample) and with a capture probe (e.g., a capture probe attached to a substrate) to identify the analyte. In some embodiments, the analyte capture agent includes an analyte binding moiety and a capture agent barcode domain. Additional description of analyte capture agents can be found in Section (II)(b)(viii) of WO 2020/176788 and/or U.S. Patent Application Publication No. 2020/0277663, each of which is incorporated by reference in its entirety.

There are at least two general methods to associate a spatial barcode with one or more neighboring cells, such that the spatial barcode identifies the one or more cells, and/or contents of the one or more cells, as associated with a particular spatial location. One general method is to promote analytes out of a cell and towards a spatially-barcoded array (e.g., including spatially-barcoded capture probes). Another general method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template), or derivatives thereof (see, e.g., Section (II)(b)(ii) of WO 2020/176788 and/or U.S. Patent Application Publication No. 2020/0277663 regarding extended capture probes, the references of which are incorporated by reference in their entireties.). For example, in some cases, the capture probes may include mRNA specific priming sequences, e.g., poly-T primer segments that allow priming and replication of mRNA in a reverse transcription reaction or other targeted priming sequences. Alternatively or additionally, random RNA priming may be carried out using random N-mer primer segments of the barcoded oligonucleotides. Reverse transcriptases (RTs) can use an RNA template and a primer complementary to the 3′ end of the RNA template to direct the synthesis of the first strand complementary DNA (cDNA). Additional variants of spatial analysis methods, including in some embodiments, an imaging step, are described in Section (II)(a) of WO 2020/176788 and/or U.S. Patent Application Publication No. 2020/0277663, each of which is incorporated by reference in its entirety. Analysis of captured analytes, for example, including sample removal, extension of capture probes, sequencing (e.g., of a cleaved extended capture probe and/or a cDNA molecule complementary to an extended capture probe), sequencing on the array (e.g., using in situ hybridization approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663, each of which is incorporated by reference in its entirety.

Typically, for spatial array-based analytical methods, a substrate functions as a support for direct or indirect attachment of capture probes to features of the array. A “feature” is an entity that acts as a support or repository for various molecular entities used in sample analysis. In some embodiments, some or all of the features in an array are functionalized for analyte capture. Exemplary substrates are described in Section (II)(c) of WO 2020/176788 and/or U.S. Patent Application Publication No. 2020/0277663, each of which is incorporated by reference in its entirety. Exemplary features and geometric attributes of an array can be found in Sections (II)(d)(i), (II)(d)(iii), and (II)(d)(iv) of WO 2020/176788 and/or U.S. Patent Application Publication No. 2020/0277663, each of which is incorporated by reference in its entirety.

Generally, analytes can be captured when contacting a biological sample with a substrate including capture probes (e.g., substrate with capture probes embedded, spotted, printed on the substrate or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663, each of which is incorporated by reference in its entirety.

In some cases, a spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be separated into single cells or cell groups for analysis. Some such methods of spatial analysis are described in Section (III) of WO 2020/176788 and/or U.S. Patent Application Publication No. 2020/0277663, each of which is incorporated by reference in its entirety.

Some exemplary particular spatial analysis workflows are described in the Exemplary Embodiments section of WO 2020/176788 and/or U.S. Patent Application Publication No. 2020/0277663, each of which is incorporated by reference in its entirety.

In some embodiments, a spatial analysis can be performed using dedicated hardware and/or software, such as any of the systems described in Sections (II)(e)(ii) and/or (V) of WO 2020/176788 and/or U.S. Patent Application Publication No. 2020/0277663, each of which is incorporated by reference in its entirety.

### II. Spatial Analysis and Brain Disorders

Provided herein are methods of identifying and measuring biomarkers that are dysregulated in a brain disorder, such as Alzheimer's Disease (AD) or a glioblastoma. This disclosure includes methods of detecting biomarkers in various locations in a sample and thereby identifying candidate drug targets for treatment of a brain disorder, methods of identifying a candidate biomarker for efficacy of treatment of a brain disorder (e.g., AD, glioblastoma), methods of diagnosing a brain disorder (e.g., AD, glioblastoma) in a subject, methods of identifying a subject with increased likelihood of developing a brain disorder (e.g., AD, glioblastoma), methods of monitoring the progression of a brain disorder (e.g., AD, glioblastoma) in a subject, and methods of determining the efficacy of a treatment for a brain disorder (e.g., AD, glioblastoma), methods for identifying a patient subpopulation for which a therapeutic treatment is effective for a brain disorder (e.g., AD, glioblastoma), and methods of modifying treatment for an AD or glioblastoma patient. Also provided herein are kits comprising antibodies to the specific candidate biomarkers identified herein.

The biomarkers for Alzheimer's Disease identified herein can be used for diagnostic, prognostic and therapeutic purposes and include but are not limited to one or more of pro-melanin concentrating hormone (PMCH), aldo-keto reductase family 1, member E1 (Akr1e1), autophagy related 4C cysteine peptidase (Atg4C), Gm14296, hypocretin neuropeptide precursor (Hcrt), transthyretin (Ttr), adenosine A2a receptor (Adora2a), and thyrotropin releasing hormone preproprotein (Trh), PRNP, PRKCK, C1QB, GLUL, PTTG1, AURKAIP1, CARTPT, C4B, MZT1, PTPN3, PHYHIP, SNHG11, RAD23B, NTNG1, SRSF5, PTPN4, NT5DC3, INSIG1, OXT, ALAD, NUDT19, GM10076, CAP1, RGCC, UBA52, PPP1R1B, PDE10A, UBE2M, HBA-A1, GSTP1, MANF, GPR88, SAP30L, ALKBH6, SNHG6, AVP, PFN1, OR TAC1. or a byproduct, a degradation product, or a precursor thereof.

Also provided herein are methods of identifying and measuring biomarkers that are dysregulated in a brain cancer, such as glioblastoma. This disclosure includes methods of detecting biomarkers in various locations in a sample and thereby identifying candidate drug targets for treatment of a brain cancer (e.g., glioblastoma), methods of identifying a candidate biomarker for efficacy of treatment of a brain cancer (e.g., glioblastoma), methods of diagnosing a brain cancer (e.g., glioblastoma) in a subject, methods of identifying a subject with increased likelihood of developing a brain cancer (e.g., glioblastoma), methods of monitoring the progression of a brain cancer (e.g., gioblastoma) in a subject, and methods of determining the efficacy of a treatment for a brain cancer (e.g., glioblastoma), methods for identifying a patient subpopulation for which a therapeutic treatment is effective for a brain cancer (e.g., glioblastoma), and methods of modifying treatment of a glioblastoma patient. Also provided herein are kits comprising antibodies to the specific candidate biomarkers identified herein.

The biomarkers for glioblastoma identified herein can be used for diagnostic, prognostic, and therapeutic purposes and include but are not limited to one or more of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, SLN, SRPX2, METTL7B, POSTN, NNMT, TIMP4, SERPINA3, KLHDC8A, NES, F2R, XIST, COL1A2, COL4A1, CA12, ANXA2, WWTR1, COL4A1, LAMB2, SPARC, FN1, TNFRSF1A, HLA-DRA, ALDH1L1, FLNA, NAMPT, VEGFA, C3, HLA-A, GRN, HLA-B, TPP1, HLA-B, HLA-DRA, LAMB2, or NAMPT or a byproduct, a degradation product, or a precursor thereof.

(a) Brain Disorders

As used herein, a brain disorder can be any appropriate brain disorder. In some embodiments, a brain disorder is a degenerative brain disorder. In some embodiments, a brain disorder can be selected from the group consisting of acquired brain injury, agenesis corpus callosum, Alzheimer's Disease, amyotrophic lateral sclerosis (ALS), aneurysm, aphasia, arteriovenous malformation, ataxia (e.g., Friedrich's ataxia), attention deficit hyperactivity disorder (ADHD), autism, Batten disease, Behcet's disease, blepharospasm, brain cancer, cerebral lupus, cerebral palsy, cervical dystonia, Charcot-Marie-Tooth disorder, Chiari malformation, chronic inflammatory demyelinating polyneuropathy, ciliopathies, coma, concussion, Creutzfeldt-Jakob disease, dementia (e.g., non-Alzheimer type), Down Syndrome, dysautonomia, dyslexia, dyspraxia, dystonia, encephalitis, epilepsy, essential tremor, Friedreich ataxia, Gaucher disease, Guillain-Barre Syndrome, headache (e.g., cluster headache, tension headache, migraine), Huntington's disease, hydrocephalus, hypoxia, intracranial hypertension, ischemia, Joubert syndrome leukodystrophy, locked-in Syndrome (LIS), lysosomal storage disorders, meningitis, meningococcal disease, metal metabolism disorders (e.g., Wilson disease), mitochondrial disorders, motor neurone disease, multiple sclerosis, multiple system atrophy, muscular dystrophy, myasthenia gravis, Parkinson's disease, peripheral neuropathy, progressive supranuclear palsy, restless legs syndrome, Rett syndrome, shy drager syndrome, sleep disorders (e.g., narcolepsy), spasmodic dysphonia, stroke, Sydenham's chorea, Tay-Sachs disease, Tourette syndrome, transient ischaemic attack, transverse myelitis, traumatic brain injury, trigeminal neuralgia, tuberous sclerosis, subarachnoid haemorrhage, vegetative state, and Von Hippel-Lindau syndrome.

In some embodiments, a brain disorder can be selected from the group consisting of Alzheimer's disease, Parkinson's disease, dementia, brain cancer, epilepsy, stroke, ataxia, and Wilson's disease. In some embodiments, a brain disorder can be selected from the group consisting of Alzheimer's disease, amyotrophic lateral sclerosis, ataxia, Batten disease, brain cancer (e.g., glioblastoma), concussion, Creutzfeldt-Jakob disease, dementia, epilepsy, Huntington's disease, hypoxia, Joubert syndrome, meningitis, Parkinson's disease, seizures, stroke, transient ischemic attack, traumatic brain injury, and Wilson's disease.

In some embodiments, a brain disorder can be Alzheimer's disease. In some embodiments, a brain disorder can be Parkinson's disease. In some embodiments, a brain disorder can be dementia. In some embodiments, a brain disorder can be brain cancer. Non-limiting examples of brain cancers include acoustic neuroma, astrocytoma, CNS lymphoma, glioblastoma, glioma (e.g., unspecified glioma), haemangioblastoma, meningioma, oligodendroglioma, and pituitary adenoma. In some embodiments, brain cancer can be glioblastoma. In some embodiments, a brain disorder can be epilepsy. In some embodiments, a brain disorder can be stroke. In some embodiments, a brain disorder can be ataxia. In some embodiments, a brain disorder can be Wilson's disease. In some embodiments, a brain disorder can be seizure. In some embodiments, a brain disorder can be transient ischemic attack. In some embodiments, a brain disorder can be hypoxia. In some embodiments, a brain disorder can be Huntington's disease. In some embodiments, a brain disorder can be traumatic brain injury. In some embodiments, a brain disorder can be concussion. In some embodiments, a brain disorder can be ALS. In some embodiments, a brain disorder can be Batten disease. In some embodiments, a brain disorder can be Creutzfeldt-Jakob disease. In some embodiments, a brain disorder can be meningitis. In some embodiments, a brain disorder can be Joubert syndrome.

(b) Animals

As used herein, an animal can be any appropriate animal. In some embodiments, an animal can be a zebrafish. In some embodiments, an animal can be selected from the group consisting of a zebrafish, a mouse, a rat, a dog, a naked mole rat, a nonhuman primate, and a human. In some embodiments, an animal can be a mammal. In some embodiments, a mammal can be selected from a mouse, a rat, a dog, a naked mole rat, a nonhuman primate, and a human. In some embodiments, a mammal can be a mouse. In some embodiments, a mammal can be a rat. In some embodiments, a mammal can be a nonhuman primate (e.g., a chimpanzee, a gorilla, an orangutan, a rhesus monkey, a cynomolgus monkey, a Taiwanese macaque, a green monkey, a squirrel monkey, tamarin, a marmoset, or a mouse lemur). In some embodiments, a mammal can be a human. In some embodiments, an animal can be an animal model of a brain disorder (e.g., any of the brain disorders described herein). In some embodiments, a mammal can be a mammalian model of a brain disorder (e.g., any of the brain disorders described herein or known in the art).

In some embodiments, an animal model of a brain disorder can express one or more human genes. Animal models of Alzheimer's disease can include one or more mutations in one or more genes, for example, in amyloid precursor protein (APP, e.g., human transgene APP models with the Swedish mutation (K670D/M671L), the Indiana mutation (V717F), the London mutation (V7171), and/or the Arctic mutation (E693G)), presenilin 1 (PSEN1), presnilin 2 (PSEN2), tau (MAPT, e.g. N279K, AK280, P301L, P301S, V337 and/or R406W), apolipoprotein E (APOE), progranulin (PGRN), TAR DNA-binding protein TDP-43 (TARDBP), valosin-containing protein (VCP). Non-limiting examples of animal models of Alzheimer's disease include mouse models (e.g., PDAPP, H6, J9, J20, Tg2576, APP23, C3-3, CRND8, ARC6/ARC48, C3-3×PSEN1, PSAPP, APP_{SL}PS1_{M146L}, 5×FAD, hBACE1/hAPP, hTau, TauP301L, Tau V337M, Tau P301S, Tau G272V, P301S, 3×Tg-AD). In some embodiments, an animal model can be the progeny of a wild-type (wt) animal and a transgenic (tg) animal.

In some embodiments, mammalian models of Parkinson's disease can include one or more mutations in one or more genes, for example leucine-rich repeat kinase 2 (LRRK2, e.g., R1441G, R1441C, N1437H, Y1699C, G2019S, and/or I2020T), alpha-synuclein (SNCA, e.g., A53T, A30P, and/or E46K), Parkin RBR E3 ubiquitin protein ligase (PRKN), DJ-1 (also called PARK7), and/or PTEN induced kinase 1 (PINK1). In some embodiments, mammalian models of Parkinson's disease can have been treated with a toxin, such as 1-methyl-4-phenyl-1,2,3,6-tetrahydropyridine or 6-hydroxydopamine.

Other animal models may be known, for example, such as those found in Dawson, et al., Nat Neurosci. 2018 October; 21(10): 1370-1379 (doi: 10.1038/s41593-018-0236-8), LaFerla and Green, Cold Spring Harb Perspect Med November 2012; 2:a006320 (doi:10.1101/cshperspect.a006320), Gotz and Ittner, Nat Rev Neurosci. 2008 July; 9(7):532-44. (doi: 10.1038/nrn2420), Nagahara et al., Nat Med. 2009 March; 15(3): 331-337. (doi: 10.1038/nm.1912), Dawson et al., Neuron. 2010 Jun. 10; 66(5): 646-661 (doi: 10.1016/j.neuron.2010.04.034), Blandini and Armentero FEBS J. 2012 April; 279(7):1156-66 (doi: 10.1111/j.1742-4658.2012.08491.x), each of which is herein incorporated by reference in its entirety.

(c) Spatial Cell-Based Analytical Methodology and Methods Involving Sorting Subsets of Nucleic Acids

Provided herein are methods for sorting subsets of nucleic acids from a biological sample into a cluster. For example, in some embodiments, such methods include contacting the biological sample with a plurality of capture probes, wherein a capture probe comprises a capture domain and a spatial barcode having a sequence; releasing nucleic acids from the biological sample, wherein members of the released nucleic acids are specifically bound by the capture domain(s); determining, for the nucleic acids that are specifically bound by the capture domain(s), (1) all or a portion of a sequence of the spatial barcode, or a complement thereof, and (2) all or a portion of a sequence of the nucleic acid or a complement thereof, and using the determined sequences of (1) and (2) to identify the location and amount of the nucleic acids in the biological sample; and comparing the determined location and amount of the nucleic acids at a plurality of different locations in the biological sample.

In some embodiments, methods of differentiating cell types in a biological sample are provided herein, e.g., the methods comprise sorting a subset of nucleic acids into a cluster based on the determined location and amount of the nucleic acids at the plurality of different locations in the biological sample, and using the cluster(s) to differentiate cell types in the biological sample. In some embodiments, methods of identifying a biological sample are provided herein, e.g., the methods comprise sorting a subset of nucleic acids into a cluster based on the determined location and amount of the nucleic acids at the plurality of different locations in the biological sample, and using the cluster(s) to identify the biological sample (e.g., the type of tissue the biological sample is from). In some embodiments, methods of generating an image of a biological sample are provided herein, e.g., the methods comprise sorting a subset of nucleic acids into a cluster based on the determined location and amount of the nucleic acids at the plurality of different locations in the biological sample, and using the cluster(s) to generate an image of the biological sample.

In some embodiments methods of molecular heterogeneity in a biological sample, e.g., the methods comprise sorting a subset of nucleic acids into a cluster based on the determined location and amount of the nucleic acids at the plurality of different locations in the biological sample, and using the cluster(s) to identify molecular heterogeneity in the biological sample relative to a reference biological sample. In some embodiments, methods of identifying a subject as having abnormal gene expression in at least one tissue, e.g., sorting a subset of nucleic acids of into a cluster based on the determined location and amount of the nucleic acids at a plurality of different locations in the biological sample, and using the cluster(s) to identify at least one region in the biological sample with abnormal gene expression relative to a reference biological sample. In some embodiments, methods of identifying a subject as having a cellular anomaly are provided herein, e.g., the methods comprise sorting a subset of nucleic acids into a cluster based on the determined location and amount of the nucleic acids at the plurality of different locations in the biological sample, and using the cluster(s) to identify at least one cellular anomaly in the biological sample. In some embodiments, methods of assessing the efficacy of a treatment or therapy in a subject are provided herein, e.g., sorting a subset of nucleic acids of into a cluster based on the determined location and amount of the nucleic acids at the plurality of different locations in the biological sample, and using the cluster(s) to identify at least one region in the biological sample having restored gene expression. In some embodiments, methods disclosed herein can be used to determine the heterogeneity of a sample, i.e., they can be used to determine the spatial or locational distribution and spatial density. For example, the methods can determine that even though the net level of a biomarker disclosed herein can be relatively the same between a normal and diseased tissue, the density or localization of that biomarker within the tissue can vary. Thus, the methods provided herein can help determine that an analyte can be expressed (even highly) in one area of a sample while not expressed in another. The expression of the analyte can be correlated with an image of the tissue.

In some embodiments, the amount of one or more nucleic acids falls outside a predetermined threshold. In some embodiments, the amount of one or more nucleic acids are elevated compared to the amount of a reference nucleic acid. In some embodiments, the amount of one or more nucleic acids are reduced compared to the amount of a reference nucleic acid.

In some embodiments, methods of comparing at least two biological samples are provided herein, e.g., the methods comprise sorting a subset of nucleic acids into a first set of clusters based on the determined location and amount of the nucleic acids at the plurality of different locations in a first biological sample; sorting a subset of nucleic acids into a second set of clusters based on the determined location and amount of the nucleic acids at the plurality of different locations in a second biological sample; and comparing the first and second sets of clusters (i.e., the clusters from the first and second biological samples).

In some embodiments, the first biological sample is from the same subject as the second biological sample. In some embodiments, there is a period of time between acquiring the first biological sample and acquiring the second biological or subsequent samples from the subject. In some embodiments, the period of time is about 1 day to about five years, e.g., about 1 day to about 10 days, about 1 day to about 1 month, about 1 day to about 6 months, about 1 day to about 1 year, about 1 day to about 1.5 years, about 1 day to about 2 years, about 1 day to about 2 years, about 1 day to about 4 years, about 4 years to about 5 years, about 3 years to about 5 years, about 2 years to about 5 years, or about 1 year to about 5 years. For example, about 1.5 years to about 2 years, about 1 year to about 2 years, about 6 months to about 2 years, about 1 to about 3 years, or about 2 to about 4 years. In some embodiments, the period of time is about 1 month, about 6 months, about 1 year, about 2 years, about 3 years, about 4 years, or about 5 years. In some embodiments, the method further comprises comparing the clusters from additional biological samples obtained from the subject before and after the period of time. In some embodiments, the first biological sample is obtained from a first subject and the second biological sample is obtained from a second subject. In some embodiments, the second biological sample is obtained from a healthy subject. In some embodiments, the first biological sample is obtained from a subject at risk (e.g., increased risk) of developing a disease.

In some embodiments, methods provided herein include sorting a subset of nucleic acids into a first set of clusters based on the determined amount and location of the nucleic acids at the plurality of different locations in the biological sample; and comparing the set of clusters to a reference set of clusters. In some embodiments, the reference set of clusters is a normalized set of clusters from more than one reference biological sample. In some embodiments, each of the more than one reference biological sample comprises the same type of tissue as the biological sample obtained from the subject.

In some embodiments, a method as described herein can further comprise identifying a subpopulation of cells in the biological sample.

In some embodiments, the biological sample comprises a nervous tissue (e.g., a brain tissue, a spinal cord tissue, a retinal tissue), blood, serum, plasma, cerebrospinal fluid, bone marrow aspirate, or a combination thereof.

In some embodiments, the biological sample is obtained from a biopsy. In some embodiments, the biological sample is obtained from a surgical excision. In some embodiments, the biological sample is obtained by venipuncture. In some embodiments, the biological sample is obtained by spinal tap.

Methods of processing biological samples (e.g., cerebrospinal fluid) for use in the methods described herein are well-known in the art. See e.g., Kansak K and Irwin D J Psychiatr Clin North Am. 2015 June; 38(2): 309-322; Paterson, R. W. et al. (2018) Alz Res Therapy 10, 32; Lewczuk P. et al., World J Biol Psychiatry. 2018 June; 19(4): 244-328, which are hereby incorporated by reference in their entirety.

(d) Locations in a Sample

As used herein, a location in a sample can be any appropriate location. For example, in some embodiments, a location can be in one or more of a basal ganglia (e.g., a striatum, a caudate nucleus, a putamen, a nucleus accumbens, an olfactory tubercle, a globus pallidus, a ventral pallidum, substantia nigra, a subthanamic nucleus, or a combination or substructure or any thereof), a brain stem (e.g., a medulla oblongata, a midbrain, a pons, or a combination or substructure of any thereof), a cerebellum, a cerebral cortex (e.g., a lobe of a cerebral cortex, an isocortex, a cortical subplate, or a combination or substructure of any thereof), a limbic system (e.g., a prefrontal cortex (e.g., a cingulate gyms, a thalamus, a hippocampus (e.g., a parahippocampal gyms and/or a subiculum)), an amygdala, a nucleus accumbens, a hypothalamus, a ventral tegmental area, a raphe nuclei, a habenular commissure, an entorhinal cortex, an olfactory bulb(s), a medial forebrain bundle, and a piriform cortex.

In some embodiments, a location can be a lobe of a cerebral cortex (e.g., a frontal lobe, a parietal lobe, a temporal lobe, or an occipital lobe). In some embodiments, a location can be in a hypothalamus (or substructure thereof). In some embodiments, a location can be in a limbic system (or substructure thereof). In some embodiments, a location can be in a hippocampus (or substructure thereof). In some embodiments, a location can be in a cerebral cortex (or substructure thereof). In some embodiments, a location can be in a brain stem (or substructure thereof). In some embodiments, a location can be in a basal ganglia (or a substructure thereof). In some embodiments, a location can be in a substantia nigra (or a substructure thereof).

In some embodiments, a location can be in one or more of nervous tissue, blood, serum, plasma and cerebrospinal fluid. In some embodiments a location can be in one or more of a brain, a spinal cord, bone marrow, and a retina. In some embodiments a location can be in one or more of a cell culture sample.

(e) Reference Levels or Reference Amounts

A reference level (“level” or “amount” for a test sample or reference sample can also be referred to as “abundance.” The terms herein are interchangeable.) of an analyte (e.g., a biomarker) can be any appropriate reference level or amount. In some embodiments, a reference level of a biomarker can be determined based on a level of the biomarker in a corresponding sample (e.g., a brain of a control animal, e.g., a control animal not diagnosed, not presenting with any of the symptoms of a brain disorder, not having a family history of a brain disorder, and not having any known risk factors of a brain disorder) at a corresponding position. In some embodiments, a reference level of a biomarker can be determined based on an amount of the biomarker in one or more other locations in a sample.

In some embodiments, a reference level can be based on a reference level as published by an appropriate body (e.g., a government agency (e.g., the United States Food and Drug Administration) or a professional organization (e.g., the American Medical Association or American Psychiatric Association)), for example, a reference level that is a threshold level for a biomarker at the location in the brain of an animal.

In some embodiments, a reference level of a biomarker can be determined based on any appropriate criteria. For example, in some embodiments, a reference level of a biomarker can come from an age-matched healthy subject. In some embodiments, a reference level of a biomarker can come from a sex-matched healthy subject or a sex-matched healthy subject population. In some embodiments, a reference level of a biomarker can come from an age-matched, sex-matched healthy subject or an age-matched, sex-matched healthy subject population. In some embodiments, a reference level of a biomarker can come from an aggregate sample (e.g., an average of 2 or more individual) of healthy subjects (e.g., that are age-matched and/or sex-matched). In some embodiments, a reference level can come from a sample (biopsy, etc) that was historically taken from a subject when that subject was deemed healthy or taken from a subject prior to associated treatment. In some embodiments, a reference level is a baseline level taken from a subject taken prior to a treatment.

A healthy subject can be any appropriate healthy subject. In some embodiments, a healthy subject has one or more of: no known brain disorder, presentation of no symptoms or no more than three (e.g., no more than two, or no more than one) of: a brain disorder, no known genetic mutations associated with risk of a brain disorder, no family medical history of a brain disorder, and no behavioral risk factors of a brain disorder.

In some cases, a level (e.g., abundance) of a biomarker can be elevated relative to a reference level. For example, a level of a biomarker can be at least 0.2-fold (e.g., at least 0.4-fold, at least 0.6-fold, at least 0.8-fold, at least 1-fold, at least 1.3-fold, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, 30-fold, 40-fold, 50-fold, or more) greater than a reference level (e.g., any of the exemplary reference levels described herein or known in the art).

In some cases, a level of a biomarker can be decreased relative to a reference level. For example, a level of a biomarker can be at least 5% less, at least 10% less, at least 15% less, at least 20% less, at least 25% less, at least 30% less, at least 35% less, at least 40% less, at least 45% less, at least 50% less, at least 55%, at least 60% less, at least 65% less, at least 70% less, at least 75% less, at least 80% less, at least 85% less, at least 90% less, at least 95% decreased (e.g., about a 5% to about a 99% decrease, about a 5% decrease to about a 80% decrease, about a 5% decrease to about a 60% decrease, about a 5% decrease to about a 40% decrease, abo0%ut a 5% decrease to about a 20% decrease, about a 20% decrease to about a 95% decrease, about a 20% decrease to about a 80% decrease, about a 20% decrease to about a 60% decrease, about a 20% decrease to about a 40% decrease, about a 40% decrease to about a 99% decrease, about a 40% decrease to about a 80% decrease, about a 40% decrease to about a 60% decrease, about a 60% decrease to about a 99% decrease, about a 60% decrease to about a 80% decrease, about a 80% decrease to about a 99% decrease) as compared to a reference level (e.g., any of the exemplary reference levels described herein). Other suitable reference levels and methods of determining the same will be apparent to those skilled in the field.

(f) Biomarkers and Candidate Biomarkers

As used herein, a biomarker can be any appropriate biomarker. In some embodiments, a biomarker can be a nucleic acid (e.g., genomic DNA (gDNA), mRNA, or rRNA (e.g., bacterial 16S rRNA)), a protein (e.g., an enzyme, a cell surface marker, a structural protein, a tumor suppressor, an antibody, a cytokine, a peptide hormone, or an identifiable fragment, precursor, or degradation product of any thereof), a lipoprotein, a fatty acid, a cell (e.g., a cell type, for example, in a location indicative of disease), or a small molecule (e.g., an enzymatic cofactor, a hormone (e.g., a steroid hormone or a eicosanoid hormone), or a metabolite). In some embodiments, a biomarker can include an alteration in a nucleic acid (e.g., an insertion, a deletion, a point mutation, and/or methylation), for example, relative to a wildtype or control nucleic acid. In some embodiments, a biomarker can include an alteration in a protein (e.g., an inserted amino acid, a deletion of an amino acid, an amino acid substitution, and/or a post-translational modification (e.g., presence, absence, or a change in, for example, acylation, isoprenylation, phosphorylation, glycosylation, methylation, hydroxylation, amidation, and/or ubiquitinylation)), for example, relative to a control or wildtype protein.

In some embodiments, a biomarker is a nucleic acid. In some embodiments, a biomarker is an mRNA molecule. In some embodiments, a biomarker is a protein. In some embodiments, a biomarker is an enzyme. In some embodiments, a biomarker is a cell surface marker.

(g) Clusters

Many methods can be used to help identify a cluster of analytes. Non-limiting examples of such methods include nonlinear dimensionality reduction methods such as t-distributed stochastic neighbor embedding (t-SNE), global t-distributed stochastic neighbor embedding (g-SNE), and uniform manifold approximation and projection (UMAP).

Any number of clusters can be identified. In some embodiments, 2 to 500 clusters can be identified using the methods as described herein. For example, 2 to 10, 2 to 20, 2 to 50, 2 to 75, 2 to 100, 2 to 150, 2 to 200, 2 to 300, 2 to 400, 400 to 500, 300 to 500, 200 to 500, 100 to 500, 75 to 500, 50 to 500, or 25 to 200 clusters can be identified. In some embodiments, 25 to 75, 50 to 100, 50 to 150, 75 to 150, or 100 to 200 clusters can be identified.

Any number of nucleic acids can be sorted into a cluster. For example, a cluster can include about 1 to about 200,000 nucleic acids. In some embodiments, a cluster can include about 1 to about 150,000, about 1 to about 100,000, about 1 to about 75,000, about 1 to about 50,000, about 100,000 to about 200,000, or about 50,000 to about 200,000 nucleic acids. In some embodiments, a cluster includes about 2 to about 25,000 nucleic acids. For example, about 2 to about 50, about 2 to about 100, about 2 to about 500, about 2 to about 1,000, about 2 to about 5,000, about 2 to about 10,000, about 2 to about 15,000, about 2 to about 20,000, about 20,000 to about 25,000, about 15,000 to about 25,000, about 10,000 to about 25,000, about 5,000 to about 25,000, about 1,000 to about 25,000, about 500 to about 25,000, or about 100 to about 25,000 nucleic acids.

In some embodiments, a nucleic acid included in a cluster is different than each of the other nucleic acids in the cluster. For example, the nucleic acid has a sequence that is not identical to any of the other nucleic acids in the cluster. In some embodiments, a nucleic acid corresponds to a gene.

(h) Identifying a Diagnostic or Prognostic Marker of a Brain Disorder or a Candidate Biomarker for Efficacy of a Treatment of a Brain Disorder

In some embodiments, provided herein are methods for identifying a diagnostic or prognostic biomarker of a brain disorder, and determining a candidate biomarker for determining efficacy of a treatment of a brain disorder. A diagnostic or prognostic biomarker is an analyte (e.g., nucleic acid, protein) that can be used to identify and/or determine the presence of a brain disorder, or determine the likelihood that a brain disorder can or will be identified in a subject. In some instances, candidate prognostic biomarker is detected and used to predict the prognosis of a subject's brain disorder. In some instances, the diagnostic or prognostic biomarker is increased relative to a reference sample. In some instances, the diagnostic or prognostic biomarker is decreased relative to a reference sample.

The methods can include (a) determining level(s) of one or more biomarker(s) in a location in a sample comprising brain tissue obtained from an animal having a brain disorder; (b) identifying: (i) one or more biomarker(s) showing elevated level(s) in the location in the sample as compared to reference level(s) of the one or more biomarker(s) and/or (ii) one or more biomarker(s) showing decreased level(s) in the location in the sample as compared to reference level(s) of the one or more biomarker(s) as diagnostic, or prognostic biomarker(s) of the brain disorder and/or or as candidate biomarker(s) for determining efficacy of a treatment of the brain disorder. In some embodiments, a reference level of the one or more biomarker(s) is a level of the one or more biomarker(s) in a corresponding location in a sample comprising brain tissue obtained from a control animal. In some embodiments, an animal can be any of the animals described herein. In some embodiments, an animal can be a mammal.

In some embodiments, the methods can include identifying one or more biomarker(s) showing elevated level(s) in the location in the sample as compared to reference level(s) of the one or more biomarker(s) as diagnostic, or prognostic biomarker(s) of the brain disorder and/or or as candidate biomarker(s) for determining efficacy of a treatment of the brain disorder. In some embodiments, the methods can include identifying one or more biomarker(s) showing decreased level(s) in the location in the sample as compared to reference level(s) of the one or more biomarker(s) (e.g., one or both of Adora2a and Trh) as diagnostic or prognostic biomarker(s) of the brain disorder, and/or or as candidate biomarker(s) for determining efficacy of a treatment of the brain disorder.

The identified diagnostic markers (identified using the methods described herein) can then be used in methods of diagnosing a brain disorder (e.g., by measuring a level of the identified diagnostic marker in a biological sample obtained from a subject (e.g., a biological sample comprising cerebrospinal fluid, blood, serum, plasma, or bone marrow aspirate), and comparing the level to a reference level (e.g., any of the exemplary reference levels described in herein (e.g., a level of the biomarker in a similar biological sample from a control subject or a population of control subjects)). For example, an increase in the level of the diagnostic biomarker (e.g., one or more of PMCH, Akr1e1, Atg4c, Gm14296, Hcrt, and Ttr) in a biological sample obtained from a subject (for a diagnostic biomarker identified as having an increased level as compared to a reference level) as compared to a level of the biomarker in a similar biological sample from a control subject or a population of control subjects indicates that the subject has the brain disorder. For example, a decrease in the level of the diagnostic biomarker (e.g., one or both of Adora2a and Trh) in a biological sample obtained from a subject (for a diagnostic biomarker identified as having an decreased level as compared to a reference level) as compared to a level of the biomarker in a similar biological sample from a control subject or a population of control subjects indicates that the subject has the brain disorder. Such methods can be used to identify patients having an early stage of a brain disorder (e.g., before the presentation of symptoms). In such methods where a patient is diagnosed as having an early stage of a brain disorder (e.g., using any of the methods described herein), the subject can be administered a treatment that allows for a decrease in the rate of progression of the brain disorder in the subject. In some embodiments, the biomarker could be used as an indicator of progression or regression of the disorder.

In some instances, a biological sample is placed on a substrate that comprises a plurality of capture probes. After permeabilization of the biological sample, analytes (e.g., mRNA molecules) migrate and hybridize to the capture probe. In some instances, the capture probe includes a capture domain that includes a poly-thymine (T) sequence that can indiscriminately hybridize to a poly(A) mRNA sequence of an analyte. Once the capture probes capture the analyte(s), first strand cDNA created by template switching and reverse transcriptase is then denatured and the second strand is then extended. The second strand cDNA is then denatured from the first strand cDNA, neutralized, and transferred to a tube. cDNA quantification and amplification can be performed using standard techniques discussed herein. The cDNA can then be subjected to library preparation and indexing, including fragmentation, end-repair, and a-tailing, and indexing PCR steps. The library preparation can optionally be quality controlled to verify the success of the library preparation methods. The cDNA fragments can then be sequenced using, for example, paired-end sequencing using TruSeq Read 1 and TruSeq Read 2 as sequencing primer sites.

In some aspects, arrays (e.g., glass slides) include a plurality of capture probes that bind to one or more specific biological targets in a sample (i.e., targeted analysis). In some aspects, the capture probes hybridize to specific analytes, e.g., under appropriate conditions where oligonucleotide capture probes can hybridize to the target nucleic acids in a sequence-specific manner. That is, the capture probe includes a sequence that is specific to an analyte of interest, and the capture probe discriminantly captures the targeted analyte. In some aspects, analytes that do not hybridize to capture probes are removed (e.g., analytes that do not interact with capture domains of the capture probes). In some embodiments, removal of analytes that did not interact with a capture probe can be accomplished by, e.g., washing the sample to remove such analytes.

In some instances, targeted capture occurs through enrichment of targets of interest after analytes are non-discriminantly captured by capture probes on an array. In this instance, analytes (e.g., mRNA) is captured by a capture probe. In some instances, the capture probe includes a sequence that hybridizes to an analyte. In some instances, the capture probe includes poly-thymine (T) sequence that hybridizes to a poly(A) sequence of an mRNA analyte. After the analytes are captured by the capture probe, the analytes are pooled and amplified. In some instances, after amplification, specific analytes of interest are enriched in the pool. In some instances, a plurality of bait oligonucleotides are added to the pool. In some instance, a bait oligonucleotide includes a capture domain that binds specifically to all or a portion of the sequence of the nucleic acid from the biological sample, or a complement thereof. In some instances, the bait oligonucleotide includes a molecular tag. In some instances, the molecular tag include a moiety such as a streptavidin molecule, an avidin molecule, a biotin molecule, or a fluorophore molecule. In some instances, the moiety can be used to isolate bait oligonucleotides that have hybridized to a target sequence of interest. After isolation of the hybridized bait oligonucleotide/target, the target can be isolated, purified, and optionally amplified using methods known in the art. In some instances, this enriched pool of a target of interest can then be sequenced to identify all or a portion of the sequence of the spatial barcode (from the initial capture probe) or the complement thereof, and all or a portion of the sequence of the nucleic acid from the biological sample, and using the determined sequences of (i) and (ii) to identify the location of the nucleic acid in the biological sample.

In some instances, a plurality of bait oligonucleotides can be designed so that each bait oligonucleotide sequence theoretically hybridizes to a unique target of interest. In some instances, the designed bait oligonucleotides are at least 40 nucleotides in length. In some instances, the bait oligonuceltoides are about 120 nucleotides in length. In some instances, the bait oligonucleotides range from about 40 to about 160 nucleotides in length. In some instances, a panel of bait oligonucleotides are used to target one analyte of interest or a plurality of analytes of interest. In some embodiments, the plurality of analytes of interest is between five genes and twenty thousand genes. In some embodiments, the plurality of analytes is between one hundred genes and ten thousand genes. In some embodiments, the plurality of analytes is between five hundred analytes and two thousand analytes. In some embodiments, the plurality of analytes is more than 10, more than 50, more than 100, more than 500, more than 1000, more than 2000, more than 5000, more than 10000, more than 15000, or more than 20000 analytes. It is appreciated that panels and bait oligonucleotides can be designed to target analytes of interest in a specific setting (e.g., for a specific tissue or for a specific pathological setting such as cancer). In some cases, spatial analysis can be performed by detecting multiple oligonucleotides that hybridize to one or more analytes. In some instances, for example, spatial analysis can be performed using RNA-templated ligation (RTL). Methods of in situ hybridization such as RTL have been described previously. See Credle et al., Nucleic Acids Res. 2017 Aug. 21; 45(14):e128. Briefly, RTL steps include hybridization of two oligonucleotides to adjacent sequences of an analyte (e.g., an RNA molecules, e.g., an mRNA molecule). In some instances, the oligonucleotides are DNA molecules. In some instances, one of the oligonucleotides includes at least two ribonucleic acid bases at the 3′ end and the other oligonucleotide includes a phosphorylated nucleotide at the 5′ end. In some instances, one of the two oligonucleotides includes a capture probe binding domain (e.g., a poly(A) sequence).

After hybridization, a ligase (e.g., T4 DNA ligase) ligates the oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. In some instances, after ligation, the ligation product is released from the analyte. In some instances, the ligation product is released using an endonuclease (e.g., RNAse H). The released ligation product can then be captured by capture probes on an array, amplified, and sequenced, thus determining the location and abundance of the analyte in the biological sample.

In some embodiments, the methods include optimizing permeabilization of a biological sample. Optimizing permeabilization can be useful for identifying intracellular analytes. Permeabilization optimization can include selection of permeabilization agents, concentration of permeabilization agents, and permeabilization duration. In general, a biological sample can be permeabilized by exposing the sample to one or more permeabilizing agents. Suitable agents for this purpose include, but are not limited to, organic solvents (e.g., acetone, ethanol, and methanol), detergents (e.g., saponin, Triton X-100™, Tween-20™, or sodium dodecyl sulfate (SDS)), and enzymes (e.g., trypsin, proteases (e.g., proteinase K). In some embodiments, the detergent is an anionic detergent (e.g., SDS or N-lauroylsarcosine sodium salt solution). In some embodiments, the biological sample can be permeabilized during any of the steps described herein (e.g., using any of the detergents described herein, e.g., SDS and/or N-lauroylsarcosine sodium salt solution) before or after enzymatic treatment (e.g., treatment with any of the enzymes described herein, e.g., trypin, proteases (e.g., pepsin and/or proteinase K)).

At any point during the methods disclosed herein, the biological sample can be imaged. For example, a region of interest can be identified in a biological sample using a variety of different techniques, e.g., expansion microscopy, bright field microscopy, dark field microscopy, phase contrast microscopy, electron microscopy, fluorescence microscopy, reflection microscopy, interference microscopy, confocal microscopy, and visual identification (e.g., by eye), and combinations thereof.

In some embodiments, this disclosure further provides devices for holding or supporting substrates for use in the methods disclosed herein. In particular, the devices include a first and second members that receive a first and second substrate, respectively. In some embodiments, the devices of the disclosure can be used for sandwiching the first and second substrates together for spatial transcriptomics applications. In some embodiments, the first substrate can support a sample (e.g., a biological substrate) on its surface. In some embodiments, the second substrate can include a plurality of barcoded probes and/or permeabilization reagents. In some instances, the biological sample is permeabilized to allow analytes to be released from the sample on the first substrate and bind (e.g., hybridize) to the capture probes attached to the second substrate. Methods and devices relating to substrates that are used in sandwiching methods are disclosed in WO 2020/123320, which in incorporated by reference in its entirety.

In some embodiments, the methods can include performing an experiment to validate whether the one or more identified candidate prognostic biomarker(s) provides for an accurate assessment of the prognosis of the brain disorder in a mammal. Non-limiting examples of experiments can include generation of a knockout (e.g., a knockout of Adora2a or Trh) or a knock-in (e.g., a knock-in of PMCH, Akr1e1, Atg4c, Gm14296, Hcrt, or Ttr) animal, and study of the prognosis of the knockout or knock-in animal. In some embodiments, the additional experiments can include following a group of patients having a brain disorder over time and assessing the level of the biomarker in the subject over time. In some embodiments, the additional experiments can include determining the level of the biomarker in an animal model of a brain disorder over time. Other experiments to validate whether the one or more identified candidate prognostic biomarker(s) will be apparent to those skilled in the field. In some embodiments, the methods can further include performing an experiment to validate whether the one or more identified candidate biomarker(s) provides for an accurate assessment of the efficacy of a treatment of the brain disorder in an animal. Non-limiting examples of experiments can include administering a treatment of a brain disorder to an animal model of the brain disorder and assessing the levels of the identified candidate biomarker in the animal model over time and assessing progression of the disease in the animal model over time). Other experiments to validate whether the one or more identified candidate biomarker(s) provides for an accurate assessment of the efficacy of a treatment of the brain disorder in an animal will be apparent to those skilled in the field.

(i) Biomarkers of Alzheimer's Disease

As described in Examples 5 and 6, PMCH, Adora2a, Trh, Akr1e1, Atg4c, Gm14296, Hcrt, and Ttr have been identified as diagnostic biomarkers of Alzheimer's disease. Further, as shown in Table 11, Prnp, Prkcd, C1qb, Glul, Pttg1, Aurkaip1, Cartpt, C4b, Mzt1, Ptpn3, Phyhip, Snhg11, Rad23b, Ntng1, Srsf5, Ptpn4, Nt5dc3, and Insig1 are significantly upregulated in the hippocampus of transgenic mice with Alzheimer's disease. As seen in Table 12, Oxt, Alad, Nudt19, Gm10076, Cap1, Rgcc, Uba52, Ppp1r1b, Pde10a, Ube2m, Hba-a1, Gstp1, Manf, Gpr88, Sap301, Alkbh6, Snhg6, Avp, Pfn1, and Tac1 are significantly downregulated in the hippocampus of transgenic mice with Alzheimer's disease. Some embodiments of any of the methods described herein can include the detection of a level of one or more of PMCH, Adora2a, Trh, Akr1e1, Atg4c, Gm14296, Hcrt, and Ttr, Prnp, Prkcd, C1qb, Glul, Pttg1, Aurkaip1, Cartpt, C4b, Mzt1, Ptpn3, Phyhip, Snhg11, Rad23b, Ntng1, Srsf5, Ptpn4, Nt5dc3, Insig1, Oxt, Alad, Nudt19, Gm10076, Cap1, Rgcc, Uba52, Ppp1r1b, Pde10a, Ube2m, Hba-a1, Gstp1, Manf, Gpr88, Sap301, Alkbh6, Snhg6, Avp, Pfn1, and Tac1, or a byproduct, a degradation product, or a precursor thereof. Some embodiments of any of the methods described herein can include the detection of a level of one or more of PMCH, Adora2a, Trh, Akr1e1, Atg4c, Gm14296, Hcrt, and Ttr, or a byproduct, a degradation product, or a precursor thereof. In some embodiments of any of the methods described herein, a biomarker can be one or more of PMCH, Adora2a, Trh, Akr1e1, Atg4c, Gm14296, Hcrt, and Ttr, or a byproduct, a degradation product, or a precursor thereof. Some embodiments of any of the methods described herein can include the detection of a level of one or more of PMCH, Hcr, Oxt, Phyhip, or a byproduct or precursor or degradation product thereof, or a byproduct, a degradation product, or a precursor thereof.

(i) PMCH and Byproducts, Degradation Products, and Precursors Thereof

Pro-melanin concentrating hormone (PMCH) is a peptide with the sequence shown as SEQ NO: 1, encoded by SEQ ID NO: 2 (Table 1). PMCH is typically expressed primarily in the hypothalamus. See e.g., Rao, C V, et al. Aging Cell. 2018; 17:e12797; and Chen W T et al., Cell 182(4), 20 Aug. 2020, Pages 976-991.e19 incorporated by reference herein in their entireties. After synthesis, PMCH can be cleaved into three chains: neuropeptide-glycine-glutamic acid (also called NGE, SEQ ID NO: 3), neuropeptide-glutamic acid-isoleucine (also called NEI, SEQ ID NO: 4), and melanin-concentrating hormone (also called MCH, SEQ ID NO: 5). In mammals, MCH is involved in the regulation of feeding behavior, mood, sleep-wake cycle, and energy balance. See e.g., Schmidt, F M, et al., PloS one, 8(5), e63136, incorporated by reference herein in its entirety. As described in Example 5, PMCH has been identified as a biomarker of Alzheimer's disease. As used herein NGE, NEI, and MCH can be considered PMCH byproducts and/or degradation products. Some embodiments of any of the methods described herein can include the detection of a level of a PMCH pseudogene. The PMCH gene is conserved in chimpanzee, dog, cow, mouse, rat, chicken and frog. At least 247 organisms have orthologs to human gene PMCH. PMCH has two pseudogenes, PMCHL1 and PMCHL2. In some embodiments of any of the methods described herein, a biomarker can be PMCH, a byproduct, degradation product, or fragment thereof, or a precursor thereof, for example, genomic DNA, mRNA, or protein. In some embodiments of any of the methods described herein, a biomarker can be a polypeptide with at least 80% sequence identity (e.g., at least 85%, 90%, 95%, 98%, or 99% sequence identity) to any one of SEQ ID NOs. 1, 3, 4, or 5. In some embodiments of any of the methods described herein, a biomarker can be a nucleic acid encoding a polypeptide with at least 80% sequence identity (e.g., at least 85%, 90%, 95%, 98% or 99% sequence identity) to any one of SEQ ID NOs. 1, 3, 4, or 5. In some embodiments of any of the methods described herein, a biomarker can be a nucleic acid with at least 80% sequence identity (e.g., at least 85%, 90%, 95%, 98% or 99% sequence identity) to SEQ ID NO: 2.

TABLE 1

| | |
|---|---|
| SEQ ID NO: 1 | MAKMNLSSYILILTFSLFSQGILLSASKSIRNLD |
| | DDMVFNTFRLGKGFQKEDTAEKSVIAPSLEQYKN |
| | DESSFMNEEENKVSKNTGSKHNFLNHGLPLNLAI |
| | KPYLALKGSVAFPAENGVQNTESTQEKREIGDEE |
| | NSAKFPIGRRDFDMLRCMLGRVYRPCWQV |
| SEQ ID NO: 2 | ATGGCAAAAATGAATCTCTCTTCCTATATATTAA |
| | TACTAACTTTTTCTTTGTTTTCTCAAGGTATTTT |
| | ACTTTCAGCATCCAAGTCCATAAGAAATTTAGAT |
| | GATGACATGGTATTTAATACATTCAGGTTGGGGA |
| | TAAGGCTTTCAGAAGGAAGACACTGCAGAAAAAT |
| | CAGTTATTGCTCCTCCCTGGAACAATATAAAAAT |
| | GATGAGAGCAGTTTCATGAACGAAGAGGAAAATA |
| | AAGTTTCAAAGAACACAGGCTCCAAACATAATTT |
| | CTTAAATCATGGTCTGCCACTGAATCTGGCTATA |
| | AAACCTTATCTTGCACTAAAAGGATCTGTAGCTT |
| | TCCCAGCTGAGAATGGAGTTCAGAATACTGAATC |
| | AACACAAGAAAAGAGAGAAATTGGGGATGAAGAA |
| | AACTCAGCTAAATTTCCTATAGGAAGGAGAGATT |
| | TTGACAGTGAGTAG |
| SEQ ID NO: 3 | GSVAFPAENGVQNTESTQE |
| SEQ ID NO: 4 | EIGDEENSAKFPI |
| SEQ ID NO: 5 | DFDMLRCMLGRVYRPCWQV |

(ii) Adenosine A2a Receptor (Adora2a) and Byproducts, Degradation Products, and Precursors Thereof

Adenosine A2a receptor (Adora2a) is a peptide with the sequence shown as SEQ NO: 6, encoded by SEQ ID NO: 7 (Table 2). Adora2a is a member of the guanine nucleotide-binding protein (G protein)-coupled receptor (GPCR) superfamily. Adora2a uses adenosine as the preferred endogenous agonist and preferentially interacts with the G(s) and G(olf) family of G proteins to increase intracellular cAMP levels. Adora2a plays a role in many biological functions, such as cardiac rhythm and circulation, cerebral and renal blood flow, immune function, pain regulation, and sleep. Adora2a has been implicated in pathophysiological conditions such as inflammatory diseases and neurodegenerative disorders.

As described in Example 6, Adora2a has been identified as a biomarker of Alzheimer's disease. Some embodiments of any of the methods described herein can include the detection of a level of Adora2a. The Adora2a gene is conserved in chimpanzee, dog, cow, mouse, rat, chicken and frog. In some embodiments of any of the methods described herein, a biomarker can be Adora2a, a byproduct, degradation product, or fragment thereof, or a precursor thereof, for example, genomic DNA, mRNA, or protein. In some embodiments of any of the methods described herein, a biomarker can be a polypeptide with at least 80% sequence identity (e.g., at least 85%, 90%, 95%, 98%, or 99% sequence identity) to any one of SEQ ID NOs: 6 or 7. In some embodiments of any of the methods described herein, a biomarker can be a nucleic acid encoding a polypeptide with at least 80% sequence identity (e.g., at least 85%, 90%, 95%, 98% or 99% sequence identity) to any one of SEQ ID NO: 6. In some embodiments of any of the methods described herein, a biomarker can be a nucleic acid with at least 80% sequence identity (e.g., at least 85%, 90%, 95%, 98% or 99% sequence identity) to SEQ ID NO: 7.

TABLE 2

| | |
|---|---|
| SEQ ID NO: 6 | MPIMGSSVYITVELAIAVLAILGNVLVCWAVWLNSNLQNVTNYFVVS |
| | LAAADIAVGVLAIPFAITISTGFCAACHGCLFIACFVLVLTQSSIFS |
| | LLAIAIDRYIAIRIPLRYNGLVTGTRAKGIIAICWVLSFAIGLTPML |
| | GWNNCGQPKEGKNHSQGCGEGQVACLFEDVVPMNYMVYFNFFACVLV |
| | PLLLMLGVYLRIFLAARRQLKQMESQPLPGERARSTLQKEVHAAKSL |
| | AIIVGLFALCWLPLHIINCFTFFCPDCSHAPLWLMYLAIVLSHTNSV |
| | VNPFIYAYRIREFRQTFRKIIRSHVLRQQEPFKAAGTSARVLAAHGS |
| | DGEQVSLRLNGHPPGVWANGSAPHPERRPNGYALGLVSGGSAQESQG |
| | NTGLPDVELLSHELKGVCPEPPGLDDPLAQDGAGVS |
| SEQ ID NO: 7 | GAAGCTCTGCCTGGGCCTCAGGGACTGTGACATGGAGCAGGAGC |
| | CGCCCCCAGCCAAGCTGCTTTCAGCACAGCGTGGGCCCCCAGCAC |
| | CTTGGTGCGGGGTGCGGCCCCTCGGAGGAGGGCTGTCAGGTGAA |
| | GCCTCGTGTGAGGGGGTGCCTCAGGAACCCTGAAGCTGGGCTGA |
| | GCCATGATGCTGCTGCCAGAACCCCTGCAGAGGGCCTGGTTTCAG |
| | GAGACTCAGAGTCCTCTGTGAAAAAGCCCTTGGAGAGCGCCCCA |
| | GCAGGGCTGCACTTGGCTCCTGTGAGGAAGGGGCTCAGGGGTCT |
| | GGGCCCCTCCGCCTGGGCCGGGCTGGGAGCCAGGCGGGCGGCTG |
| | GGCTGCAGCAATGGACCGTGAGCTGGCCCAGCCCGCGTCCGTGCT |
| | GAGCCTGCCTGTCGTCTGTGGCCATGCCCATCATGGGCTCCTCGG |
| | TGTACATCACGGTGGAGCTGGCCATTGCTGTGCTGGCCATCCTGG |
| | GCAATGTGCTGGTGTGCTGGGCCGTGTGGCTCAACAGCAACCTGC |
| | AGAACGTCACCAACTACTTTGTGGTGTCACTGGCGGCGGCCGACA |
| | TCGCAGTGGGTGTGCTCGCCATCCCCTTTGCCATCACCATCAGCA |
| | CCGGGTTCTGCGCTGCCTGCCACGGCTGCCTCTTCATTGCCTGCTT |
| | CGTCCTGGTCCTCACGCAGAGCTCCATCTTCAGTCTCCTGGCCATC |
| | GCCATTGACCGCTACATTGCCATCCGCATCCCGCTCCGGTACAAT |
| | GGCTTGGTGACCGGCACGAGGGCTAAGGGCATCATTGCCATCTGC |
| | TGGGTGCTGTCGTTTGCCATCGGCCTGACTCCCATGCTAGGTTGG |
| | AACAACTGCGGTCAGCCAAAGGAGGGCAAGAACCACTCCCAGGG |
| | CTGCGGGGAGGGCCAAGTGGCCTGTCTCTTTGAGGATGTGGTCCC |
| | CATGAACTACATGGTGTACTTCAACTTCTTTGCCTGTGTGCTGGTG |
| | CCCCTGCTGCTCATGCTGGGTGTCTATTTGCGGATCTTCCTGGCGG |
| | CGCGACGACAGCTGAAGCAGATGGAGAGCCAGCCTCTGCCGGGG |
| | GAGCGGGCACGGTCCACACTGCAGAAGGAGGTCCATGCTGCCAA |
| | GTCACTGGCCATCATTGTGGGGCTCTTTGCCCTCTGCTGGCTGCCC |
| | CTACACATCATCAACTGCTTCACTTTCTTCTGCCCCGACTGCAGCC |
| | ACGCCCCTCTCTGGCTCATGTACCTGGCCATCGTCCTCTCCCACAC |
| | CAATTCGGTTGTGAATCCCTTCATCTACGCCTACCGTATCCGCGA |
| | GTTCCGCCAGACCTTCCGCAAGATCATTCGCAGCCACGTCCTGAG |
| | GCAGCAAGAACCTTTCAAGGCAGCTGGCACCAGTGCCCGGGTCTT |
| | GGCAGCTCATGGCAGTGACGGAGAGCAGGTCAGCCTCCGTCTCA |
| | ACGGCCACCCGCCAGGAGTGTGGGCCAACGGCAGTGCTCCCCAC |
| | CCTGAGCGGAGGCCCAATGGCTATGCCCTGGGGCTGGTGAGTGG |
| | AGGGAGTGCCCAAGAGTCCCAGGGGAACACGGGCCTCCCAGACG |
| | TGGAGCTCCTTAGCCATGAGCTCAAGGGAGTGTGCCCAGAGCCCC |
| | CTGGCCTAGATGACCCCCTGGCCCAGGATGGAGCAGGAGTGTCCT |
| | GATGATTCATGGAGTTTGCCCCTTCCTAAGGGAAGGAGATCTTTA |
| | TCTTTCTGGTTGGCTTGACCAGTCACGTTGGGAGAAGAGAGAGAG |
| | TGCCAGGAGACCCTGAGGGCAGCCGGTTCCTACTTTGGACTGAGA |
| | GAAGGGAGCCCCAGGCTGGAGCAGCATGAGGCCCAGCAAGAAG |
| | GGCTTGGGTTCTGAGGAAGCAGATGTTTCATGCTGTGAGGCCTTG |
| | CACCAGGTGGGGGCCACAGCACCAGCAGCATCTTTGCTGGGCAG |
| | GGCCCAGCCCTCCACTGCAGAAGCATCTGGAAGCACCACCTTGTC |
| | TCCACAGAGCAGCTTGGGCACAGCAGACTGGCCTGGCCCTGAGA |
| | CTGGGGAGTGGCTCCAACAGCCTCCTGCCACCCACACACCACTCT |
| | CCCTAGACTCTCCTAGGGTTCAGGAGCTGCTGGGCCCAGAGGTGA |
| | CATTTGACTTTTTTCCAGGAAAAATGTAAGTGTGAGGAAACCCTT |
| | TTTATTTTATTACCTTTCACTCTCTGGCTGCTGGGTCTGCCGTCGG |
| | TCCTGCTGCTAACCTGGCACCAGAGCCTCTGCCCGGGGAGCCTCA |
| | GGCAGTCCTCTCCTGCTGTCACAGCTGCCATCCACTTCTCAGTCCC |
| | AGGGCCATCTCTTGGAGTGACAAAGCTGGGATCAAGGACAGGGA |
| | GTTGTAACAGAGCAGTGCCAGAGCATGGGCCCAGGTCCCAGGGG |
| | AGAGGTTGGGGCTGGCAGGCCACTGGCATGTGCTGAGTAGC GCA |
| | GAGCTACCCAGTGAGAGGCCTTGTCTAACTGCCTTTCCTTCTAAA |
| | GGGAATGTTTTTTTCTGAGATAAAATAAAAACGAGCCACATCGTG |
| | TTTTAAGCTTGTCCAAATGA |

(iii) Thyrotropin Releasing Hormone Preproprotein (Trh) and Byproducts, Degradation Products, and Precursors Thereof

Thyrotropin releasing hormone preproprotein (Trh) is a peptide with the sequence shown as SEQ NO: 8, encoded by SEQ ID NO: 9 (Table 3). Trh encodes a member of the thyrotropin-releasing hormone family. Cleavage of the encoded proprotein releases mature thyrotropin-releasing hormone, which is a tripeptide hypothalamic regulatory hormone. The human proprotein contains six thyrotropin-releasing hormone tripeptides. Thyrotropin-releasing hormone is involved in the regulation and release of thyroid-stimulating hormone, as well as prolactin. Deficiency of this hormone has been associated with hypothalamic hypothyroidism.

As described in Example 6, Trh has been identified as a biomarker of Alzheimer's disease. Some embodiments of any of the methods described herein can include the detection of a level of Trh. In some embodiments of any of the methods described herein, a biomarker can be Trh, a byproduct, degradation product, or fragment thereof, or a precursor thereof, for example, genomic DNA, mRNA, or protein. In some embodiments of any of the methods described herein, a biomarker can be a polypeptide with at least 80% sequence identity (e.g., at least 85%, 90%, 95%, 98%, or 99% sequence identity) to any one of SEQ ID NOs: 8 or 9. In some embodiments of any of the methods described herein, a biomarker can be a nucleic acid encoding a polypeptide with at least 80% sequence identity (e.g., at least 85%, 90%, 95%, 98% or 99% sequence identity) to any one of SEQ ID NO: 8. In some embodiments of any of the methods described herein, a biomarker can be a nucleic acid with at least 80% sequence identity (e.g., at least 85%, 90%, 95%, 98% or 99% sequence identity) to SEQ ID NO: 9.

TABLE 3

| | |
|---|---|
| SEQ ID NO: 8 | NIPGPWLLLALALTLNLTGVPGGRAQPEAAQQEAVTAAEHPGLDDF |
| | LRQVERLLFLRENIQRLQGDQGEHSASQIFQSDWLSKRQHPGKREE |
| | EEEEGVEEEEEEEGGAVGPHKRQHPGRREDEASWSVDVTQHKRQHP |
| | GRRSPWLAYAVPKRQHPGRRLADPKAQRSWEEEEEEEEREEDLMPE |
| | KRQHPGKRALGGPCGPQGAYGQAGLLLGLLDDLSRSQGAEEKRQHP |
| | GRRAAWVREPLEE |
| SEQ ID NO: 9 | CGGCAGGCGCCCGGGGTCCTCAGCGCTGCAGACTCCTGACCTGCC |
| | GACTGCGGATCCCGAGTCCCCGGATCCCGGACCCATCCTGTGGAG |
| | CCCACTCCTGGCAGACGCCGCGATGCCCGGCCCTTGGTTGCTGCT |
| | CGCTCTGGCTTTGACCCTGAACCTGACCGGTGTCCCCGGCGGCCG |
| | TGCTCAGCCAGAGGCGGCCCAGCAGGAGGCAGTGACGGCCGCGG |
| | AGCATCCGGGCCTGGATGACTTCCTGCGCCAGGTGGAGCGCCTCC |
| | TCTTCCTCCGGGAAAACATCCAGCGGCTGCAAGGGGACCAGGGT |
| | GAGCACTCCGCGTCCCAGATCTTTCAATCTGACTGGCTCTCCAA |
| | ACGTCAGCATCCAGGCAAAAGAGAGGAGGAGGAGGAAGAGGGAG |
| | TTGAAGAAGAGGAAGAGGAAGAAGGGGGGGCTGTGGGACCCCA |
| | CAAACGGCAGCACCCTGGCCGACGAGAAGATGAGGCTTCATGGT |
| | CAGTCGATGTAACCCAGCACAAGCGGCAGCATCCTGGCCGGCGC |
| | TCCCCCTGGCTTGCATATGCTGTCCCGAAGCGGCAGCACCCAGG |
| | CAGAAGGCTGGCAGATCCCAAGGCTCAAAGGAGCTGGGAAGAAG |
| | AGGAGGAGGAGGAAGAGAGAGAGGAAGACCTGATGCCTGAAAAA |
| | CGCCAGCATCCGGGCAAGAGGGCCCTGGGAGGCCCCTGTGGGCC |
| | CCAGGGAGCCTATGGTCAAGCGGGCCTTCTGCTGGGGCTCCTGGA |
| | TGACCTGAGTAGGAGCCAGGGAGCTGAGGAAAAGCGGCAGCACC |
| | CTGGTCGGCGGGCAGCCTGGGTCAGAGAGCCCCTGGAGGAGTGAA |
| | CCCAGTTTTCCCTGAAGTCGAGTTTGTGGTCTAAGGATGTCTTGA |
| | GCCCTGTGTGCCCCACCATTCATGACCTCTGTATTCTCTAGTTAG |
| | ATCCCTGACCATAAGCCTGAGCCCCTCCCTCCCAGCCCCATATTC |
| | ACACACATCCCAGCCCCTGGCCTTGCCCTCTTCCTTTAGGCATGTG |
| | AGAAAATCAGCCTAGCAGTTTAAACCCCACTTTCCTCCACTTAGC |
| | ACCATAGGCAAGGGGGCAGATCCCAGAGCCCCTCTCACCCCCCCC |
| | ACCACAGGCCTGCTCCTTCCTTAGCCTTGGCTAAGATGGTCCTTC |
| | TGTGTCTTGCAAAGACTCCCCAAGTGGGACAGGGAGCCCCTGGGA |
| | GGGCAGCCAGTGAGGGTGGGGTGGGACTGAAGCGTTGTGTGCAAA |
| | TCCAGCTTCCATCCCCTCCCCAACCTGGCAGGATTCTCCATGTGT |
| | AAACTTCACCCCCAGGACCCAGGATCTTCTCCTTTCTGGGCATCC |
| | CTTTGTGGGTGGGCAGAGCCCTGACCCACAGCTGTGTTACTGCTT |
| | GGAGAAGCATATGTAGGGGCATACCCTGTGGTGTTGTGCTGTGTC |
| | TGGCTGTGGGATAAATGTGTGTGGGAATATTGAAACATCGCCTAG |
| | GAATTGTGGTTTGTATATAACCCTCTAAGCCCCTATCCCTTGTCGA |
| | TGACAGTCATCCTAATGATAATAAAACCTGCATCCAGATAA |

(iv) Aldo-Keto Reductase Family 1, Member E1 (Akr1e1) and Byproducts, Degradation Products, and Precursors Thereof

Aldo-keto reductase family 1, member E1 (Akr1e1) is a peptide with the sequence shown as SEQ NOs: 10-12, encoded by SEQ ID NOs: 13-15, respectively (Table 4). Akr1e1 is a member of the aldo-keto reductase superfamily. Members in this family are characterized by their structure (evolutionarily highly conserved TIM barrel) and function (NAD(P)H-dependent oxido-reduction of carbonyl groups). Transcripts of this gene have been reported in specimens of human testis.

As described in Example 6, Akr1e1 has been identified as a biomarker of Alzheimer's disease. Some embodiments of any of the methods described herein can include the detection of a level of Akr1e1. In some embodiments of any of the methods described herein, a biomarker can be Akr1e1, a byproduct, degradation product, or fragment thereof, or a precursor thereof, for example, genomic DNA, mRNA, or protein. In some embodiments of any of the methods described herein, a biomarker can be a polypeptide with at least 80% sequence identity (e.g., at least 85%, 90%, 95%, 98%, or 99% sequence identity) to any one of SEQ ID NOs. 10, 11, 12, 13, 14, or 15. In some embodiments of any of the methods described herein, a biomarker can be a nucleic acid encoding a polypeptide with at least 80% sequence identity (e.g., at least 85%, 90%, 95%, 98% or 99% sequence identity) to any one of SEQ ID NOs: 10-12. In some embodiments of any of the methods described herein, a biomarker can be a nucleic acid with at least 80% sequence identity (e.g., at least 85%, 90%, 95%, 98% or 99% sequence identity) to SEQ ID NOs: 13-15.

TABLE 4

| | |
|---|---|
| SEQ ID NO: | MGDIPAVGLSSWKASPGKVTEAVKEAIDAGYRHFDCAYFYHNEREV |
| 10 (Isoform-1) | GAGIRCKIKEGAVRREDLFIATKLWCTCHKKSLVETACRKSLKALK |
| | LNYLDLYLIHWPMGFKPPHPEVVIMSCSELSFCLSHPRVQDLPLDE |
| | SNMVIPSDTDFLDTWEAMEDLVITGLVKNIGVSNFNHEQLERLLNK |
| | PGLRFKPLTNQIECHPYLTQKNLISFCQSRDVSVTAYRPLGGSCEG |
| | VDLIDNPVIKRIAKEHGKSPAQILIRFQIQRNVIVIPGSITPSHIK |
| | ENIQVFDFELTQHDMDNILSLNRNLRLAMFPITKNHKDYPFHIEY |
| SEQ ID NO: | MGDIPAVGLSSWKASPGKVTEAVKEAIDAGYRHFDCAYFYHNEREV |
| 11 (Isoform-2) | GAGIRCKIKEGAVRREDLFIATKLWCTCHKKSLVETACRKSLKALK |
| | LNYLDLYLIHWPMGFKPPHPEVVIMSCSELSFCLSHPRVQDLPLDE |
| | SNMVIPSDTDFLDTWEAMEDLVITGLVKNIGVSNFNHEQLERLLNK |
| | PGLRFKPLTNQILIRFQIQRNVIVIPGSITPSHIKENIQVFDFELT |
| | QHDMDNILSLNRNLRLAMFPITKNHKDYPFHIEY |
| SEQ ID NO: | MGDIPAVGLSSWKASPGKVTEAVKEAIDAGYRHFDCAYFYHNEREV |
| 12 (Isoform-3) | GAGIRCKIKEGAVRREDLFIATKLWCTCHKKSLVETACRKSLKALK |
| | LNYLDLYLIHVVPMGFKPPHPEWIMSCSELSFCLSHPRVQDLPLDE |
| | SNMVIPSDTDFLDTWEILIRFQIQRNVIVIPGSITPSHIKENIQVF |
| | DFELTQHDMDNILSLNRNLRLAMFPITKNHKDYPFHIEY |
| SEQ ID NO: | GTCACAAGGCACTTCCAGCCAGTCGCAACGGCGGGTCGCCAGCGCC |
| 13 (Isoform-1) | GCAGTAGCTCGCGCGGTGCCTGTCGGTAGTCGCGTGCGGGGCGGCG |
| | GGGCGGCGGGGCGGCCGGCGGCGGCCATGGGAGATATCCCAGCCGT |
| | GGGCCTCAGCTCCTGGAAGGCTTCTCCAGGGAAAGTGACCGAGGCA |
| | GTGAAAGAGGCCATTGACGCAGGGTACCGGCACTTCGACTGTGCTT |
| | ACTTTTACCACAATGAGAGGGAGGTTGGAGCAGGGATCCGTTGCAA |
| | GATCAAGGAAGGCGCTGTAAGACGGGAGGATCTGTTCATTGCCACT |
| | AAGCTGTGGTGCACCTGCCATAAGAAGTCCTTGGTGGAAACAGCAT |
| | GCAGAAAGAGTCTCAAGGCCTTGAAGCTGAACTATTTGGACCTCTA |
| | CCTCATACACTGGCCCATGGGTTTCAAGCCTCCTCATCCAGAATGG |
| | ATCATGAGCTGCAGTGAACTTTCCTTCTGCCTCTCACATCCTCGAG |
| | TGCAGGACTTGCCTCTGGACGAGAGCAACATGGTTATTCCCAGTGA |
| | CACGGACTTCCTGGACACGTGGGAGGCCAT |
| | GGAGGACCTGGTGATCACCGGGCTGGTGAAGAACATCGGGGTGT |
| | CAAACTTCAACCATGAACAGCTTGAGAGGCTTTTGAATAAGCCT |
| | GGGTTGAGGTTCAAGCCACTAACCAACCAGATTGAGTGCCACCC |
| | ATATCTTACTCAGAAGAATCTGATCAGTTTTTGCCAATCCAGAG |
| | ATGTGTCCGTGACTGCTTACCGTCCTCTTGGTGGCTCGTGTGAG |
| | GGGGTTGACCTGATAGACAACCCTGTGATCAAGAGGATTGCAAA |
| | GGAGCACGGCAAGTCTCCTGCTCAGATTTTGATCCGATTTCAAA |
| | TCCAGAGGAATGTGATAGTGATCCCCGGATCTATCACCCCAAGT |
| | CACATTAAAGAGAATATCCAGGTGTTTGATTTTGAATTAACACA |
| | GCACGATATGGATAACATCCTCAGCCTAAACAGGAATCTCCGAC |
| | TGGCCATGTTCCCCATAACTAAAAATCACAAAGACTATCCTTTC |
| | CACATAGAATACTGAGGACGCTTCCCCTTCCTTGTTTCTGCTCA |
| | GCCCAGATGCACAGACACTATTGGCAATGTTGACCCTCCTCTGT |
| | CATCACAGCGCCAGGGCAGCTGTGCCTGGGACAGGAGCCACACA |
| | GTCAGAGGGGGATGTAAGAGCCACCTTCTCTGACAAATCTGGAG |
| | AATTGAGTGTGTTCTAAGTGAAGGCAATGGGGTTTCTCCAAGAC |
| | AGCCTGTGTGGCCTCTACTCTGAACAAATACACTGATGAGTCAT |
| | CAGTGAAATTTGCCTTCACATTTTAAGAAAACTTTATCTTATGG |
| | AGTTATTTAAGCCATCTACAGAGCTGAGGAAACAGTGTAATGTG |
| | TCTCTGCCCCATTGCGCAGCTCCACCCATTGTGCCCCAGGCCAG |
| | CCCGCGTCACCTACACTTCCTTCTGTGCCCTGCCAGTGACCCCC |
| | AGGTTATTCTAAAGCAGAGTCCTTCCCTTCCCCCAGTGAGAAGG |
| | AAAATGGGATAAGTCTGGGACACTGTTTCAGTTCAATAAAGAGG |
| | CTTTTTTCTTCCTTAAAAA |
| SEQ ID NO: | ATGCTTCCAGCCATTGTCGGAGTGTCAGCCGTCACAAGGCACTT |
| 14 (Isoform-2) | CCAGCCAGTCGCAACGGCGGGTCGCCAGCGCCGCAGTAGCTCGC |
| | GCGGTGCCTGTCGGTAGTCGCGTGCGGGGCGGCGGGGCGGCGGG |
| | GCGGCCGGCGGCGGCCATGGGAGATATCCCAGCCGTGGGCCTCA |
| | GCTCCTGGAAGGCTTCTCCAGGGAAAGTGACCGAGGCAGTGAAA |
| | GAGGCCATTGACGCAGGGTACCGGCACTTCGACTGTGCTTACTT |
| | TTACCACAATGAGAGGGAGGTTGGAGCAGGGATCCGTTGCAAGA |
| | TCAAGGAAGGCGCTGTAAGACGGGAGGATCTGTTCATTGCCACTA |
| | AGCTGTGGTGCACCTGCCATAAGAAGTCCTTGGTGGAAACAGCAT |
| | GCAGAAAGAGTCTCAAGGCCTTGAAGCTGAACTATTTGGACCTCT |
| | ACCTCATACACTGGCCCATGGGTTTCAAGCCTCCTCATCCAGAAT |
| | GGATCATGAGCTGCAGTGAACTTTCCTTCTGCCTCTCACATCCTC |
| | GAGTGCAGGACTTGCCTCTGGACGAGAGCAACATGGTTATTCCCA |
| | GTGACACGGACTTCCTGGACACGTGGGAGGCCATGGAGGACCTGG |
| | TGATCACCGGGCTGGTGAAGAACATCGGGGTGTCAAACTTCAACC |
| | ATGAACAGCTTGAGAGGCTTTTGAATAAGCCTGGGTTGAGGTTCA |
| | AGCCACTAACCAACCAGATTTTGATCCGATTTCAAATCCAGAGGA |
| | ATGTGATAGTGATCCCCGGATCTATCACCCCAAGTCACATTAAAG |
| | AGAATATCCAGGTGTTTGATTTTGAATTAACACAGCACGATATGG |
| | ATAACATCCTCAGCCTAAACAGGAATCTCCGACTGGCCATGTTCC |
| | CCATAACTAAAAATCACAAAGACTATCCTTTCCACATAGAATACT |
| | GAGGACGCTTCCCCTTCCTTGTTTCTGCTCAGCCCAGATGCACAG |
| | ACACTATTGGCAATGTTGACCCTCCTCTGTCATCACAGCGCCAGG |
| | GCAGCTGTGCCTGGGACAGGAGCCACACAGTCAGAGGGGGATGT |
| | AAGAGCCACCTTCTCTGACAAATCTGGAGAATTGAGTGTGTTCTA |
| | AGTGAAGGCAATGGGGTTTCTCCAAGACAGCCTGTGTGGCCTCTA |
| | CTCTGAACAAATACACTGATGAGTCATCAGTGAAATTTGCCTTCA |
| | CATTTTAAGAAAACTTTATCTTATGGAGTTATTTAAGCCATCTAC |
| | AGAGCTGAGGAAACAGTGTAATGTGTCTCTGCCCCATTGCGCAGC |
| | TCCACCCATTGTGCCCCAGGCCAGCCCGCGTCACCTACACTTCCT |
| | TCTGTGCCCTGCCAGTGACCCCCAGGTTATTCTAAAGCAGAGTCC |
| | TTCCCTTCCCCCAGTGAGAAGGAAAATGGGATAAGTCTGGGACAC |
| | TGTTTCAGTTCAATAAAGAGGCTTTTTTCTTCCTTAAAAAAAAAA |
| | AAAAAAAA |
| SEQ ID NO: | ATGCTTCCAGCCATTGTCGGAGTGTCAGCCGTCACAAGGCACTTC |
| 15 (Isoform-3) | CAGCCAGTCGCAACGGCGGGTCGCCAGCGCCGCAGTAGCTCGCG |
| | CGGTGCCTGTCGGTAGTCGCGTGCGGGGCGGCGGGGCGGCGGGG |
| | CGGCCGGCGGCGGCCATGGGAGATATCCCAGCCGTGGGCCTCAG |
| | CTCCTGGAAGGCTTCTCCAGGGAAAGTGACCGAGGCAGTGAAAG |
| | AGGCCATTGACGCAGGGTACCGGCACTTCGACTGTGCTTACTTTT |
| | ACCACAATGAGAGGGAGGTTGGAGCAGGGATCCGTTGCAAGATCA |
| | AGGAAGGCGCTGTAAGACGGGAGGATCTGTTCATTGCCACTAAGC |
| | TGTGGTGCACCTGCCATAAGAAGTCCTTGGTGGAAACAGCATGCA |
| | GAAAGAGTCTCAAGGCCTTGAAGCTGAACTATTTGGACCTCTACC |
| | TCATACACTGGCCCATGGGTTTCAAGCCTCCTCATCCAGAATGGA |
| | TCATGAGCTGCAGTGAACTTTCCTTCTGCCTCTCACATCCTCGAG |
| | TGCAGGACTTGCCTCTGGACGAGAGCAACATGGTTATTCCCAGTG |
| | ACACGGACTTCCTGGACACGTGGGAGATTTTGATCCGATTTCAAA |
| | TCCAGAGGAATGTGATAGTGATCCCCGGATCTATCACCCCAAGTC |
| | ACATTAAAGAGAATATCCAGGTGTTTGATTTTGAATTAACACAGC |
| | ACGATATGGATAACATCCTCAGCCTAAACAGGAATCTCCGACTGG |
| | CCATGTTCCCCATAACTAAAAATCACAAAGACTATCCTTTCCACA |
| | TAGAATACTGAGGACGCTTCCCCTTCCTTGTTTCTGCTCAGCCCA |
| | GATGCACAGACACTATTGGCAATGTTGACCCTCCTCTGTCATCAC |
| | AGCGCCAGGGCAGCTGTGCCTGGGACAGGAGCCACACAGTCAGAG |
| | GGGGATGTAAGAGCCACCTTCTCTGACAAATCTGGAGAATTGAGT |
| | GTGTTCTAAGTGAAGGCAATGGGGTTTCTCCAAGACAGCCTGTGT |
| | GGCCTCTACTCTGAACAAATACACTGATGAGTCATCAGTGAAATT |
| | TGCCTTCACATTTTAAGAAAACTTTATCTTATGGAGTTATTTAAG |
| | CCATCTACAGAGCTGAGGAAACAGTGTAATGTGTCTCTGCCCCAT |
| | TGCGCAGCTCCACCCATTGTGCCCCAGGCCAGCCCGCGTCACCTA |
| | CACTTCCTTCTGTGCCCTGCCAGTGACCCCCAGGTTATTCTAAAG |
| | CAGAGTCCTTCCCTTCCCCCAGTGAGAAGGAAAATGGGATAAGTC |
| | TGGGACACTGTTTCAGTTCAATAAAGAGGCTTTTTTCTTCCTTAA |
| | AAAAAAAAAAAAAAAA |

(v) Autophagy Related 4C Cysteine Peptidase (Atg4c) and Byproducts, Degradation Products, and Precursors Thereof

Autophagy related 4C cysteine peptidase (Atg4c) is a peptide with the sequence shown as SEQ NOs: 16-18, encoded by SEQ ID NO: 19-21, respectively (Table 5). Autophagy is the process by which endogenous proteins and damaged organelles are destroyed intracellularly. Autophagy is postulated to be essential for cell homeostasis and cell remodeling during differentiation, metamorphosis, non-apoptotic cell death, and aging. Reduced levels of autophagy have been described in some malignant tumors, and a role for autophagy in controlling the unregulated cell growth linked to cancer has been proposed. Atg4c encodes a member of the autophagin protein family. The encoded protein is also designated as a member of the C-54 family of cysteine proteases.

As described in Example 6, Atg4c has been identified as a biomarker of Alzheimer's disease. Some embodiments of any of the methods described herein can include the detection of a level of Atg4c. In some embodiments of any of the methods described herein, a biomarker can be Atg4c, a byproduct, degradation product, or fragment thereof, or a precursor thereof, for example, genomic DNA, mRNA, or protein. In some embodiments of any of the methods described herein, a biomarker can be a polypeptide with at least 80% sequence identity (e.g., at least 85%, 90%, 95%, 98%, or 99% sequence identity) to any one of SEQ ID NOs: 16-21. In some embodiments of any of the methods described herein, a biomarker can be a nucleic acid encoding a polypeptide with at least 80% sequence identity (e.g., at least 85%, 90%, 95%, 98% or 99% sequence identity) to any one of SEQ ID NOs: 16-18. In some embodiments of any of the methods described herein, a biomarker can be a nucleic acid with at least 80% sequence identity (e.g., at least 85%, 90%, 95%, 98% or 99% sequence identity) to SEQ ID NOs: 19-21.

TABLE 5

| | |
|---|---|
| SEQ ID NO: | MEATGTDEVDKLKTKFISAWNNMKYSWVLKTKTYFSRNSPVLLLGK |
| 16 (Isoform-1) | CYHFKYEDEDKTLPAESGCTIEDHVIAGNVEEFRKDFISRIWLTYR |
| | EEFPQIEGSALTTDCGWGCTLRTGQMLLAQGLILHFLGRAWTWPDA |
| | LNIENSDSESWTSHTVKKFTASFEASLSGEREFKTPTISLKETIGK |
| | YSDDHEMRNEVYHRKIISWFGDSPLALFGLHQLIEYGKKSGKKAGD |
| | WYGPAVVAHILRKAVEEARHPDLQGITIYVAQDCTVYNSDVIDKQS |
| | ASMTSDNADDKAVIILVPVRLGGERTNTDYLEFVKGILSLEYCVGI |
| | IGGKPKQSYYFAGFQDDSLIYMDPHYCQSFVDVSIKDFPLETFHCP |
| | SPKKMSFRKMDPSCTIGFYCRNVQDFKRASEEITKMLKFSSKEKYP |
| | LFTFVNGHSRDYDFTSTTTNEEDLFSEDEKKQLKRFSTEEFVLL |
| SEQ ID NO: | MEATGTDEVDKLKTKFISAWNNMKYSWVLKTKTYFSRNSPVLLLGK |
| 17 (Isoform-2) | CYHFKYEDEDKTLPAESGCTIEDHVIAGNVEEFRKDFISRIWLTYR |
| | EEFPQIEGSALTTDCGWGCTLRTGQMLLAQGLILHFLGRAWTWPDA |
| | LNIENSDSESWTSHTVKKFTASFEASLSGEREFKTPTISLKETIGK |
| | YSDDHEMRNEVYHRKIISWFGDSPLALFGLHQLIEYGKKSGKKAGD |
| | WYGPAVVAHILRKAVEEARHPDLQGITIYVAQDCTVYNSDVIDKQS |
| | ASMTSDNADDKAVIILVPVRLGGERTNTDYLEFVKGILSLEYCVGI |
| | IGGKPKQSYYFAGFQDDSLIYMDPHYCQSFVDVSIKDFPLEMLKFS |
| | SKEKYPLFTFVNGHSRDYDFTSTTTNEEDLFSEDEKKQLKRFSTEE |
| | FVLL |
| SEQ ID NO: | MEATGTDEVDKLKTKFISAWNNMKYSWVLKTKTYFSRNSPVLLLGK |
| 18 (Isoform-3) | CYHFKYEDEDKTLPAESGCTIEDHVIAGNVEEFRKDFISRIWLTYR |
| | EEFPQIEGSALTTDCGWGCTLRTGQMLLAQGLILHFLGRAWTWPDA |
| | LNIENSDSESWTSHTVKKFTASFEASLSGEREFKTPTISLKETIGK |
| | YSDDHEMRNEVYHRKIISWFGDSPLALFGLHQLIEYGKKSGKKAGD |
| | WYGPAVVAHILRKAVEEARHPDLQGITIYVAQDCTVYNSDVIDKQS |
| | ASMTSDNADDKAVIILVPVRLGGERTNTDYLEFVKGILSLEYCVGI |
| | IGGKPKQSYYFAGFQDDSLIYMDPHYCQSFVDVSIKDFPLEMLKFS |
| | SKEKYPLFTFVNGHSRDYDFTSTTTNEEDLFSEDEKKQLKRFSTEE |
| | FVLL |
| SEQ ID NO: | CCGCAATGCATGCTGGATCTTGCAGTTCCTACGCCAGAGGCAAGGG |
| 19 (Isoform-1) | CGTGATGCTGCATGGCCTGCTGGTAGTTGAAGTCCAAGTGCTCGAG |
| | AGGAATTTATGGCAGCCGTAATGTGGTTTGTCTATCAGAGAGACAA |
| | TGCTCACAGACGGATACTTTCTCTTGGTTCTGCGCTTGCTGCATTG |
| | GTGGATTAGCGGCTTGGGGTGGCGAACAGTCAGTATAAAAGATTAA |
| | ACTCTACAGAAGAATGCAATCAAGTGATGGCTTTTCCTTTAGAATT |
| | TGAATATGGAGGCTACAGGAACAGATGAAGTTGACAAGCTAAAAAC |
| | CAAATTTATATCTGCTTGGAACAACATGAAATATAGTTGGGTGTTG |
| | AAAACAAAGACGTATTTTAGTAGAAATTCTCCTGTATTATTGCTTG |
| | GAAAATGTTACCATTTTAAATATGAAGATGAAGATAAAACGTTACC |
| | TGCAGAGTCGGGATGTACAATAGAGGATCACGTAATTGCAGGAAAT |
| | GTAGAAGAATTTCGTAAAGATTTCATTTCTAGAATATGGCTGACCT |
| | ACAGGGAAGAATTCCCTCAAATAGAAGGCTCAGCTTTGACAACAGA |
| | CTGTGGGTGGGGCTGCACATTGAGAACTGGCCAGATGCTCTTGGCT |
| | CAAGGACTCATACTACACTTTCTTGGTAGAGCTTGGACCTGGCCTG |
| | ATGCTTTGAATATTGAAAATTCAGACTCTGAATCATGGACTTCCCA |
| | CACTGTCAAAAAATTTACTGCATCATTTGAAGCATCACTTTCAGGG |
| | GAAAGAGAATTCAAAACCCCAACAATTTCTCTGAAGGAAACAATTG |
| | GGAAATATTCTGATGATCATGAAATGCGAAATGAAGTTTATCATAG |
| | GAAAATCATCTCTTGGTTTGGTGATTCCCCCTTGGCTCTTTTTGGC |
| | TTACATCAACTAATAGAATATGGAAAGAAGTCTGGGAAAAAAGCAG |
| | GAGATTGGTATGGACCAGCTGTGGTTGCTCACATTTTAAGAAAAGC |
| | AGTTGAAGAAGCAAGGCATCCTGATTTACAAGGAATAACTATTTAT |
| | GTTGCACAAGATTGTACAGTTTACAATTCTGATGTAATTGATAAAC |
| | AGAGTGCTTCCATGACTTCTGATAATGCAGATGACAAAGCTGTTAT |
| | TATTCTAGTTCCTGTTAGACTTGGTGGAGAAAGAACCAACACCGAC |
| | TACTTAGAATTTGTGAAGGGTATTTTAAGCCTGGAATATTGTGTGG |
| | GTATTATTGGTGGCAAACCTAAACAGTCATATTACTTTGCTGGATT |
| | TCAAGATGACAGTTTGATTTACATGGATCCTCATTACTGCCAATCT |
| | TTTGTAGATGTCAGCATAAAGGATTTCCCTCTTGAGACATTCCACT |
| | GCCCTTCTCCCAAAAAGATGTCTTTTCGAAAAATGGATCCCAGCTG |
| | TACAATAGGATTTTACTGTCGAAATGTTCAGGACTTCAAACGAGCT |
| | TCTGAAGAAATCACCAAGATGCTGAAATTTTCTTCTAAGGAGAAAT |
| | ATCCCTTATTTACTTTTGTAAATGGTCATTCCAGAGACTATGATTT |
| | TACATCTACTACAACCAATGAAGAAGACCTTTTTTCAGAGGATGAA |
| | AAGAAACAATTAAAAAGATTTAGCACGGAAGAGTTTGTCTTGCTTT |
| | AAAGATTAGCACATTTGTGCTTGATAAGAAGAATTCCATTGAAAGG |
| | GGAAAAATGAAGAGAAACAAGTATATCTGAAATGTTTATTTTCACA |
| | AATATCTTAATTTTATATGTTCTTTAAAAAAGAACATTTGAAAATA |
| | TAACAGTTAAAGATATTTTTCTAAAAGAGAAATGATTTAATGAATC |
| | TTGCTTTCTAATAAATAAATTGAGTGATTCTGGTTGCATTCCTATT |
| | TCCCTAAGATCTACTAGTGATAATTCTACCTTAACTGTAAGCCTTT |
| | TAGTCTTCAAAGTCTTCCACCTGAGCCCATTGTTCTCATGGAGGTT |
| | TTGTGATATTAACCCTCCCCCAAAGACTGGGATCACCAAATAGTTT |
| | CAAAATTCTCAGTTTGTACTGAAGACCAGAAGATCAGAGAAGGAAA |
| | CTTTAATGCTGTCTAGCCTCCTGCTATTAATGCAATCAAAGAATAC |
| | TTTTGCATATGTCTTGATAATTAAATAGTATTTGTTAACTGTGATA |
| | TGCATACACTTATATAAGCAGAATTATGAGTTAAAGTAATACTTAG |
| | CAATATGATTTTATAATGGCTCCTCATTATGCTTGCTGTTGAACCT |
| | TTTATGAGGAGTGAATATAAAGTATTGGTTTTCCCTCACAAATTTA |
| | AAGATTATGTTATTAATACTATTATAACTGCATCAATCAAGTCAGA |
| | TAAAGGCAACTATAAAATAGTAGTAGTGTTTGTTTCCTATCTCAAG |
| | GGCGAAATTTTATGGGAACTCAATTTATTATGCAGTTTTTAAGTTT |
| | AAAATACCAAGAAAGATGTCACTAGATTCTCTTCTATGTGATTTTT |
| | GTTTTTTATATAAAGCAGTGTAGTGGTGTTTAGAAGCTGAGGCCAC |
| | CTGTAAGGCAAATCTGCCTTAAGTGTATTATGTGTTACTTAAAGGC |
| | AAATTTGTGATCTAAAAGTACAAGAGTGATTTTTGAGCTAGGATTA |
| | TAAAATACATAATAAAGATGTGAGAAGATAAAA |
| SEQ ID NO: | GGTGGCCGGGGTGCTCAAAGTACCTGTAGCTGCGGCGCTGAGGTCG |
| 20 (Isoform-2) | GAACGTCTGCGTGTGTGCGGGCTGGTTTTGTGGCGGCTGCTGCTAG |
| | AGCTGGAGCATTTGCCGGTCAGTATAAAAGATTAAACTCTACAGAA |
| | GAATGCAATCAAGTGATGGCTTTTCCTTTAGAATTTGAATATGGAG |
| | GCTACAGGAACAGATGAAGTTGACAAGCTAAAAACCAAATTTATAT |
| | CTGCTTGGAACAACATGAAATATAGTTGGGTGTTGAAAACAAAGAC |
| | GTATTTTAGTAGAAATTCTCCTGTATTATTGCTTGGAAAATGTTAC |
| | CATTTTAAATATGAAGATGAAGATAAAACGTTACCTGCAGAGTCGG |
| | GATGTACAATAGAGGATCACGTAATTGCAGGAAATGTAGAAGAATT |
| | TCGTAAAGATTTCATTTCTAGAATATGGCTGACCTACAGGGAAGAA |
| | TTCCCTCAAATAGAAGGCTCAGCTTTGACAACAGACTGTGGGTGGG |
| | GCTGCACATTGAGAACTGGCCAGATGCTCTTGGCTCAAGGACTCAT |
| | ACTACACTTTCTTGGTAGAGCTTGGACCTGGCCTGATGCTTTGAAT |
| | ATTGAAAATTCAGACTCTGAATCATGGACTTCCCACACTGTCAAAA |
| | AATTTACTGCATCATTTGAAGCATCACTTTCAGGGGAAAGAGAATT |
| | CAAAACCCCAACAATTTCTCTGAAGGAAACAATTGGGAAATATTCT |
| | GATGATCATGAAATGCGAAATGAAGTTTATCATAGGAAAATCATCT |
| | CTTGGTTTGGTGATTCCCCCTTGGCTCTTTTTGGCTTACATCAACT |
| | AATAGAATATGGAAAGAAGTCTGGGAAAAAAGCAGGAGATTGGTAT |
| | GGACCAGCTGTGGTTGCTCACATTTTAAGAAAAGCAGTTGAAGAAG |
| | CAAGGCATCCTGATTTACAAGGAATAACTATTTATGTTGCACAAGA |
| | TTGTACAGTTTACAATTCTGATGTAATTGATAAACAGAGTGCTTCC |
| | ATGACTTCTGATAATGCAGATGACAAAGCTGTTATTATTCTAGTTC |
| | CTGTTAGACTTGGTGGAGAAAGAACCAACACCGACTACTTAGAATT |
| | TGTGAAGGGTATTTTAAGCCTGGAATATTGTGTGGGTATTATTGGT |
| | GGCAAACCTAAACAGTCATATTACTTTGCTGGATTTCAAGATGACA |
| | GTTTGATTTACATGGATCCTCATTACTGCCAATCTTTTGTAGATGT |
| | CAGCATAAAGGATTTCCCTCTTGAGATGCTGAAATTTTCTTCTAAG |
| | GAGAAATATCCCTTATTTACTTTTGTAAATGGTCATTCCAGAGACT |
| | ATGATTTTACATCTACTACAACCAATGAAGAAGACCTTTTTTCAGA |
| | GGATGAAAAGAAACAATTAAAAAGATTTAGCACGGAAGAGTTTGTC |
| | TTGCTTTAAAGATTAGCACATTTGTGCTTGATAAGAAGAATTCCAT |
| | TGAAAGGGGAAAAATGAAGAGAAACAAGTATATCTGAAATGTTTAT |
| | TTTCACAAATATCTTAATTTTATATGTTCTTTAAAAAAGAACATTT |
| | GAAAATATAACAGTTAAAGATATTTTTCTAAAAGAGAAATGATTTA |
| | ATGAATCTTGCTTTCTAATAAATAAATTGAGTGATTCTGGTTGCAT |
| | TCCTATTTCCCTAAGATCTACTAGTGATAATTCTACCTTAACTGTA |
| | AGCCTTTTAGTCTTCAAAGTCTTCCACCTGAGCCCATTGTTCTCAT |
| | GGAGGTTTTGTGATATTAACCCTCCCCCAAAGACTGGGATCACCAA |
| | ATAGTTTCAAAATTCTCAGTTTGTACTGAAGACCAGAAGATCAGAG |
| | AAGGAAACTTTAATGCTGTCTAGCCTCCTGCTATTAATGCAATCAA |
| | AGAATACTTTTGCATATGTCTTGATAATTAAATAGTATTTGTTAAC |
| | TGTGATATGCATACACTTATATAAGCAGAATTATGAGTTAAAGTAA |
| | TACTTAGCAATATGATTTTATAATGGCTCCTCATTATGCTTGCTGT |
| | TGAACCTTTTATGAGGAGTGAATATAAAGTATTGGTTTTCCCTCAC |
| | AAATTTAAAGATTATGTTATTAATACTATTATAACTGCATCAATCA |
| | AGTCAGATAAAGGCAACTATAAAATAGTAGTAGTGTTTGTTTCCTA |
| | TCTCAAGGGCGAAATTTTATGGGAACTCAATTTATTATGCAGTTTT |
| | TAAGTTTAAAATACCAAGAAAGATGTCACTAGATTCTCTTCTATGT |
| | GATTTTTGTTTTTTATATAAAGCAGTGTAGTGGTGTTTAGAAGCTG |
| | AGGCCACCTGTAAGGCAAATCTGCCTTAAGTGTATTATGTGTTACT |
| | TAAAGGCAAATTTGTGATCTAAAAGTACAAGAGTGATTTTTGAGCT |
| | AGGATTATAAAATACATAATAAAGATGTGAGAAGATAAAATGCTTT |
| | TGTTTTGGTTTTAATGTTGGGATTATTTTAATCCTTTCATTTGAAA |
| | AATCAGTGTCTCAAATGAATTCTGTTCATTTATAATAAATGCATAT |
| | ATTGCTCTGAAAA |
| SEQ ID NO: | AAAGTACCTGTAGCTGCGGCGCTGAGGTCGGAACGTCTGCGTGTGT |
| 21 (Isoform-3) | GCGGGCTGGTTTTGTGGCGGCTGCTGCTAGAGCTGGAGCATTTGCC |
| | GGGTTGGTGGCTCCTGCACATTTTTACAGTTCTCCAGTCCTTCTCT |
| | TTCGTCAGTATAAAAGATTAAACTCTACAGAAGAATGCAATCAAGT |
| | GATGGCTTTTCCTTTAGAATTTGAATATGGAGGCTACAGGAACAGA |
| | TGAAGTTGACAAGCTAAAAACCAAATTTATATCTGCTTGGAACAAC |
| | ATGAAATATAGTTGGGTGTTGAAAACAAAGACGTATTTTAGTAGAA |
| | ATTCTCCTGTATTATTGCTTGGAAAATGTTACCATTTTAAATATGA |
| | AGATGAAGATAAAACGTTACCTGCAGAGTCGGGATGTACAATAGAG |
| | GATCACGTAATTGCAGGAAATGTAGAAGAATTTCGTAAAGATTTCA |
| | TTTCTAGAATATGGCTGACCTACAGGGAAGAATTCCCTCAAATAGA |
| | AGGCTCAGCTTTGACAACAGACTGTGGGTGGGGCTGCACATTGAGA |
| | ACTGGCCAGATGCTCTTGGCTCAAGGACTCATACTACACTTTCTTG |
| | GTAGAGCTTGGACCTGGCCTGATGCTTTGAATATTGAAAATTCAGA |
| | CTCTGAATCATGGACTTCCCACACTGTCAAAAAATTTACTGCATCA |
| | TTTGAAGCATCACTTTCAGGGGAAAGAGAATTCAAAACCCCAACAA |
| | TTTCTCTGAAGGAAACAATTGGGAAATATTCTGATGATCATGAAAT |
| | GCGAAATGAAGTTTATCATAGGAAAATCATCTCTTGGTTTGGTGAT |
| | TCCCCCTTGGCTCTTTTTGGCTTACATCAACTAATAGAATATGGAA |
| | AGAAGTCTGGGAAAAAAGCAGGAGATTGGTATGGACCAGCTGTGGT |
| | TGCTCACATTTTAAGAAAAGCAGTTGAAGAAGCAAGGCATCCTGAT |
| | TTACAAGGAATAACTATTTATGTTGCACAAGATTGTACAGTTTACA |
| | ATTCTGATGTAATTGATAAACAGAGTGCTTCCATGACTTCTGATAA |
| | TGCAGATGACAAAGCTGTTATTATTCTAGTTCCTGTTAGACTTGGT |
| | GGAGAAAGAACCAACACCGACTACTTAGAATTTGTGAAGGGTATTT |
| | TAAGCCTGGAATATTGTGTGGGTATTATTGGTGGCAAACCTAAACA |
| | GTCATATTACTTTGCTGGATTTCAAGATGACAGTTTGATTTACATG |
| | GATCCTCATTACTGCCAATCTTTTGTAGATGTCAGCATAAAGGATT |
| | TCCCTCTTGAGATGCTGAAATTTTCTTCTAAGGAGAAATATCCCTT |
| | ATTTACTTTTGTAAATGGTCATTCCAGAGACTATGATTTTACATCT |
| | ACTACAACCAATGAAGAAGACCTTTTTTCAGAGGATGAAAAGAAAC |
| | AATTAAAAAGATTTAGCACGGAAGAGTTTGTCTTGCTTTAAAGATT |
| | AGCACATTTGTGCTTGATAAGAAGAATTCCATTGAAAGGGGAAAAA |
| | TGAAGAGAAACAAGTATATCTGAAATGTTTATTTTCACAAATATCT |
| | TAATTTTATATGTTCTTTAAAAAAGAACATTTGAAAATATAACAGT |
| | TAAAGATATTTTTCTAAAAGAGAAATGATTTAATGAATCTTGCTTT |
| | CTAATAAATAAATTGAGTGATTCTGGTTGCATTCCTATTTCCCTAA |
| | GATCTACTAGTGATAATTCTACCTTAACTGTAAGCCTTTTAGTCTT |
| | CAAAGTCTTCCACCTGAGCCCATTGTTCTCATGGAGGTTTTGTGAT |
| | ATTAACCCTCCCCCAAAGACTGGGATCACCAAATAGTTTCAAAATT |
| | CTCAGTTTGTACTGAAGACCAGAAGATCAGAGAAGGAAACTTTAAT |
| | GCTGTCTAGCCTCCTGCTATTAATGCAATCAAAGAATACTTTTGCA |
| | TATGTCTTGATAATTAAATAGTATTTGTTAACTGTGATATGCATAC |
| | ACTTATATAAGCAGAATTATGAGTTAAAGTAATACTTAGCAATATG |
| | ATTTTATAATGGCTCCTCATTATGCTTGCTGTTGAACCTTTTATGA |
| | GGAGTGAATATAAAGTATTGGTTTTCCCTCACAAATTTAAAGATTA |
| | TGTTATTAATACTATTATAACTGCATCAATCAAGTCAGATAAAGGC |
| | AACTATAAAATAGTAGTAGTGTTTGTTTCCTATCTCAAGGGCGAAA |
| | TTTTATGGGAACTCAATTTATTATGCAGTTTTTAAGTTTAAAATAC |
| | CAAGAAAGATGTCACTAGATTCTCTTCTATGTGATTTTTGTTTTTT |
| | ATATAAAGCAGTGTAGTGGTGTTTAGAAGCTGAGGCCACCTGTAAG |
| | GCAAATCTGCCTTAAGTGTATTATGTGTTACTTAAAGGCAAATTTG |
| | TGATCTAAAAGTACAAGAGTGATTTTTGAGCTAGGATTATAAAATA |
| | CATAATAAAGATGTGAGAAGATAAAATGCTTTTGTTTTGGTTTTAA |
| | TGTTGGGATTATTTTAATCCTTTCATTTGAAAAATCAGTGTCTCAA |
| | ATGAATTCTGTTCATTTATAATAAATGCATATATTGCTCTGAAAA |

(vi) Gm14296 and Byproducts, Degradation Products, and Precursors Thereof

Gm14296 is a peptide with the sequence shown as SEQ NOs: 22-23, encoded by SEQ ID NO: 24-25, respectively (Table 6). Gm14296 also comprises the human orthologous sequences to SEQ ID NOs: 22-25. Gm14296 has broad expression in cortex and cerebellum.

As described in Example 6, Gm14296 has been identified as a biomarker of Alzheimer's disease. Some embodiments of any of the methods described herein can include the detection of a level of Gm14296. In some embodiments of any of the methods described herein, a biomarker can be Gm14296, a byproduct, degradation product, or fragment thereof, or a precursor thereof, for example, genomic DNA, mRNA, or protein. In some embodiments of any of the methods described herein, a biomarker can be a polypeptide with at least 80% sequence identity (e.g., at least 85%, 90%, 95%, 98%, or 99% sequence identity) to any one of SEQ ID NOs: 22-25. In some embodiments of any of the methods described herein, a biomarker can be a nucleic acid encoding a polypeptide with at least 80% sequence identity (e.g., at least 85%, 90%, 95%, 98% or 99% sequence identity) to any one of SEQ ID NOs: 22-23. In some embodiments of any of the methods described herein, a biomarker can be a nucleic acid with at least 80% sequence identity (e.g., at least 85%, 90%, 95%, 98% or 99% sequence identity) to SEQ ID NOs: 24-25.

TABLE 6

| | |
|---|---|
| SEQ ID NO: | MDLVTYDDVHVNFTQEEWALLDPSQKSLYKGVMLETYRNLTAIGY |
| 22 (Isoform-1) | IWEEHTIEDHFQTSRSHGRHERSCTAEQPSEFIQCGKAFAYESHS |
| | QMHQIKHTGEKHYDCNQCGKAFKRRSDLQIHKRTHTGEKPYECK |
| SEQ ID NO: | MDLVTYDDVHVNFTQEEWALLDPSQKSLYKGVMLETYRNLTAIGY |
| 23 (Isoform-2) | IWEEHTIEDHFQTSRSHGRHERSCTAEQPSEFIQCGKAFAYESHS |
| | QMHQIKHTGEKHYDCNQCGKAFKRRSDLQIHKRTHTGEKPYECK |
| SEQ ID NO: | GGTCAAGCTTGCAAGCTCCTTTCTGTAACTCAGCAGTCATGGGAG |
| 24 (Isoform-1) | TTTCTGTGTGATGTGTTCTGCGTGACTCTACTGGGTGTTGTGAGA |
| | GGAGGTTAGTCAAGCTCAGAACTCCAAACATATGTCTTCGCTGGA |
| | TGCTGTGAATTTAGCTCAAGCAAGCTCAGAACTTCACACATGGAT |
| | TTAGTCACCTATGATGACGTGCATGTGAACTTCACTCAGGAAGAG |
| | TGGGCTTTGCTGGATCCTTCTCAGAAGAGTCTCTATAAAGGTGTG |
| | ATGCTAGAGACCTATAGGAATCTCACAGCTATAGGTTACATTTGG |
| | GAAGAACATACAATTGAAGACCATTTCCAAACTTCTAGAAGTCAT |
| | GGAAGGCATGAAAGAAGTTGTACTGCAGAGCAACCCTCTGAGTTT |
| | ATTCAATGTGGTAAAGCCTTTGCATATGAGAGTCATAGTCAAATG |
| | CATCAAATTAAACATACTGGAGAGAAACACTATGACTGTAACCAA |
| | TGTGGTAAAGCTTTTAAAAGAAGGAGTGACCTCCAAATACATAAG |
| | CGAACACATACAGGAGAGAAACCCTATGAATGTAAATGATGTGGT |
| | AAAGCCTTTGCAAGAAGCAGTCATCTCCGAATACATAAGCGAAC |
| | ACATACAGGAGAGAAACCCTATGAATGTAAACGATGTGGTAAAG |
| | CCTTTGCAAGAAGTGATAACCTCCAACAACATAAACGAACACATA |
| | CAGGAGAGAAACCCTATGAATGTAAACAATGTGGTAAAGCCTTTT |
| | CACAAAGCAGTCATCTCCGAATACATAAGCGAATACATACAGGA |
| | GAGAAACCCTATGAATGTAACCAATGTGGAAAAGCCTTTACAAG |
| | AAGTGGTGACCTCCAAAAACATAAACGAACACATACAGGAGAGA |
| | AACCCTATAAATGTAAACAATGTGGTAAAGCCTTTGCACAAAGCA |
| | GTAATCTCCGATTACATAAGCAAACACATACAGGAGAGAGACCC |
| | TATCAATGTAACTAGCTTTGCAGGAAGCAGGGGTCTCCAATGCCA |
| | TAAAAGATCACATACTGGAGAGACACTATATGAATGTAACCAAG |
| | GTGGTAAACCCTTTGCAGGAAGGAATGACCTTTAAAGAGAAAAG |
| | ACCATACAGGAGAGAAACTCTATGAAGGTATCTAATGTTATAAAG |
| | CCTTTGCAGCAAGCATTGATCTCCAAGTACATAAATGAACACATA |
| | CCAGAGAGGAACCTTTTAAATGAAACCAATGTGGTAAAACTTTTT |
| | CGAAATCCAATAGTCTTTACACACATAAAAGCACATATAGTTGAG |
| | AGAAATGCTTTGATTTTAACCAATGTGGTTAAACTTTTTTCAAAG |
| | GGCAGTCATCAACAAAATCATAAAAGAACACATATAGGAGAGAGA |
| | TTTTTGAATGTTAGCAATGGTGCTTTTACACCTTTTTCATGAAGC |
| | AGTGGCCTCCAAATATATAAAGGAACACATACAAGAGAGAAACCC |
| | TATGAATATTGGCAATGTTTTAAAGCACTTCTACAGCACAATCAT |
| | CAAATCCATAAAGAACATGAGGTGGAGAGAAATCCTCTGAACAA |
| | CACTAATTAGGGAAAGCAATTGTACAAAAAAGTCATCTCCCAACA |
| | CAAAAGTATACACACTGGATAGAAACCCTATGAATATAGGCAGT |
| | GTGGTAAATATTTTTTAAGTCACAGTAGTTCCTAAGTAAATAAAA |
| | GGAGACACAGTTGAGAGAAACCCTATGAAAGTTATCAAATGAAC |
| | AAAGCCTTGAAAGTTAAAATCTTCAAATGCATAAAAGAAGAAAT |
| | AGAGGTGAGAAACCCTATGTTTGCAAGCCATGTGGTATTGCTTTC |
| | ATTTGTCAGTCATATTCAAAGACACAAAAGAAAACATAATGTAGA |
| | CAAAATGTTGAGTGTATTTAATATGGTGAAGATTTTTGAATATTT |
| | CTAGAGTCTTCACAATCAAACAATCCATACTGCAAAGAAATTTAG |
| | AAATATAATATGGTGAAACCTTTTCAGATCCCAAATCTTCATCAT |
| | TATGAAAAAGAAACCTATGAATGCATTCAATATGGGGAAGCCTTT |
| | AAACATTTCTAATTCTTCATCATCATGAAAATAATCATACAAAGG |
| | AGATCAAGGGGATACAAGGCATGATTCTTATTGTCTTGCTTGAGT |
| | TACAGTCCTGACATCCTTTGGTGATGAATATCAGTGTGGAAAGTG |
| | TGAGCTGGATTAAACTCTTTCCTCCCCAACTGCTTCTTCGTCATG |
| | TTTATGCAGGAATAGAAACCCTGACTAAGACAAATTGGTATGAGG |
| | ATTGGGGTATTCCAGTGACAACCTGACCATGTTTTGGGAGGACTA |
| | AAAGGACTTTGGAACTTTGGGCTAGAAGATCCATTCTGTGTTAAG |
| | AACTCTGTGGGATGTTGTGTAGGAGCTTGGAAGATAATGTTGAGA |
| | ACAGTGGAGAAGATGAAGGCCTCACTTTTGAAATTTCACAGGAAA |
| | ATTAAAGACTCTTTTGAGGTCCATTGCTGTTTTGGATTGTGAAGA |
| | TTCTGTGGTTCTGTGTAGCTAGGGCTGAAGAATCAGCTGTGATGA |
| | ACAAGATACCAGAACTTCTAGAGTGAAAACTTTGCATTACTGGGA |
| | CTATTGATGCTGGTTAGCTGGAGCTAAGAAATTAGCAGTGATTAA |
| | GAA |
| | GAGACCAGCATCATTGAGGTGGCATCTTCTGGGAAGTGTTTTCT |
| | GAGAGCACAGAGGCAGTGTTCTAGAGATAGCCAAGGTTGTACCT |
| | TGTGCTGTGCCTGGACTTGGTAATGTGTTAGAGTTACCCACATG |
| | GTACTGGTTTTGAAGGCCTGAAGGGATAATGTAGAGCAGCTGAT |
| | GCCCTGCCCTGTGAGAGGCCATGGAAGGCCATTGGTGAAGGTGC |
| | AAACTCAGGTGAAATTGATGTCCCAGTACTGAAGAGATCATGCA |
| | GTGTTTTGGAGTTGCTAGGTCCATGAATGACCAAGAACAGCAGC |
| | AACAGTAAAGTACAGGCATCTGGAGCCTAGAGGACAAGCTGTGT |
| | GCTACAAAGGACAGGGCTGGAGAAGTGACCCAAGCCCTTGGAGG |
| | AGCCCAATAAATGGTGATTTTGAACCCAGACATTGGACAGTTGG |
| | AGTTTAATTTTTGCTTTTGATTGTGACTGTACACTAATATTTTT |
| | CCCTCTTGAAGGGAGAAAATATTTTAGTGGATTCCACAGTTAAG |
| | AGACTTTTAGTTGTAAAAGGCTTTGGATTTTAAAGGATATTGGA |
| | TATTTTAAAAGGATTGAACTTTTACTATGTAAAGACTGTGGGAC |
| | TTTCAAAGTTTCTTAGAACTTAGGGATGAATAAGAAACTAAGGG |
| | TTAAGGCTTAATTGTGATGTGCTTGTGTGTCAAGTTGACAAGGG |
| | GTCAATTGTACTGGCTAGTTTTCTGTCAACTTGACACAAGCTGG |
| | AGTTATCACAGAGAAAGGAGCTTCATTGGGGGAAATGTCTCCAT |
| | GAGATCCATCTGTAAGACATTTTCTCAATTTGTGATCAAGTGGA |
| | AAGGCCCCTTGTGGGTGGGACCATCTCTCTGGGCTGGTAGTCTT |
| | GGGTTCTATAAGAGAGCAGTCTGAGAAAGCCAGGTGAAGCAAAC |
| | CAGTAAAGAAAGATCCCTCCATGGCCTCTGCATCAGCTCCTGCT |
| | TCTTGACCTGCTTGAGTTCCAGTCCTGACTTCCTTGGTGATGAA |
| | CAGCAAGCAGTATGAAAGTATAAACTGAACAAACCCTTTCCTCC |
| | CCAACTTGCTTCTTGGTCATGTTGTTTGTGCACGAATAGAAACC |
| | CTGAATAATACAAGGGGTATAAGGTGTGGATTGTAAAAAAAGAG |
| | ACTAGACAATAAAGAAATGAAGAAATAAAATTATTTTAAAAAAA |
| | A |
| SEQ ID NO: | GGTCAAGCTTGCAAGCTCCTTTCTGTAACTCAGCAGTCATGGGA |
| 25 (Isoform-2) | GTTTCTGTGTGATGTGTTCTGCGTGACTCTACTGGGTGTTGTGA |
| | GAGGAGGTTAGTCAAGCTCAGAACTCCAAACATATGTCTTCGCT |
| | GGATGCTGTGAATTTAGCTCAAGCAAGCTCAGAACTTCACACAT |
| | GGATTTAGTCACCTATGATGACGTGCATGTGAACTTCACTCAGGA |
| | AGAGTGGGCTTTGCTGGATCCTTCTCAGAAGAGTCTCTATAAAGG |
| | TGTGATGCTAGAGACCTATAGGAATCTCACAGCTATAGGTTACAT |
| | TTGGGAAGAACATACAATTGAAGACCATTTCCAAACTTCTAGAAG |
| | TCATGGAAGGCATGAAAGAAGTTGTACTGCAGAGCAACCCTCTGA |
| | GTTTATTCAATGTGGTAAAGCCTTTGCATATGAGAGTCATAGTCA |
| | AATGCATCAAATTAAACATACTGGAGAGAAACACTATGACTGTAA |
| | CCAATGTGGTAAAGCTTTTAAAAGAAGGAGTGACCTCCAAATACA |
| | TAAGCGAACACATACAGGAGAGAAACCCTATGAATGTAAATGATG |
| | TGGTAAAGCCTTTGCAAGAAGCAGTCATCTCCGAATACATAAGCG |
| | AACACATACAGGAGAGAAACCCTATGAATGTAAACGATGTGGTAA |
| | AGCCTTTGCAAGAAGTGATAACCTCCAACAACATAAACGAACACA |
| | TACAGGAGAGAAACCCTATGAATGTAAACAATGTGGTAAAGCCTT |
| | TTCACAAAGCAGTCATCTCCGAATACATAAGCGAATACATACAGG |
| | AGAGAAACCCTATGAATGTAACCAATGTGGAAAAGCCTTTACAAG |
| | AAGTGGTGACCTCCAAAAACATAAACGAACACATACAGGAGAGAA |
| | ACCCTATAAATGTAAACAATGTGGTAAAGCCTTTGCACAAAGCAG |
| | TAATCTCCGATTACATAAGCAAACACATACAGGAGAGAGACCCTA |
| | TCAATGTAACTAGCTTTGCAGGAAGCAGGGGTCTCCAATGCCATA |
| | AAAGATCACATACTGGAGAGACACTATATGAATGTAACCAAGGTG |
| | GTAAACCCTTTGCAGGAAGGAATGACCTTTAAAGAGAAAAGACCA |
| | TACAGGAGAGAAACTCTATGAAGGTATCTAATGTTATAAAGCCTT |
| | TGCAGCAAGCATTGATCTCCAAGTACATAAATGAACACATACCAG |
| | AGAGGAACCTTTTAAATGAAACCAATGTGGTAAAACTTTTTCGAA |
| | ATCCAATAGTCTTTACACACATAAAAGCACATATAGTTGAGAGAA |
| | ATGCTTTGATTTTAACCAATGTGGTTAAACTTTTTTCAAAGGGCA |
| | GTCATCAACAAAATCATAAAAGAACACATATAGGAGAGAGATTTT |
| | TGAATGTTAGCAATGGTGCTTTTACACCTTTTTCATGAAGCAGTG |
| | GCCTCCAAATATATAAAGGAACACATACAAGAGAGAAACCCTATG |
| | AATATTGGCAATGTTTTAAAGCACTTCTACAGCACAATCATCAAA |
| | TCCATAAAGAACATGAGGTGGAGAGAAATCCTCTGAACAACACTA |
| | ATTAGGGAAAGCAATTGTACAAAAAAGTCATCTCCCAACACAAAA |
| | GTATACACACTGGATAGAAACCCTATGAATATAGGCAGTGTGGTA |
| | AATATTTTTTAAGTCACAGTAGTTCCTAAGTAAATAAAAGGAGAC |
| | ACAGTTGAGAGAAACCCTATGAAAGTTATCAAATGAACAAAGCCT |
| | TGAAAGTTAAAATCTTCAAATGCATAAAAGAAGAAATAGAGGTGA |
| | GAAACCCTATGTTTGCAAGCCATGTGGTATTGCTTTCATTTGTCA |
| | GTCATATTCAAAGACACAAAAGAAAACATAATGTAGACAAAATGT |
| | TGAGTGTATTTAATATGGTGAAGATTTTTGAATATTTCTAGAGTC |
| | TTCACAATCAAACAATCCATACTGCAAAGAAATTTAGAAATATAA |
| | TATGGTGAAACCTTTTCAGATCCCAAATCTTCATCATTATGAAAA |
| | AGAAACCTATGAATGCATTCAATATGGGGAAGCCTTTAAACATTT |
| | CTAATTCTTCATCATCATGAAAATAATCATACAAAGGAGATCAAG |
| | GGGATACAAGGCATGATTCTTATTGTCTTGCTTGAGTTACAGTCC |
| | TGACATCCTTTGGTGATGAATATCAGTGTGGAAAGTGTGAGCTGG |
| | ATTAAACTCTTTCCTCCCCAACTGCTTCTTCGTCATGTTTATGCA |
| | GGAATAGAAACCCTGACTAAGACAAATTGGTATGAGGATTGGGGT |
| | ATTCCAGTGACAACCTGACCATGTTTTGGGAGGACTAAAAGGACT |
| | TTGGAACTTTGGGCTAGAAGATCCATTCTGTGTTAAGAACTCTGT |
| | GGGATGTTGTGTAGGAGCTTGGAAGATAATGTTGAGAACAGTGGA |
| | GAAGATGAAGGCCTCACTTTTGAAATTTCACAGGAAAATTAAAGA |
| | CTCTTTTGAGGTCCATTGCTGTTTTGGATTGTGAAGATTCTGTGG |
| | TTCTGTGTAGCTAGGGCTGAAGAATCAGCTGTGATGAACAAGATA |
| | CCAGAACTTCTAGAGTGAAAACTTTGCATTACTGGGACTATTGAT |
| | GCTGGTTAGCTGGAGCTAAGAAATTAGCAGTGATTAAGAAGAGAC |
| | CAGCATCATTGAGGTGGCATCTTCTGGGAAGTGTTTTCTGAGAGC |
| | ACAGAGGCAGTGTTCTAGAGATAGCCAAGGTTGTACCTTGTGCTG |
| | TGCCTGGACTTGGTAATGTGTTAGAGTTACCCACATGGTACTGGT |
| | TTTGAAGGCCTGAAGGGATAATGTAGAGCAGCTGATGCCCTGCCC |
| | TGTGAGAGGCCATGGAAGGCCATTGGTGAAGGTGCAAACTCAGGT |
| | GAAATTGATGTCCCAGTACTGAAGAGATCATGCAGTGTTTTGGAG |
| | TTGCTAGGTCCATGAATGACCAAGAACAGCAGCAACAGTAAAGTA |
| | CAGGCATCTGGAGCCTAGAGGACAAGCTGTGTGCTACAAAGGACA |
| | GGGCTGGAGAAGTGACCCAAGCCCTTGGAGGAGCCCAATAAATGG |
| | TGATTTTGAACCCAGACATTGGACAGTTGGAGTTTAATTTTTGCT |
| | TTTGATTGTGACTGTACACTAATATTTTTCCCTCTTGAAGGGAGA |
| | AAATATTTTAGTGGATTCCACAGTTAAGAGACTTTTAGTTGTAAA |
| | AGGCTTTGGATTTTAAAGGATATTGGATATTTTAAAAGGATTGAA |
| | CTTTTACTATGTAAAGACTGTGGGACTTTCAAAGTTTCTTAGAAC |
| | TTAGGGATGAATAAGAAACTAAGGGTTAAGGCTTAATTGTGATGT |
| | GCTTGTGTGTCAAGTTGACAAGGGGTCAATTGTACTGGCTAGTTT |
| | TCTGTCAACTTGACACAAGCTGGAGTTATCACAGAGAAAGGAGCT |
| | TCATTGGGGGAAATGTCTCCATGAGATCCATCTGTAAGACATTTT |
| | CTCAATTTGTGATCAAGTGGAAAGGCCCCTTGTGGGTGGGACCAT |
| | CTCTCTGGGCTGGTAGTCTTGGGTTCTATAAGAGAGCAGTCTGAG |
| | AAAGCCAGGTGAAGCAAACCAGTAAAGAAAGATCCCTCCATGGCC |
| | TCTGCATCAGCTCCTGCTTCTTGACCTGCTTGAGTTCCAGTCCTG |
| | ACTTCCTTGGTGATGAACAGCAAGCAGTATGAAAGTATAAACTGA |
| | ACAAACCCTTTCCTCCCCAACTTGCTTCTTGGTCATGTTGTTTGT |
| | GCACGAATAGAAACCCTGAATAATACAAGGGGTATAAGGTGTGGA |
| | TTGTAAAAAAAGAGACTAGACAATAAAGAAATGAAGAAATAAAAT |
| | TATTTTAAAAAAAA |

(vii) Hypocretin Neuropeptide Precursor (Hcrt) and Byproducts, Degradation Products, and Precursors Thereof

Hypocretin neuropeptide precursor (Hcrt) is a peptide with the sequence shown as SEQ NOs: 26, encoded by SEQ ID NO: 27, respectively (Table 7). Hcrt encodes a hypothalamic neuropeptide precursor protein that gives rise to two mature neuropeptides, orexin A and orexin B, by proteolytic processing. Orexin A and orexin B, which bind to orphan G-protein coupled receptors HCRTR1 and HCRTR2, function in the regulation of sleep and arousal. This neuropeptide arrangement may also play a role in feeding behavior, metabolism, and homeostasis.

As described in Example 6, Hcrt has been identified as a biomarker of Alzheimer's disease. Some embodiments of any of the methods described herein can include the detection of a level of Hcrt. In some embodiments of any of the methods described herein, a biomarker can be Hcrt, a byproduct, degradation product, or fragment thereof, or a precursor thereof, for example, genomic DNA, mRNA, or protein. In some embodiments of any of the methods described herein, a biomarker can be a polypeptide with at least 80% sequence identity (e.g., at least 85%, 90%, 95%, 98%, or 99% sequence identity) to any one of SEQ ID NOs: 26-27. In some embodiments of any of the methods described herein, a biomarker can be a nucleic acid encoding a polypeptide with at least 80% sequence identity (e.g., at least 85%, 90%, 95%, 98% or 99% sequence identity) to any one of SEQ ID NO: 26. In some embodiments of any of the methods described herein, a biomarker can be a nucleic acid with at least 80% sequence identity (e.g., at least 85%, 90%, 95%, 98% or 99% sequence identity) to SEQ ID NO: 27.

TABLE 7

| | |
|---|---|
| SEQ ID | MNLPSTKVSWAAVTLLLLLLLLPPALLSSGAAAQPLPDC |
| NO: 26 | CRQKTCSCRLYELLHGAGNHAAGILTLGKRRSGPPGLQG |
| | RLQRLLQASGNHAAGILTMGRRAGAEPAPRPCLGRRCSA |
| | PAAASVAPGGQSGI |
| SEQ ID | CACAATTGACAGCCTCAAGGTTCCTGGCTTTTTGAACCA |
| NO: 27 | CCACAGACATCTCCTTTCCCGGCTACCCCACCCTGAGCG |
| | CCAGACACCATGAACCTTCCTTCCACAAAGGTCTCCTGG |
| | GCCGCCGTGACGCTACTGCTGCTGCTGCTGCTGCTGCCG |
| | CCCGCGCTGTTGTCGTCCGGGGCGGCTGCACAGCCCCTG |
| | CCCGACTGCTGTCGTCAAAAGACTTGCTCTTGCCGCCTC |
| | TACGAGCTGCTGCACGGCGCGGGCAATCACGCGGCCGGC |
| | ATCCTCACGCTGGGCAAGCGGAGGTCCGGGCCCCCGGGC |
| | CTCCAGGGTCGGCTGCAGCGCCTCCTGCAGGCCAGCGGC |
| | AACCACGCCGCGGGCATCCTGACCATGGGCCGCCGCGCA |
| | GGCGCAGAGCCAGCGCCGCGCCCCTGCCTCGGGCGCCGC |
| | TGTTCCGCCCCGGCCGCCGCCTCCGTCGCGCCCGGAGGA |
| | CAGTCCGGGATCTGAGTCGTTCTTCGGGCCCTGTCCTGG |
| | CCCAGGCCTCTGCCCTCTGCCCACCCAGCGTCAGCCCCC |
| | AGAAAAAAGGCAATAAAGACGAGTCTCCATT |

(viii) Transthyretin (Ttr) and Byproducts, Degradation Products, and Precursors Thereof

Transthyretin (Ttr) is a peptide with the sequence shown as SEQ NOs: 28, encoded by SEQ ID NO: 29, respectively (Table 8).

Ttr encodes one of the three prealbumins, which include alpha-1-antitrypsin, transthyretin and orosomucoid. Ttr is a homo-tetrameric carrier protein, which transports thyroid hormones in the plasma and cerebrospinal fluid. It is also involved in the transport of retinol (vitamin A) in the plasma by associating with retinol-binding protein. Ttr may also be involved in other intracellular processes including proteolysis, nerve regeneration, autophagy, and glucose homeostasis. Mutations in Ttr are associated with amyloid deposition, predominantly affecting peripheral nerves or the heart, while a small percentage of the gene mutations are non-amyloidogenic. The mutations are implicated in the etiology of several diseases, including amyloidotic polyneuropathy, euthyroid hyperthyroxinaemia, amyloidotic vitreous opacities, cardiomyopathy, oculoleptomeningeal amyloidosis, meningocerebrovascular amyloidosis and carpal tunnel syndrome.

As described in Example 6, Ttr has been identified as a biomarker of Alzheimer's disease. Some embodiments of any of the methods described herein can include the detection of a level of Ttr. In some embodiments of any of the methods described herein, a biomarker can be Ttr, a byproduct, degradation product, or fragment thereof, or a precursor thereof, for example, genomic DNA, mRNA, or protein. In some embodiments of any of the methods described herein, a biomarker can be a polypeptide with at least 80% sequence identity (e.g., at least 85%, 90%, 95%, 98%, or 99% sequence identity) to any one of SEQ ID NOs: 28-29. In some embodiments of any of the methods described herein, a biomarker can be a nucleic acid encoding a polypeptide with at least 80% sequence identity (e.g., at least 85%, 90%, 95%, 98% or 99% sequence identity) to any one of SEQ ID NO: 28. In some embodiments of any of the methods described herein, a biomarker can be a nucleic acid with at least 80% sequence identity (e.g., at least 85%, 90%, 95%, 98% or 99% sequence identity) to SEQ ID NO: 29.

TABLE 8

| | |
|---|---|
| SEQ ID | MASHRLLLLCLAGLVFVSEAGPTGTGESKCPLMVKVLDA |
| NO: 28 | VRGSPAINVAVHVFRKAADDTWEPFASGKTSESGELHGL |
| | TTEEEFVEGIYKVEIDTKSYWKALGISPFHEHAEVVFTA |
| | NDSGPRRYTIAALLSPYSYSTTAVVTNPKE |
| SEQ ID | GTTGACTAAGTCAATAATCAGAATCAGCAGGTTTGCAGT |
| NO: 29 | CAGATTGGCAGGGATAAGCAGCCTAGCTCAGGAGAAGTG |
| | AGTATAAAAGCCCCAGGCTGGGAGCAGCCATCACAGAAG |
| | TCCACTCATTCTTGGCAGGATGGCTTCTCATCGTCTGCT |
| | CCTCCTCTGCCTTGCTGGACTGGTATTTGTGTCTGAGGC |
| | TGGCCCTACGGGCACCGGTGAATCCAAGTGTCCTCTGAT |
| | GGTCAAAGTTCTAGATGCTGTCCGAGGCAGTCCTGCCAT |
| | CAATGTGGCCGTGCATGTGTTCAGAAAGGCTGCTGATGA |
| | CACCTGGGAGCCATTTGCCTCTGGGAAAACCAGTGAGTC |
| | TGGAGAGCTGCATGGGCTCACAACTGAGGAGGAATTTGT |
| | AGAAGGGATATACAAAGTGGAAATAGACACCAAATCTTA |
| | CTGGAAGGCACTTGGCATCTCCCCATTCCATGAGCATGC |
| | AGAGGTGGTATTCACAGCCAACGACTCCGGCCCCCGCCG |
| | CTACACCATTGCCGCCCTGCTGAGCCCCTACTCCTATTC |
| | CACCACGGCTGTCGTCACCAATCCCAAGGAATGAGGGAC |
| | TTCTCCTCCAGTGGACCTGAAGGACGAGGGATGGGATTT |
| | CATGTAACCAAGAGTATTCCATTTTTACTAAAGCAGTGT |
| | TTTCACCTCATATGCTATGTTAGAAGTCCAGGCAGAGAC |
| | AATAAAACATTCCTGTGAAAGGCACTTTTCATTCCACTT |
| | TAACTTGATTTTTTAAATTCCCTTATTGTCCCTTCCAAA |
| | AAAAAGAGAATCAAAATTTTACAAAGAATCAAAGGAATT |
| | CTAGAAAGTATCTGGGCAGAACGCTAGGAGAGATCCAAA |
| | TTTCCATTGTCTTGCAAGCAAAGCACGTATTAAATATGA |
| | TCTGCAGCCATTAAAAAGACACATTCTGTAAAAAAAAAA |
| | AA |

(j) Methods of (i) Diagnosing Alzheimer's Disease, (ii) Identifying Increased Likelihood of Developing Alzheimer's Disease, (iii) Monitoring the Progression of Alzheimer's Disease, and (iv) Determining the Efficacy of a Treatment for Alzheimer's Disease in a Subject

Provided herein are methods of diagnosing a subject as having Alzheimer's disease. Also provided herein are methods of identifying a subject as having an increased likelihood of developing Alzheimer's disease. Further provided herein are methods of monitoring the progression of Alzheimer's Disease. Also provided herein are methods for determining the efficacy of a treatment for Alzheimer's Disease in a subject.

In any of these methods, a biological sample can be any appropriate biological sample obtained from the subject. In some embodiments, a biological sample can be a sample comprising cerebrospinal fluid. In some embodiments, a biological sample can be a sample comprising blood, serum, or plasma. In some embodiments, a biological sample can be a cell culture sample. In some embodiments, the cell culture sample is derived from a cell obtained from the subject. In some embodiments, the method can further include obtaining the sample from the subject. In some embodiments, the method can further include obtaining first and second biological samples from the subject.

(i) Methods of Diagnosing Alzheimer's Disease

In some embodiments, the methods can include (a) determining an elevated level of one or more diagnostic biomarkers described herein (e.g., one or more of PMCH, Akr1e1, Atg4c, Gm14296, Hcrt, and Ttr, or a byproduct or precursor or degradation product or fragment thereof), in a biological sample from a subject; and (b) identifying a subject having an elevated level the one or more diagnostic biomarkers in the biological sample as compared to a reference level of the one or more diagnostic biomarkers, as having Alzheimer's disease. In some embodiments, the methods can include (a) determining an decreased level of one or more diagnostic biomarkers described herein (e.g., one or both of Adora2a and Trh, or a byproduct or precursor or degradation product or fragment thereof), in a biological sample from a subject; and (b) identifying a subject having an decreased level the one or more diagnostic biomarkers in the biological sample as compared to a reference level of the one or more diagnostic biomarkers, as having Alzheimer's disease.

In some embodiments wherein the subject has been diagnosed with Alzheimer's disease, the methods can further include confirming a diagnosis of Alzheimer's disease in the subject. Non-limiting examples of ways to confirm a diagnosis of Alzheimer's disease include obtaining an image of the subject's brain (e.g., a CT, MRI, or PET scan), detecting a genetic mutation associated with Alzheimer's disease, determining the levels of other biomarkers of Alzheimer's disease, or performing cognitive testing on the subject (e.g., neuropsychological/memory testing).

Non-limiting examples of confirmatory tests can include detecting hallmarks of amyloid beta (Aβ) deposition, such as low cerebrospinal fluid Aβ42 and/or uptake of amyloid PET tracers and/or hallmarks of neurodegeneration, such as elevation of total tau (t-tau) and/or phosphorylated tau (p-tau) in cerebrospinal fluid, hypometabolism on a fluorodeoxyglucose (FDG) PET scan, and/or atrophy (e.g., hippocampal atrophy) on structural MRI.

Non-limiting examples of genetic tests that can be used to confirm a diagnosis of Alzheimer's disease include determination of the apolipoprotein E allele (APOE) in the subject, where the APOE e4 allele is associated with an increased risk of Alzheimer's disease; or mutations in APP, PSEN1, or PSEN2, which can be associated with early-onset Alzheimer's disease. Other methods of confirming a diagnosis of Alzheimer's disease will be apparent to one skilled in the field. In some embodiments, the methods can further include selecting a treatment for the subject. In some embodiments, the methods can further include administering a treatment of Alzheimer's disease to the subject. In some embodiments, a treatment of Alzheimer's disease can be a treatment that reduces the rate of progression of Alzheimer's disease or decreases the likelihood of developing Alzheimer's disease. In some embodiments, a method of a treatment or prophylaxis for Alzheimer's disease can include, but is not limited to treatment with cholinesterase inhibitor, an N-methyl-D-aspartate (NMDA) inhibitor, an antipsychotic, a tricyclic antidepressant, a benzodiazepine, insulin, and tacrine hydrochloride. In some embodiments, a treatment of Alzheimer's disease can include a cholinesterase inhibitor, such as galantamine, rivastigmine, or donepezil. In some embodiments, a treatment of Alzheimer's disease can include an N-methyl D-aspartate (NMDA) inhibitor, such as memantine. In some embodiments, a treatment of Alzheimer's disease can include a supportive treatment, such as a sleep aid, a benzodiazepine, an anti-anxiety medication, an anti-depressant, an anticonvulsant, an antipsychotic, or a combination thereof. In some embodiments, the antipsychotic agent used in the treatment of Alzheimer's disease is aripiprazole, risperidone, olanzapine, quetiapine, or haloperidol; the benzodiazepine is lorazepam, oxazepam or temazepam; and the tricyclic antidepressant is nortriptyline. Nonlimiting examples of agents that may be used to treat Alzheimer's Disease include but are not limited to and Aricept®, Exelon®, Razadyne®, Namenda®.

In some embodiments, the methods can further include updating the subject's clinical record with the diagnosis of Alzheimer's disease. In some embodiments, the methods can further include enrolling the subject in a clinical trial. In some embodiments, the methods can further include informing the subject's family of the diagnosis. In some embodiments, the methods can further include assessing or referring the subject for enrollment in a supportive care plan or care facility. In some embodiments, the methods can further include monitoring the subject more frequently.

(ii) Methods of Identifying Increased Likelihood of Developing Alzheimer's Disease

In some embodiments wherein the subject has been identified as having increased likelihood of developing Alzheimer's disease, the methods can further comprise monitoring the identified subject for the development of symptoms of Alzheimer's disease. In some embodiments, the methods can further include recording in the identified subject's clinical record that the subject has an increased likelihood of developing Alzheimer's disease. In some embodiments, the methods can further include notifying the subject's family that the subject has an increased likelihood or susceptibility of developing Alzheimer's disease. In some embodiments, the subject can be tested for the presence of genetic mutations known to be associated with risk for Alzheimer's disease. In some embodiments, the subject can be advised to avoid behavioral risk factors for Alzheimer's disease.

In some embodiments, the methods can further include enrolling the subject in a clinical trial (e.g., for the early treatment and/or prevention of Alzheimer's disease). In some embodiments, the methods can further include informing the subject's family of the subject's likelihood of developing Alzheimer's disease. In some embodiments, the methods can further include monitoring the subject more frequently.

In some embodiments, the methods can further include performing one or more tests to further determine the subject's risk of developing Alzheimer's disease. Non-limiting examples of more tests to further determine the subject's risk of developing Alzheimer's disease include taking a family history (e.g., where a family history of Alzheimer's disease is indicative of an increased risk of developing Alzheimer's disease), detecting a genetic mutation associated with Alzheimer's disease, determining the levels of other biomarkers (e.g., in cerebrospinal fluid or in blood or a component thereof) indicative an increased risk of developing Alzheimer's disease, and taking a health history (e.g., where a history of head injury and/or heart conditions are indicative of an increased risk of developing Alzheimer's disease, or the subject's behavior indicate an increased risk of developing Alzheimer's disease).

Non-limiting examples of genetic tests include determination of the apolipoprotein E (APOE) allele in the subject, where the APOE e4 allele is associated with an increased risk of Alzheimer's disease; or mutations in APP, PSEN1, or PSEN2, which can be associated with early-onset Alzheimer's disease.

In some embodiments, the methods can further include administering a treatment of Alzheimer's disease to the subject. Exemplary treatments are described herein.

(iii) Methods of Monitoring the Progression of Alzheimer's Disease

In some embodiments, the methods described in this section can be used to monitor progression of Alzheimer's disease in a subject over time. Accordingly, in some embodiments, the methods can include (a) determining a first level of one or more diagnostic biomarkers described herein (e.g., one or more of PMCH, Akr1e1, Atg4c, Gm14296, Hcrt, and Ttr, or a byproduct or precursor or degradation product or fragment thereof) in a first biological sample obtained from a subject at a first time point; (b) determining a second level of the one or more diagnostic biomarkers described herein (e.g., one or more of PMCH, Akr1e1, Atg4c, Gm14296, Hcrt, and Ttr, or a byproduct or precursor or degradation product or fragment thereof) in a second biological sample obtained from the subject at a second time point; (c) identifying: (i) a subject having an increased second level of the one or more diagnostic biomarkers described herein (e.g., one or more of PMCH, Akr1e1, Atg4c, Gm14296, Hcrt, and Ttr, or a byproduct or precursor or degradation product or fragment thereof) as compared to the first level of the one or more diagnostic biomarkers described herein (e.g., one or more of PMCH, Akr1e1, Atg4c, Gm14296, Hcrt, and Ttr, or a byproduct or precursor or degradation product or fragment thereof), as having progressing Alzheimer's disease, or (ii) a subject having about the same or a decreased second level of the one or more diagnostic biomarkers described herein (e.g., one or more of PMCH, Akr1e1, Atg4c, Gm14296, Hcrt, and Ttr, or a byproduct or precursor or degradation product or fragment thereof) as compared to the first level of the one or more diagnostic biomarkers described herein (e.g., one or more of PMCH, Akr1e1, Atg4c, Gm14296, Hcrt, and Ttr, or a byproduct or precursor or degradation product or fragment thereof), as having static or regressing Alzheimer's disease. In some embodiments, the methods can include identifying a subject having an increased second level the one or more diagnostic biomarkers described herein (e.g., one or more of PMCH, Akr1e1, Atg4c, Gm14296, Hcrt, and Ttr, or a byproduct or precursor or degradation product or fragment thereof) as compared to the first level of the one or more diagnostic biomarkers described herein (e.g., one or more of PMCH, Akr1e1, Atg4c, Gm14296, Hcrt, and Ttr, or a byproduct or precursor or degradation product or fragment thereof), as having progressing Alzheimer's disease. In some embodiments, the methods can include identifying a subject having about the same or a decreased second level of the one or more diagnostic biomarkers described herein (e.g., one or more of PMCH, Akr1e1, Atg4c, Gm14296, Hcrt, and Ttr, or a byproduct or precursor or degradation product or fragment thereof) as compared to the first level of the one or more diagnostic biomarkers described herein (e.g., one or more of PMCH, Akr1e1, Atg4c, Gm14296, Hcrt, and Ttr, or a byproduct or precursor or degradation product or fragment thereof), as having static or regressing Alzheimer's disease.

In some embodiments, the methods can include (a) determining a first level of one or more diagnostic biomarkers described herein (e.g., one or both of Adora2a and Trh, or a byproduct or precursor or degradation product or fragment thereof) in a first biological sample obtained from a subject at a first time point; (b) determining a second level of the one or more diagnostic biomarkers described herein (e.g., one or both of Adora2a and Trh, or a byproduct or precursor or degradation product or fragment thereof) in a second biological sample obtained from the subject at a second time point; (c) identifying: (i) a subject having about the same or a decreased second level of the one or more diagnostic biomarkers described herein (e.g., one or both of Adora2a and Trh, or a byproduct or precursor or degradation product or fragment thereof) as compared to the first level of the one or more diagnostic biomarkers described herein, as having progressing Alzheimer's disease, or (ii) a subject having an increased second level of the one or more diagnostic biomarkers described herein (e.g., one or both of Adora2a and Trh, or a byproduct or precursor or degradation product or fragment thereof) as compared to the first level of the one or more diagnostic biomarkers described herein (e.g., one or both of Adora2a and Trh, or a byproduct or precursor or degradation product or fragment thereof), as having static or regressing Alzheimer's disease. In some embodiments, the methods can include identifying a subject having an increased second level the one or more diagnostic biomarkers described herein (e.g., one or both of Adora2a and Trh, or a byproduct or precursor or degradation product or fragment thereof) as compared to the first level of the one or more diagnostic biomarkers described herein (e.g., one or both of Adora2a and Trh, or a byproduct or precursor or degradation product or fragment thereof), as having static or regressing Alzheimer's disease. In some embodiments, the methods can include identifying a subject having about the same or a decreased second level of the one or more diagnostic biomarkers described herein (e.g., one or both of Adora2a and Trh, or a byproduct or precursor or degradation product or fragment thereof) as compared to the first level of the one or more diagnostic biomarkers described herein (e.g., one or both of Adora2a and Trh, or a byproduct or precursor or degradation product or fragment thereof), as having progressing Alzheimer's disease.

In some embodiments, when the methods include identifying a subject as having progressing Alzheimer's disease, the methods can further include administering a treatment for Alzheimer's disease to the subject or increasing the dose of a previously administered treatment for Alzheimer's disease to the subject. In some embodiments, the methods can further include selecting a treatment for Alzheimer's disease for the subject. In some embodiments, the methods can further include administering a treatment of Alzheimer's disease to the subject. In some embodiments, a treatment for Alzheimer's disease can be a treatment that reduces the rate of progression of Alzheimer's disease.

In some embodiments, a method of a treatment of Alzheimer's disease can include, but is not limited to treatment with cholinesterase inhibitor, an N-methyl-D-aspartate (NMDA) inhibitor, an antipsychotic, a tricyclic antidepressant, a benzodiazepine, insulin, and tacrine hydrochloride. In some embodiments, a treatment of Alzheimer's disease can include a cholinesterase inhibitor, such as galantamine, rivastigmine, or donepezil. In some embodiments, a treatment of Alzheimer's disease can include an N-methyl D-aspartate (NMDA) inhibitor, such as memantine. In some embodiments, a treatment of Alzheimer's disease can include a supportive treatment, such as a sleep aid, a benzodiazepine, an anti-anxiety medication, an anti-depressant, an anticonvulsant, an antipsychotic, or a combination thereof. In some embodiments, the antipsychotic agent used in the treatment of Alzheimer's disease is aripiprazole, risperidone, olanzapine, quetiapine, or haloperidol; the benzodiazepine is lorazepam, oxazepam or temazepam; and the tricyclic antidepressant is nortriptyline. Nonlimiting examples of agents that may be used to treat Alzheimer's Disease include but are not limited to and Aricept®, Exelon®, Razadyne®, Namenda®.

In some embodiments, the methods can further include updating the subject's clinical record that the subject has progressing Alzheimer's disease. In some embodiments, the methods can further include enrolling the subject in a clinical trial. In some embodiments, the methods can further include informing the subject's family of the progression of the disease. In some embodiments, the methods can further include assessing or referring the subject for enrollment in a supportive care plan or care facility. In some embodiments, the methods can further include monitoring the subject more frequently.

In some embodiments, when the methods include identifying a subject as having static or regressing Alzheimer's disease, the methods can include recording in the subject's clinical record that the subject has static or regressing Alzheimer's disease. In some embodiments, the methods can further include the methods can further include maintaining the dose or lowering the dose of a treatment for Alzheimer's disease to be administered to the subject or ceasing administration of a treatment for Alzheimer's disease to the subject. In some embodiments, the methods can further include assessing or referring the subject to be discharged from a care facility.

(iv) Methods of Determining the Efficacy of a Treatment for Alzheimer's Disease in a Subject

In some embodiments, the methods described in this section can be used to determine the efficacy of treatment of a treatment for Alzheimer's Disease in a subject. Accordingly, in some embodiments, the method can include (a) determining a first level of one or more diagnostic biomarkers described herein (e.g., one or more of PMCH, Akr1e1, Atg4c, Gm14296, Hcrt, and Ttr, or a byproduct or precursor or degradation product or fragment thereof) in a first biological sample obtained from a subject at a first time point; (b) determining a second level of the one or more diagnostic biomarkers described herein (e.g., one or more of PMCH, Akr1e1, Atg4c, Gm14296, Hcrt, and Ttr, or a byproduct or precursor or degradation product or fragment thereof) in a second biological sample obtained from the subject at a second time point, wherein the subject is administered one or more doses of a therapeutic treatment between the first and second time points; (c) identifying: (i) the therapeutic treatment as being effective in a subject having about the same or a decreased second level of the one or more diagnostic biomarkers described herein (e.g., one or more of PMCH, Akr1e1, Atg4c, Gm14296, Hcrt, and Ttr, or a byproduct or precursor or degradation product or fragment thereof), as compared to the first level of the one or more diagnostic biomarkers described herein (e.g., one or more of PMCH, Akr1e1, Atg4c, Gm14296, Hcrt, and Ttr, or a byproduct or precursor or degradation product or fragment thereof), or (ii) the therapeutic treatment as not being effective in a subject having an increased second level of the one or more diagnostic biomarkers described herein (e.g., one or more of PMCH, Akr1e1, Atg4c, Gm14296, Hcrt, and Ttr, or a byproduct or precursor or degradation product or fragment thereof), as compared to the first level of the one or more diagnostic biomarkers described herein (e.g., one or more of PMCH, Akr1e1, Atg4c, Gm14296, Hcrt, and Ttr, or a byproduct or precursor or degradation product or fragment thereof). In some embodiments, the method can include (a) determining a first level of one or more diagnostic biomarkers described herein (e.g., one or more of Adora2a and Trh, or a byproduct or precursor or degradation product or fragment thereof) in a first biological sample obtained from a subject at a first time point; (b) determining a second level of the one or more diagnostic biomarkers described herein (e.g., one or more of Adora2a and Trh, or a byproduct or precursor or degradation product or fragment thereof) in a second biological sample obtained from the subject at a second time point, wherein the subject is administered one or more doses of a therapeutic treatment between the first and second time points; (c) identifying: (i) the therapeutic treatment as being effective in a subject having an increased second level of the one or more diagnostic biomarkers described herein (e.g., one or more of Adora2a and Trh, or a byproduct or precursor or degradation product or fragment thereof), as compared to the first level of the one or more diagnostic biomarkers described herein (e.g., one or more of Adora2a and Trh, or a byproduct or precursor or degradation product or fragment thereof), or (ii) the therapeutic treatment as not being effective in a subject having about the same or a decreased second level of the one or more diagnostic biomarkers described herein (e.g., one or more of Adora2a and Trh, or a byproduct or precursor or degradation product or fragment thereof), as compared to the first level of the one or more diagnostic biomarkers described herein (e.g., one or more of Adora2a and Trh, or a byproduct or precursor or degradation product or fragment thereof).

In some embodiments, the methods include identifying the therapeutic treatment as being effective in the subject. In some embodiments, the methods can further include selecting additional doses of the therapeutic treatment for the subject. In some embodiments, the methods can further include administering additional doses of the therapeutic treatment to the subject. In some embodiments, the methods can further include recording in the subject's clinical record that the therapeutic treatment is effective in the subject.

In some embodiments, the methods include identifying the therapeutic treatment as not being effective in the subject. In some embodiments, the methods can further include selecting a different therapeutic treatment for the subject. In some embodiments, the methods can further include administering a different therapeutic treatment to the subject. In some embodiments, the methods can further include increasing the dose of the therapeutic treatment to be administered to the subject. In some embodiments, the methods can include administering one or more additional doses of the therapeutic treatment to the subject in combination with an additional therapeutic treatment. In some embodiments, the methods can further include ceasing administration of the therapeutic treatment to the subject. In some embodiments, the methods can further include recording in the subject's clinical record that the therapeutic treatment is not effective in the subject. In some embodiments, the methods can further include referring the patient for enrollment in a clinical trial of a different therapeutic agent.

In some embodiments, the methods can further include additional assessments of the efficacy of the therapeutic treatment. Non-limiting examples of ways to assess efficacy of the therapeutic treatment include obtaining an image of the subject's brain (e.g., a CT, MRI, or PET scan), testing of other biomarkers, and performing cognitive testing on the subject (e.g., neuropsychological testing). Non-limiting examples of additional assessments can include hallmarks of amyloid beta (Aβ) deposition, such as low cerebrospinal Aβ42 and/or uptake of amyloid PET tracers and/or hallmarks of neurodegeneration, such as elevation of total tau (t-tau) and/or phosphorylated tau (p-tau) in cerebrospinal fluid, hypometabolism on a fluorodeoxyglucose (FDG) PET scan, and/or atrophy (e.g., hippocampal atrophy) on structural MRI, where each of these hallmarks is indicative of the therapeutic treatment not being effective in the subject.

(k) Identifying a Candidate Drug Target for Alzheimer's Disease

In some embodiments, provided herein are methods for identifying a candidate drug target for treatment of a brain disorder (e.g., AD). A candidate drug target as used herein refers to a biomarker that has been identified as dysregulated (e.g., upregulated or downregulated) in a brain tissue (e.g., as described herein). Treatment of a drug or therapeutic molecule restores the levels (e.g., abundance) of the dysregulated biomarker. The methods can include (a) determining level(s) of one or more biomarker(s) in a location in a sample comprising brain tissue obtained from an animal having a brain disorder, (b) identifying: (i) one or more biomarker(s) (e.g., one or more of PMCH, Akr1e1, Atg4c, Gm14296, Hcrt, and Ttr) showing elevated level(s) in the location in the sample as compared to reference level(s), and/or (ii) one or more biomarker(s) (e.g., one or both of Adora2a and Trh) showing decreased level(s) in the location in the sample as compared to reference level(s), as candidate drug target(s) for treatment of the brain disorder. In some embodiments, a reference level of the one or more biomarker(s) is a level of the one or more biomarker(s) in a corresponding location in a sample comprising brain tissue obtained from a control animal. In some embodiments, an animal can be any of the exemplary animals described herein. In some embodiments, an animal can be a mammal.

In some embodiments, the methods can include identifying one or more biomarker(s) (e.g., one or more of PMCH, Akr1e1, Atg4c, Gm14296, Hcrt, and Ttr) showing elevated level(s) in the location in the sample as compared to reference level(s) as candidate drug target(s) for treatment of the brain disorder. In some embodiments, the methods further include testing the ability of an inhibitor of the expression and/or activity of the one or more identified candidate drug target(s) (e.g., one or more of PMCH, Akr1e1, Atg4c, Gm14296, Hcrt, and Ttr) to treat the brain disorder in an animal (e.g., using a clinical trial, enzymatic assays, assessment of cell signaling activity, in vitro assays, ex vivo assays, or an animal model of the brain disorder (e.g., any of the exemplary animal models of brain disorders described herein or known in the art).

In some embodiments, the methods can include identifying one or more biomarker(s) showing decreased level(s) (e.g., one of both of Adora2a and Trh) in the location in the sample as compared to reference level(s) of the one or more biomarker(s) as candidate drug target(s) for treatment of the brain disorder. In some embodiments, the method can further include testing the ability of an agent that increases the expression and/or activity of the one or more identified candidate drug target(s) (e.g., one or both of Adora2a and Trh) to treat the brain disorder in an animal (e.g., using a clinical trial, enzymatic assays, assessment of cell signaling activity, in vitro assays, ex vivo assays, or an animal model of the brain disorder (e.g., any of the exemplary animal models of brain disorders described herein or known in the art).

In some embodiments, the methods can further include additional studies to further validate a candidate drug target. Non-limiting examples of additional studies can include generation of a knockout (e.g., a PMCH, Akr1e1, Atg4c, Gm14296, Hcrt, or Ttr knockout) or a knock-in (e.g., a Adora2a or Trh knock-in) animal, administration of an agent that activates (e.g., activates Adora2a or Trh) and/or inhibits (e.g., inhibits one or more of PMCH, Akr1e1, Atg4c, Gm14296, Hcrt, and Ttr) the candidate drug target (or a protein or nucleic acid that is downstream of the activity of the candidate drug target in a cell).

Some embodiments of these methods can further include screening for a molecule that inhibits the expression and/or at least one activity of a candidate drug target (for a candidate drug target that has a level that is elevated at a location in the brain as compared to a reference level) (e.g., one or more of PMCH, Akr1e1, Atg4c, Gm14296, Hcrt, and Ttr). Some embodiments of these methods can further include screening for a molecule that increases the expression and/or at least one activity of a candidate drug target (for a candidate drug target that has a level that is decreased at a location in the brain as compared to a reference level) (e.g., one or both of Adora2a and Trh).

Other studies to further validate a candidate drug target will be apparent to those skilled in the field.

(l) Biomarkers of Glioblastoma

Non-limiting biomarkers of glioblastoma include COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, SLN, SRPX2, METTL7B, POSTN, NNMT, TIMP4, SERPINA3, KLHDC8A, NES, F2R, XIST, COL1A2, COL4A1, CA12, ANXA2, WWTR1, COL4A1, LAMB2, SPARC, FN1, TNFRSF1A, HLA-DRA, ALDH1L1, FLNA, NAMPT, VEGFA, C3, HLA-A, GRN, HLA-B, TPP1, HLA-B, HLA-DRA, LAMB2, and NAMPT.

TABLE 9
Exemplary Biomarkers of Glioblastoma

| | Protein PubMed | cDNA PubMed |
|---|---|---|
| Biomarker | Accession No. | Accession No. |
| COL1A1 | NP_000079.2 | NM_000088.4 |
| COL3A1 | NP_000081.2 | NM_000090.4 |
| COL8A1 | NP_065084.2 | NM_020351 |
| WEE1 | NP_003381.1 | NM_003390.4 |
| CHI3L1 | NP_001267.2 | NM_001276.4 |
| MGP | NP_000891.2 | NM_000900.5 |
| SRPX | NP_006298.1 | NM_006307.5 |
| SERPINE1 | NP_000593.1 | NM_000602.5 |
| COL1A2 | NP_000080.2 | NM_000089.4 |
| TIMP1 | NP_003245.1 | NM_003254.3 |
| ANXA1 | NP_000691.1 | NM_000700.3 |
| COL6A2 | NP_001840.3 | NM_001849.4 |
| CAV1 | NP_001744.2 | NM_001753.5 |
| PLIN2 | NP_001113.2 | NM_001122.4 |
| CD44 | NP_000601.3 | NM_000610.4 |
| APOC1 | NP_001636.1 | NM_001645.5 |
| IGFBP2 | NP_000588.3 | NM_000597.3 |
| PDPN | NP_006465.3 | NM_006474.4 |
| VIM | NP_003371.2 | NM_003380.5 |
| LGALS3 | NP_002297.2 | NM_002306.4 |
| VEGFA | NP_003367.4 | NM_003376.6 |
| IGFBP5 | NP_000590.1 | NM_000599.4 |
| CTGF | NP_001892.1 | NM_001901.3 |
| EMP1 | NP_001414.1 | NM_001423.3 |
| EMP3 | NP_001416.1 | NM_001425.3 |
| IGFBP3 | NP_001013416.1 | NM_001013398.2 |
| A2M | NP_000005.3 | NM_000014.6 |
| ANXA2 | NP_004030.1 | NM_004039.3 |
| FLNA | NP_001104026.1 | NM_001110556.2 |
| IFGBP7 | NP_001544.1 | NM_001553.3 |
| S100A11 | NP_005611.1 | NM_005620.2 |
| ADM | NP_001115.1 | NM_001124.3 |
| FN1 | NP_997647.1 | NM_212482.3 |
| SERPING1 | NP_000053.2 | NM_000062.3 |
| MT2A | NP_005944.1 | NM_005953.5 |
| S100A10 | NP_002957.1 | NM_002966.3 |
| SPARC | NP_003109.1 | NM_003118.4 |
| ITGB1 | NP_002202.2 | NM_002211.4 |
| SLC5A3 | NP_008864.4 | NM_006933.7 |
| FABP7 | NP_001437.1 | NM_001446.5 |
| YBX3 | NP_003642.3 | NM_003651.5 |
| IFITM2 | NP_006426.2 | NM_006435.2 |
| TAGLN2 | NP_003555.1 | NM_003564.3 |
| COL6A1 | NP_001839.2 | NM_001848.3 |
| HLA-A | NP_002107.3 | NM_002116.8 |
| LGALS3BP | NP_005558.1 | NM_005567.4 |
| ANXA5 | NP_001145.1 | NM_001154.4 |
| APOE | NP_000032.1 | NM_000041.4 |
| GADD45A | NP_001915.1 | NM_001924.4 |
| TPM4 | NP_003281.1 | NM_003290.3 |
| SPP1 | NP_001035147.1 | NM_001040058.2 |
| GABRA1 | NP_001121116.1 | NM_001127644.2 |
| CCK | NP_000720.1 | NM_000729.6 |
| SLC17A7 | NP_064705.1 | NM_020309.4 |
| CHGA | NP_001266.1 | NM_001275.4 |
| STMN2 | NP_008960.2 | NM_007029.4 |
| CALY | NP_056537.1 | NM_015722.4 |
| EEF1A2 | NP_001949.1 | NM_001958.5 |
| CABP1 | NP_001028849.1 | NM_001033677.1 |
| NRGN | NP_006167.1 | NM_006176.3 |
| SNAP25 | NP_570824.1 | NM_130811.4 |
| ATP2B2 | NP_001001331.1 | NM_001001331.4 |
| SYN1 | NP_008881.2 | NM_006950.3 |
| NECAB1 | NP_071746.1 | NM_022351.5 |
| MBP | NP_001020272.1 | NM_001025101.2 |
| PHYHIP | NP_055574.3 | NM_014759.5 |
| BASP | NP_006308.3 | NM_006317.5 |
| CPLX1 | NP_006642.1 | NM_006651.4 |
| VSNL1 | NP_003376.2 | NM_003385.5 |
| TAGLN3 | NP_001008273.1 | NM_001008272.2 |
| ENC1 | NP_003624.1 | NM_003633.3 |
| FBXL16 | NP_699181.2 | NM_153350.4 |
| CHN1 | NP_001813.1 | NM_001822.7 |
| KIF5A | NP_004975.2 | NM_004984.4 |
| PLP1 | NP_000524.3 | NM_000533.5 |
| OLFM1 | NP_001269540.1 | NM_001282611.2 |
| SNCB | NP_003076.1 | NM_003085.5 |
| STXBP1 | NP_001027392.1 | NM_001032221.6 |
| ATP1B1 | NP_001668.1 | NM_001677.4 |
| DNM1 | NP_004399.2 | NM_004408.4 |
| SERPINI1 | NP_001116224.1 | NM_001122752.1 |
| PRKAR1B | NP_001158232.1 | NM_001164760.2 |
| MEF2C | NP_002388.2 | NM_002397.5 |
| MTURN | NP_690006.2 | NM_152793.3 |
| NSF | NP_006169.2 | NM_006178.4 |
| SYT1 | NP_005630.1 | NM_005639.3 |
| MAP2 | NP_001362434.1 | NM_001375505.1 |
| MT-ATP8 | YP_003024030.1 | |
| MAP1A | NP_002364.5 | NM_002373.6 |
| UCHL1 | NP_004172.2 | NM_004181.5 |
| FAIM2 | NP_036438.2 | NM_012306.4 |
| STMN1 | NP_005554.1 | NM_005563.4 |
| APLP1 | NP_001019978.1 | NM_001024807.3 |
| NCDN | NP_055099.1 | NM_014284.3 |
| STMN3 | NP_056978.2 | NM_015894.4 |
| MT-ND4L | YP_003024034.1 | |
| BEX1 | NP_060946.3 | NM_018476.4 |
| MT-ND2 | YP_003024027.1 | |
| PPP3CA | NP_000935.1 | NM_000944.5 |
| CPLX2 | NP_006641.1 | NM_006650.4 |
| ST8SIA3 | NP_056963.2 | NM_015879.3 |
| GABRG2 | NP_944494.1 | NM_198904.3 |
| KCNC2 | NP_631875.1 | NM_139137.4 |
| MT-ND5 | YP_003024036.1 | |

Some embodiments of any of the methods described herein can include the detection of a level of one or more of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, SLN, SRPX2, METTL7B, POSTN, NNMT, TIMP4, SERPINA3, KLHDC8A, NES, F2R, XIST, COL1A2, COL4A1, CA12, ANXA2, WWTR1, COL4A1, LAMB2, SPARC, FN1, TNFRSF1A, HLA-DRA, ALDH1L1, FLNA, NAMPT, VEGFA, C3, HLA-A, GRN, HLA-B, TPP1, HLA-B, HLA-DRA, LAMB2, or NAMPT or a byproduct, a degradation product, or a precursor thereof

(m) Methods of (i) Diagnosing Glioblastoma, (ii) Identifying Increased Likelihood of Developing Glioblastoma, (iii) Monitoring the Progression of Glioblastoma, and (iv) Determining the Efficacy of a Treatment for Glioblastoma in a Subject

Also provided herein are methods of diagnosing a subject as having glioblastoma. Also provided herein are methods of identifying a subject as having an increased likelihood of developing glioblastoma. In some embodiments, the methods can include (a) determining a level of one or more of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, SLN, SRPX2, METTL7B, POSTN, NNMT, TIMP4, SERPINA3, KLHDC8A, NES, F2R, XIST, COL1A2, COL4A1, CA12, ANXA2, WWTR1, COL4A1, LAMB2, SPARC, FN1, TNFRSF1A, HLA-DRA, ALDH1L1, FLNA, NAMPT, VEGFA, C3, HLA-A, GRN, HLA-B, TPP1, HLA-B, HLA-DRA, LAMB2, or NAMPT or a byproduct or precursor or degradation product or fragment thereof, in a biological sample from a subject; and (b) identifying a subject having an elevated level of one or more of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, and SPP1, or a byproduct or precursor or degradation product or fragment thereof, as compared to a reference level, as having glioblastoma. In some embodiments, the methods can include (a) determining a level of one or more of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, and MT-ND5, or a byproduct or precursor or degradation product or fragment thereof, in a biological sample from a subject; and (b) identifying a subject having an decreased level of one or more of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, and MT-ND5, or a byproduct or precursor or degradation product or fragment thereof, in the biological sample as compared to a reference level, as having glioblastoma.

A biological sample can be any appropriate biological sample. In some embodiments, a biological sample can be a sample comprising brain tissue or cerebrospinal fluid. In some embodiments, the method can further include obtaining the sample from the subject.

In some embodiments, the methods can further include confirming a diagnosis of glioblastoma in the subject. Non-limiting examples of ways to confirm a diagnosis of glioblastoma include obtaining an image of the subject's brain (e.g., a CT, MM, or PET scan), detecting a genetic mutation associated with glioblastoma (e.g., a mutation associated with neurofibromatosis type 1, Turcot syndrome or Li Fraumeni syndrome), determining the levels of other biomarkers of glioblastoma, or performing neurological testing on the subject (e.g., vision, hearing, balance, coordination, strength and reflexes testing). Other methods of confirming a diagnosis of glioblastoma will be apparent to one skilled in the field.

In some embodiments, the methods can further include selecting a treatment for the subject. In some embodiments, the methods can further include administering a treatment of glioblastoma to the subject. In some embodiments, a treatment of glioblastoma can be a treatment that reduces the rate of progression of glioblastoma. In some embodiments, a treatment of glioblastoma can include surgery, radiation therapy, chemotherapy, targeted drug therapy, and tumor treating fields (TTF) therapy.

In some instances, the methods disclosed herein include treating a subject having glioblastoma with one or more therapeutic agents. Examples of therapeutic agents include, but are not limited to, e.g., chemotherapeutic agents, growth inhibitory agents, cytotoxic agents, agents used in radiation therapy, anti-angiogenesis agents, cancer immunotherapeutic agents, apoptotic agents, anti-tubulin agents, and other-agents (e.g., antibodies) to treat cancer, such as anti-HER-2 antibodies, anti-CD20 antibodies, an epidermal growth factor receptor (EGFR) antagonist (e.g., a tyrosine kinase inhibitor), HER1/EGFR inhibitor (e.g., erlotinib (Tarceva®), platelet derived growth factor inhibitors (e.g., Gleevec® (Imatinib Mesylate)), a COX-2 inhibitor (e.g., celecoxib), interferons, CTLA-4 inhibitors (e.g., anti-CTLA antibody ipilimumab (YERVOY®)), PD-1 inhibitors (e.g., anti-PD-1 antibodies, BMS-936558), PD-L1 inhibitors (e.g., anti-PD-L1 antibodies, MPDL3280A), PD-L2 inhibitors (e.g., anti-PD-L2 antibodies), TIM3 inhibitors (e.g., anti-TIM3 antibodies), cytokines, antagonists (e.g., neutralizing antibodies) that bind to one or more of the following targets ErbB2, ErbB3, ErbB4, PDGFR-beta, BlyS, APRIL, BCMA, PD-1, PD-L1, PD-L2, CTLA-4, TIM3, or VEGF receptor(s), TRAIL/Apo2, and other bioactive and organic chemical agents, etc. In some instances, the therapy or treatment includes surgery, chemotherapeutic agents, growth inhibitory agents, cytotoxic agents, agents used in radiation therapy, anti-angiogenesis agents, cancer immunotherapeutic agents, apoptotic agents, anti-tubulin agents, or a combination thereof.

In some instances, chemotherapeutic agents are provided as a therapy to a subject having glioblastoma. Nonlimiting exemplary chemotherapeutic agents include anti-hormonal agents that act to regulate or inhibit hormone action on cancers such as anti-estrogens and selective estrogen receptor modulators (SERMs), including, for example, tamoxifen (including Nolvadex® tamoxifen), raloxifene, droloxifene, 4-hydroxytamoxifen, trioxifene, keoxifene, LY117018, onapristone, and Fareston® toremifene; aromatase inhibitors that inhibit the enzyme aromatase, which regulates estrogen production in the adrenal glands, such as, for example, 4(5)-imidazoles, aminoglutethimide, Megase® megestrol acetate, Aromasin® exemestane, formestanie, fadrozole, Rivisor® vorozole, Femara® letrozole, and Arimidex® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., Angiozyme® ribozyme) and a HER2 expression inhibitor; vaccines such as gene therapy vaccines, for example, Allovectin® vaccine, Leuvectin® vaccine, and Vaxid® vaccine; Proleukin® rIL-2; Lurtotecan® topoisomerase 1 inhibitor; Abarelix® rmRH; and pharmaceutically acceptable salts, acids or derivatives of any of the above.

In some embodiments, radiation therapy is administered locally to a tumor lesion to enhance the local immunogenicity of a subject's tumor (adjuvinating radiation) and/or to kill tumor cells (ablative radiation). In some instances, radiation therapy is administered systemically to a subject. In some instances, the radiation therapy is tomotherapy, stereotactic radiation, intensity-modulated radiation therapy (IMRT), hypofractionated radiotherapy, hypoxia-guided radiotherapy, and/or proton therapy. In some instances, radiation is followed by administration of a second therapy (e.g., chemotherapy, immunotherapy). In some instances, radiation is provided concurrently with administration of a second therapy (e.g., chemotherapy, immunotherapy).

In some instances, any of the above therapeutic agents are provided before, substantially contemporaneous with, or after other modes of treatment, for example, surgery, chemotherapy, radiation therapy, or the administration of a biologic, such as another therapeutic antibody. In some embodiments, the cancer has recurred or progressed following a therapy selected from surgery, chemotherapy, and radiation therapy, or a combination thereof.

In some instances, for treatment of cancer, as discussed herein, the antibodies are administered in conjunction with one or more additional anti-cancer agents, such as the chemotherapeutic agent, growth inhibitory agent, anti-angiogenesis agent and/or anti-neoplastic composition. Nonlimiting examples of chemotherapeutic agent, growth inhibitory agent, anti-angiogenesis agent, anti-cancer agent and anti-neoplastic composition.

In some embodiments, the methods can further include updating the subject's clinical record with the diagnosis of glioblastoma. In some embodiments, the methods can further include enrolling the subject in a clinical trial. In some embodiments, the methods can further include informing the subject's family of the diagnosis. In some embodiments, the methods can further include assessing or referring the subject for enrollment in a supportive care plan or care facility. In some embodiments, the methods can further include monitoring the subject more frequently.

In some embodiments, the methods can further comprise monitoring the identified subject for the development of symptoms of glioblastoma. In some embodiments, the methods can further include recording in the identified subject's clinical record that the subject has an increased likelihood of developing glioblastoma. In some embodiments, the methods can further include notifying the subject's family that the subject has an increased likelihood or susceptibility of developing glioblastoma.

In some embodiments, the methods can further include administering to the subject a treatment for decreasing the rate of progression or decreasing the likelihood of developing glioblastoma. In some embodiments, a treatment of glioblastoma can include surgery, radiation therapy, chemotherapy, surgery, radiation therapy, chemotherapy, targeted drug therapy, and tumor treating fields (TTF) therapy. In some embodiments, the subject can be tested for the presence of genetic mutations known to be associated with risk for glioblastoma.

In some embodiments, the methods can further include performing one or more tests to further determine the subject's risk of developing glioblastoma. Non-limiting examples of more tests to further determine the subject's risk of developing glioblastoma include, detecting a genetic mutation associated with glioblastoma (e.g., a mutation associated with neurofibromatosis type 1, Turcot syndrome, or Li Fraumeni syndrome), and determining the levels of other biomarkers (e.g., in brain tissue, cerebrospinal fluid, or in blood or a component thereof) indicative an increased risk of developing glioblastoma are indicative of an increased risk of developing glioblastoma.

In some embodiments, the methods can further include updating the subject's clinical record to indicate an increased risk of developing glioblastoma. In some embodiments, the methods can further include enrolling the subject in a clinical trial (e.g., for the early treatment and/or prevention of glioblastoma). In some embodiments, the methods can further include informing the subject's family of the subject's likelihood of developing glioblastoma. In some embodiments, the methods can further include monitoring the subject more frequently.

In some embodiments, provided herein are methods of monitoring progression of glioblastoma in a subject over time. In some embodiments, the methods can include (a) determining a first level of one or more of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, and SPP, or a byproduct or precursor or degradation product or fragment thereof, in a first biological sample obtained from a subject at a first time point; (b) determining a second level of one or more of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, and SPP, or a byproduct or precursor or degradation product or fragment thereof, in a second biological sample obtained from the subject at a second time point; (c) identifying: (i) a subject having an increased second level as compared to the first level, as having progressing glioblastoma, or (ii) a subject having about the same or a decreased second level as compared to the first level, as having static or regressing glioblastoma.

In some embodiments, the methods can include (a) determining a first level of one or more of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, and MT-ND5, or a byproduct or precursor or degradation product or fragment thereof, in a first biological sample obtained from a subject at a first time point; (b) determining a second level of one or more of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, and MT-ND5, or a byproduct or precursor or degradation product or fragment thereof, in a second biological sample obtained from the subject at a second time point; (c) identifying: (i) a subject having about the same or a decreased second level as compared to the first level of the one or more diagnostic biomarkers described herein, as having progressing glioblastoma, or (ii) a subject having an increased second level as compared to the first level, as having static or regressing glioblastoma.

A biological sample can be any appropriate biological sample. In some embodiments, a biological sample can comprise brain tissue or cerebrospinal fluid. In some embodiments, the method can further include obtaining the first and second biological samples from the subject.

In some embodiments, when the methods include identifying a subject as having progressing glioblastoma, the methods can further include administering a treatment for glioblastoma to the subject or increasing the dose of a previously administered treatment for glioblastoma to the subject. In some embodiments, the methods can further include selecting a treatment for glioblastoma for the subject. In some embodiments, the methods can further include administering a treatment of glioblastoma to the subject. In some embodiments, a treatment for glioblastoma can be a treatment that reduces the rate of progression of glioblastoma. In some embodiments, a treatment of glioblastoma can include surgery, radiation therapy, chemotherapy, targeted drug therapy, and tumor treating fields (TTF) therapy. In some embodiments, a treatment of glioblastoma can include palliative care. In some embodiments, the methods can further include updating the subject's clinical record that the subject has progressing glioblastoma. In some embodiments, the methods can further include enrolling the subject in a clinical trial. In some embodiments, the methods can further include informing the subject's family of the progression of the disease. In some embodiments, the methods can further include assessing or referring the subject for enrollment in a supportive care plan. In some embodiments, the methods can further include monitoring the subject more frequently.

In some embodiments, when the methods include identifying a subject as having static or regressing glioblastoma, the methods can include recording in the subject's clinical record that the subject has static or regressing glioblastoma. In some embodiments, the methods can further include the methods can further include maintaining the dose or lowering the dose of a treatment for glioblastoma to be administered to the subject or ceasing administration of a treatment for glioblastoma to the subject.

In some embodiments, provided herein are methods of determining efficacy of treatment of a treatment for glioblastoma in a subject. In some embodiments, the method can include (a) determining a first level of one or more of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, and SPP1, or a byproduct or precursor or degradation product or fragment thereof, in a first biological sample obtained from a subject at a first time point; (b) determining a second level of one or more of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, and SPP1, or a byproduct or precursor or degradation product or fragment thereof, in a second biological sample obtained from the subject at a second time point, wherein the subject is administered one or more doses of a therapeutic treatment between the first and second time points; (c) identifying: (i) the therapeutic treatment as being effective in a subject having about the same or a decreased second level as compared to the first level, or (ii) the therapeutic treatment as not being effective in a subject having an increased second level as compared to the first level.

In some embodiments, the method can include (a) determining a first level of one or more of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, and MT-ND5, or a byproduct or precursor or degradation product or fragment thereof, in a first biological sample obtained from a subject at a first time point; (b) determining a second level of one or more of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, and MT-ND5, or a byproduct or precursor or degradation product or fragment thereof, in a second biological sample obtained from the subject at a second time point, wherein the subject is administered one or more doses of a therapeutic treatment between the first and second time points; (c) identifying: (i) the therapeutic treatment as being effective in a subject having an increased second level as compared to the first level, or (ii) the therapeutic treatment as not being effective in a subject having about the same or a decreased second level as compared to the first level.

In some embodiments, the methods include identifying the therapeutic treatment as being effective in the subject. In some embodiments, the methods can further include selecting additional doses of the therapeutic treatment for the subject. In some embodiments, the methods can further include administering additional doses of the therapeutic treatment to the subject. In some embodiments, the methods can further include recording in the subject's clinical record that the therapeutic treatment is effective in the subject.

In some embodiments, the methods include identifying the therapeutic treatment as not being effective in the subject. In some embodiments, the methods can further include selecting a different therapeutic treatment for the subject. In some embodiments, the methods can further include administering a different therapeutic treatment to the subject. In some embodiments, the methods can further include increasing the dose of the therapeutic treatment to be administered to the subject. In some embodiments, the methods can include administering one or more additional doses of the therapeutic treatment to the subject in combination with an additional therapeutic treatment. In some embodiments, the methods can further include ceasing administration of the therapeutic treatment to the subject. In some embodiments, the methods can further include recording in the subject's clinical record that the therapeutic treatment is not effective in the subject. In some embodiments, the methods can further include referring the patient for enrollment in a clinical trial of a different therapeutic agent.

The biological samples can be any appropriate biological sample. In some embodiments, the biological samples can include brain tissue or cerebrospinal fluid. In some embodiments, the method can further include obtaining the first and second biological samples from the subject.

In some embodiments, the methods can further include additional assessments of the efficacy of the therapeutic treatment. Non-limiting examples of ways to assess efficacy of the therapeutic treatment include obtaining an image of the subject's brain (e.g., a CT, MRI, or PET scan), testing of other biomarkers, and performing neurological testing on the subject (e.g., vision, hearing, balance, coordination, strength and reflexes testing).

(n) Methods of Detecting Biomarker(s) in a Location in a Sample

Any of the exemplary methods described herein can be used to determine a level and/or at least one activity of one or more biomarkers (e.g., one or more of PMCH, Akr1e1, Atg4c, Gm14296, Hcrt, Ttr, Adora2a, and Trh, or a byproduct, degradation product, or fragment, or precursor thereof) in a location in a sample (e.g., a brain tissue sample, or a cell culture sample). In some embodiments, determining a level and/or an activity of one or more biomarkers (e.g., one or more of PMCH, Akr1e1, Atg4c, Gm14296, Hcrt, Ttr, Adora2a, and Trh) can include any of the workflows described herein.

In some embodiments, the methods can include contacting the sample with a binding agent that specifically binds to a biomarker (e.g., one of PMCH, Akr1e1, Atg4c, Gm14296, Hcrt, Ttr, Adora2a, and Trh, or a byproduct, degradation product, or fragment, or precursor thereof) (e.g., gDNA, mRNA, a protein, or a byproduct, degradation product, or fragment, or precursor thereof), wherein the binding agent further comprises an oligonucleotide having a sequence; and sequencing all or a portion of the sequence of the oligonucleotide or a complement thereof, from a probe specifically bound to the biomarker (e.g., one of PMCH, Akr1e1, Atg4c, Gm14296, Hcrt, Ttr, Adora2a, and Trh, or a byproduct, degradation product, or fragment, or precursor thereof) in the location of the sample, to determine the level of the biomarker (e.g., PMCH, Akr1e1, Atg4c, Gm14296, Hcrt, Ttr, Adora2a, or Trh, or a byproduct, degradation product, or fragment, or precursor thereof) in the location in the sample.

In some embodiments, the methods can include delivering a plurality of probes to a sample (e.g., a tissue sample, for instance, affixed to a support), wherein a probe of the plurality of probes includes a protein that specifically binds to a biomarker (e.g., one of PMCH, Akr1e1, Atg4c, Gm14296, Hcrt, Ttr, Adora2a, and Trh, or a byproduct, degradation product, or fragment, or precursor thereof) (e.g., a protein, or a byproduct, degradation product, or fragment, or precursor thereof) in the tissue sample, wherein the protein is conjugated to an oligonucleotide having a sequence, and separating the probe specifically bound to the biomarker at the location of the tissue sample from the plurality of probes not specifically bound to the biomarker at the location of the tissue sample; and sequencing all or a portion of the sequence of the oligonucleotide or a complement thereof, from the specifically bound probe, and using the determined sequence to associate presence or abundance of the biomarker with the location of the tissue sample.

In some embodiments, the methods can include delivering a plurality of probes to a sample (e.g., a tissue sample, for instance, affixed to a support), wherein a probe of the plurality of probes includes a first oligonucleotide that specifically binds to a biomarker (e.g., one of PMCH, Akr1e1, Atg4c, Gm14296, Hcrt, Ttr, Adora2a, and Trh, or a byproduct, degradation product, or fragment, or precursor thereof) (e.g., gDNA, mRNA, or a byproduct, degradation product, or fragment, or precursor thereof) in the tissue sample, wherein the first oligonucleotide is conjugated to a second oligonucleotide having a sequence, and separating the probe specifically bound to the biomarker at the location of the tissue sample from the plurality of probes not specifically bound to the biomarker at the location of the tissue sample; and sequencing all or a portion of the sequence of the second oligonucleotide or a complement thereof, from the specifically bound probe, and using the determined sequence to associate presence or abundance of the biomarker with the location of the tissue sample.

In some embodiments, the methods can include delivering a plurality of probes to a tissue sample, wherein at least one probe of the plurality of probes comprises a protein that specifically binds to a biomarker (e.g., one of PMCH, Akr1e1, Atg4c, Gm14296, Hcrt, Ttr, Adora2a, and Trh, or a byproduct, degradation product, or fragment, or precursor thereof) (e.g., a protein, or a byproduct, degredation product, precursor, or fragment of any thereof) in the tissue sample, wherein the protein is conjugated to an oligonucleotide having a sequence, and wherein (i) each of the at least one probe comprises a protein that specifically binds a different biomarker of the tissue sample, and (ii) the protein of each of the at least one probe is conjugated to a different oligonucleotide having a sequence; imaging the tissue sample to identify a location of interest of the tissue sample; and sequencing all or a portion of the sequence(s) of the oligonucleotide(s) or a complement thereof, from the at least one probe specifically bound to the biomarker in the location of interest of the tissue sample, and using the determined sequence(s) to associate presence or abundance of the biomarker with the location of interest of the tissue sample.

In some embodiments, the methods can include delivering a plurality of probes to a tissue sample, wherein at least one probe of the plurality of probes comprises a first oligonucleotide that specifically binds a biomarker (e.g., one of PMCH, Akr1e1, Atg4c, Gm14296, Hcrt, Ttr, Adora2a, and Trh, or a byproduct, degradation product, or fragment, or precursor thereof) (e.g., gDNA, mRNA, or a byproduct, degradation product, or fragment, or precursor thereof) in the tissue sample, wherein the first oligonucleotide is conjugated to a second oligonucleotide having a sequence, and wherein (i) each of the at least one probe comprises a first oligonucleotide that specifically binds a different biomarker of the tissue sample, and (ii) the first oligonucleotide of each of the at least one probe is conjugated to a different second oligonucleotide having a sequence; imaging the tissue sample to identify a location of interest of the tissue sample; and sequencing all or a portion of the sequence(s) of the second oligonucleotide(s) or a complement thereof, from the at least one probe specifically bound to the biomarker in the location of interest of the tissue sample, and using the determined sequence(s) to associate presence or abundance of the biomarker with the location of interest of the tissue sample.

Any of the exemplary methods described herein can be used to determine a level and/or at least one activity of one or more biomarkers (e.g., one or more of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, and MT-ND5, or a byproduct, degradation product, or fragment, or precursor thereof) in a sample (e.g., a brain tissue sample or cerebrospinal fluid) or at a location in a sample (e.g., a brain tissue sample). In some embodiments, determining a level and/or an activity of one or more biomarkers (e.g., one or more of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, and MT-ND5) can include any of the workflows described herein.

In some embodiments, the methods can include contacting the sample with a binding agent that specifically binds to a biomarker (e.g., one of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, or MT-ND5, or a byproduct, degradation product, or fragment, or precursor thereof) (e.g., gDNA, mRNA, a protein, or a byproduct, degradation product, or fragment, or precursor thereof), wherein the binding agent further comprises an oligonucleotide having a sequence; and sequencing all or a portion of the sequence of the oligonucleotide or a complement thereof, from a probe specifically bound to the biomarker (e.g., one of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, or MT-ND5, or a byproduct, degradation product, or fragment, or precursor thereof), to determine the level of the biomarker (e.g., COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, or MT-ND5, or a byproduct, degradation product, or fragment, or precursor thereof) in the sample (e.g., cerebrospinal fluid or brain tissue) or at a location in the sample (e.g., brain tissue).

In some embodiments, the methods can include delivering a plurality of probes to a sample (e.g., a tissue sample, for instance, affixed to a support), wherein a probe of the plurality of probes includes a protein that specifically binds to a biomarker (e.g., one of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, or MT-ND5, or a byproduct, degradation product, or fragment, or precursor thereof) (e.g., a protein, or a byproduct, degradation product, or fragment, or precursor thereof) in the tissue sample, wherein the protein is conjugated to an oligonucleotide having a sequence, and separating the probe specifically bound to the biomarker at the location of the tissue sample from the plurality of probes not specifically bound to the biomarker at the location of the tissue sample; and sequencing all or a portion of the sequence of the oligonucleotide or a complement thereof, from the specifically bound probe, and using the determined sequence to determining the level of the biomarker in a sample (e.g., cerebrospinal fluid or brain tissue) or to associate presence or abundance of the biomarker with the location of the tissue sample (e.g., brain tissue).

In some embodiments, the methods can include delivering a plurality of probes to a sample (e.g., a tissue sample, for instance, affixed to a support), wherein a probe of the plurality of probes includes a first oligonucleotide that specifically binds to a biomarker (e.g., one of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, or MT-ND5, or a byproduct, degradation product, or fragment, or precursor thereof) (e.g., gDNA, mRNA, or a byproduct, degradation product, or fragment, or precursor thereof) in the tissue sample, wherein the first oligonucleotide is conjugated to a second oligonucleotide having a sequence, and separating the probe specifically bound to the biomarker at the location of the tissue sample from the plurality of probes not specifically bound to the biomarker at the location of the tissue sample; and sequencing all or a portion of the sequence of the second oligonucleotide or a complement thereof, from the specifically bound probe, and using the determined sequence to determine the presence or level of the biomarker in the sample (e.g., brain tissue or cerebrospinal fluid) or to determine the presence or level of the biomarker at the location in the sample (e.g., brain tissue).

In some embodiments, the methods can include delivering a plurality of probes to a tissue sample, wherein at least one probe of the plurality of probes comprises a protein that specifically binds to a biomarker (e.g., one of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, and MT-ND5, or a byproduct, degradation product, or fragment, or precursor thereof) (e.g., a protein, or a byproduct, degredation product, precursor, or fragment of any thereof) in the tissue sample, wherein the protein is conjugated to an oligonucleotide having a sequence, and wherein (i) each of the at least one probe comprises a protein that specifically binds a different biomarker of the tissue sample, and (ii) the protein of each of the at least one probe is conjugated to a different oligonucleotide having a sequence; imaging the tissue sample to identify a location of interest of the tissue sample; and sequencing all or a portion of the sequence(s) of the oligonucleotide(s) or a complement thereof, from the at least one probe specifically bound to the biomarker in the location of interest of the tissue sample, and using the determined sequence(s) to determine the presence or level of the biomarker in the sample (e.g., brain tissue or cerebrospinal fluid) or to determine the presence or level of the biomarker at the location in the sample (e.g., brain tissue).

In some embodiments, the methods can include delivering a plurality of probes to a tissue sample, wherein at least one probe of the plurality of probes comprises a first oligonucleotide that specifically binds a biomarker (e.g., one of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, and MT-ND5, or a byproduct, degradation product, or fragment, or precursor thereof) (e.g., gDNA, mRNA, or a byproduct, degradation product, or fragment, or precursor thereof) in the tissue sample, wherein the first oligonucleotide is conjugated to a second oligonucleotide having a sequence, and wherein (i) each of the at least one probe comprises a first oligonucleotide that specifically binds a different biomarker of the tissue sample, and (ii) the first oligonucleotide of each of the at least one probe is conjugated to a different second oligonucleotide having a sequence; imaging the tissue sample to identify a location of interest of the tissue sample; and sequencing all or a portion of the sequence(s) of the second oligonucleotide(s) or a complement thereof, from the at least one probe specifically bound to the biomarker in the location of interest of the tissue sample, and using the determined sequence(s) to determine the presence or level of the biomarker in the sample (e.g., brain tissue or cerebrospinal fluid) or to determine the presence or level of the biomarker at the location in the sample (e.g., brain tissue).

A biological sample can be any appropriate biological sample. In some embodiments, a biological sample can be a sample comprising blood, serum, or plasma. In some embodiments, a biological sample can comprise cerebrospinal fluid. In some embodiments, a biological sample can be a cell culture sample. In some embodiments, the method can further include obtaining the first and second biological samples from the subject.

(o) Kits

In some embodiments, also provided herein are kits that include one or more reagents to detect a level of one or more of any of the biomarkers and/or candidate biomarkers described herein (e.g., PMCH, Akr1e1, Atg4c, Gm14296, Hcrt, Ttr, Adora2a, or Trh, or a byproduct or precursor or degradation product or fragment thereof).

In some embodiments, also provided herein are kits that include one or more reagents to detect a level of one or more of any of the biomarkers and/or candidate biomarkers described herein (e.g., COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, or MT-ND5, or a byproduct or precursor or degradation product or fragment thereof).

In some embodiments, reagents can include one or more antibodies (and/or antigen-binding antibody fragments), labeled hybridization probes, and primers. For example, in some embodiments, an antibody (and/or antigen-binding antibody fragment) can be used for visualizing one or more features of a tissue sample (e.g., by using immunofluorescence or immunohistochemistry). In some embodiments, an antibody (and/or antigen-binding antibody fragment) can be an analyte binding moiety, for example, as part of an analyte capture agent. For example, in some embodiments, a kit can include an anti-PMCH antibody, such as Product No. HPA046055 (Atlas Antibodies), Cat. Nos. PA5-25442, PA5-84521, PA5-83802 (ThermoFisher Scientific), or Product No. AV13054 (MilliporeSigma). Other useful commercially available antibodies will be apparent to one skilled in the art.

In some embodiments, labeled hybridization probes can be used for in situ sequencing of one or more biomarkers and/or candidate biomarkers. In some embodiments, primers can be used for amplification (e.g., clonal amplification) of a captured oligonucleotide analyte.

In some embodiments, a kit can further include instructions for performing any of the methods or steps provided herein. In some embodiments, a kit can include a substrate with one or more capture probes comprising a spatial barcode and a capture domain that binds to a biological analyte from a tissue sample, and reagents to detect a biological analyte, wherein the biological analyte is any of the biomarkers of this disclosure. In some embodiments, the kit further includes but is not limited to one or more antibodies (and/or antigen-binding antibody fragments), labeled hybridization probes, primers, or any combination thereof for visualizing one or more features of a tissue sample.

### III. Embodiments

The following embodiments are provided herein.

Embodiment (“E” following by a number; e.g., E1, E2, E3, etc.)

E1. A method for identifying a candidate drug target for treatment of a brain disorder, the method comprising:
(a) determining level(s) of one or more biomarker(s) in a location in a sample comprising brain tissue obtained from an animal having a brain disorder;
(b) identifying:
(i) one or more biomarker(s) showing elevated level(s) in the location in the sample as compared to reference level(s) of the one or more biomarker(s) in a corresponding location in a sample comprising brain tissue obtained from a control animal, and/or
(ii) one or more biomarker(s) showing decreased level(s) in the location in the sample as compared to reference level(s) of the one or more biomarker(s) in a corresponding location in a sample comprising brain tissue obtained from a control animal, as candidate drug target(s) for treatment of the brain disorder.

E2. The method of E1, wherein the method comprises identifying one or more biomarker(s) showing elevated level(s) in the location in the sample as compared to reference level(s) of the one or more biomarker(s) in a corresponding location in a sample comprising brain tissue obtained from a control animal as candidate drug target(s) for treatment of the brain disorder.

E3. The method of E2, wherein the method further comprises testing the ability of an inhibitor of the expression and/or activity of the one or more identified candidate drug target(s) to treat the brain disorder in an animal.

E4. The method of E1, wherein the method comprises identifying one or more biomarker(s) showing decreased level(s) in the location in the sample as compared to reference level(s) of the one or more biomarker(s) in a corresponding location in a sample comprising brain tissue obtained from a control animal as candidate drug target(s) for treatment of the brain disorder.

E5. The method of E4, wherein the method further comprises testing the ability of an agent that increases the expression and/or activity of the one or more identified candidate drug target(s) to treat the brain disorder in an animal.

E6. A method for identifying a diagnostic biomarker of a brain disorder, the method comprising:
(a) determining level(s) of one or more biomarker(s) in a location in a sample comprising brain tissue obtained from an animal having a brain disorder;
(b) identifying:
(i) one or more biomarker(s) showing elevated level(s) in the location in the sample as compared to reference level(s) of the one or more biomarker(s) in a corresponding location in a sample comprising brain tissue obtained from a control animal, and/or
(ii) one or more biomarker(s) showing decreased level(s) in the location in the sample as compared to reference level(s) of the one or more biomarker(s) in a corresponding location in a sample comprising brain tissue obtained from a control animal, as diagnostic biomarker(s) of the brain disorder.

E7. The method of E6, wherein the method comprises identifying one or more biomarker(s) showing elevated level(s) in the location in the sample as compared to reference level(s) of the one or more biomarker(s) in a corresponding location in a sample comprising brain tissue obtained from a control animal as diagnostic biomarker(s) of the brain disorder.

E8. The method of E6, wherein the method comprises identifying one or more biomarker(s) showing decreased level(s) in the location in the sample as compared to reference level(s) of the one or more biomarker(s) in a corresponding location in a sample comprising brain tissue obtained from a control animal, as diagnostic biomarker(s) of the brain disorder.

E9. A method for identifying a candidate prognostic biomarker of a brain disorder, the method comprising:
(a) determining level(s) of one or more biomarker(s) in a location in a sample comprising brain tissue obtained from an animal having a brain disorder;
(b) identifying:
(i) one or more biomarker(s) showing elevated level(s) in the location in the sample as compared to reference level(s) of the one or more biomarker(s) in a corresponding location in a sample comprising brain tissue obtained from a control animal, and/or
(ii) one or more biomarker(s) showing decreased level(s) in the location in the sample as compared to reference level(s) of the one or more biomarker(s) in a corresponding location in a sample comprising brain tissue obtained from a control animal, as candidate prognostic biomarker(s) of the brain disorder.

E10. The method of E9, wherein the method comprises identifying one or more biomarker(s) showing elevated level(s) in the location in the sample as compared to reference level(s) of the one or more biomarker(s) in a corresponding location in a sample comprising brain tissue obtained from a control animal, as candidate prognostic biomarker(s) of the brain disorder.

E11. The method of E10, wherein the method further comprises performing an experiment to validate whether the one or more identified candidate prognostic biomarker(s) provides for an accurate assessment of the prognosis of the brain disorder in an animal.

E12. The method of E9, wherein the method comprises identifying one or more biomarker(s) showing decreased level(s) in the location in the sample as compared to reference level(s) of the one or more biomarker(s) in a corresponding location in a sample comprising brain tissue obtained from a control animal, as candidate prognostic biomarker(s) of the brain disorder.

E13. The method of E12, wherein the method further comprises performing an experiment to validate whether the one or more identified candidate prognostic biomarker(s) provides for an accurate assessment of the prognosis of the brain disorder in an animal.

E14. A method for determining a candidate biomarker for determining efficacy of a treatment of a brain disorder, wherein the method comprises:
(a) determining level(s) of one or more biomarker(s) in a location in a sample comprising brain tissue obtained from an animal having a brain disorder;
(b) identifying:
(i) one or more biomarker(s) showing elevated level(s) in the location in the sample as compared to reference level(s) of the one or more biomarker(s) in a corresponding location in a sample comprising brain tissue obtained from a control animal, and/or
(ii) one or more biomarker(s) showing decreased level(s) in the location in the sample as compared to reference level(s) of the one or more biomarker(s) in a corresponding location in a sample comprising brain tissue obtained from a control animal, as a candidate biomarker for determining efficacy of a treatment of the brain disorder.

E15. The method of E14, wherein the method comprises identifying one or more biomarker(s) showing elevated level(s) in the location in the sample as compared to reference level(s) of the one or more biomarker(s) in a corresponding location in a sample comprising brain tissue obtained from a control animal as candidate biomarker(s) for determining efficacy of a treatment of the brain disorder.

E16. The method of E15, wherein the method further comprises performing an experiment to validate whether the one or more identified candidate biomarker(s) provides for an accurate assessment of the efficacy of a treatment of the brain disorder in an animal.

E17. The method of E14, wherein the method comprises identifying one or more biomarker(s) showing decreased level(s) in the location in the sample as compared to reference level(s) of the one or more biomarker(s) in a corresponding location in a sample comprising brain tissue obtained from a control animal, as candidate biomarker(s) for determining efficacy of a treatment of a brain disorder.

E18. The method of E17, wherein the method further comprises performing an experiment to validate whether the one or more identified candidate biomarker(s) provides for an accurate assessment of the efficacy of a treatment of the brain disorder in an animal.

E19. The method of any one of E1-E18, wherein the location is in the hypothalamus, thalamus, hypothalamus, pineal gland, caudoputamen, cerebrum, pituitary gland, cerebellum, medulla oblongata, pons, midbrain, substantia nigra, lateral ventricles, olfactory bulb, entorhinal cortex, prefrontal cortex, temporal cortex, amygdala, and spinal cord.

E20. The method of any one of E1-E19, wherein the brain disorder is selected from the group consisting of: Alzheimer's disease, Parkinson's disease, dementia, brain cancer, epilepsy, seizures, stroke, transient ischemic attack, hypoxia, ataxia, Wilson's disease, Huntington's disease, traumatic brain injury, concussion, amyotrophic lateral sclerosis, Batten disease, Creutzfeldt-Jakob disease, meningitis, ciliopathies, and Joubert syndrome.

E21. The method of E20, wherein the brain cancer is glioblastoma. E22. The method of any one of E1-E21, wherein the animal is a mouse, a rat, a human, a nonhuman primate, a zebrafish, a naked mole rat, or a dog.

E23. The method of any one of E1-E22, wherein the control animal is a sex-matched, age-matched healthy animal or a population of sex-matched, age-matched healthy animals.

E24. The method of any one of E1-E23, wherein the one or more biomarkers is a nucleic acid.

E25. The method of E24, wherein the nucleic acid is DNA.

E26. The method of E24, wherein the nucleic acid is RNA.

E27. The method of E26, wherein the RNA is mRNA.

E28. The method of any one of E24-E27, wherein step (a) comprises:
delivering a probe to the sample, wherein the sample is affixed to a support, and the probe comprises (i) a sequence that binds specifically to one of the one or more biomarkers and (ii) a spatial barcode that identifies the location where the probe interacted with the sample; and
determining all or a portion of (i) the spatial barcode or a complement thereof, and (ii) the sequence that binds specifically to one of the biomarkers or a complement thereof, to determine the level of the biomarker in the location in the sample.

E29. The method of E28, wherein the determining step comprises sequencing.

E30. The method of E29, wherein the sequencing comprises high throughput sequencing.

E31. The method of any one of E1-E23, wherein the one or more biomarkers is a protein, a peptide, or a combination thereof.

E32. The method of E31, wherein step (a) comprises:
contacting the sample with a binding agent that specifically binds to one of the one or more biomarker(s), wherein the binding agent further comprises an oligonucleotide having a sequence; and
sequencing all or a portion of the sequence of the oligonucleotide or a complement thereof, from a probe specifically bound to the biomarker in the location of the sample, to determine the level of the biomarker in the location in the sample.

E33. The method of E32, wherein the binding agent comprises an antibody or an antigen-binding antibody fragment.

E34. The method of E32 or E33, wherein the sequencing comprises high throughput sequencing.

E35. A method of diagnosing a subject as having Alzheimer's disease, wherein the method comprises:
(a) determining a level of pro-melanin concentrating hormone (PMCH), or a byproduct or precursor or degradation product thereof, in a biological sample from a subject; and
(b) identifying a subject having an elevated level of PMCH, or a byproduct or precursor or degradation product thereof, in the biological sample as compared to a reference level of PMCH, or a byproduct or precursor or degradation product thereof, as having Alzheimer's disease.

E36. A method of diagnosing a subject as having Alzheimer's disease, wherein the method comprises:
(a) determining a level of aldo-keto reductase family 1, member E1 (Akr1e1), or a byproduct or precursor or degradation product thereof, in a biological sample from a subject; and
(b) identifying a subject having an elevated level of Akr1e1, or a byproduct or precursor or degradation product thereof, in the biological sample as compared to a reference level of Akr1e1, or a byproduct or precursor or degradation product thereof, as having Alzheimer's disease.

E37. A method of diagnosing a subject as having Alzheimer's disease, wherein the method comprises:
(a) determining a level of autophagy related 4C cysteine peptidase (Atg4c), or a byproduct or precursor or degradation product thereof, in a biological sample from a subject; and
(b) identifying a subject having an elevated level of Atg4c, or a byproduct or precursor or degradation product thereof, in the biological sample as compared to a reference level of Atg4c, or a byproduct or precursor or degradation product thereof, as having Alzheimer's disease.

E38. A method of diagnosing a subject as having Alzheimer's disease, wherein the method comprises:
(a) determining a level of Gm14296, or a byproduct or precursor or degradation product thereof, in a biological sample from a subject; and
(b) identifying a subject having an elevated level of Gm14296, or a byproduct or precursor or degradation product thereof, in the biological sample as compared to a reference level of Gm14296, or a byproduct or precursor or degradation product thereof, as having Alzheimer's disease.

E39. A method of diagnosing a subject as having Alzheimer's disease, wherein the method comprises:
(a) determining a level of hypocretin neuropeptide precursor (Hcrt), or a byproduct or precursor or degradation product thereof, in a biological sample from a subject; and
(b) identifying a subject having an elevated level of Hcrt, or a byproduct or precursor or degradation product thereof, in the biological sample as compared to a reference level of Hcrt, or a byproduct or precursor or degradation product thereof, as having Alzheimer's disease.

E40. A method of diagnosing a subject as having Alzheimer's disease, wherein the method comprises:
(a) determining a level of transthyretin (Ttr), or a byproduct or precursor or degradation product thereof, in a biological sample from a subject; and
(b) identifying a subject having an elevated level of Ttr, or a byproduct or precursor or degradation product thereof, in the biological sample as compared to a reference level of Ttr, or a byproduct or precursor or degradation product thereof, as having Alzheimer's disease.

E41. A method of diagnosing a subject as having Alzheimer's disease, wherein the method comprises:
(a) determining a level of adenosine A2a receptor (Adora2a), or a byproduct or precursor or degradation product thereof, in a biological sample from a subject; and
(b) identifying a subject having about the same or a decreased level of Adora2a, or a byproduct or precursor or degradation product thereof, in the biological sample as compared to a reference level of Adora2a, or a byproduct or precursor or degradation product thereof, as having Alzheimer's disease.

E42. A method of diagnosing a subject as having Alzheimer's disease, wherein the method comprises:
(a) determining a level of thyrotropin releasing hormone preproprotein (Trh), or a byproduct or precursor or degradation product thereof, in a biological sample from a subject; and
(b) identifying a subject having about the same or a decreased level of Trh, or a byproduct or precursor or degradation product thereof, in the biological sample as compared to a reference level of Trh, or a byproduct or precursor or degradation product thereof, as having Alzheimer's disease.

E43. The method of any one of E35-E42, wherein the method further comprises confirming a diagnosis of Alzheimer's disease in the subject by obtaining an image of the subject's brain, detecting a genetic mutation associated with Alzheimer's disease, or performing cognitive testing on the subject.

E44. The method of any one of E35-E43, wherein the method further comprises administering a treatment of Alzheimer's disease to the subject.

E45. A method of identifying a subject as having an increased likelihood of developing Alzheimer's disease, wherein the method comprises:
(a) determining a level of PMCH, or a byproduct or precursor or degradation product thereof, in a biological sample from a subject; and
(b) identifying a subject having an elevated level of PMCH, or a byproduct or precursor or degradation product thereof, in the biological sample as compared to a reference level of PMCH, or a byproduct or precursor or degradation product thereof, as having an increased likelihood of developing Alzheimer's disease.

E46. A method of identifying a subject as having an increased likelihood of developing Alzheimer's disease, wherein the method comprises:
(a) determining a level of Akr1e1, or a byproduct or precursor or degradation product thereof, in a biological sample from a subject; and
(b) identifying a subject having an elevated level of Akr1e1, or a byproduct or precursor or degradation product thereof, in the biological sample as compared to a reference level of Akr1e1, or a byproduct or precursor or degradation product thereof, as having an increased likelihood of developing Alzheimer's disease.

E47. A method of identifying a subject as having an increased likelihood of developing Alzheimer's disease, wherein the method comprises:
(a) determining a level of Atg4c, or a byproduct or precursor or degradation product thereof, in a biological sample from a subject; and
(b) identifying a subject having an elevated level of Atg4c, or a byproduct or precursor or degradation product thereof, in the biological sample as compared to a reference level of Atg4c, or a byproduct or precursor or degradation product thereof, as having an increased likelihood of developing Alzheimer's disease.

E48. A method of identifying a subject as having an increased likelihood of developing Alzheimer's disease, wherein the method comprises:
(a) determining a level of Gm14296, or a byproduct or precursor or degradation product thereof, in a biological sample from a subject; and
(b) identifying a subject having an elevated level of Gm14296, or a byproduct or precursor or degradation product thereof, in the biological sample as compared to a reference level of Gm14296, or a byproduct or precursor or degradation product thereof, as having an increased likelihood of developing Alzheimer's disease.

E49. A method of identifying a subject as having an increased likelihood of developing Alzheimer's disease, wherein the method comprises:
(a) determining a level of Hcrt, or a byproduct or precursor or degradation product thereof, in a biological sample from a subject; and
(b) identifying a subject having an elevated level of Hcrt, or a byproduct or precursor or degradation product thereof, in the biological sample as compared to a reference level of Hcrt, or a byproduct or precursor or degradation product thereof, as having an increased likelihood of developing Alzheimer's disease.

E50. A method of identifying a subject as having an increased likelihood of developing Alzheimer's disease, wherein the method comprises:
(a) determining a level of Ttr, or a byproduct or precursor or degradation product thereof, in a biological sample from a subject; and
(b) identifying a subject having an elevated level of Ttr, or a byproduct or precursor or degradation product thereof, in the biological sample as compared to a reference level of Ttr, or a byproduct or precursor or degradation product thereof, as having an increased likelihood of developing Alzheimer's disease.

E51. A method of identifying a subject as having an increased likelihood of developing Alzheimer's disease, wherein the method comprises:
(a) determining a level of Adora2a, or a byproduct or precursor or degradation product thereof, in a biological sample from a subject; and
(b) identifying a subject having about the same or a decreased level of Adora2a, or a byproduct or precursor or degradation product thereof, in the biological sample as compared to a reference level of Adora2a, or a byproduct or precursor or degradation product thereof, as having an increased likelihood of developing Alzheimer's disease.

E52. A method of identifying a subject as having an increased likelihood of developing Alzheimer's disease, wherein the method comprises:
(a) determining a level of Trh, or a byproduct or precursor or degradation product thereof, in a biological sample from a subject; and
(b) identifying a subject having about the same or a decreased level of Trh, or a byproduct or precursor or degradation product thereof, in the biological sample as compared to a reference level of Trh, or a byproduct or precursor or degradation product thereof, as having an increased likelihood of developing Alzheimer's disease.

E53. The method of any one of E35-E52, wherein the method further comprises monitoring the identified subject for the development of symptoms of Alzheimer's disease.

E54. The method of any one of E35-E52, wherein the method further comprises recording in the identified subject's clinical record that the subject has an increased likelihood or susceptibility of developing Alzheimer's disease.

E55. The method of any one of E35-E52, wherein the method further comprises notifying the subject's family that the subject has an increased likelihood or susceptibility of developing Alzheimer's disease.

E56. The method of any one of E35-E55, wherein the method further comprises administering to the subject a treatment for decreasing the rate of progression or decreasing the likelihood or susceptibility of developing Alzheimer's disease.

E57. The method of any one of E35-E56, wherein the biological sample comprises cerebrospinal fluid.

E58. The method of any one of E35-E57, further comprising obtaining the biological sample from the subject.

E59. The method of any one of E35-E58, wherein the level is a level of protein or a byproduct or precursor or degradation product thereof.

E60. The method of any one of E35-E58, wherein the level is a level of mRNA or a fragment thereof.

E61. A method of monitoring progression of Alzheimer's disease in a subject over time, wherein the method comprises:
(a) determining a first level of PMCH, or a byproduct or precursor or degradation product thereof, in a first biological sample obtained from a subject at a first time point;
(b) determining a second level of PMCH, or a byproduct or precursor or degradation product thereof, in a second biological sample obtained from the subject at a second time point;
(c) identifying:
(i) a subject having an increased second level of PMCH, or a byproduct or precursor or degradation product thereof, as compared to the first level of PMCH, or a byproduct or precursor or degradation product thereof, as having progressing Alzheimer's disease, or
(ii) a subject having about the same or a decreased second level of PMCH, or a byproduct or precursor or degradation product thereof as compared to the first level of PMCH, or a byproduct or precursor or degradation product thereof, as having static or regressing Alzheimer's disease.

E62. A method of monitoring progression of Alzheimer's disease in a subject over time, wherein the method comprises:
(a) determining a first level of Akr1e1, or a byproduct or precursor or degradation product thereof, in a first biological sample obtained from a subject at a first time point;
(b) determining a second level of Akr1e1, or a byproduct or precursor or degradation product thereof, in a second biological sample obtained from the subject at a second time point;
(c) identifying:
(i) a subject having an increased second level of Akr1e1, or a byproduct or precursor or degradation product thereof, as compared to the first level of Akr1e1, or a byproduct or precursor or degradation product thereof, as having progressing Alzheimer's disease, or
(ii) a subject having about the same or a decreased second level of Akr1e1, or a byproduct or precursor or degradation product thereof as compared to the first level of Akr1e1, or a byproduct or precursor or degradation product thereof, as having static or regressing Alzheimer's disease.

E63. A method of monitoring progression of Alzheimer's disease in a subject over time, wherein the method comprises:
(a) determining a first level of Atg4c, or a byproduct or precursor or degradation product thereof, in a first biological sample obtained from a subject at a first time point;
(b) determining a second level of Atg4c, or a byproduct or precursor or degradation product thereof, in a second biological sample obtained from the subject at a second time point;
(c) identifying:
(i) a subject having an increased second level of Atg4c, or a byproduct or precursor or degradation product thereof, as compared to the first level of Atg4c, or a byproduct or precursor or degradation product thereof, as having progressing Alzheimer's disease, or
(ii) a subject having about the same or a decreased second level of Atg4c, or a byproduct or precursor or degradation product thereof as compared to the first level of Atg4c, or a byproduct or precursor or degradation product thereof, as having static or regressing Alzheimer's disease.

E64. A method of monitoring progression of Alzheimer's disease in a subject over time, wherein the method comprises:
(a) determining a first level of Gm14296, or a byproduct or precursor or degradation product thereof, in a first biological sample obtained from a subject at a first time point;
(b) determining a second level of Gm14296, or a byproduct or precursor or degradation product thereof, in a second biological sample obtained from the subject at a second time point;
(c) identifying:
(i) a subject having an increased second level of Gm14296, or a byproduct or precursor or degradation product thereof, as compared to the first level of Gm14296, or a byproduct or precursor or degradation product thereof, as having progressing Alzheimer's disease, or
(ii) a subject having about the same or a decreased second level of Gm14296, or a byproduct or precursor or degradation product thereof as compared to the first level of Gm14296, or a byproduct or precursor or degradation product thereof, as having static or regressing Alzheimer's disease.

E65. A method of monitoring progression of Alzheimer's disease in a subject over time, wherein the method comprises:
(a) determining a first level of Hcrt, or a byproduct or precursor or degradation product thereof, in a first biological sample obtained from a subject at a first time point;
(b) determining a second level of Hcrt, or a byproduct or precursor or degradation product thereof, in a second biological sample obtained from the subject at a second time point;
(c) identifying:
(i) a subject having an increased second level of Hcrt, or a byproduct or precursor or degradation product thereof, as compared to the first level of Hcrt, or a byproduct or precursor or degradation product thereof, as having progressing Alzheimer's disease, or
(ii) a subject having about the same or a decreased second level of Hcrt, or a byproduct or precursor or degradation product thereof as compared to the first level of Hcrt, or a byproduct or precursor or degradation product thereof, as having static or regressing Alzheimer's disease.

E66. A method of monitoring progression of Alzheimer's disease in a subject over time, wherein the method comprises:
(a) determining a first level of Ttr, or a byproduct or precursor or degradation product thereof, in a first biological sample obtained from a subject at a first time point;
(b) determining a second level of Ttr, or a byproduct or precursor or degradation product thereof, in a second biological sample obtained from the subject at a second time point;
(c) identifying:
(i) a subject having an increased second level of Ttr, or a byproduct or precursor or degradation product thereof, as compared to the first level of Ttr, or a byproduct or precursor or degradation product thereof, as having progressing Alzheimer's disease, or
(ii) a subject having about the same or a decreased second level of Ttr, or a byproduct or precursor or degradation product thereof as compared to the first level of Ttr, or a byproduct or precursor or degradation product thereof, as having static or regressing Alzheimer's disease.

E67. A method of monitoring progression of Alzheimer's disease in a subject over time, wherein the method comprises:
(a) determining a first level of Adora2a, or a byproduct or precursor or degradation product thereof, in a first biological sample obtained from a subject at a first time point;
(b) determining a second level of Adora2a, or a byproduct or precursor or degradation product thereof, in a second biological sample obtained from the subject at a second time point;
(c) identifying:
(i) a subject having about the same or a decreased second level of Adora2a, or a byproduct or precursor or degradation product thereof, as compared to the first level of Adora2a, or a byproduct or precursor or degradation product thereof, as having progressing Alzheimer's disease, or
(ii) a subject having an increased second level of Adora2a, or a byproduct or precursor or degradation product thereof as compared to the first level of Adora2a, or a byproduct or precursor or degradation product thereof, as having static or regressing Alzheimer's disease.

E68. A method of monitoring progression of Alzheimer's disease in a subject over time, wherein the method comprises:
(a) determining a first level of Trh, or a byproduct or precursor or degradation product thereof, in a first biological sample obtained from a subject at a first time point;
(b) determining a second level of Trh, or a byproduct or precursor or degradation product thereof, in a second biological sample obtained from the subject at a second time point;
(c) identifying:
(i) a subject having about the same or a decreased second level of Trh, or a byproduct or precursor or degradation product thereof, as compared to the first level of Trh, or a byproduct or precursor or degradation product thereof, as having progressing Alzheimer's disease, or
(ii) a subject having an increased second level of Trh, or a byproduct or precursor or degradation product thereof as compared to the first level of Trh, or a byproduct or precursor or degradation product thereof, as having static or regressing Alzheimer's disease.

E69. The method of any one of E61-E68, wherein the method comprises identifying a subject as having progressing Alzheimer's disease.

E70. The method of E69, wherein the method further comprises administering a treatment for Alzheimer's disease to the subject or increasing the dose of a treatment for Alzheimer's disease to be administered to the subject.

E71. The method of E69, wherein the method further comprises assessing the subject for admittance into a care facility.

E72. The method of E69, wherein the method further comprises recording in the subject's clinical record that the subject has progressing Alzheimer's disease.

E73. The method of any one of E61-68, wherein the method comprises identifying a subject as having static or regressing Alzheimer's disease.

E74. The method of E73, wherein the method further comprises recording in the subject's clinical record that the subject has static or regressing Alzheimer's disease.

E75. A method of determining efficacy of treatment of a treatment for Alzheimer's disease in a subject, wherein the method comprises:
(a) determining a first level of PMCH, or a byproduct or precursor or degradation product thereof, in a first biological sample obtained from a subject at a first time point;
(b) determining a second level of PMCH, or a byproduct or precursor or degradation product thereof, in a second biological sample obtained from the subject at a second time point, wherein the subject is administered one or more doses of a therapeutic treatment between the first and second time points;
(c) identifying:
(i) the therapeutic treatment as being effective in a subject having about the same or a decreased second level of PMCH, or a byproduct or precursor or degradation product thereof, as compared to the first level of PMCH, or a byproduct or precursor or degradation product thereof, or
(ii) the therapeutic treatment as not being effective in a subject having an increased second level of PMCH, or a byproduct or precursor or degradation product thereof, as compared to the first level of PMCH, or a byproduct or precursor or degradation product thereof.

E76. A method of determining efficacy of treatment of a treatment for Alzheimer's disease in a subject, wherein the method comprises:
(a) determining a first level of Akr1e1, or a byproduct or precursor or degradation product thereof, in a first biological sample obtained from a subject at a first time point;
(b) determining a second level of Akr1e1, or a byproduct or precursor or degradation product thereof, in a second biological sample obtained from the subject at a second time point, wherein the subject is administered one or more doses of a therapeutic treatment between the first and second time points;
(c) identifying:
(i) the therapeutic treatment as being effective in a subject having about the same or a decreased second level of Akr1e1, or a byproduct or precursor or degradation product thereof, as compared to the first level of Akr1e1, or a byproduct or precursor or degradation product thereof, or
(ii) the therapeutic treatment as not being effective in a subject having an increased second level of Akr1e1, or a byproduct or precursor or degradation product thereof, as compared to the first level of Akr1e1, or a byproduct or precursor or degradation product thereof.

E77. A method of determining efficacy of treatment of a treatment for Alzheimer's disease in a subject, wherein the method comprises:
(a) determining a first level of Atg4c, or a byproduct or precursor or degradation product thereof, in a first biological sample obtained from a subject at a first time point;
(b) determining a second level of Atg4c, or a byproduct or precursor or degradation product thereof, in a second biological sample obtained from the subject at a second time point, wherein the subject is administered one or more doses of a therapeutic treatment between the first and second time points;
(c) identifying:
(i) the therapeutic treatment as being effective in a subject having about the same or a decreased second level of Atg4c, or a byproduct or precursor or degradation product thereof, as compared to the first level of Atg4c, or a byproduct or precursor or degradation product thereof, or
(ii) the therapeutic treatment as not being effective in a subject having an increased second level of Atg4c, or a byproduct or precursor or degradation product thereof, as compared to the first level of Atg4c, or a byproduct or precursor or degradation product thereof.

E78. A method of determining efficacy of treatment of a treatment for Alzheimer's disease in a subject, wherein the method comprises:
(a) determining a first level of Gm14296, or a byproduct or precursor or degradation product thereof, in a first biological sample obtained from a subject at a first time point;
(b) determining a second level of Gm14296, or a byproduct or precursor or degradation product thereof, in a second biological sample obtained from the subject at a second time point, wherein the subject is administered one or more doses of a therapeutic treatment between the first and second time points;
(c) identifying:
(i) the therapeutic treatment as being effective in a subject having about the same or a decreased second level of Gm14296, or a byproduct or precursor or degradation product thereof, as compared to the first level of Gm14296, or a byproduct or precursor or degradation product thereof, or
(ii) the therapeutic treatment as not being effective in a subject having an increased second level of Gm14296, or a byproduct or precursor or degradation product thereof, as compared to the first level of Gm14296, or a byproduct or precursor or degradation product thereof.

E79. A method of determining efficacy of treatment of a treatment for Alzheimer's disease in a subject, wherein the method comprises:
(a) determining a first level of Hcrt, or a byproduct or precursor or degradation product thereof, in a first biological sample obtained from a subject at a first time point;
(b) determining a second level of Hcrt, or a byproduct or precursor or degradation product thereof, in a second biological sample obtained from the subject at a second time point, wherein the subject is administered one or more doses of a therapeutic treatment between the first and second time points;
(c) identifying:
(i) the therapeutic treatment as being effective in a subject having about the same or a decreased second level of Hcrt, or a byproduct or precursor or degradation product thereof, as compared to the first level of Hcrt, or a byproduct or precursor or degradation product thereof, or
(ii) the therapeutic treatment as not being effective in a subject having an increased second level of Hcrt, or a byproduct or precursor or degradation product thereof, as compared to the first level of Hcrt, or a byproduct or precursor or degradation product thereof.

E80. A method of determining efficacy of treatment of a treatment for Alzheimer's disease in a subject, wherein the method comprises:
(a) determining a first level of Ttr, or a byproduct or precursor or degradation product thereof, in a first biological sample obtained from a subject at a first time point;
(b) determining a second level of Ttr, or a byproduct or precursor or degradation product thereof, in a second biological sample obtained from the subject at a second time point, wherein the subject is administered one or more doses of a therapeutic treatment between the first and second time points;
(c) identifying:
(i) the therapeutic treatment as being effective in a subject having about the same or a decreased second level of Ttr, or a byproduct or precursor or degradation product thereof, as compared to the first level of Ttr, or a byproduct or precursor or degradation product thereof, or
(ii) the therapeutic treatment as not being effective in a subject having an increased second level of Ttr, or a byproduct or precursor or degradation product thereof, as compared to the first level of Ttr, or a byproduct or precursor or degradation product thereof.

E81. A method of determining efficacy of treatment of a treatment for Alzheimer's disease in a subject, wherein the method comprises:
(a) determining a first level of Adora2a, or a byproduct or precursor or degradation product thereof, in a first biological sample obtained from a subject at a first time point;
(b) determining a second level of Adora2a, or a byproduct or precursor or degradation product thereof, in a second biological sample obtained from the subject at a second time point, wherein the subject is administered one or more doses of a therapeutic treatment between the first and second time points;
(c) identifying:
(i) the therapeutic treatment as being effective in a subject having an increased level of Adora2a, or a byproduct or precursor or degradation product thereof, as compared to the first level of Adora2a, or a byproduct or precursor or degradation product thereof, or
(ii) the therapeutic treatment as not being effective in a subject having about the same or a decreased second level of Adora2a, or a byproduct or precursor or degradation product thereof, as compared to the first level of Adora2a, or a byproduct or precursor or degradation product thereof.

E82. A method of determining efficacy of treatment of a treatment for Alzheimer's disease in a subject, wherein the method comprises:
(a) determining a first level of Trh, or a byproduct or precursor or degradation product thereof, in a first biological sample obtained from a subject at a first time point;
(b) determining a second level of Trh, or a byproduct or precursor or degradation product thereof, in a second biological sample obtained from the subject at a second time point, wherein the subject is administered one or more doses of a therapeutic treatment between the first and second time points;
(c) identifying:
(i) the therapeutic treatment as being effective in a subject having an increased level of Trh, or a byproduct or precursor or degradation product thereof, as compared to the first level of Trh, or a byproduct or precursor or degradation product thereof, or
(ii) the therapeutic treatment as not being effective in a subject having about the same or a decreased second level of Trh, or a byproduct or precursor or degradation product thereof, as compared to the first level of Trh, or a byproduct or precursor or degradation product thereof.

E83. The method of any one of E75-E82, wherein the method comprises identifying the therapeutic treatment as being effective in the subject.

E84. The method of E83, wherein the method further comprises selecting additional doses of the therapeutic treatment for the subject.

E85. The method of E83, wherein the method further comprises administering additional doses of the therapeutic treatment to the subject.

E86. The method of E83, wherein the method further comprises recording in the subject's clinical record that the therapeutic treatment is effective in the subject.

E87. The method of any one of E75-82, wherein the method comprises identifying the therapeutic treatment as not being effective in the subject.

E88. The method of E87, wherein the method further comprises selecting a different therapeutic treatment for the subject.

E89. The method of E87, wherein the method further comprises administering a different therapeutic treatment to the subject.

E90. The method of E87, wherein the method further comprises increasing the dose of the therapeutic treatment to be administered to the subject.

E91. The method of E87, wherein the method further comprises administering one or more additional doses of the therapeutic treatment to the subject in combination with an additional therapeutic treatment.

E92. The method of any one of E61-E91, wherein the first and second biological samples comprise cerebrospinal fluid.

E93. The method of any one of E61-E92, further comprising obtaining the first and second biological samples from the subject.

E94. The method of any one of E61-E93, wherein each of the first and second level is a level of protein or a byproduct or precursor or degradation product thereof.

E95. The method of any one of E61-E93, wherein each of the first and second level is a level of mRNA or a fragment thereof.

E96. A kit comprising:

an antibody that binds specifically to PMCH, Adora2a, Trh, Akr1e1, Atg4c, Gm14296, Hcrt, Ttr, or a byproduct or precursor or degradation product thereof; and

instructions for performing the method of any one of E35-E95.

E97. The method of any one of E1, E6, E9, E14, E35-E42, and E45-E52, wherein the sample comprises at least one tissue section.

E98. The method of any one of E1, E6, E9, E14, E35-E42, and E45-E52, wherein the sample comprises serial tissue sections.

E99. The method of any one of E1, E6, E9, E14, E35-E42, and E45-E52, wherein the sample comprises a tissue structure.

E100. A method of identifying a patient subpopulation for which a therapeutic treatment is effective for Alzheimer's disease, the method comprising:
(a) administering a therapeutic treatment for Alzheimer's disease to a patient subpopulation;
(b) determining (i) a first level of PMCH, or a byproduct or precursor or degradation product thereof, in first biological samples obtained from a patient subpopulation at a first time point and (ii) a second level of PMCH, or a byproduct, precursor, or degradation product thereof, in second biological samples obtained from the patient population at a second time point, wherein the patient subpopulation is administered one or more doses of a therapeutic treatment between the first and second time points;
(c) determining a correlation between efficacy of the therapeutic treatment and the second level in samples from the patient subpopulation as compared a sample obtained from an untreated patient, wherein a lower second level in the samples from the patient subpopulation as compared to the sample from the untreated patient is indicative that the therapeutic treatment is effective for Alzheimer's disease in the patient subpopulation.

E101. The method of E100, wherein the therapeutic treatment is an antagonist of PMCH, or a byproduct or precursor or degradation product thereof.

E102. A method of identifying a patient subpopulation for which a therapeutic treatment is effective for Alzheimer's disease, the method comprising:
(a) administering a therapeutic treatment for Alzheimer's disease to a patient subpopulation;
(b) determining (i) a first level of Akr1e1, or a byproduct or precursor or degradation product thereof, in first biological samples obtained from a patient subpopulation at a first time point and (ii) a second level of Akr1e1, or a byproduct, precursor, or degradation product thereof, in second biological samples obtained from the patient population at a second time point, wherein the patient subpopulation is administered one or more doses of a therapeutic treatment between the first and second time points;
(c) determining a correlation between efficacy of the therapeutic treatment and the second level in samples from the patient subpopulation as compared a sample obtained from an untreated patient, wherein a lower second level in the samples from the patient subpopulation as compared to the sample from the untreated patient is indicative that the therapeutic treatment is effective to Alzheimer's disease in the patient subpopulation.

E103. The method of E102, wherein the therapeutic treatment is an antagonist of Akr1e1, or a byproduct or precursor or degradation product thereof.

E104. A method of identifying a patient subpopulation for which a therapeutic treatment is effective for Alzheimer's disease, the method comprising:
(a) administering a therapeutic treatment for Alzheimer's disease to a patient subpopulation;
(b) determining (i) a first level of Atg4c, or a byproduct or precursor or degradation product thereof, in first biological samples obtained from a patient subpopulation at a first time point and (ii) a second level of Atg4c, or a byproduct, precursor, or degradation product thereof, in second biological samples obtained from the patient population at a second time point, wherein the patient subpopulation is administered one or more doses of a therapeutic treatment between the first and second time points;
(c) determining a correlation between efficacy of the therapeutic treatment and the second level in samples from the patient subpopulation as compared a sample obtained from an untreated patient, wherein a lower second level in the samples from the patient subpopulation as compared to the sample from the untreated patient is indicative that the therapeutic treatment is effective to Alzheimer's disease in the patient subpopulation.

E105. The method of E104, wherein the therapeutic treatment is an antagonist of Atg4c, or a byproduct or precursor or degradation product thereof.

E106. A method of identifying a patient subpopulation for which a therapeutic treatment is effective for Alzheimer's disease, the method comprising:
(a) administering a therapeutic treatment for Alzheimer's disease to a patient subpopulation;
(b) determining (i) a first level of Gm14296, or a byproduct or precursor or degradation product thereof, in first biological samples obtained from a patient subpopulation at a first time point and (ii) a second level of Gm14296, or a byproduct, precursor, or degradation product thereof, in second biological samples obtained from the patient population at a second time point, wherein the patient subpopulation is administered one or more doses of a therapeutic treatment between the first and second time points;
(c) determining a correlation between efficacy of the therapeutic treatment and the second level in samples from the patient subpopulation as compared a sample obtained from an untreated patient, wherein a lower second level in the samples from the patient subpopulation as compared to the sample from the untreated patient is indicative that the therapeutic treatment is effective to Alzheimer's disease in the patient subpopulation.

E107. The method of E104, wherein the therapeutic treatment is an antagonist of Gm14296, or a byproduct or precursor or degradation product thereof.

E108. A method of identifying a patient subpopulation for which a therapeutic treatment is effective for Alzheimer's disease, the method comprising:
(a) administering a therapeutic treatment for Alzheimer's disease to a patient subpopulation;
(b) determining (i) a first level of Hcrt, or a byproduct or precursor or degradation product thereof, in first biological samples obtained from a patient subpopulation at a first time point and (ii) a second level of Hcrt, or a byproduct, precursor, or degradation product thereof, in second biological samples obtained from the patient population at a second time point, wherein the patient subpopulation is administered one or more doses of a therapeutic treatment between the first and second time points;
(c) determining a correlation between efficacy of the therapeutic treatment and the second level in samples from the patient subpopulation as compared a sample obtained from an untreated patient, wherein a lower second level in the samples from the patient subpopulation as compared to the sample from the untreated patient is indicative that the therapeutic treatment is effective to Alzheimer's disease in the patient subpopulation.

E109. The method of E108, wherein the therapeutic treatment is an antagonist of Hcrt, or a byproduct or precursor or degradation product thereof.

E110. A method of identifying a patient subpopulation for which a therapeutic treatment is effective for Alzheimer's disease, the method comprising:
(a) administering a therapeutic treatment for Alzheimer's disease to a patient subpopulation;
(b) determining (i) a first level of Ttr, or a byproduct or precursor or degradation product thereof, in first biological samples obtained from a patient subpopulation at a first time point and
(ii) a second level of Ttr, or a byproduct, precursor, or degradation product thereof, in second biological samples obtained from the patient population at a second time point, wherein the patient subpopulation is administered one or more doses of a therapeutic treatment between the first and second time points;
(c) determining a correlation between efficacy of the therapeutic treatment and the second level in samples from the patient subpopulation as compared a sample obtained from an untreated patient, wherein a lower second level in the samples from the patient subpopulation as compared to the sample from the untreated patient is indicative that the therapeutic treatment is effective to Alzheimer's disease in the patient subpopulation.

E111. The method of E110, wherein the therapeutic treatment is an antagonist of Ttr, or a byproduct or precursor or degradation product thereof.

E112. A method of identifying a patient subpopulation for which a therapeutic treatment is effective for Alzheimer's disease, the method comprising:
(a) administering a therapeutic treatment for Alzheimer's disease to a patient subpopulation;
(b) determining (i) a first level of Adora2a, or a byproduct or precursor or degradation product thereof, in first biological samples obtained from a patient subpopulation at a first time point and (ii) a second level of Adora2a, or a byproduct, precursor, or degradation product thereof, in second biological samples obtained from the patient population at a second time point, wherein the patient subpopulation is administered one or more doses of a therapeutic treatment between the first and second time points;
(c) determining a correlation between efficacy of the therapeutic treatment and the second level in samples from the patient subpopulation as compared a sample obtained from an untreated patient, wherein about the same or an elevated second level in the samples from the patient subpopulation as compared to the sample from the untreated patient is indicative that the therapeutic treatment is effective to Alzheimer's disease in the patient subpopulation.

E113. The method of E112, wherein the therapeutic treatment is an agonist of Adora2a, or a byproduct or precursor or degradation product thereof.

E114. A method of identifying a patient subpopulation for which a therapeutic treatment is effective for Alzheimer's disease, the method comprising:
(a) administering a therapeutic treatment for Alzheimer's disease to a patient subpopulation;
(b) determining (i) a first level of Trh, or a byproduct or precursor or degradation product thereof, in first biological samples obtained from a patient subpopulation at a first time point and (ii) a second level of Trh, or a byproduct, precursor, or degradation product thereof, in second biological samples obtained from the patient population at a second time point, wherein the patient subpopulation is administered one or more doses of a therapeutic treatment between the first and second time points;
(c) determining a correlation between efficacy of the therapeutic treatment and the second level in samples from the patient subpopulation as compared a sample obtained from an untreated patient, wherein about the same or an elevated second level in the samples from the patient subpopulation as compared to the sample from the untreated patient is indicative that the therapeutic treatment is effective to Alzheimer's disease in the patient subpopulation.

E115. The method of E114, wherein the therapeutic treatment is an agonist of Trh, or a byproduct or precursor or degradation product thereof.

E116. A method of modifying treatment of an Alzheimer's patient with a therapeutic treatment, the method comprising:
(a) administering a therapeutic treatment to an Alzheimer's patient;
(b) determining (i) a pre-treatment level of PMCH, or a byproduct or precursor or degradation product thereof, in a pre-treatment sample obtained from the Alzheimer's patient before treatment and (ii) a post-treatment level of PMCH, or a byproduct or precursor or degradation product thereof, in a post-treatment sample obtained from the Alzheimer's patient after treatment, wherein a higher level of PMCH, or a byproduct, precursor, or degradation product thereof, in the post-treatment sample, as compared to a level of PMCH, or a byproduct, precursor, or degradation product thereof, in a pre-treatment sample, is indicative of the responsiveness to treatment with the therapeutic treatment; and
(c) increasing the amount of the therapeutic treatment administered to the patient based on the higher level of PMCH, or a byproduct, precursor, or degradation product.

E117. The method of E116, wherein the therapeutic treatment is an antagonist of PMCH, or a byproduct or precursor or degradation product thereof.

E118. A method of modifying treatment of an Alzheimer's patient with a therapeutic treatment, the method comprising:
(a) administering a therapeutic treatment to an Alzheimer's patient;
(b) determining (i) a pre-treatment level of Akr1e1, or a byproduct or precursor or degradation product thereof, in a pre-treatment sample obtained from the Alzheimer's patient before treatment and (ii) a post-treatment level of Akr1e1, or a byproduct or precursor or degradation product thereof, in a post-treatment sample obtained from the Alzheimer's patient after treatment, wherein a higher level of Akr1e1, or a byproduct, precursor, or degradation product thereof, in the post-treatment sample, as compared to a level of Akr1e1, or a byproduct, precursor, or degradation product thereof, in a pre-treatment sample, is indicative of the responsiveness to treatment with the therapeutic treatment; and
(c) increasing the amount of the therapeutic treatment administered to the patient based on the higher level of Akr1e1 or a byproduct, precursor, or degradation product.

E119. The method of E118, wherein the therapeutic treatment is an antagonist of Akr1e1, or a byproduct or precursor or degradation product thereof.

E120. A method of modifying treatment of an Alzheimer's patient with a therapeutic treatment, the method comprising:
(a) administering a therapeutic treatment to an Alzheimer's patient;
(b) determining (i) a pre-treatment level of Atg4c, or a byproduct or precursor or degradation product thereof, in a pre-treatment sample obtained from the Alzheimer's patient before treatment and (ii) a post-treatment level of Atg4c, or a byproduct or precursor or degradation product thereof, in a post-treatment sample obtained from the Alzheimer's patient after treatment, wherein a higher level of Atg4c, or a byproduct, precursor, or degradation product thereof, in the post-treatment sample, as compared to a level of Atg4c, or a byproduct, precursor, or degradation product thereof, in a pre-treatment sample, is indicative of the responsiveness to treatment with the therapeutic treatment; and
(c) increasing the amount of the therapeutic treatment administered to the patient based on the higher level of Atg4c or a byproduct, precursor, or degradation product.

E121. The method of E120, wherein the therapeutic treatment is an antagonist of Atg4c, or a byproduct or precursor or degradation product thereof.

E122. A method of modifying treatment of an Alzheimer's patient with a therapeutic treatment, the method comprising:
(a) administering a therapeutic treatment to an Alzheimer's patient;
(b) determining (i) a pre-treatment level of Gm14296, or a byproduct or precursor or degradation product thereof, in a pre-treatment sample obtained from the Alzheimer's patient before treatment and (ii) a post-treatment level of Gm14296, or a byproduct or precursor or degradation product thereof, in a post-treatment sample obtained from the Alzheimer's patient after treatment, wherein a higher level of Gm14296, or a byproduct, precursor, or degradation product thereof, in the post-treatment sample, as compared to a level of Gm14296, or a byproduct, precursor, or degradation product thereof, in a pre-treatment sample, is indicative of the responsiveness to treatment with the therapeutic treatment; and
(c) increasing the amount of the therapeutic treatment administered to the patient based on the higher level of Gm14296 or a byproduct, precursor, or degradation product.

E123. The method of E122, wherein the therapeutic treatment is an antagonist of Gm14296, or a byproduct or precursor or degradation product thereof.

E124. A method of modifying treatment of an Alzheimer's patient with a therapeutic treatment, the method comprising:
(a) administering a therapeutic treatment to an Alzheimer's patient;
(b) determining (i) a pre-treatment level of Hcrt, or a byproduct or precursor or degradation product thereof, in a pre-treatment sample obtained from the Alzheimer's patient before treatment and (ii) a post-treatment level of Hcrt, or a byproduct or precursor or degradation product thereof, in a post-treatment sample obtained from the Alzheimer's patient after treatment, wherein a higher level of Hcrt, or a byproduct, precursor, or degradation product thereof, in the post-treatment sample, as compared to a level of Hcrt, or a byproduct, precursor, or degradation product thereof, in a pre-treatment sample, is indicative of the responsiveness to treatment with the therapeutic treatment; and
(c) increasing the amount of the therapeutic treatment administered to the patient based on the higher level of Hcrt or a byproduct, precursor, or degradation product.

E125. The method of E124, wherein the therapeutic treatment is an antagonist of Hcrt, or a byproduct or precursor or degradation product thereof.

E126. A method of modifying treatment of an Alzheimer's patient with a therapeutic treatment, the method comprising:
(a) administering a therapeutic treatment to an Alzheimer's patient;
(b) determining (i) a pre-treatment level of Ttr, or a byproduct or precursor or degradation product thereof, in a pre-treatment sample obtained from the Alzheimer's patient before treatment and (ii) a post-treatment level of Ttr, or a byproduct or precursor or degradation product thereof, in a post-treatment sample obtained from the Alzheimer's patient after treatment, wherein a higher level of Ttr, or a byproduct, precursor, or degradation product thereof, in the post-treatment sample, as compared to a level of Ttr, or a byproduct, precursor, or degradation product thereof, in a pre-treatment sample, is indicative of the responsiveness to treatment with the therapeutic treatment; and
(c) increasing the amount of the therapeutic treatment administered to the patient based on the higher level of Ttr or a byproduct, precursor, or degradation product.

E127. The method of E126, wherein the therapeutic treatment is an antagonist of Ttr, or a byproduct or precursor or degradation product thereof.

E128. A method of modifying treatment of an Alzheimer's patient with a therapeutic treatment, the method comprising:
(a) administering a therapeutic treatment to an Alzheimer's patient;
(b) determining (i) a pre-treatment level of Adora2a, or a byproduct or precursor or degradation product thereof, in a pre-treatment sample obtained from the Alzheimer's patient before treatment and (ii) a post-treatment level of Adora2a, or a byproduct or precursor or degradation product thereof, in a post-treatment sample obtained from the Alzheimer's patient after treatment, wherein a decreased level of Adora2a, or a byproduct, precursor, or degradation product thereof, in the post-treatment sample, as compared to a level of Adora2a, or a byproduct, precursor, or degradation product thereof, in a pre-treatment sample, is indicative of the responsiveness to treatment with the therapeutic treatment; and
(c) increasing the amount of the therapeutic treatment administered to the patient based on the decreased level of Adora2a or a byproduct, precursor, or degradation product.

E129. The method of E128, wherein the therapeutic treatment is an agonist of Adora2a, or a byproduct or precursor or degradation product thereof.

E130. A method of modifying treatment of an Alzheimer's patient with a therapeutic treatment, the method comprising:
(a) administering a therapeutic treatment to an Alzheimer's patient;
(b) determining (i) a pre-treatment level of Trh, or a byproduct or precursor or degradation product thereof, in a pre-treatment sample obtained from the Alzheimer's patient before treatment and (ii) a post-treatment level of Trh, or a byproduct or precursor or degradation product thereof, in a post-treatment sample obtained from the Alzheimer's patient after treatment, wherein a decreased level of Trh, or a byproduct, precursor, or degradation product thereof, in the post-treatment sample, as compared to a level of Trh, or a byproduct, precursor, or degradation product thereof, in a pre-treatment sample, is indicative of the responsiveness to treatment with the therapeutic treatment; and
(c) increasing the amount of the therapeutic treatment administered to the patient based on the decreased level of Trh or a byproduct, precursor, or degradation product.

E131. The method of E130, wherein the therapeutic treatment is an agonist of Trh, or a byproduct or precursor or degradation product thereof.

E132. A method of differentiating cell types in a biological sample comprising:
(a) contacting the biological sample with a plurality of capture probes, wherein a capture probe comprises a capture domain and a spatial barcode having a sequence;
(b) releasing nucleic acids from the biological sample, wherein members of the released nucleic acids are specifically bound by the capture domain(s);
(c) determining, for the nucleic acids that are specifically bound by the capture domain(s), (1) all or a portion of a sequence of the spatial barcode, or a complement thereof, and (2) all or a portion of a sequence of the nucleic acid, or a complement thereof, and using the determined sequences of (1) and (2) to identify the location and amount of the nucleic acid(s) in the biological sample;
(d) comparing the determined location and amount of the nucleic acids at a plurality of different locations in the biological sample; and
(e) sorting a subset of the nucleic acids of (d) into a cluster based on the determined location and amount of the nucleic acids at the plurality of different locations in the biological sample, and using the cluster(s) to differentiate cell types in the biological sample.

E133. A method of generating an image of a biological sample comprising:
(a) contacting the biological sample with a plurality of capture probes, wherein a capture probe comprises a capture domain and a spatial barcode having a sequence;
(b) releasing nucleic acids from the biological sample, wherein members of the released nucleic acids are specifically bound by the capture domain(s);
(c) determining, for the nucleic acids that are specifically bound by the capture domain(s), (1) all or a portion of a sequence of the spatial barcode, or a complement thereof, and (2) all or a portion of a sequence of the nucleic acid, or a complement thereof, and using the determined sequences of (1) and (2) to identify the location and amount of the nucleic acids in the biological sample;
(d) comparing the determined location and amount of the nucleic acids at a plurality of different locations in the biological sample; and
(e) sorting a subset of the nucleic acids of (d) into a cluster based on the determined location and amount of the nucleic acids at the plurality of different locations in the biological sample, and using the cluster(s) to biological sample to generate an image of the biological sample.

E134. A method of detecting molecular heterogeneity in a biological sample comprising:
(a) contacting a biological sample from the subject with a plurality of capture probes, wherein a capture probe comprises a capture domain and a spatial barcode having a sequence;
(b) releasing nucleic acids from the biological sample, wherein members of the released nucleic acids are specifically bound by the capture domain(s);
(c) determining, for the nucleic acids that are specifically bound by the capture domain(s), (1) all or a portion of a sequence of the spatial barcode, or a complement thereof, and (2) all or a portion of a sequence of the nucleic acid or a complement thereof, and using the determined sequences of (1) and (2) to identify the location and amount of the nucleic acids in the biological sample;
(d) comparing the determined location and amount of the nucleic acids at a plurality of different locations in the biological sample; and
(e) sorting a subset of the nucleic acids of (d) into a cluster based on the determined location and amount of the nucleic acids at the plurality of different locations in the biological sample, and using the cluster(s) to identify molecular heterogeneity in the biological sample relative to a reference biological sample.

E135. A method of identifying a subject as having abnormal gene expression in at least one tissue comprising:
(a) contacting a biological sample obtained from the subject with a plurality of capture probes, wherein a capture probe comprises a capture domain and a spatial barcode having a sequence;
(b) releasing nucleic acids from the biological sample, wherein members of the released nucleic acids are specifically bound by the capture domain(s);
(c) determining, for the nucleic acids that are specifically bound by the capture domain(s), (1) all or a portion of a sequence of the spatial barcode, or a complement thereof, and (2) all or a portion of a sequence of the nucleic acid, or a complement thereof, and using the determined sequences of (1) and (2) to identify the location and amount of the nucleic acids in the biological sample;
(d) comparing the determined location and amount of the nucleic acids at a plurality of different locations in the biological sample; and
(e) sorting a subset of the nucleic acids of (d) into a cluster based on the determined location and amount of the nucleic acids at the plurality of different locations in the biological sample, and using the cluster(s) to identify at least one region in the biological sample with abnormal gene expression relative to a reference biological sample.

E136. The method of E134 or E135, wherein the amount of one or more nucleic acids falls outside a predetermined threshold.

E137. A method of identifying a subject as having a cellular anomaly comprising:
(a) contacting a biological sample from the subject with a plurality of capture probes, wherein a capture probe comprises a capture domain and a spatial barcode having a sequence;
(b) releasing nucleic acids from the biological sample, wherein members of the released nucleic acids are specifically bound by the capture domain(s);
(c) determining, for the nucleic acids that are specifically bound by the capture domain(s), (1) all or a portion of a sequence of the spatial barcode, or a complement thereof, and (2) all or a portion of a sequence of the nucleic acid, or a complement thereof, and using the determined sequences of (1) and (2) to identify the location and amount of the nucleic acids in the biological sample;
(d) comparing the determined location and amount of the nucleic acids at a plurality of different locations in the biological sample; and
(e) sorting a subset of the nucleic acids of (d) into a cluster based on the determined location and amounts of the nucleic acids at the plurality of different locations in the biological sample, and using the cluster(s) to identify at least one cellular anomaly in the biological sample.

E138. A method of assessing the efficacy of a treatment or therapy in a subject comprising:
(a) contacting a biological sample from the subject with a plurality of capture probes, wherein a capture probe comprises a capture domain and a spatial barcode having a sequence;
(b) releasing nucleic acids from the biological sample, wherein members of the released nucleic acids are specifically bound by the capture domain(s);
(c) determining, for the nucleic acids that are specifically bound by the capture domain(s), (1) all or a portion of a sequence of the spatial barcode, or a complement thereof, and (2) all or a portion of a sequence of the nucleic acid, or a complement thereof, and using the determined sequences of (1) and (2) to identify the location and amount of the nucleic acids in the biological sample;
(d) comparing the determined location and amount of the nucleic acids at a plurality of different locations in the biological sample; and
(e) sorting a subset of the nucleic acids of (d) into a cluster based on the determined location and amount of the nucleic acids at the plurality of different locations in the biological sample, and using the cluster(s) to identify at least one region in the biological sample having restored gene expression.

E139. A method of comparing at least two biological samples comprising:
(a) contacting the first biological sample with a plurality of capture probes, wherein a capture probe comprises a capture domain and a spatial barcode having a sequence;
(b) releasing nucleic acids from the biological sample, wherein members of the released nucleic acids are specifically bound by the capture domain(s);
(c) determining, for the nucleic acids that are specifically bound by the capture domain(s), (1) all or a portion of a sequence of the spatial barcode, or a complement thereof, and (2) all or a portion of a sequence of the nucleic acid, or a complement thereof, and using the determined sequences of (1) and (2) to identify the location and amount of the nucleic acids in the biological sample;
(d) comparing the determined location and amount of nucleic acids at a plurality of different locations in the biological sample;
(e) sorting a subset of the nucleic acids of (d) into a first set of clusters based on the determined location and amount of nucleic acids at the plurality of different locations in the biological sample, and using the clusters to differentiate cell types in the biological sample;
(f) performing steps (a) to (e) on a second biological sample to identify a second set of clusters; and
(g) comparing the two sets of clusters.

E140. The method of E139, wherein the first biological sample is from the same subject as the second biological sample.

E141. The method of E140, wherein there is a period of time between acquiring the first biological sample and acquiring the second biological or subsequent samples from the subject.

E142. The method of E141, wherein the period of time is about 1 month to about two years.

E143. The method of 142, wherein the period of time is about 1 year.

E144. The method of any one of E139-E143, wherein the method further comprises comparing the clusters from additional biological samples obtained from the subject before and after the period of time.

E145. The method of E138, wherein the first biological sample is obtained from a first subject and the second biological sample is obtained from a second subject.

E146. The method of E145, wherein the second biological sample is obtained from a healthy subject.

E147. The method of E145 or E146, wherein the first biological sample is obtained from a subject at risk of developing a disease.

E148. A method comprising:
(a) contacting a biological sample obtained from a subject with a plurality of capture probes, wherein a capture probe comprises a capture domain and a spatial barcode having a sequence;
(b) releasing nucleic acids from the biological sample, wherein members of the released nucleic acids are specifically bound by the capture domain(s);
(c) determining, for the nucleic acids that are specifically bound by the capture domain(s), (1) all or a portion of a sequence of the spatial barcode, or a complement thereof, and (2) all or a portion of a sequence of the nucleic acid, or a complement thereof, and using the determined sequences of (1) and (2) to identify the location and amount of the nucleic acids in the biological sample;
(d) comparing the determined location and amount of nucleic acids at a plurality of different locations in the biological sample;
(e) sorting a subset of the nucleic acids of (d) into a set of clusters based on the determined location and amount of nucleic acids at the plurality of different locations in the biological sample, and using the clusters to differentiate cell types in the biological sample; and
(f) comparing the set of clusters to a reference set of clusters.

E149. The method of E148, wherein the reference set of clusters is a normalized set of clusters from more than one reference biological sample.

E150. The method of E149, wherein each of the more than one reference biological sample comprises the same type of tissue as the biological sample obtained from the subject.

E151. The method of any one of E133-E150, wherein the cluster is identified using nonlinear dimensionality reduction.

E152. The method of any one of E133-E150, wherein the cluster is identified using t-distributed stochastic neighbor embedding (t-SNE).

E153. The method of any one of E133-E150, wherein the cluster is identified using global t-distributed stochastic neighbor embedding (g-SNE).

E154. The method of any one of E133-E150, wherein the cluster is identified using uniform manifold approximation and projection (UMAP).

E155. The method of any one of E133-E154, wherein 2 to 200 clusters are identified.

E156. The method of any one of E133-E154, wherein 2 to 10 clusters are identified.

E157. The method of any one of E133-E156, wherein a cluster consists of about 2 to about 25,000 genes.

E158. The method of any one of E133-E157, wherein the method further comprises identifying a subpopulation of cells in the biological sample.

E159. The method of any one of E133-E158, wherein the biological sample comprises epithelial tissue, a connective tissue, a muscle tissue, an adipose tissue, a nervous tissue, an embryonic tissue, or a combination thereof.

E160. The method of any one of E133-E158, wherein the biological sample comprises brain tissue, a spinal cord tissue, a skin tissue, an adipose tissue, an intestinal tissue, a colon tissue, a cervical tissue, a vaginal tissue, a muscle tissue, a cardiac tissue, a liver tissue, a pancreatic tissue, a kidney tissue, a spleen tissue, a lymph node tissue, a bone marrow tissue, a cartilage tissue, a retinal tissue, a corneal tissue, a breast tissue, a prostate tissue, a bladder tissue, a tracheal tissue, a lung tissue, a uterine tissue, a stomach tissue, a thyroid tissue, a thymus tissue, or a combination thereof.

E161. The method of any one of E133-E160, wherein the biological sample is obtained from a biopsy.

E162. The method of any one of E133-E160, wherein the biological sample is obtained from a surgical excision.

E163. The method of E159 or E160, wherein the biological sample was collected during an endoscopy or colposcopy.

E164. The method of any one of E133-E163, wherein the biological sample is a frozen tissue sample.

E165. The method of any one of E133-E163, wherein the biological sample is a formalin-fixed, paraffin-embedded (FFPE) sample.

E166. The method of any one of E133-E165, wherein the nucleic acid is DNA.

E167. The method of E166, wherein the DNA is genomic DNA.

E168. The method of E166, wherein the DNA is mitochondrial DNA.

E169. The method of any one of E133-E165, wherein the nucleic acid is RNA.

E170. The method of E169, wherein the RNA is mRNA.

E171. A method of diagnosing a subject as having glioblastoma, wherein the method comprises:
(a) determining a level of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, or SPP1, or a byproduct or precursor or degradation product thereof, in a biological sample from a subject; and
(b) identifying a subject having an elevated level of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, or SPP1, in the biological sample as compared to a reference level, as having glioblastoma.

E172. A method of diagnosing a subject as having glioblastoma, wherein the method comprises:
(a) determining a level of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, or MT-ND5, or a byproduct or precursor or degradation product thereof, in a biological sample from a subject; and
(b) identifying a subject having a decreased level of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, and MT-ND5, or a byproduct or precursor or degradation product thereof, in the biological sample as compared to a reference level, as having glioblastoma.

E173. The method of E171 or E172, wherein the method further comprises confirming a diagnosis of gliobastoma in the subject by obtaining an image of the subject's brain or performing neurological testing on the subject.

E174. The method of any one of E171-173, wherein the method further comprises administering a treatment of glioblastoma to the subject.

E175. A method of identifying a subject as having an increased likelihood of developing glioblastoma, wherein the method comprises:
(a) determining a level of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, or SPP1, or a byproduct or precursor or degradation product thereof, in a biological sample from a subject; and
(b) identifying a subject having an elevated level of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, or SPP1, or a byproduct or precursor or degradation product thereof, in the biological sample as compared to a reference level, as having an increased likelihood of developing glioblastoma.

E176. A method of identifying a subject as having an increased likelihood of developing glioblastoma, wherein the method comprises:
(a) determining a level of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, or MT-ND5, or a byproduct or precursor or degradation product thereof, in a biological sample from a subject; and
(b) identifying a subject having a decreased level of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, or MT-ND5, or a byproduct or precursor or degradation product thereof, in the biological sample as compared to a reference level, as having an increased likelihood of developing glioblastoma.

E177. The method of E175 or E176, wherein the method further comprises monitoring the identified subject for the development of symptoms of glioblastoma.

E178. The method of any one of E175-E177, wherein the method further comprises recording in the identified subject's clinical record that the subject has an increased likelihood or susceptibility of developing glioblastoma.

E179. The method of any one of E175-E178, wherein the method further comprises notifying the subject's family that the subject has an increased likelihood or susceptibility of developing glioblastoma.

E180. The method of any one of E175-E179, wherein the method further comprises administering to the subject a treatment for decreasing the rate of progression or decreasing the likelihood or susceptibility of developing glioblastoma.

E181. The method of any one of E175-E177, wherein the biological sample comprises brain tissue or cerebrospinal fluid.

E182. The method of any one of E175-E181, further comprising obtaining the biological sample from the subject.

E183. The method of any one of E175-E182, wherein the level is a level of protein or a byproduct or precursor or degradation product thereof.

E184. The method of any one of E175-E182, wherein the level is a level of mRNA or a fragment thereof.

E185. A method of monitoring progression of glioblastoma in a subject over time, wherein the method comprises:
(a) determining a first level of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, or SPP1, or a byproduct or precursor or degradation product thereof, in a first biological sample obtained from a subject at a first time point;
(b) determining a second level of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, or SPP1, or a byproduct or precursor or degradation product thereof, in a second biological sample obtained from the subject at a second time point;
(c) identifying:
(i) a subject having an increased second level, as compared to the first level, as having progressing glioblastoma, or
(ii) a subject having about the same or a decreased second level as compared to the first level, as having static or regressing glioblastoma.

E186. A method of monitoring progression of glioblastoma in a subject over time, wherein the method comprises:
(a) determining a first level of: GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, or MT-ND5, or a byproduct or precursor or degradation product thereof, in a first biological sample obtained from a subject at a first time point;
(b) determining a second level of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, or MT-ND5, or a byproduct or precursor or degradation product thereof, in a second biological sample obtained from the subject at a second time point;
(c) identifying:
(i) a subject having a decreased second level, as compared to the first level, as having progressing glioblastoma, or
(ii) a subject having about the same or a increased second level as compared to the first level, as having static or regressing glioblastoma.

E187. The method of E185 or E186, wherein the method comprises identifying a subject as having progressing glioblastoma.

E188. The method of E187, wherein the method further comprises administering a treatment for glioblatoma to the subject or increasing the dose of a treatment for glioblastoma to be administered to the subject.

E189. The method of E188, wherein the method further comprises recording in the subject's clinical record that the subject has progressing glioblastoma.

E190. The method of any one of E185-E188, wherein the method comprises identifying a subject as having static or regressing glioblastoma.

E191. The method of E190, wherein the method further comprises recording in the subject's clinical record that the subject has static or regressing glioblastoma.

E192. A method of determining efficacy of treatment of a treatment for glioblastoma in a subject, wherein the method comprises:
(a) determining a first level of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, or SPP1, or a byproduct or precursor or degradation product thereof, in a first biological sample obtained from a subject at a first time point;
(b) determining a second level of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, or SPP1, or a byproduct or precursor or degradation product thereof, in a second biological sample obtained from the subject at a second time point, wherein the subject is administered one or more doses of a therapeutic treatment between the first and second time points;
(c) identifying:
(i) the therapeutic treatment as being effective in a subject having about the same or a decreased second level, as compared to the first level, or
(ii) the therapeutic treatment as not being effective in a subject having an increased second level, as compared to the first level.

E193. A method of determining efficacy of treatment of a treatment for glioblastoma in a subject, wherein the method comprises:
(a) determining a first level of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, or MT-ND5, or a byproduct or precursor or degradation product thereof, in a first biological sample obtained from a subject at a first time point;
(b) determining a second level of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, or MT-ND5, or a byproduct or precursor or degradation product thereof, in a second biological sample obtained from the subject at a second time point, wherein the subject is administered one or more doses of a therapeutic treatment between the first and second time points;
(c) identifying:
(i) the therapeutic treatment as being effective in a subject having an increased second level as compared to the first level, or
(ii) the therapeutic treatment as not being effective in a subject having about the same or a decreased second level as compared to the first level.

E194. The method of E182 or E193, wherein the method comprises identifying the therapeutic treatment as being effective in the subject.

E195. The method of E194, wherein the method further comprises selecting additional doses of the therapeutic treatment for the subject.

E196. The method of E194, wherein the method further comprises administering additional doses of the therapeutic treatment to the subject.

E197. The method of E194, wherein the method further comprises recording in the subject's clinical record that the therapeutic treatment is effective in the subject.

E198. The method of E192 or E193, wherein the method comprises identifying the therapeutic treatment as not being effective in the subject.

E199. The method of E198, wherein the method further comprises selecting a different therapeutic treatment for the subject.

E200. The method of E198, wherein the method further comprises administering a different therapeutic treatment to the subject.

E201. The method of E198, wherein the method further comprises increasing the dose of the therapeutic treatment to be administered to the subject.

E202. The method of E198, wherein the method further comprises administering one or more additional doses of the therapeutic treatment to the subject in combination with an additional therapeutic treatment.

E203. The method of any one of E185-E202, wherein the first and second biological samples comprise brain tissue or cerebrospinal fluid.

E204. The method of any one of E185-E203, further comprising obtaining the first and second biological samples from the subject.

E205. The method of any one of E185-E204, wherein each of the first and second level is a level of protein or a byproduct or precursor or degradation product thereof.

E206. The method of any one of E185-E205, wherein each of the first and second level is a level of mRNA or a fragment thereof.

E207. A kit comprising:
an antibody that binds specifically to COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, or MT-ND5 or a byproduct or precursor or degradation product thereof; and
instructions for performing the method of any one of E171-E206.

E208. A method of identifying a patient subpopulation for which a therapeutic treatment is effective for glioblastoma, the method comprising:
(a) administering a therapeutic treatment for glioblastoma to a patient subpopulation;
(b) determining (i) a first level of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, or SPP1, or a byproduct or precursor or degradation product thereof, in first biological samples obtained from a patient subpopulation at a first time point and (ii) a second level of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, or SPP1, or a byproduct or precursor or degradation product thereof, in second biological samples obtained from the patient population at a second time point, wherein the patient subpopulation is administered one or more doses of a therapeutic treatment between the first and second time points;
(c) determining a correlation between efficacy of the therapeutic treatment and the second level in samples from the patient subpopulation as compared to the level in a sample obtained from an untreated patient, wherein a lower second level in the samples from the patient subpopulation as compared to the level in the sample from the untreated patient is indicative that the therapeutic treatment is effective for glioblastoma in the patient subpopulation.

E209. The method of E208, wherein the therapeutic treatment is an antagonist of the gene, or a byproduct or precursor or degradation product thereof.

E210. A method of identifying a patient subpopulation for which a therapeutic treatment is effective for glioblastoma, the method comprising:
(a) administering a therapeutic treatment for glioblastoma to a patient subpopulation;
(b) determining (i) a first level of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, or MT-ND5, or a byproduct or precursor or degradation product thereof, in first biological samples obtained from a patient subpopulation at a first time point and (ii) a second level of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, or MT-ND5, or a byproduct or precursor or degradation product thereof, in second biological samples obtained from the patient population at a second time point, wherein the patient subpopulation is administered one or more doses of a therapeutic treatment between the first and second time points;
(c) determining a correlation between efficacy of the therapeutic treatment and the second level in samples from the patient subpopulation as compared to a level in a sample obtained from an untreated patient, wherein about the same or an elevated second level in the samples from the patient subpopulation as compared to the level in the sample from the untreated patient is indicative that the therapeutic treatment is effective for glioblastoma in the patient subpopulation.

E211. The method of E210, wherein the therapeutic treatment is an agonist of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, or MT-ND5, or a byproduct or precursor or degradation product thereof.

E212. A method of modifying treatment of a glioblastoma patient with a therapeutic treatment, the method comprising:
(a) administering a therapeutic treatment to a glioblastoma patient;
(b) determining (i) a pre-treatment level of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, or SPP1, or a byproduct or precursor or degradation product thereof, in a pre-treatment sample obtained from the glioblastoma patient before treatment and (ii) a post-treatment level of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, or SPP1, or a byproduct or precursor or degradation product thereof, in a post-treatment sample obtained from the glioblastoma patient after treatment, wherein a higher post-treatment level, as compared to the pre-treatment level, is indicative of the responsiveness to treatment with the therapeutic treatment; and
(c) increasing the amount of the therapeutic treatment administered to the patient based on the higher post-treatment level as compared to the pre-treatment level.

E213. The method of E212, wherein the therapeutic treatment is an antagonist of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, or SPP1, or a byproduct or precursor or degradation product thereof.

E213. A method of modifying treatment of a glioblastoma patient with a therapeutic treatment, the method comprising:
(a) administering a therapeutic treatment to an glioblastoma patient;
(b) determining (i) a pre-treatment level of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, or MT-ND5, or a byproduct or precursor or degradation product thereof, in a pre-treatment sample obtained from the glioblastoma patient before treatment and (ii) a post-treatment level of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, or MT-ND5, or a byproduct or precursor or degradation product thereof, in a post-treatment sample obtained from the glioblastoma patient after treatment, wherein a decreased post-treatment level as compared to the pre-treatment level, is indicative of the responsiveness to treatment with the therapeutic treatment; and
(c) increasing the amount of the therapeutic treatment administered to the patient based on the decreased post-treatment level as compared to the pre-treatment level.

E214. The method of any one of E35-E42, E45-E52, E61-E68, E75-E82, E100, E102, E104, E106, E108, E110, E112, E114, E116, E118, E120, E122, E124, E126, E128, and E130, wherein the sample is a cell culture sample.

E215. The method of any one of E35-E42, E45-E52, E61-E68, E75-E82, E100, E102, E104, E106, E108, E110, E112, E114, E116, E118, E120, E122, E124, E126, E128, and E130, wherein the levels of at least two biomarkers of the group consisting of PMCH, Akr1e1, Atg4c, Gm14296, Hcr, Ttr, Adora2a, Trh, and byproducts, precursors and degradation products thereof thereof are determined.

E216. The method of any one of E35-E42, E45-E52, E61-E68, E75-E82, E100, E102, E104, E106, E108, E110, E112, E114, E116, E118, E120, E122, E124, E126, E128, and E130, wherein the levels of at least three biomarkers of the group consisting of PMCH, Akr1e1, Atg4c, Gm14296, Hcr, Ttr, Adora2a, Trh, and byproducts, precursors and degradation products thereof are determined.

E217. The method of any one of E35-E42, E45-E52, E61-E68, E75-E82, E100, E102, E104, E106, E108, E110, E112, E114, E116, E118, E120, E122, E124, E126, E128, and E130, wherein the levels of at least four biomarkers of the group consisting of PMCH, Akr1e1, Atg4c, Gm14296, Hcr, Ttr, Adora2a, Trh, and byproducts, precursors and degradation products thereof are determined.

E218. A kit comprising:
(a) a substrate with one or more capture probes, wherein each capture probe comprises a spatial barcode and a capture domain, wherein each capture probe binds to a biological analyte from a biological sample, and (b) reagents to detect the biological analyte, wherein the biological analyte is a biomarker selected from a group consisting of PMCH, Akr1e1, Atg4c, Gm14296, Hcr, Ttr, Adora2a, Trh.

E219. A kit comprising:
(a) a substrate with one or more capture probes, wherein each capture probe comprises a spatial barcode and a capture domain, wherein each capture probe binds to a biological analyte from a biological sample, and (b) reagents to detect the biological analyte, wherein the biological analyte is a biomarker selected from a group consisting of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, or SPP1.

E220. The kit of E218 or E219, wherein the kit further comprises one or more reagents selected from a group consisting of one or more antibodies, one or more antigen-binding antibody fragments, labeled hybridization probes, primers, or any combination thereof, wherein the one or more reagents enable visualizing one or more features of the biological sample.

E221. The kit of E218 or E219, wherein the biological sample is a tissue sample.

E222. The kit of E218 or E219, wherein the biological sample is a cell culture sample.

E223. A method of assessing expression levels in a subject, comprising: a) obtaining a biological sample from the subject; and b) determining an expression level of one or more analytes selected from the group consisting of pro-melanin concentrating hormone (PMCH), aldo-keto reductase family 1, member E1 (Akr1e1), autophagy related 4C cysteine peptidase (Atg4c), Gm14296, hypocretin neuropeptide precursor (Hcrt), transthyretin (Ttr), adenosine A2a receptor (Adora2a), thyrotropin releasing hormone preproprotein (Trh), prion protein (Prnp), protein kinase C theta (Prkck), complement C1q B chain (C1qb), glutamate-ammonia ligase (Glul), pituitary tumor-transforming gene 1 (Pttg1), aurora kinase A interacting protein 1 (Aurkaip1), cocaine- and amphetamine-regulated transcript protein (Cartpt), complement component 4B (C4b), mitotic spindle organizing protein 1 (Mzt1), tyrosine-protein phosphatase non-receptor type 3 (Ptpn3), phytanoyl-CoA 2-hydroxylase interacting protein (Phyhip), small nucleolar RNA host gene 11 (Snhg11), RAD23 homolog B (Rad23b), netrin G1 (Ntng1), serine/arginine-rich splicing factor 5 (Srsf5), tyrosine-protein phosphatase non-receptor type 4 (Ptpn4), 5′-nucleotidase domain containing 3 (Nt5dc3), insulin induced gene 1 (Insig1), oxytocin (Oxt), delta-aminolevulinate dehydratase (Alad), nudix hydrolase 19 (Nudt19), Gm10076 ribosomal protein L41 pseudogene (Gm10076), cyclase associated actin cytoskeleton regulatory protein 1 (Cap1), regulator of cell cycle (Rgcc), ubiquitin A-52 residue ribosomal protein fusion product 1 (Uba52), protein phosphatase 1 regulatory inhibitor subunit 1B (Ppp1r1b), phosphodiesterase 10A (Pde10a), Ubiquitin Conjugating Enzyme E2 M (Ube2m), hemoglobin alpha, adult chain 1 (Hba-a1), glutathione S-transferase pi gene (Gstp1), Mesencephalic Astrocyte Derived Neurotrophic Factor (Manf), G Protein-Coupled Receptor 88 (Gpr88), Sin3A-associated protein (Sap301), alkB homolog 6 (Alkbh6), Small nucleolar RNA host gene 6 (Snhg6), Arginine Vasopressin (Avp), Profilin-1 (Pfn1), tachykinin, precursor 1 (Tac1), byproducts, precursors and degradation products thereof, in the biological sample obtained from the subject.

E224. The method of E223, wherein (a) comprises serially obtaining a biological sample from the subject at a plurality of time points and (b) comprises determining the expression levels in the serially obtained biological samples from the subject.

E225. A method of diagnosing a subject as having Alzheimer's disease or having an increased likelihood of developing Alzheimer's disease, wherein the method comprises: (a)
determining an expression level of one or more analytes selected from the group consisting of PMCH, Akr1e1, Atg4c, Gm14296, Hcrt, Ttr, Adora2a, and Trh, and byproducts, precursors and degradation products thereof, in a biological sample from the subject; and (b) identifying the subject having: (1) an elevated expression level of the one or more analytes PMCH, Akr1e1, Atg4c, Gm14296, Hcrt, and Ttr, and byproducts, precursors and degradation products thereof, of step (a), or (2) about the same or a decreased expression level of the one or more analytes Adora2a, Trh, byproducts, precursors and degradation products thereof, of step (a) as having Alzheimer's disease or having an increased likelihood of developing Alzheimer's disease.

E226. A method of diagnosing a subject as having Alzheimer's disease or having an increased likelihood of developing Alzheimer's disease, wherein the method comprises: (a)
determining an expression level of one or more analytes selected from the group consisting of PMCH, Akr1e1, Atg4c, Gm14296, Hcrt, Ttr, Adora2a, Trh, Prnp, Prkck, C1qb, Glul, Pttg1, Aurkaip1, Cartpt, C4b, Mzt1, Ptpn3, Phyhip, Snhg11, Rad23b, Ntng1, Srsf5, Ptpn4, Nt5dc3, Insig1, Oxt, Alad, Nudt19, Gm10076, Cap1, Rgcc, Uba52, Ppp1r1b, Pde10a, Ube2m, Hba-a1, Gstp1, Manf, Gpr88, Sap301, Alkbh6, Snhg6, Avp, Pfn1, Tac1, byproducts, precursors and degradation products thereof, in a biological sample from the subject; and (b) identifying the subject having: (1) an elevated expression level of the one or more analytes PMCH, Akr1e1, Atg4c, Gm14296, Hcrt, Ttr, Prnp, Prkck, C1qb, Glul, Pttg1, Aurkaip1, Cartpt, C4b, Mzt1, Ptpn3, Phyhip, Snhg11, Rad23b, Ntng1, Srsf5, Ptpn4, Nt5dc3, Insig1, and byproducts, precursors and degradation products thereof, of step (a), or (2) about the same or a decreased expression level of the one or more analytes Adora2a, Trh, Oxt, Alad, Nudt19, Gm10076, Cap1, Rgcc, Uba52, Ppp1r1b, Pde10a, Ube2m, Hba-a1, Gstp1, Manf, Gpr88, Sap301, Alkbh6, Snhg6, Avp, Pfn1, Tac1, byproducts, precursors and degradation products thereof, of step (a) as having Alzheimer's disease or having an increased likelihood of developing Alzheimer's disease.

E227. The method of E225 or E226, further comprising obtaining the biological sample from the subject.

E228. A method of monitoring progression of Alzheimer's disease in a subject over time, wherein the method comprises: (a) determining a first expression level of one or more analytes selected from the group consisting of PMCH, Akr1e1, Atg4c, Gm14296, Hcrt, Ttr, Adora2a, Trh, Prnp, Prkck, C1qb, Glul, Pttg1, Aurkaip1, Cartpt, C4b, Mzt1, Ptpn3, Phyhip, Snhg11, Rad23b, Ntng1, Srsf5, Ptpn4, Nt5dc3, Insig1, Oxt, Alad, Nudt19, Gm10076, Cap1, Rgcc, Uba52, Ppp1r1b, Pde10a, Ube2m, Hba-a1, Gstp1, Manf, Gpr88, Sap301, Alkbh6, Snhg6, Avp, Pfn1, Tac1, byproducts, precursors and degradation products thereof, in a first biological sample obtained from the subject at a first time point; (b) determining a second expression level of the one or more analytes of step (a); and (c) identifying the subject as having: (1) an increased second expression level of the one or more analytes of the PMCH, Akr1e1, Atg4c, Gm14296, Hcrt, Ttr, Prnp, Prkck, C1qb, Glul, Pttg1, Aurkaip1, Cartpt, C4b, Mzt1, Ptpn3, Phyhip, Snhg11, Rad23b, Ntng1, Srsf5, Ptpn4, Nt5dc3, Insig1, and byproducts, precursors and degradation products thereof, of step (a), or (2) about the same or a decreased second expression level of the one or more of the Adora2a, Trh, Oxt, Alad, Nudt19, Gm10076, Cap1, Rgcc, Uba52, Ppp1r1b, Pde10a, Ube2m, Hba-a1, Gstp1, Manf, Gpr88, Sap301, Alkbh6, Snhg6, Avp, Pfn1, Tac1, byproducts, precursors and degradation products thereof, of step (a), as having progressing Alzheimer's disease; or (3) about the same or a decreased second level of the PMCH, Akr1e1, Atg4c, Gm14296, Hcrt, Ttr, Prnp, Prkck, C1qb, Glul, Pttg1, Aurkaip1, Cartpt, C4b, Mzt1, Ptpn3, Phyhip, Snhg11, Rad23b, Ntng1, Srsf5, Ptpn4, Nt5dc3, Insig1, or a byproduct or precursor or degradation product thereof, of step (a), or (4) an increased second expression level of the Adora2a, Trh, Oxt, Alad, Nudt19, Gm10076, Cap1, Rgcc, Uba52, Ppp1r1b, Pde10a, Ube2m, Hba-a1, Gstp1, Manf, Gpr88, Sap301, Alkbh6, Snhg6, Avp, Pfn1, Tac1, byproducts, precursors and degradation products thereof, of step (a) as having static or regressing Alzheimer's disease.

E229. The method of any one of E223-E228, wherein the method further comprises administering a treatment for Alzheimer's disease to the subject.

E230. A method of determining efficacy of treatment of a treatment for Alzheimer's disease in a subject, wherein the method comprises: (a) determining a first expression level of one or more analytes selected from the group consisting of PMCH, Akr1e1, Atg4c, Gm14296, Hcrt, Ttr, Adora2a, Trh, Prnp, Prkck, C1qb, Glul, Pttg1, Aurkaip1, Cartpt, C4b, Mzt1, Ptpn3, Phyhip, Snhg11, Rad23b, Ntng1, Srsf5, Ptpn4, Nt5dc3, Insig1, Oxt, Alad, Nudt19, Gm10076, Cap1, Rgcc, Uba52, Ppp1r1b, Pde10a, Ube2m, Hba-a1, Gstp1, Manf, Gpr88, Sap301, Alkbh6, Snhg6, Avp, Pfn1, Tac1, byproducts, precursors and degradation products thereof, in a first biological sample obtained from a subject at a first time point; (b) determining a second expression level of the one or more analytes of step 9(a) in a second biological sample obtained from the subject at a second time point, wherein the subject is administered one or more doses of a therapeutic treatment between the first and second time points; and (c) identifying: (1a) the therapeutic treatment as being effective in the subject having about the same or a decreased second expression level of the PMCH, Akr1e1, Atg4c, Gm14296, Hcrt, Ttr, Adora2a, Trh, Prnp, Prkck, C1qb, Glul, Pttg1, Aurkaip1, Cartpt, C4b, Mzt1, Ptpn3, Phyhip, Snhg11, Rad23b, Ntng1, Srsf5, Ptpn4, Nt5dc3, Insig1, Oxt, Alad, Nudt19, Gm10076, Cap1, Rgcc, Uba52, Ppp1r1b, Pde10a, Ube2m, Hba-a1, Gstp1, Manf, Gpr88, Sap301, Alkbh6, Snhg6, Avp, Pfn1, Tac1, byproducts, precursors and degradation products thereof, of step (a), or (1b) the therapeutic treatment as being effective in a subject having an increased second expression level of the one or more or Adora2a, Trh, and byproducts, precursors and degradation products thereof, of step (a); or (2a) the therapeutic treatment as not being effective in a subject having an increased second expression level of the one or more of PMCH, Akr1e1, Atg4c, Gm14296, Hcrt, Ttr, Prnp, Prkck, C1qb, Glul, Pttg1, Aurkaip1, Cartpt, C4b, Mzt1, Ptpn3, Phyhip, Snhg11, Rad23b, Ntng1, Srsf5, Ptpn4, Nt5dc3, and Insig1, and byproducts, precursors and degradation products thereof, of step (a), or (2b) the therapeutic treatment as not being effective in a subject having about the same or a decreased second expression level of the one or more of Adora2a, Trh, Oxt, Alad, Nudt19, Gm10076, Cap1, Rgcc, Uba52, Ppp1r1b, Pde10a, Ube2m, Hba-a1, Gstp1, Manf, Gpr88, Sap301, Alkbh6, Snhg6, Avp, Pfn1, Tac1, byproducts, precursors and degradation products thereof, of step (a).

E231. A method of identifying a patient subpopulation for which a therapeutic treatment is effective for Alzheimer's disease, the method comprising: (a) determining: (1) a first expression level of one or more analytes selected from the group consisting of PMCH, Akr1e1, Atg4c, Gm14296, Hcrt, Ttr, Adora2a, Trh, Prnp, Prkck, C1qb, Glul, Pttg1, Aurkaip1, Cartpt, C4b, Mzt1, Ptpn3, Phyhip, Snhg11, Rad23b, Ntng1, Srsf5, Ptpn4, Nt5dc3, Insig1, Oxt, Alad, Nudt19, Gm10076, Cap1, Rgcc, Uba52, Ppp1r1b, Pde10a, Ube2m, Hba-a1, Gstp1, Manf, Gpr88, Sap301, Alkbh6, Snhg6, Avp, Pfn1, Tac1, byproducts, precursors and degradation products thereof, in first biological samples obtained from a patient subpopulation at a first time point; and (2) a second expression level of the one or more analytes of step (a)(1) in second biological samples obtained from the patient population at a second time point, wherein the patient subpopulation is administered one or more doses of a therapeutic treatment for Alzheimer's Disease between the first and second time points; (b) determining a correlation between efficacy of the therapeutic treatment and the second level in the biological samples from the patient subpopulation as compared a biological sample obtained from an untreated patient, wherein (1) a lower second expression level of the one or more analytes PMCH, Akr1e1, Atg4c, Gm14296, Hcrt, Ttr, Prnp, Prkck, C1qb, Glul, Pttg1, Aurkaip1, Cartpt, C4b, Mzt1, Ptpn3, Phyhip, Snhg11, Rad23b, Ntng1, Srsf5, Ptpn4, Nt5dc3, and Insig1, and byproducts, precursors and degradation products thereof, or (2) about the same or an elevated second expression level of the one or more analytes Adora2a, Trh, Oxt, Alad, Nudt19, Gm10076, Cap1, Rgcc, Uba52, Ppp1r1b, Pde10a, Ube2m, Hba-a1, Gstp1, Manf, Gpr88, Sap301, Alkbh6, Snhg6, Avp, Pfn1, Tac1, byproducts, precursors and degradation products thereof, in the biological samples from the patient subpopulation as compared to the biological sample from the untreated patient is indicative that the therapeutic treatment is effective for Alzheimer's disease in the patient subpopulation.

E232. The method of any one of claims E228-E231, further comprising obtaining the first and second biological samples from the subject.

E233. The method of any one of E223-E232, wherein the levels of at least two of the analytes are determined.

E234. The method of any one of E223-E233, wherein the levels of at least three of the analytes are determined.

E235. The method of any one of E223-E234, wherein the levels of at least four of the analytes are determined.

E236. The method of E235, wherein the at least four of the analytes are pro-melanin concentrating hormone (PMCH), hypocretin neuropeptide precursor (Hcrt), oxytocin (Oxt), and phytanoyl-CoA 2-hydroxylase interacting protein (Phyhip), and byproducts, precursors and degradation products thereof.

E237. The method of any one of E223-E236, wherein the levels of each of the analytes are determined.

E238. The method of any one of E223-E237, further comprising administering a treatment or prophylaxis of Alzheimer's disease to the subject, adjusting a dosage of a treatment or prophylaxis of Alzheimer's disease for the subject, or adjusting a treatment or prophylaxis of Alzheimer's disease for the subject.

E239. The method of E238, wherein the treatment or prophylaxis comprises: (a) an antagonist of one or more of the analytes selected from the group consisting of PMCH, Akr1e1, Atg4c, Gm14296, Hcrt, Ttr, Prnp, Prkck, C1qb, Glul, Pttg1, Aurkaip1, Cartpt, C4b, Mzt1, Ptpn3, Phyhip, Snhg11, Rad23b, Ntng1, Srsf5, Ptpn4, Nt5dc3, and Insig1, and byproducts, precursors and degradation products thereof; or (b) an agonist of one or more of the analytes selected from the group consisting of Adora2a, Trh, Oxt, Alad, Nudt19, Gm10076, Cap1, Rgcc, Uba52, Ppp1r1b, Pde10a, Ube2m, Hba-a1, Gstp1, Manf, Gpr88, Sap301, Alkbh6, Snhg6, Avp, Pfn1, Tac1, and byproducts, precursors and degradation products thereof.

E240. The method of E238, wherein the treatment or prophylaxis comprises administering one or more agents selected from the group consisting of a cholinesterase inhibitor, an N-methyl-D-aspartate (NMDA) inhibitor, an antipsychotic, a tricyclic antidepressant, a benzodiazepine, insulin, and tacrine hydrochloride.

E241. The method of E240, wherein the cholinesterase inhibitor is galantamine, rivastigmine, or donepezil; the NMDA inhibitor is memantine; the antipsychotic agent is aripiprazole, risperidone, olanzapine, quetiapine, or haloperidol; the benzodiazepine is lorazepam, oxazepam or temazepam; and the tricyclic antidepressant is nortriptyline.

E242. A kit comprising: (a) an antibody that binds specifically to one or more analytes selected from the group consisting of PMCH, AKR1E1, ATG4C, GM14296, HCR, TTR, ADORA2A, TRH, PRNP, PRKCK, C1QB, GLUL, PTTG1, AURKAIP1, CARTPT, C4B, MZT1, PTPN3, PHYHIP, SNHG11, RAD23B, NTNG1, SRSF5, PTPN4, NT5DC3, INSIG1, OXT, ALAD, NUDT19, GM10076, CAP1, RGCC, UBA52, PPP1R1B, PDE10A, UBE2M, HBA-A1, GSTP1, MANF, GPR88, SAP30L, ALKBH6, SNHG6, AVP, PFN1, TAC1, byproducts, precursors and degradation products thereof; and (b) instructions for performing the method of any one of the preceding claims.

E243. A method of assessing expression levels in a subject, comprising: (a) obtaining a biological sample from the subject; and (b) determining an expression level of one or more analytes selected from the group consisting of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, SLN, SRPX2, METTL7B, POSTN, NNMT, TIMP4, SERPINA3, KLHDC8A, NES, F2R, XIST, COL1A2, COL4A1, CA12, ANXA2, WWTR1, COL4A1, LAMB2, SPARC, FN1, TNFRSF1A, HLA-DRA, ALDH1L1, FLNA, NAMPT, VEGFA, C3, HLA-A, GRN, HLA-B, TPP1, HLA-B, HLA-DRA, LAMB2, NAMPT, byproducts, precursors, and degradation products thereof in the biological sample obtained from the subject.

E244. The method of E243, wherein (a) comprises serially obtaining a biological sample from the subject at a plurality of time points and (b) comprises determining the expression levels in the serially obtained biological samples from the subject.

E245. A method of diagnosing a subject as having glioblastoma, or having an increased likelihood of developing glioblastoma, wherein the method comprises: (a) determining an expression level of one or more analytes selected from the group consisting of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, and byproducts, precursors, degradation and degradation products thereof, in a biological sample from a subject; and (b) identifying a subject having an elevated expression level of the one or more analytes of step (a), in the biological sample as compared to a reference level, as having glioblastoma, or having an increased likelihood of developing glioblastoma.

E246. A method of diagnosing a subject as having glioblastoma, or having an increased likelihood of developing glioblastoma, wherein the method comprises: (a) determining an expression level of one or more analytes selected from the group consisting of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, SLN, SRPX2, METTL7B, POSTN, NNMT, TIMP4, SERPINA3, KLHDC8A, NES, F2R, XIST, COL1A2, COL4A1, CA12, ANXA2, WWTR1, COL4A1, LAMB2, SPARC, FN1, TNFRSF1A, HLA-DRA, ALDH1L1, FLNA, NAMPT, VEGFA, C3, HLA-A, GRN, HLA-B, TPP1, HLA-B, HLA-DRA, LAMB2, and NAMPT and byproducts, precursors, degradation and degradation products thereof, in a biological sample from a subject; and (b) identifying a subject having an elevated expression level of the one or more analytes of step (a), in the biological sample as compared to a reference level, as having glioblastoma, or having an increased likelihood of developing glioblastoma.

E247. A method of diagnosing a subject as having glioblastoma, or having an increased likelihood of developing glioblastoma, wherein the method comprises: (a) determining an expression level of one or more analytes selected from the group consisting of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, byproducts, precursor and degradation products thereof, in a biological sample from a subject; and (b) identifying a subject having a decreased expression level of the one or more analytes of step (a), in the biological sample as compared to a reference level, as having glioblastoma, or having an increased likelihood of developing glioblastoma.

E248. A method of monitoring progression of glioblastoma in a subject over time, wherein the method comprises: (a) determining a first expression level of (1) one or more analytes selected from the group consisting of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, SLN, SRPX2, METTL7B, POSTN, NNMT, TIMP4, SERPINA3, KLHDC8A, NES, F2R, XIST, COL1A2, COL4A1, CA12, ANXA2, WWTR1, COL4A1, LAMB2, SPARC, FN1, TNFRSF1A, HLA-DRA, ALDH1L1, FLNA, NAMPT, VEGFA, C3, HLA-A, GRN, HLA-B, TPP1, HLA-B, HLA-DRA, LAMB2, and NAMPT, and byproducts, precursors, and degradation products thereof; or (2) one or more analytes selected from the group consisting of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, and MT-ND5, byproducts, precursors, and degradation products thereof, in a first biological sample obtained from a subject at a first time point; (b) determining a second expression level of the one or more analytes from step (a)(1) or from step (a)(2) in a second biological sample obtained from the subject at a second time point; and (c) identifying: (1) a subject having an increased second expression level of the one or more analytes from step (a)(1) or a decreased second expression level of the one or more analytes from step (a)(2), as compared to the first expression level, as having progressing glioblastoma, or (2) a subject having about the same or a decreased second level of the one or more analytes of step (a)(1) as compared to the first expression level, or about the same or an increased second expression level of the one or more analytes of step (a)(2), as having static or regressing glioblastoma.

E249. A method of determining efficacy of treatment of a treatment for glioblastoma in a subject, wherein the method comprises: (a) determining a first expression level of: (1) one or more analytes selected from the group consisting of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, SLN, SRPX2, METTL7B, POSTN, NNMT, TIMP4, SERPINA3, KLHDC8A, NES, F2R, XIST, COL1A2, COL4A1, CA12, ANXA2, WWTR1, COL4A1, LAMB2, SPARC, FN1, TNFRSF1A, HLA-DRA, ALDH1L1, FLNA, NAMPT, VEGFA, C3, HLA-A, GRN, HLA-B, TPP1, HLA-B, HLA-DRA, LAMB2, NAMPT, byproducts, precursors, and degradation products thereof; or (2) one or more analytes selected from the group consisting of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, byproducts, precursors, and degradation products thereof in a first biological sample obtained from a subject at a first time point; (b) determining a second expression level of the one or more analytes of step (a)(1) or step (a)(2), in a second biological sample obtained from the subject at a second time point, wherein the subject is administered one or more doses of a therapeutic treatment between the first and second time points; and (c) identifying: (i) the therapeutic treatment as being effective in a subject having about the same or a decreased second expression level of the one or more analytes of step (a)(1), or an increased second expression level of the one or more analytes of step (a)(2), as compared to the first expression level, or (ii) the therapeutic treatment as not being effective in a subject having an increased second expression level of the one or more analytes of step (a)(1), or about the same or a decreased second level of the one or more analytes of step (a)(2), as compared to the first level.

E250. A kit comprising: an antibody that binds specifically to one or more analytes selected from the group consisting of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, SLN, SRPX2, METTL7B, POSTN, NNMT, TIMP4, SERPINA3, KLHDC8A, NES, F2R, XIST, COL1A2, COL4A1, CA12, ANXA2, WWTR1, COL4A1, LAMB2, SPARC, FN1, TNFRSF1A, HLA-DRA, ALDH1L1, FLNA, NAMPT, VEGFA, C3, HLA-A, GRN, HLA-B, TPP1, HLA-B, HLA-DRA, LAMB2, NAMPT and byproducts, precursors, and degradation products thereof; and
instructions for performing the method of any one of E243-246.

E251. A method of identifying a patient subpopulation for which a therapeutic treatment is effective for glioblastoma, the method comprising: (a) determining: (1) a first expression level of (i) one or more analytes selected from the group consisting of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, SLN, SRPX2, METTL7B, POSTN, NNMT, TIMP4, SERPINA3, KLHDC8A, NES, F2R, XIST, COL1A2, COL4A1, CA12, ANXA2, WWTR1, COL4A1, LAMB2, SPARC, FN1, TNFRSF1A, HLA-DRA, ALDH1L1, FLNA, NAMPT, VEGFA, C3, HLA-A, GRN, HLA-B, TPP1, HLA-B, HLA-DRA, LAMB2, NAMPT, byproducts, precursors, and degradation products thereof; or (ii) one or more analytes selected from the group consisting of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, byproducts, precursors, and degradation products thereof in first biological samples obtained from a patient subpopulation at a first time point and (2) a second expression level of the one or more analytes of step (a)(1)(i) or step (a)(1)(ii), in second biological samples obtained from the patient population at a second time point, wherein the patient subpopulation is administered one or more doses of a therapeutic treatment for glioblastoma between the first and second time points; and (b) determining a correlation between efficacy of the therapeutic treatment and the second expression level in biological samples from the patient subpopulation as compared to the level in a biological sample obtained from an untreated patient, wherein a lower second expression level of the one or more analytes of step (a)(1)(i) or about the same or an elevated second level of the one or more analytes of step (a)(1)(ii) in the biological samples from the patient subpopulation as compared to the level in the biological sample from the untreated patient is indicative that the therapeutic treatment is effective for glioblastoma in the patient subpopulation.

E252. The method of any one of E246-E251, further comprising obtaining the biological sample or the first biological sample and the second biological sample from the subject.

E253. The method of any one of E243-E252, wherein the expression levels of at least two analytes are determined.

E254. The method of any one of E243-E253, wherein the expression levels of at least three analytes are determined.

E255. The method of any one of E243-E254, wherein the expression levels of at least four analytes are determined.

E256. The method of E255, wherein the at least four of the analytes are selected from a group consisting of ADM, CD44, FN1, HLA-A, HLA-B, HLA-DRA, LAMB2, NAMPT, NES, SPARC, SPP1, and VEGFA and byproducts, precursors and degradation products thereof.

E257. The method of any one of E243-E256, wherein the expression levels of each of the analytes are determined.

E258. The method of any one of E243-E257, further comprising administering a therapeutic treatment for glioblastoma to the subject, adjusting a dosage of a treatment for glioblastoma for the subject, or adjusting a treatment for glioblastoma for the subject.

E259. The method of E258, wherein the therapeutic treatment is: (a) an antagonist of one or more analytes selected from the group consisting of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, SLN, SRPX2, METTL7B, POSTN, NNMT, TIMP4, SERPINA3, KLHDC8A, NES, F2R, XIST, COL1A2, COL4A1, CA12, ANXA2, WWTR1, COL4A1, LAMB2, SPARC, FN1, TNFRSF1A, HLA-DRA, ALDH1L1, FLNA, NAMPT, VEGFA, C3, HLA-A, GRN, HLA-B, TPP1, HLA-B, HLA-DRA, LAMB2, NAMPT, byproducts, precursors, and degradation products thereof; or (b) an agonist of one or more analytes selected from the group consisting of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, byproducts, precursors, and degradation products thereof.

E260. The method of any one of E243-E259, further comprising administering a treatment or glioblastoma to the subject, wherein the treatment comprises surgery, chemotherapeutic agents, growth inhibitory agents, cytotoxic agents, agents used in radiation therapy, anti-angiogenesis agents, cancer immunotherapeutic agents, apoptotic agents, anti-tubulin agents, or a combination thereof.

E261. The method of any one of E223-E260, wherein the determining step(s) comprises: (a) contacting the biological sample with an substrate comprising a plurality of attached capture probes, wherein a capture probe of the plurality comprises (i) the spatial barcode and (ii) a capture domain that binds specifically to a sequence present in the analyte; (b) extending a 3′ end of the capture probe using the analyte that is specifically bound to the capture domain as a template to generate an extended capture probe; (c) amplifying the extended capture probe to produce the nucleic acid; (d) determining (i) all or a portion of the sequence of the spatial barcode or the complement thereof, and (ii) all or a portion of the sequence of the analyte from the biological sample; and (e) using the determined sequences of (i) and (ii) in step (d) to identify the location of the analyte in the biological sample.

E262. The method of any one of E223-E261, wherein the biological sample or first and second biological samples comprise cerebrospinal fluid, whole blood, plasma, and/or serum.

E263. The method of any one of E223-E262, wherein the biological sample or first and second biological samples comprise a tissue section.

E264. The method of any one of E223-E263, wherein the biological sample comprises serial tissue sections.

E265. The method of any one of E223-E264, wherein the biological sample comprises a tissue structure.

E266. The method of any one of E223-E265, wherein the expression level is a level of protein or a byproduct or precursor or degradation product thereof.

E267. The method of any one of E223-E266, wherein the expression level is a level of mRNA or a fragment thereof.

E268. The method of any one of E223-E241, E243-E249, and E251-E262, wherein the biological sample is a cell culture sample.

E269. A kit comprising: (a) a substrate with one or more capture probes, wherein each capture probe comprises a spatial barcode and a capture domain, wherein each capture probe binds to a biological analyte from a biological sample; and (b) reagents to detect the biological analyte, wherein the biological analyte is a analyte selected from a group consisting of PMCH, AKR1E1, ATG4C, GM14296, HCR, TTR, ADORA2A, TRH, PRNP, PRKCK, C1QB, GLUL, PTTG1, AURKAIP1, CARTPT, C4B, MZT1, PTPN3, PHYHIP, SNHG11, RAD23B, NTNG1, SRSF5, PTPN4, NT5DC3, INSIG1, OXT, ALAD, NUDT19, GM10076, CAP1, RGCC, UBA52, PPP1R1B, PDE10A, UBE2M, HBA-A1, GSTP1, MANF, GPR88, SAP30L, ALKBH6, SNHG6, AVP, PFN1, OR TAC1.

E270. A kit comprising: (a) a substrate with one or more capture probes, wherein each capture probe comprises a spatial barcode and a capture domain, wherein each capture probe binds to a biological analyte from a biological sample; and (b) reagents to detect the biological analyte, wherein the biological analyte is a biomarker selected from a group consisting of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, SLN, SRPX2, METTL7B, POSTN, NNMT, TIMP4, SERPINA3, KLHDC8A, NES, F2R, XIST, COL1A2, COL4A1, CA12, ANXA2, WWTR1, COL4A1, LAMB2, SPARC, FN1, TNFRSF1A, HLA-DRA, ALDH1L1, FLNA, NAMPT, VEGFA, C3, HLA-A, GRN, HLA-B, TPP1, HLA-B, HLA-DRA, LAMB2, and NAMPT.

E271. The kit of E269 or E270, wherein the kit further comprises one or more reagents selected from a group consisting of one or more antibodies, one or more antigen-binding antibody fragments, labeled hybridization probes, primers, or any combination thereof, wherein the one or more reagents enable visualizing one or more features of the biological sample.

E272. The kit of E269 or E270, wherein the biological sample is a tissue sample.

E273. The kit of E269 or E270, wherein the biological sample is a cell culture sample.

E274. The method of E253, wherein the at least two of the analytes are selected from a group consisting of ADM, CD44, FN1, HLA-A, HLA-B, HLA-DRA, LAMB2, NAMPT, NES, SPARC, SPP1, and VEGFA and byproducts, precursors and degradation products thereof.

Additional embodiments are disclosed in WO 2020/123305 A1, WO 2020/123320 A1, and WO 2020/123316 A1, each of which is incorporated by reference in its entirety.

### EXAMPLES

Identifying individual cells and their genetic makeup can be important for understanding their roles in how the central nervous system (CNS) physiologically functions, develops, and organizes; as well as how these modalities are altered in diseased states. The Examples described herein demonstrate, e.g., the ability to do one or more of the following: (1) examine histological and transcriptome profiles from the same tissue section at a much higher resolution, better sensitivity, and shorter time; (2) obtain unbiased and high-throughput gene expression analysis for intact tissue sections across different brain regions from both rodent specimens and human patients; (3) generate spatial clustering that reliably correlates with the neuroanatomy; and or (4) demonstrate the ability to discover novel targets and/or pathways with unbiased analysis.

### Example 1. Spatial Gene Expression Characterization Across the Mouse Hippocampal and Various Brain Regions

Spatial analysis was performed on a mouse brain section.

FIG. 1A shows spatial clustering of the hippocampus and neighboring areas using the spatial gene expression technology (the inset shows H&E staining reference). FIG. 1B shows spatially-resolved clustering and expression in the mouse brain (WT) based on total differentially expressed genes. The top genes that are more highly expressed in the hippocampus (than any other cluster) are shown (expanded and annotated to the right). These data show that spatial clustering (e.g., FIG. 1A) and t-SNE plots (FIG. 1C) can identify brain regions in an unbiased manner. In addition, restriction of gene expression was in specifically demonstrated in hippocampal areas (FIG. 2). Similarly, FIG. 3 shows spatial clustering and gene expression anteriorly within the olfactory bulb, and FIG. 4 shows spatial clustering and gene expression posteriorly within the cerebellum. (Scale bars=1.0 mm)

### Example 2. Spatial Clustering and Gria1 Expression Across the Sagittal Mouse Brain

Spatial analysis was performed on a mouse brain section.

FIG. 5A shows spatially-resolved clustering of the anterior and posterior regions of the mouse brain split across two capture areas. FIG. 5B shows an examination of Gria1 (Glutamate Ionotropic Receptor AMPA type Subunit 1) expression of across multiple regions of the brain.

### Example 3. Spatial Data Correlates with Single Nuclear RNA-Seq

FIG. 6A shows that serial sections between different capture areas demonstrate strong correlation with the examination of 10,000 randomly selected genes. FIG. 6B shows that obtained spatial sequencing data correlate with single-nuclear 3′ RNA sequencing from the consecutive sections collected the same brain sample.

### Example 4. Compatibility with Rat and Human Neuronal Tissue

Spatial analysis was performed on both a rat brain tissue section and a human brain tissue section.

FIG. 7A shows spatially-resolved clustering of the Sprague-Dawley rat olfactory bulb with a demonstration of Gabral expression corresponding to the external plexiform layer (inset). FIG. 7B shows spatially-resolved clustering of cerebellar tissue from a female patient (BioIVT: Asterand; Case ID 9917; Specimen ID 1215079F) with a demonstration of Neurod1 expression corresponding to the granule cell layer (inset).

### Example 5. Application of Spatial Gene Expression on the APPSWE [Tg2576] Model of Familial Alzheimer's Disease

Spatial gene expression technology was applied to a 12-month-old male APPSWE [Tg2576] mouse model of familial Alzheimer's disease (Taconic Biosciences). FIG. 8A illustrates differential gene expression between wt/wt and tg/wt mice, highlighting elevated expression of pro-melanin concentrating hormone (PMCH). This scatter plot of genes across replicates of litter mate controls (wt/wt) and transgenic APPSWE [Tg2576] (tg/wt) mice, highlights differential gene expression with Pmch demonstrating elevated expression. FIG. 8B illustrates neurogranin (Nrgn) expression and demonstrates a similar spatial pattern between wt/wt and tg/wt mice. However, spatial examination of PMCH demonstrated increased expression within the hypothalamic region, as well as other areas within tg/wt mice (FIG. 8C).

### Example 6: Further Application of Spatial Gene Expression on the APPSWE [Tg2576] Model of Familial Alzheimer's Disease

Additional genetic markers were evaluated in the APPSWE model as discussed above. In brief, spatial gene expression technology was applied to a 12-month-old male APPSWE [Tg2576] mouse model of familial Alzheimer's disease (Taconic Biosciences). As shown in Table 10, compared to wild-type control mice, tg/wt mice showed differential expression of multiple genes.

TABLE 10
Top differentially expressed genes in hippocampus of wt/wt and tg/wt mice

| Condition | | | | | | | | |
|---|---|---|---|---|---|---|---|---|
| Type | Condition1 | Condition2 | Feature_ID | Feature_name | Mean1 | Mean2 | Adj_p_value | Log2FC |
| description | V19L01-027 B1_ | V19L01- | ENSMUSG00000035383 | Pmch | 0.088 | 7.400 | 0.000 | −6.384 |
| | Control_ | 029_81 | | | | | | |
| | 1349M_wt_wt_ | Alzheimer_ | | | | | | |
| | Hippocampal | 1349M_tg_wt_ | | | | | | |
| | | Hippocampal | | | | | | |
| description | V19L01-027 B1_ | V19L01- | ENSMUSG00000045471 | Hcrt | 0.024 | 0.793 | 0.000 | −5.057 |
| | Control_ | 029_81 | | | | | | |
| | 1349M_wt_wt_ | Alzheimer_ | | | | | | |
| | Hippocampal | 1349M_tg_wt_ | | | | | | |
| | | Hippocampal | | | | | | |
| description | V19L01-027 B1_ | V19L01- | ENSMUSG00000028550 | Atg4c | 0.102 | 0.372 | 0.000 | −1.859 |
| | Control_ | 029_81 | | | | | | |
| | 1349M_wt_wt_ | Alzheimer_ | | | | | | |
| | Hippocampal | 1349M_tg_wt_ | | | | | | |
| | | Hippocampal | | | | | | |
| description | V19L01-027 B1_ | V19L01- | ENSMUSG00000045410 | Ark1e1 | 0.125 | 0.413 | 0.000 | −1.727 |
| | Control_ | 029_81 | | | | | | |
| | 1349M_wt_wt_ | Alzheimer_ | | | | | | |
| | Hippocampal | 1349M_tg_wt_ | | | | | | |
| | | Hippocampal | | | | | | |
| description | V19L01-027 B1_ | V19L01- | ENSMUSG00000020178 | Adora2a | 0.437 | 0.142 | 0.000 | 1.614 |
| | Control_ | 029_81 | | | | | | |
| | 1349M_wt_wt_ | Alzheimer_ | | | | | | |
| | Hippocampal | 1349M_tg_wt_ | | | | | | |
| | | Hippocampal | | | | | | |
| description | V19L01-027 C1_ | V19L01- | ENSMUSG00000035383 | Pmch | 0.095 | 7.916 | 0.000 | −6.377 |
| | Control_ | 029_81 | | | | | | |
| | 1349M_wt_wt_ | Alzheimer_ | | | | | | |
| | Hippocampal | 1349M_tg_wt_ | | | | | | |
| | | Hippocampal | | | | | | |
| description | V19L01-027 C1_ | V19L01- | ENSMUSG00000045471 | Hcrt | 0.020 | 0.848 | 0.000 | −5.361 |
| | Control_ | 029_81 | | | | | | |
| | 1349M_wt_wt_ | Alzheimer_ | | | | | | |
| | Hippocampal | 1349M_tg_wt_ | | | | | | |
| | | Hippocampal | | | | | | |
| description | V19L01-027 C1_ | V19L01- | ENSMUSG00000079941 | Gm11273 | 0.135 | 0.631 | 0.000 | −2.224 |
| | Control_ | 029_81 | | | | | | |
| | 1349M_wt_wt_ | Alzheimer_ | | | | | | |
| | Hippocampal | 1349M_tg_wt_ | | | | | | |
| | | Hippocampal | | | | | | |
| description | V19L01-027 C1_ | V19L01- | ENSMUSG00000005892 | Trh | 0.905 | 0.272 | 0.000 | 1.733 |
| | Control_ | 029_81 | | | | | | |
| | 1349M_wt_wt_ | Alzheimer_ | | | | | | |
| | Hippocampal | 1349M_tg_wt | | | | | | |
| | | Hippocampal | | | | | | |
| description | V19L01-027 C1_ | V19L01- | ENSMUSG00000045410 | Akr1e1 | 0.138 | 0.442 | 0.000 | −1.675 |
| | Control_ | 029_81 | | | | | | |
| | 1349M_wt_wt_ | Alzheimer_ | | | | | | |
| | Hippocampal | 1349M_tg_wt_ | | | | | | |
| | | Hippocampal | | | | | | |
| description | V19L01-027 B1_ | V19L01- | ENSMUSG00000058443 | Rpl10-ps3 | 0.696 | 0.273 | 0.000 | 1.352 |
| | Alzheimer_ | 029_C1_ | | | | | | |
| | 1349M_tg_wt_ | Alzheimer_ | | | | | | |
| | Hippocampal | 1349M_tg_wt | | | | | | |
| | | Hippocampal | | | | | | |
| description | V19L01-027 B1_ | V19L01- | ENSMUSG00000027301 | Oxt | 3.765 | 1.710 | 0.000 | 1.139 |
| | Alzheimer_ | 029_C1_ | | | | | | |
| | 1349M_tg_wt_ | Alzheimer_ | | | | | | |
| | Hippocampal | 1349M_tg_wt_ | | | | | | |
| | | Hippocampal | | | | | | |
| description | V19L01-027 B1_ | V19L01- | ENSMUSG00000021913 | Ogdhl | 1.026 | 0.478 | 0.000 | 1.101 |
| | Alzheimer_ | 029_C1_ | | | | | | |
| | 1349M_tg_wt_ | Alzheimer_ | | | | | | |
| | Hippocampal | 1349M_tg_wt_ | | | | | | |
| | | Hippocampal | | | | | | |
| description | V19L01-027 B1_ | V19L01- | ENSMUSG00000054256 | Msi1 | 0.317 | 0.156 | 0.000 | 1.022 |
| | Alzheimer_ | 029_C1_ | | | | | | |
| | 1349M_tg_wt_ | Alzheimer_ | | | | | | |
| | Hippocampal | 1349M_tg_wt_ | | | | | | |
| | | Hippocampal | | | | | | |
| description | V19L01-027 B1_ | V19L01- | ENSMUSG00000003469 | Phyhip | 1.551 | 0.769 | 0.000 | 1.011 |
| | Alzheimer_ | 029_C1_ | | | | | | |
| | 1349M_tg_wt_ | Alzheimer_ | | | | | | |
| | Hippocampal | 1349M_tg_wt | | | | | | |
| | | Hippocampal | | | | | | |

Scatter plots of genes across replicates of litter mate controls (wt/wt) and transgenic APPSWE [Tg2576] (tg/wt) mice highlight differential gene expression in various genes demonstrating decreased or elevated expression. See FIG. 10. Spatial examination of Adora2a (FIG. 11) and Trh (FIG. 12) demonstrated decreased expression within the hypothalamic region, as well as other areas within tg/wt mice. Spatial examination of Akr1e1 (FIG. 13), Atg4c (FIG. 14) Gm14296 (FIG. 15), Hcrt (FIG. 16), and Ttr (FIG. 17) demonstrated increased expression within the hypothalamic region, as well as other areas within tg/wt mice.

Sequence Listing
Synthetic PURAMATRIX® polypeptide sequence
SEQ ID NO: 30
RADARADARADARADA
Synthetic EAK16 polypeptide sequence
SEQ ID NO: 31
AEAEAKAKAEAEAKAK
Synthetic KLD12 polypeptide sequence
SEQ ID NO: 32
KLDLKLDLKLDL
18s Probe 1 (P1)
SEQ ID NO: 33
GAGGAATTCCCAGTAAGT
18s Probe 2 (P2)
SEQ ID NO: 34
GAGATTGAGCAATAACAG
18s Probe 3 (P3)
SEQ ID NO: 35
GTAGTTCCGACCATAAAC
18s Probe 4 (P4)
SEQ ID NO: 36
GGTGACTCTAGATAACCT

### Example 7: Further Application of Spatial Gene Expression on the APPSWE [Tg2576] Model of Familial Alzheimer's Disease

Additional genetic markers were evaluated in the APPSWE model as discussed above. In brief, spatial gene expression technology was applied to a 12-month-old male APPSWE [Tg2576] mouse model of familial Alzheimer's disease (Taconic Biosciences).

In order to identify genes whose expression is specific to each cluster, analysis pipelines were used to test, for each gene and each cluster, whether the in-cluster mean differs from the out-of-cluster mean. In order to find differentially expressed genes between groups of cells, the pipeline used the sSeq method which employs a negative binomial exact test. The sSeq method is described in Yu, D., Huber, W. & Vitek, O., Shrinkage estimation of dispersion in Negative Binomial models for RNA-seq experiments with small sample size, Bioinformatics 29, 1275-1282 (2013), which is hereby incorporated by reference in it entirety. When the counts become large, the pipeline switched to the fast asymptotic beta test used in edgeR. The fast asymptotic beta test used in edgeR is described in Robinson, M. D. & Smyth, G. K., Small-sample estimation of negative binomial dispersion, with applications to SAGE data, Biostatistics 9, 321-332 (2007), which is hereby incorporated by reference in it entirety. For each cluster, the algorithm was run on that cluster versus all other cells, yielding a list of genes that are differentially expressed in that cluster relative to the rest of the sample. The pipeline computed relative library size as the total UMI counts for each cell divided by the median UMI counts per cell. Prior to differential expression analysis, replicates and conditions were passed through an aggregator pipeline that normalizes and aggregates the matrices across samples.

As shown in the Tables below, compared to wild-type control mice, tg/wt mice showed differential expression of multiple genes. The Tables were generated using Cell Ranger and Loupe Browser (10× Genomics, Pleasanton, Calif.).

TABLE 11
Top differentially expressed genes in hippocampus of wt/wt and tg/wt
mice (upregulated genes in tg/wt mice)

| | | Hippocampus_ | Hippocampus_ | Hippocampus_ |
|---|---|---|---|---|
| | | Alzheimers | Alzheimers Log2 | Alzheimers |
| FeatureID | FeatureName | Average | Fold Change | P-Value |
| ENSMUSG00000035383 | Pmch | 9.87459958 | 6.63014796 | 4.24E−111 |
| ENSMUSG00000045471 | Hcrt | 1.13151336 | 5.55732919 | 5.37E−24 |
| ENSMUSG00000079037 | Prnp | 35.1160266 | 1.41316398 | 5.63E−18 |
| ENSMUSG00000021948 | Prkcd | 2.57479493 | 1.23327617 | 2.11E−12 |
| ENSMUSG00000036905 | Clqb | 1.6134692 | 1.05147806 | 1.18E−09 |
| ENSMUSG00000026473 | Glul | 7.94968541 | 1.03088145 | 1.48E−09 |
| ENSMUSG00000020415 | Pttg1 | 1.34162154 | 0.88725232 | 6.58E−07 |
| ENSMUSG00000065990 | Aurkaip1 | 2.34205972 | 0.86694549 | 1.11E−06 |
| ENSMUSG00000021647 | Cartpt | 1.68021895 | 0.94174206 | 2.47E−06 |
| ENSMUSG00000073418 | C4b | 1.72207897 | 0.85060935 | 4.53E−06 |
| ENSMUSG00000033186 | Mzt1 | 1.41483616 | 0.80377754 | 1.11E−05 |
| ENSMUSG00000038764 | Ptpn3 | 1.17709067 | 0.80916623 | 1.60E−05 |
| ENSMUSG00000003469 | Phyhip | 1.22008204 | 0.75529427 | 5.68E−05 |
| ENSMUSG00000044349 | Snhg11 | 5.71623546 | 0.72059154 | 0.00012752 |
| ENSMUSG00000028426 | Rad23b | 2.05566611 | 0.72020576 | 0.00013992 |
| ENSMUSG00000059857 | Ntng1 | 1.17919175 | 0.6999172 | 0.00041466 |
| ENSMUSG00000021134 | Srsf5 | 2.60905873 | 0.67909106 | 0.00041749 |
| ENSMUSG00000026384 | Ptpn4 | 1.69331031 | 0.68754071 | 0.00045081 |
| ENSMUSG00000054027 | Nt5dc3 | 1.31188315 | 0.67486621 | 0.00056268 |
| ENSMUSG00000045294 | Insig1 | 1.09611821 | 0.66212879 | 0.00077751 |
| ENSMUSG00000033768 | Nrxn2 | 2.44792192 | 0.6488397 | 0.00095387 |
| ENSMUSG00000052949 | Rnf157 | 2.03918069 | 0.64616114 | 0.00103143 |
| ENSMUSG00000022594 | Lynx1 | 2.56542087 | 0.64468131 | 0.00105315 |
| ENSMUSG00000028926 | Cdk14 | 2.26755212 | 0.641981 | 0.00114251 |
| ENSMUSG00000028760 | Eif4g3 | 1.65306651 | 0.63162745 | 0.00153968 |
| ENSMUSG00000026335 | Pam | 1.20068744 | 0.6354298 | 0.00154409 |
| ENSMUSG00000040612 | Ildr2 | 1.13086687 | 0.63867232 | 0.00155168 |
| ENSMUSG00000096768 | Gm47283 | 2.30100781 | 0.6287651 | 0.00156704 |
| ENSMUSG00000026463 | Atp2b4 | 1.17838364 | 0.6194991 | 0.00243697 |
| ENSMUSG00000021978 | Extl3 | 1.15624148 | 0.61378066 | 0.00253559 |
| ENSMUSG00000039883 | Lrrc17 | 1.55706323 | 0.61097256 | 0.00262576 |
| ENSMUSG00000029328 | Hnmpdl | 2.44501273 | 0.60497905 | 0.00274079 |
| ENSMUSG00000038807 | Rap1gap2 | 1.35293506 | 0.60495919 | 0.0030478 |
| ENSMUSG00000090071 | Cdk5r2 | 1.34000532 | 0.60415658 | 0.00307196 |
| ENSMUSG00000015165 | Hnmpl | 1.45055455 | 0.60152017 | 0.00316468 |
| ENSMUSG00000036896 | C1qc | 1.05587441 | 0.60601087 | 0.00329788 |
| ENSMUSG00000059003 | Grin2a | 1.88127632 | 0.59610013 | 0.00356702 |
| ENSMUSG00000027350 | Chgb | 6.74754338 | 0.59219371 | 0.00356728 |
| ENSMUSG00000032452 | Clstn2 | 1.02338846 | 0.598116 | 0.00368233 |
| ENSMUSG00000031762 | Mt2 | 4.25695334 | 0.58602705 | 0.00402678 |
| ENSMUSG00000030729 | Pgm2l1 | 3.12269242 | 0.58443211 | 0.00428113 |
| ENSMUSG00000035390 | Brsk1 | 1.52506214 | 0.58606279 | 0.00436223 |
| ENSMUSG00000020396 | Nefh | 1.59827676 | 0.59084825 | 0.00448374 |
| ENSMUSG00000025266 | Gnl3l | 1.68232003 | 0.57980633 | 0.00490353 |
| ENSMUSG00000050965 | Prkca | 1.39770426 | 0.58493724 | 0.00494354 |
| ENSMUSG00000030795 | Fus | 1.66389516 | 0.5786349 | 0.00498227 |
| ENSMUSG00000062257 | Opcml | 1.84264874 | 0.57635317 | 0.00522 |
| ENSMUSG00000022351 | Sqle | 1.11826038 | 0.57868798 | 0.00529962 |
| ENSMUSG00000056602 | Fry | 1.50065727 | 0.57484867 | 0.00543022 |
| ENSMUSG00000070802 | Pnmal2 | 4.86626706 | 0.56858439 | 0.005793 |
| ENSMUSG00000052727 | Map1b | 7.49100309 | 0.56629853 | 0.00588621 |
| ENSMUSG00000052684 | Jun | 1.14185715 | 0.57407829 | 0.00592542 |
| ENSMUSG00000028289 | Epha7 | 1.02904522 | 0.56611123 | 0.00767726 |
| ENSMUSG00000030209 | Grin2b | 1.10436092 | 0.55551102 | 0.00890302 |
| ENSMUSG00000096847 | Tmem151b | 1.05555117 | 0.55376294 | 0.00904167 |
| ENSMUSG00000021750 | Fam107a | 2.40848623 | 0.54399951 | 0.01000758 |
| ENSMUSG00000058624 | Gda | 1.61831785 | 0.54530773 | 0.01056643 |
| ENSMUSG00000039943 | Plcb4 | 1.27697287 | 0.54806802 | 0.01090433 |
| ENSMUSG00000018340 | Anxa6 | 1.33192424 | 0.54111802 | 0.01130033 |
| ENSMUSG00000039270 | Megf9 | 1.35309668 | 0.53883766 | 0.01198635 |
| ENSMUSG00000039218 | Srrm2 | 1.64611678 | 0.53611817 | 0.01209207 |
| ENSMUSG00000020524 | Gria1 | 2.66352523 | 0.53304477 | 0.01263211 |
| ENSMUSG00000026824 | Kcnj3 | 1.07074361 | 0.53475148 | 0.01372188 |
| ENSMUSG00000018909 | Arrb1 | 1.32335829 | 0.53017527 | 0.01400557 |
| ENSMUSG00000023845 | Lnpep | 1.02419657 | 0.52552307 | 0.01620938 |
| ENSMUSG00000018411 | Mapt | 2.16799317 | 0.52003772 | 0.01637085 |
| ENSMUSG00000022055 | Nefl | 5.59372623 | 0.51782223 | 0.01640064 |
| ENSMUSG00000040724 | Kcna2 | 1.88499362 | 0.52096335 | 0.01653253 |
| ENSMUSG00000032238 | Rora | 1.1720804 | 0.52724022 | 0.01731721 |
| ENSMUSG00000037111 | Setd7 | 1.38396642 | 0.5192551 | 0.01736129 |
| ENSMUSG00000029212 | Gabrb1 | 1.04617711 | 0.52138736 | 0.01743747 |
| ENSMUSG00000024897 | Apba1 | 1.44748374 | 0.51560304 | 0.0185627 |
| ENSMUSG00000016349 | Eef1a2 | 12.3575934 | 0.50794372 | 0.01936138 |
| ENSMUSG00000045038 | Prkce | 2.62570576 | 0.50927868 | 0.02006948 |
| ENSMUSG00000067889 | Sptbn2 | 3.96651919 | 0.50820679 | 0.02016986 |
| ENSMUSG00000037217 | Syn1 | 7.24986355 | 0.50582988 | 0.02042361 |
| ENSMUSG00000046093 | Hpcal4 | 4.58536859 | 0.5076774 | 0.02050425 |
| ENSMUSG00000020440 | Arf5 | 2.44937651 | 0.50715516 | 0.02071024 |
| ENSMUSG00000115783 | Bc1 | 16.2843537 | 0.50261206 | 0.02179054 |
| ENSMUSG00000030500 | Slc17a6 | 1.73694816 | 0.51312084 | 0.02180683 |
| ENSMUSG00000022982 | Sod1 | 6.06889396 | 0.50130985 | 0.02190372 |
| ENSMUSG00000069917 | Hba-a2 | 3.32358816 | 0.58029984 | 0.02322812 |
| ENSMUSG00000030257 | Srgap3 | 1.48562645 | 0.50204531 | 0.02343925 |
| ENSMUSG00000070304 | Scn2b | 2.63006954 | 0.4996594 | 0.02361173 |
| ENSMUSG00000022421 | Nptxr | 6.03446854 | 0.50101166 | 0.02368384 |
| ENSMUSG00000025092 | Hspa12a | 1.57888216 | 0.49971978 | 0.02432261 |
| ENSMUSG00000021273 | Fdft1 | 2.05582773 | 0.49469629 | 0.02567376 |
| ENSMUSG00000038467 | Chmp4b | 2.10657693 | 0.49180599 | 0.02664021 |
| ENSMUSG00000040907 | Atp1a3 | 11.8074332 | 0.48805539 | 0.02684495 |
| ENSMUSG00000018548 | Trim37 | 2.41769866 | 0.48416042 | 0.03008108 |
| ENSMUSG00000022332 | Khdrbs3 | 2.53988465 | 0.48496574 | 0.03008108 |
| ENSMUSG00000013033 | Adgrl1 | 3.13255134 | 0.48224787 | 0.03060174 |
| ENSMUSG00000058297 | Spock2 | 3.45498658 | 0.48007942 | 0.0317683 |
| ENSMUSG00000020361 | Hspa4 | 1.62203515 | 0.48224031 | 0.0317683 |
| ENSMUSG00000021670 | Hmgcr | 1.06896577 | 0.48563722 | 0.0317683 |
| ENSMUSG00000025867 | Cplx2 | 4.76299081 | 0.47991069 | 0.03234834 |
| ENSMUSG00000022048 | Dpysl2 | 7.38805009 | 0.47655488 | 0.03291409 |
| ENSMUSG00000033392 | Clasp2 | 1.55172972 | 0.47891849 | 0.03419796 |
| ENSMUSG00000040118 | Cacna2d1 | 1.40627021 | 0.47774383 | 0.03554646 |
| ENSMUSG00000022212 | Cpne6 | 2.84211718 | 0.47119322 | 0.03922686 |

TABLE 12
Top differentially expressed genes in hippocampus of wt/wt and tg/wt mice
(downregulated genes in tg/wt mice)

| | | Hippocampus_ | Hippocampus_ | Hippocampus_ |
|---|---|---|---|---|
| | | Alzheimers | Alzheimers Log2 | Alzheimers |
| FeatureID | FeatureName | Average | Fold Change | P-Value |
| ENSMUSG00000027301 | Oxt | 2.86361287 | −2.0512914 | 4.38E−15 |
| ENSMUSG00000028393 | Alad | 0.45577313 | −1.2882098 | 3.74E−14 |
| ENSMUSG00000034875 | Nudt19 | 0.77643053 | −1.1310002 | 4.61E−11 |
| ENSMUSG00000060143 | Gm10076 | 1.25822475 | −1.1054725 | 1.09E−10 |
| ENSMUSG00000028656 | Cap1 | 0.90411166 | −1.0826511 | 3.64E−10 |
| ENSMUSG00000022018 | Rgcc | 0.55209965 | −0.9271875 | 2.41E−07 |
| ENSMUSG00000090137 | Uba52 | 3.77564399 | −0.908434 | 2.71E−07 |
| ENSMUSG00000061718 | Ppp1r1b | 1.81969846 | −0.8165306 | 1.53E−05 |
| ENSMUSG00000023868 | Pde10a | 0.60042453 | −0.8008587 | 3.62E−05 |
| ENSMUSG00000005575 | Ube2m | 2.96252533 | −0.7237655 | 1.34E−04 |
| ENSMUSG00000069919 | Hba-a1 | 4.52023505 | −0.8360175 | 1.43E−04 |
| ENSMUSG00000060803 | Gstp1 | 1.10565389 | −0.7310775 | 1.46E−04 |
| ENSMUSG00000032575 | Manf | 1.22493069 | −0.6998419 | 2.88E−04 |
| ENSMUSG00000068696 | Gpr88 | 0.95486087 | −0.7093435 | 0.00050323 |
| ENSMUSG00000020519 | Sap30l | 0.65359806 | −0.6756029 | 0.00063551 |
| ENSMUSG00000042831 | Alkbh6 | 0.64082995 | −0.6708863 | 0.00071801 |
| ENSMUSG00000098234 | Snhg6 | 1.00948899 | −0.6253886 | 0.00199594 |
| ENSMUSG00000037727 | Avp | 8.29830337 | −0.9588796 | 0.00219137 |
| ENSMUSG00000018293 | Pfn1 | 2.72785066 | −0.6068787 | 0.00279371 |
| ENSMUSG00000061762 | Tac1 | 1.1586658 | −0.6338359 | 0.00281841 |
| ENSMUSG00000060636 | Rpl35a | 21.2142997 | −0.583893 | 0.00431092 |
| ENSMUSG00000008668 | Rps18 | 5.92941445 | −0.5788915 | 0.00488602 |
| ENSMUSG00000037563 | Rps16 | 12.7514654 | −0.5747596 | 0.00520105 |
| ENSMUSG00000039105 | Atp6v1g1 | 4.02341002 | −0.5644062 | 0.00660847 |
| ENSMUSG00000048240 | Gng7 | 0.77772351 | −0.56605 | 0.008779 |
| ENSMUSG00000032766 | Gng11 | 1.00819602 | −0.5546753 | 0.01034799 |
| ENSMUSG00000062997 | Rpl35 | 9.7457871 | −0.5370146 | 0.01148443 |
| ENSMUSG00000007944 | Ttc9b | 3.15954216 | −0.536717 | 0.01251601 |
| ENSMUSG00000022427 | Tomm22 | 1.69767409 | −0.5344789 | 0.01278578 |
| ENSMUSG00000047945 | Marcksl1 | 0.94225438 | −0.537552 | 0.0135835 |
| ENSMUSG00000030615 | Tmem126a | 0.81473487 | −0.5344193 | 0.01404564 |
| ENSMUSG00000008682 | Rpl10 | 7.75121399 | −0.5269604 | 0.01430142 |
| ENSMUSG00000050708 | Ftl1 | 8.4901483 | −0.5267301 | 0.01431562 |
| ENSMUSG00000061477 | Rps7 | 12.0844528 | −0.5163 | 0.01736129 |
| ENSMUSG00000061983 | Rps12 | 14.9729554 | −0.5153296 | 0.01778727 |
| ENSMUSG00000053565 | Eif3k | 2.72494147 | −0.5157138 | 0.01804875 |
| ENSMUSG00000047215 | Rpl9 | 19.5917796 | −0.5004509 | 0.02317704 |
| ENSMUSG00000032060 | Cryab | 4.88533842 | −0.5082585 | 0.02320074 |
| ENSMUSG00000028234 | Rps20 | 15.6958891 | −0.499441 | 0.02343925 |
| ENSMUSG00000045573 | Penk | 4.05589598 | −0.5243615 | 0.02458827 |
| ENSMUSG00000037843 | Vstm2l | 1.77169682 | −0.4987025 | 0.02567376 |
| ENSMUSG00000022132 | Cldn10 | 1.22008204 | −0.4973158 | 0.02627959 |
| ENSMUSG00000068523 | Gng5 | 2.11708234 | −0.4915584 | 0.02799174 |
| ENSMUSG00000036781 | Rps27l | 1.31931775 | −0.4925671 | 0.02815556 |
| ENSMUSG00000063480 | Snu13 | 2.56235006 | −0.4848905 | 0.03036092 |
| ENSMUSG00000025408 | Ddit3 | 0.79647162 | −0.4871521 | 0.03175543 |
| ENSMUSG00000041453 | Rpl21 | 21.1549845 | −0.4677535 | 0.03948733 |
| ENSMUSG00000020460 | Rps27a | 20.0076322 | −0.4659113 | 0.04072789 |
| ENSMUSG00000020484 | Xbp1 | 1.1318366 | −0.4703695 | 0.04090211 |
| ENSMUSG00000062328 | Rpl17 | 17.5661752 | −0.4608213 | 0.04404804 |
| ENSMUSG00000025362 | Rps26 | 13.9494053 | −0.4596175 | 0.04512285 |
| ENSMUSG00000027199 | Gatm | 1.653713 | −0.468694 | 0.04548188 |
| ENSMUSG00000058600 | Rpl30 | 14.8950537 | −0.4549598 | 0.04913874 |
| ENSMUSG00000071415 | Rpl23 | 15.6782724 | −0.454004 | 0.04953793 |
| ENSMUSG00000014301 | Pam16 | 2.09494017 | −0.4562433 | 0.04998985 |
| ENSMUSG00000047675 | Rps8 | 19.7593813 | −0.4519425 | 0.05091112 |
| ENSMUSG00000079641 | Rpl39 | 15.5856631 | −0.4497488 | 0.0527836 |
| ENSMUSG00000027562 | Car2 | 4.10244302 | −0.455716 | 0.0535349 |
| ENSMUSG00000041841 | Rpl37 | 31.2619961 | −0.4480622 | 0.05392483 |
| ENSMUSG00000090733 | Rps27 | 19.7136424 | −0.441018 | 0.05993373 |
| ENSMUSG00000028936 | Rpl22 | 5.24365368 | −0.4412601 | 0.06006023 |
| ENSMUSG00000001313 | Rnd2 | 2.16055857 | −0.4314959 | 0.06982624 |
| ENSMUSG00000079435 | Rpl36a | 7.99073732 | −0.4287553 | 0.07033693 |
| ENSMUSG00000078427 | Sarnp | 0.9834679 | −0.4322818 | 0.07172687 |
| ENSMUSG00000024646 | Cyb5a | 1.70203788 | −0.4291996 | 0.07241252 |
| ENSMUSG00000040952 | Rps19 | 14.9959057 | −0.4226163 | 0.07587754 |
| ENSMUSG00000020022 | Ndufa12 | 6.45759409 | −0.4206885 | 0.07814587 |
| ENSMUSG00000007872 | Id3 | 0.94273924 | −0.4321917 | 0.07915615 |
| ENSMUSG00000034424 | Gcsh | 0.8365538 | −0.426073 | 0.07915615 |
| ENSMUSG00000023089 | Ndufa5 | 7.78725563 | −0.4193806 | 0.07935164 |
| ENSMUSG00000039001 | Rps21 | 41.5985104 | −0.4174095 | 0.08114339 |
| ENSMUSG00000046727 | Cystm1 | 2.0959099 | −0.4183165 | 0.08335454 |
| ENSMUSG00000041607 | Mbp | 29.4014073 | −0.4183188 | 0.08733257 |
| ENSMUSG00000025290 | Rps24 | 25.5460838 | −0.4110906 | 0.08865811 |
| ENSMUSG00000012405 | Rpl15 | 8.51907858 | −0.4110663 | 0.08878557 |
| ENSMUSG00000026830 | Ermn | 0.79954243 | −0.4213257 | 0.0923022 |
| ENSMUSG00000007892 | Rplp1 | 42.0546068 | −0.4064774 | 0.09337864 |
| ENSMUSG00000059291 | Rpl11 | 15.4240415 | −0.4046862 | 0.09592558 |
| ENSMUSG00000029359 | Tesc | 1.41160372 | −0.4093749 | 0.09794219 |
| ENSMUSG00000008683 | Rps15a | 12.2305588 | −0.4021629 | 0.09925154 |
| ENSMUSG00000012848 | Rps5 | 10.2814013 | −0.4012341 | 0.10058586 |
| ENSMUSG00000046364 | Rpl27a | 18.371536 | −0.4002955 | 0.10113278 |
| ENSMUSG00000041120 | Nbl1 | 0.78968351 | −0.4113762 | 0.10231987 |
| ENSMUSG00000049517 | Rps23 | 15.544288 | −0.3994971 | 0.10231987 |
| ENSMUSG00000041881 | Ndufa7 | 5.89563552 | −0.3994508 | 0.10231987 |
| ENSMUSG00000035885 | Cox8a | 35.9661566 | −0.3984874 | 0.10254119 |
| ENSMUSG00000017778 | Cox7c | 23.6869497 | −0.3977921 | 0.10360034 |
| ENSMUSG00000076439 | Mog | 1.28376098 | −0.409943 | 0.10485842 |
| ENSMUSG00000060036 | Rpl3 | 11.575991 | −0.3962732 | 0.10653102 |
| ENSMUSG00000062006 | Rpl34 | 17.1299583 | −0.3952945 | 0.10763329 |
| ENSMUSG00000022820 | Ndufb4 | 7.6595745 | −0.3944297 | 0.10931541 |
| ENSMUSG00000052146 | Rps10 | 14.0976124 | −0.3924381 | 0.1122188 |
| ENSMUSG00000054091 | 1810037I17Rik | 3.05416482 | −0.3927535 | 0.11352313 |
| ENSMUSG00000027679 | Dnajc19 | 1.43439238 | −0.392651 | 0.11704727 |
| ENSMUSG00000026888 | Grb14 | 0.7985727 | −0.4007208 | 0.11844098 |
| ENSMUSG00000038803 | Ost4 | 2.40185974 | −0.3896925 | 0.11854097 |
| ENSMUSG00000106918 | Mrpl33 | 2.64364577 | −0.3891803 | 0.118869 |
| ENSMUSG00000002768 | Mea1 | 1.92232822 | −0.3892585 | 0.11952483 |
| ENSMUSG00000047557 | Lxn | 0.91251599 | −0.3923498 | 0.12000574 |
| ENSMUSG00000006412 | Pfdn2 | 2.766155 | −0.3878486 | 0.12012273 |

### Example 8: Human Brain Analyses

Spatial analysis was performed on an unspecified human cerebral cortex sample cord (FIGS. 18A and 18B), a temporal human cerebral cortex sample (FIGS. 19A and 19B), and human neuronal samples from spinal cord (FIGS. 20A and 20B) and cerebellum (FIGS. 21A and 21B).

Multiple neuronal samples, i.e., cerebellum, cerebrum (non-specific), cerebrum (temporal), and spinal cord, were compared using t-SNE and UMAP plots (see FIGS. 22A and 22B). The t-SNE plot and UMAP plots demonstrated distinct cell type clustering and relationships, respectively. A scatter plot demonstrated the differential expression of genes captured using the methods described herein (FIG. 23). Comparison of different brain regions exhibited larger spread, compared to similar regions (e.g., cerebral tissues).

Cerebral tissues from different region/sources (BioIVT nonspecific, BioIVT temporal, secondary source 1, secondary source 2, and secondary source 3) were compared (FIGS. 24A and 24B). A t-SNE plot and UMAP plot demonstrated distinct cell type clustering and relationships, respectively. A scatter plot demonstrated the differential expression of genes captured using the methods described herein (FIG. 25).

Healthy and glioblastoma samples from the cerebral cortex were also compared. A t-SNE plot and UMAP plot demonstrated distinct cell type clustering and relationships, respectively (FIGS. 26A and 26B).

### Example 9: Comparison Between Healthy and Glioblastoma Samples

Spatial analysis was performed on healthy (nonspecific & temporal) and glioblastoma samples from different patients. Two tissue sections each taken from a glioblastoma sample and a healthy control sample were used. A scatter plot demonstrated the differential expression of genes captured using the methods described herein (FIG. 27B). Decreased expression of GABRA1, CPLX2, ST8SIA3, GABRG2, and KCNC2 was observed in glioblastoma compared to healthy sample (FIG. 27A).

A scatter plot demonstrated the differential expression of genes captured using the methods described herein (FIG. 28B). Differentially expressed genes were observed. For example, overexpression of CHI3L1, TIMP1, PLIN2, and CD44 and underexpression of MBP within the glioblastoma sample were observed (FIG. 28A).

Comparison of between healthy and glioblastoma samples also showed other genes were differentially expressed, for example, the genes shown in FIGS. 29A and 29B as well as Tables 13 and 14.

TABLE 13
Top overexpressed genes in glioblastoma relative to normal

| | | Glioblastoma | | | Normal | |
|---|---|---|---|---|---|---|
| Feature | Glioblastoma | Log2 Fold | Glioblastoma | Normal | Log2 Fold | Normal |
| Name | Average | Change | P-Value | Average | Change | P-Value |
| COL1A1 | 1.23999706 | 10.0638561 | 0 | 0.00110818 | −10.063856 | 0 |
| COL3A1 | 1.06297501 | 9.90576698 | 0 | 0.00105781 | −9.905767 | 0 |
| COL8A1 | 1.09405673 | 8.82181405 | 0 | 0.00236748 | −8.8218141 | 0 |
| WEE1 | 1.66304555 | 8.51904123 | 0 | 0.0044831 | −8.5190412 | 0 |
| CHI3L1 | 18.0404354 | 7.04026861 | 0 | 0.13701146 | −7.0402686 | 0 |
| MGP | 4.4401254 | 7.03962288 | 0 | 0.03369877 | −7.0396229 | 0 |
| SRPX | 1.09653534 | 6.89434132 | 0 | 0.00916768 | −6.8943413 | 0 |
| SERPINE1 | 3.52695734 | 6.63398859 | 0 | 0.03546179 | −6.6339886 | 0 |
| COL1A2 | 1.26151136 | 6.62923995 | 0 | 0.01269371 | −6.62924 | 0 |
| TIMP1 | 8.95669102 | 6.48071995 | 0 | 0.10024 | −6.48072 | 0 |
| ANXA1 | 3.15377841 | 6.32864809 | 0 | 0.03918931 | −6.3286481 | 0 |
| COL6A2 | 1.06297501 | 5.85344594 | 0 | 0.01833536 | −5.8534459 | 0 |
| CAV1 | 1.20608973 | 5.56157824 | 0 | 0.02548816 | −5.5615782 | 0 |
| PLIN2 | 2.85624653 | 5.45801383 | 0 | 0.06492933 | −5.4580138 | 0 |
| CD44 | 2.4537209 | 5.41720583 | 0 | 0.05737355 | −5.4172058 | 0 |
| APOC1 | 5.78253847 | 5.3730687 | 0 | 0.13947968 | −5.3730687 | 0 |
| IGFBP2 | 4.66914861 | 5.36192666 | 0 | 0.1134878 | −5.3619267 | 0 |
| PDPN | 1.03333089 | 5.35572835 | 0 | 0.02518593 | −5.3557283 | 0 |
| VIM | 25.9392573 | 5.23652741 | 0 | 0.68797886 | −5.2365274 | 0 |
| LGALS3 | 6.94574832 | 4.90982887 | 0 | 0.23100535 | −4.9098289 | 0 |
| VEGFA | 5.15530241 | 4.75663587 | 0 | 0.19065749 | −4.7566359 | 0 |
| IGFBP5 | 2.99698179 | 4.62036276 | 0 | 0.12179916 | −4.6203628 | 0 |
| CTGF | 1.18611217 | 4.55321848 | 0 | 0.0504726 | −4.5532185 | 0 |
| EMP1 | 1.07288944 | 4.35483735 | 0 | 0.05238673 | −4.3548374 | 0 |
| EMP3 | 1.07353388 | 4.35155197 | 0 | 0.05253785 | −4.351552 | 0 |
| IGFBP3 | 1.07958168 | 4.28689993 | 0 | 0.05525793 | −4.2868999 | 0 |
| A2M | 4.80264633 | 4.26503459 | 0 | 0.24974368 | −4.2650346 | 0 |
| ANXA2 | 1.02544892 | 4.21795198 | 0 | 0.05505644 | −4.217952 | 0 |
| FLNA | 1.36933073 | 4.14183216 | 3.01E−306 | 0.07752229 | −4.1418322 | 3.01E−306 |
| IGFBP7 | 7.97159381 | 4.12851759 | 0 | 0.45571421 | −4.1285176 | 0 |
| S100A11 | 1.60574018 | 4.0907128 | 1.96E−302 | 0.09419538 | −4.0907128 | 1.96E−302 |
| ADM | 2.30331908 | 4.0587042 | 1.38E−299 | 0.13817001 | −4.0587042 | 1.38E−299 |
| FN1 | 1.64138253 | 4.03850975 | 6.14E−292 | 0.09983703 | −4.0385097 | 6.14E−292 |
| SERPING1 | 2.00553934 | 3.98757791 | 1.69E−291 | 0.126383 | −3.9875779 | 1.69E−291 |
| MT2A | 47.8913793 | 3.97755744 | 2.62E−295 | 3.04009285 | −3.9775574 | 2.62E−295 |
| S100A10 | 2.19525186 | 3.78345289 | 3.35E−266 | 0.15937656 | −3.7834529 | 3.35E−266 |
| SPARC | 5.0779699 | 3.77129812 | 8.99E−269 | 0.37184507 | −3.7712981 | 8.99E−269 |
| ITGB1 | 1.24768074 | 3.72288566 | 3.20E−257 | 0.09444724 | −3.7228857 | 3.20E−257 |
| SLC5A3 | 1.06183486 | 3.7215987 | 2.00E−255 | 0.08044386 | −3.7215987 | 2.00E−255 |
| FABP7 | 1.92766154 | 3.62028615 | 1.98E−247 | 0.15670686 | −3.6202862 | 1.98E−247 |
| YBX3 | 1.44121031 | 3.58458058 | 3.83E−242 | 0.12008651 | −3.5845806 | 3.83E−242 |
| IFITM2 | 1.64420815 | 3.57344722 | 8.11E−243 | 0.13806927 | −3.5734472 | 8.11E−243 |
| TAGLN2 | 1.1312854 | 3.55871738 | 6.46E−238 | 0.09595839 | −3.5587174 | 6.46E−238 |
| COL6A1 | 1.06917153 | 3.55021661 | 4.43E−238 | 0.09122344 | −3.5502166 | 4.43E−238 |
| HLA-A | 5.10359869 | 3.51334628 | 4.82E−240 | 0.44689914 | −3.5133463 | 4.82E−240 |
| LGALS3BP | 1.08384488 | 3.31576455 | 1.13E−211 | 0.10880322 | −3.3157645 | 1.13E−211 |
| ANXA5 | 1.56201757 | 3.29340226 | 4.49E−211 | 0.15927582 | −3.2934023 | 4.49E−211 |
| APOE | 23.4428053 | 3.2832261 | 4.26E−215 | 2.40797638 | −3.2832261 | 4.26E−215 |
| GADD45A | 1.28461197 | 3.24716836 | 8.61E−204 | 0.13524844 | −3.2471684 | 8.61E−204 |
| TPM4 | 1.83877873 | 3.15405038 | 5.04E−197 | 0.20652462 | −3.1540504 | 5.04E−197 |
| SPP1 | 17.4107207 | 3.13518575 | 1.38E−198 | 1.98162896 | −3.1351858 | 1.38E−198 |

TABLE 14
Top underexpressed genes in glioblastoma relative to normal

| | | Glioblastoma | | | Normal | |
|---|---|---|---|---|---|---|
| Feature | Glioblastoma | Log2 Fold | Glioblastoma | Normal | Log2 Fold | Normal |
| Name | Average | Change | P-Value | Average | Change | P-Value |
| GABRA1 | 0 | −14.352183 | 0 | 1.03670324 | 14.3521834 | 0 |
| CCK | 0.00074358 | −11.865242 | 0 | 2.95899415 | 11.8652417 | 0 |
| SLC17A7 | 0.00039658 | −11.586549 | 0 | 1.37202871 | 11.5865489 | 0 |
| CHGA | 0.00104101 | −10.202205 | 0 | 1.28473428 | 10.2022052 | 0 |
| STMN2 | 0.00143759 | −9.8028619 | 0 | 1.32830594 | 9.80286189 | 0 |
| CALY | 0.00223075 | −9.0017361 | 0 | 1.168879 | 9.00173611 | 0 |
| EEF1A2 | 0.00307347 | −8.7977703 | 0 | 1.38980998 | 8.79777031 | 0 |
| CABP1 | 0.00257775 | −8.6400874 | 0 | 1.04813766 | 8.64008738 | 0 |
| NRGN | 0.0184904 | −8.0782977 | 0 | 5.01089186 | 8.07829775 | 0 |
| SNAP25 | 0.03618765 | −7.9643021 | 0 | 9.04995943 | 7.96430206 | 0 |
| ATP2B2 | 0.00475892 | −7.7395272 | 0 | 1.02758594 | 7.73952723 | 0 |
| SYN1 | 0.00758453 | −7.2364604 | 0 | 1.15114811 | 7.23646035 | 0 |
| NECAB1 | 0.00822897 | −6.9445903 | 0 | 1.01967756 | 6.94459029 | 0 |
| MBP | 0.06583178 | −6.8916979 | 0 | 7.82290092 | 6.8916979 | 0 |
| PHYHIP | 0.01090587 | −6.8331796 | 0 | 1.24912138 | 6.83317959 | 0 |
| BASP1 | 0.01913484 | −6.6459909 | 0 | 1.9212331 | 6.64599094 | 0 |
| CPLX1 | 0.01229389 | −6.505835 | 0 | 1.12168057 | 6.50583504 | 0 |
| VSNL1 | 0.04342518 | −6.505552 | 0 | 3.95001012 | 6.50555202 | 0 |
| TAGLN3 | 0.02201002 | −5.7653121 | 0 | 1.19980733 | 5.76531209 | 0 |
| ENC1 | 0.04322689 | −5.7367194 | 0 | 2.30763564 | 5.7367194 | 0 |
| FBXL16 | 0.02909884 | −5.2660351 | 0 | 1.12157983 | 5.26603506 | 0 |
| CHN1 | 0.1102484 | −5.2245807 | 0 | 4.12399452 | 5.22458072 | 0 |
| KIF5A | 0.04218588 | −5.1231949 | 0 | 1.47196648 | 5.12319487 | 0 |
| PLP1 | 0.12987896 | −5.0483235 | 0 | 4.2992886 | 5.04832352 | 0 |
| OLFM1 | 0.10008611 | −4.9282871 | 0 | 3.04890792 | 4.92828714 | 0 |
| SNCB | 0.05185244 | −4.838491 | 0 | 1.48491205 | 4.838491 | 0 |
| STXBP1 | 0.07178043 | −4.7219566 | 0 | 1.89559382 | 4.72195662 | 0 |
| ATP1B1 | 0.12283972 | −4.7048967 | 0 | 3.20495994 | 4.70489675 | 0 |
| DNM1 | 0.06513777 | −4.6966958 | 0 | 1.69042924 | 4.69669583 | 0 |
| SERPINI1 | 0.05502506 | −4.5038462 | 0 | 1.24947398 | 4.50384622 | 0 |
| PRKAR1B | 0.06434461 | −4.3941476 | 0 | 1.35394522 | 4.39414762 | 0 |
| MEF2C | 0.05338918 | −4.3447482 | 0 | 1.08576544 | 4.34474823 | 0 |
| MTURN | 0.06578221 | −4.266778 | 0 | 1.26720487 | 4.26677801 | 0 |
| NSF | 0.06107285 | −4.2061486 | 2.57E−308 | 1.12812817 | 4.20614862 | 2.57E−308 |
| SYT1 | 0.15193855 | −4.1835448 | 0 | 2.76168758 | 4.18354475 | 0 |
| MAP2 | 0.0757462 | −4.1097179 | 7.11E−302 | 1.3085098 | 4.10971787 | 7.11E−302 |
| MT-ATP8 | 0.16096068 | −4.0814395 | 1.19E−307 | 2.72572207 | 4.08143951 | 1.19E−307 |
| MAP1A | 0.09770665 | −4.0064399 | 1.21E−292 | 1.57104793 | 4.00643992 | 1.21E−292 |
| UCHL1 | 0.23298897 | −3.8907615 | 2.44E−285 | 3.45666813 | 3.89076146 | 2.44E−285 |
| FAIM2 | 0.08541277 | −3.8188601 | 2.44E−271 | 1.20600306 | 3.81886007 | 2.44E−271 |
| STMN1 | 0.21395328 | −3.6921083 | 1.89E−260 | 2.76596919 | 3.69210828 | 1.89E−260 |
| APLP1 | 0.1050929 | −3.5845635 | 1.70E−242 | 1.26131137 | 3.58456347 | 1.70E−242 |
| NCDN | 0.11808079 | −3.5523504 | 2.66E−241 | 1.3858306 | 3.55235042 | 2.66E−241 |
| STMN3 | 0.11198342 | −3.2329552 | 6.54E−204 | 1.05327559 | 3.2329552 | 6.54E−204 |
| MT-ND4L | 0.59382445 | −3.1987417 | 5.70E−207 | 5.45265307 | 3.19874171 | 5.70E−207 |
| BEX1 | 0.17335371 | −3.1870431 | 7.04E−201 | 1.57920817 | 3.18704311 | 7.04E−201 |
| MT-ND2 | 5.0047023 | −3.1332228 | 8.11E−201 | 43.9113161 | 3.13322283 | 8.11E−201 |
| PPP3CA | 0.20225426 | −3.1272765 | 5.75E−196 | 1.7676493 | 3.12727649 | 5.75E−196 |
| MT-ND5 | 1.25843789 | −3.0810993 | 1.52E−194 | 10.6500215 | 3.08109927 | 1.52E−194 |

Spatial analysis of glioblastoma samples demonstrated regional distribution of overexpressed genes in glioblastoma (FIG. 30).

### Example 10: Comparison Between Healthy and Glioblastoma Samples

Spatial analysis was performed on healthy (nonspecific & temporal) and glioblastoma samples from different patients. Four tissue sections from 1 glioblastoma sample and a total of nine tissue sections from two control samples (4 sections from one control sample and 5 sections from the second control sample) were used to create whole transcriptome sequencing libraries. Whole transcriptome sequencing and analysis showed that genes were differentially expressed, for example, the genes shown in Table 15.

TABLE 15
top overexpressed genes in glioblastoma relative to normal, whole
transcriptome results.

| | | | Glioblastoma | |
|---|---|---|---|---|
| | | Glioblastoma | Log2 Fold | Glioblastoma |
| FeatureID | FeatureName | Average | Change | P-Value |
| ENSG00000170290 | SLN | 1.79572485 | 7.11452788 | 0 |
| ENSG00000102359 | SRPX2 | 1.2604062 | 5.6423875 | 1.14E−255 |
| ENSG00000170439 | METTL7B | 2.04773939 | 5.3733812 | 7.59E−237 |
| ENSG00000133110 | POSTN | 2.83567896 | 5.23122146 | 1.81E−227 |
| ENSG00000166741 | NNMT | 1.19618979 | 5.23563833 | 1.62E−225 |
| ENSG00000157150 | TIMP4 | 1.08558475 | 5.10925969 | 1.21E−217 |
| ENSG00000196136 | SERPINA3 | 5.4768289 | 4.72097273 | 4.10E−193 |
| ENSG00000162873 | KLHDC8A | 1.13643032 | 4.71148824 | 1.94E−192 |
| ENSG00000132688 | NES | 3.26063514 | 4.61622082 | 2.71E−187 |
| ENSG00000134531 | EMP1 | 3.15206151 | 4.42972224 | 1.66E−174 |
| ENSG00000181104 | F2R | 1.23857625 | 4.41003053 | 1.46E−172 |
| ENSG00000229807 | XIST | 1.61098898 | 4.39884917 | 2.13E−171 |
| ENSG00000168542 | COL3A1 | 1.17496623 | 4.41375537 | 2.09E−166 |
| ENSG00000113140 | SPARC | 19.3375981 | 4.20699279 | 4.10E−161 |
| ENSG00000164692 | COL1A2 | 1.58994734 | 4.11304809 | 4.54E−150 |
| ENSG00000187498 | COL4A1 | 1.45266127 | 4.1109279 | 9.21E−149 |
| ENSG00000074410 | CA12 | 1.35700368 | 4.03323633 | 9.49E−148 |
| ENSG00000182718 | ANXA2 | 2.17859775 | 3.9971458 | 1.41E−146 |
| ENSG00000018408 | WWTR1 | 2.44183045 | 3.99077399 | 2.26E−146 |
| ENSG00000102265 | TIMP1 | 15.495953 | 3.95925361 | 1.73E−144 |
| ENSG00000179431 | FJX1 | 1.95811535 | 3.85754333 | 8.75E−138 |
| ENSG00000144810 | COL8A1 | 1.74218086 | 3.77807497 | 3.24E−132 |
| ENSG00000159403 | C1R | 1.50108129 | 3.7627829 | 7.79E−132 |
| ENSG00000163565 | IFI16 | 1.30015488 | 3.66606153 | 8.00E−126 |
| ENSG00000182326 | C1S | 1.2276916 | 3.60670229 | 3.59E−122 |
| ENSG00000115414 | FN1 | 2.86026797 | 3.60549473 | 2.98E−120 |
| ENSG00000172037 | LAMB2 | 1.48695247 | 3.54299336 | 8.10E−119 |
| ENSG00000134668 | SPOCD1 | 1.30752248 | 3.49230713 | 2.04E−114 |
| ENSG00000131435 | PDLIM4 | 1.15774484 | 3.38491192 | 4.87E−109 |
| ENSG00000110492 | MDK | 1.28423721 | 3.18784985 | 1.52E−97 |
| ENSG00000185201 | IFITM2 | 2.2629159 | 3.16213754 | 9.48E−97 |
| ENSG00000245532 | NEAT1 | 5.94474762 | 3.11898334 | 1.16E−94 |
| ENSG00000149131 | SERPING1 | 2.81002878 | 3.09823979 | 1.84E−93 |
| ENSG00000142156 | COL6A1 | 2.1866627 | 3.06636705 | 1.22E−91 |
| ENSG00000164434 | FABP7 | 4.107273 | 3.05359799 | 4.48E−91 |
| ENSG00000204287 | HLA-DRA | 1.4910759 | 3.00103364 | 5.91E−88 |
| ENSG00000101955 | SRPX | 1.19876693 | 3.02158944 | 7.46E−88 |
| ENSG00000175899 | A2M | 6.9004139 | 2.97943542 | 2.34E−87 |
| ENSG00000138448 | ITGAV | 2.72458882 | 2.96132092 | 2.74E−86 |
| ENSG00000105855 | ITGB8 | 2.14930925 | 2.95931109 | 3.68E−86 |
| ENSG00000118785 | SPP1 | 19.7822315 | 2.90867342 | 1.84E−83 |
| ENSG00000026025 | VIM | 28.2939982 | 2.8893192 | 1.60E−82 |
| ENSG00000162493 | PDPN | 1.15159001 | 2.88555631 | 3.51E−81 |
| ENSG00000110799 | VWF | 1.01976142 | 2.89038134 | 7.21E−81 |
| ENSG00000115380 | EFEMP1 | 1.93540615 | 2.86314337 | 7.67E−81 |
| ENSG00000164111 | ANXA5 | 2.8731537 | 2.85432725 | 1.16E−80 |
| ENSG00000026508 | CD44 | 2.20100376 | 2.85568845 | 4.17E−80 |
| ENSG00000163191 | S100A11 | 1.72347381 | 2.83125421 | 1.77E−78 |
| ENSG00000060982 | BCAT1 | 1.20486112 | 2.80431383 | 1.26E−77 |
| ENSG00000080493 | SLC4A4 | 2.76451942 | 2.78461039 | 5.17E−77 |
| ENSG00000181722 | ZBTB20 | 1.32422845 | 2.76771188 | 7.81E−76 |
| ENSG00000163584 | RPL22L1 | 1.03422376 | 2.77109136 | 1.29E−75 |
| ENSG00000106278 | PTPRZ1 | 5.56918168 | 2.75134542 | 1.50E−75 |
| ENSG00000162430 | SELENON | 1.45572353 | 2.7580052 | 2.84E−75 |
| ENSG00000163453 | IGFBP7 | 8.54184352 | 2.73694606 | 1.35E−74 |
| ENSG00000114115 | RBP1 | 1.12181639 | 2.72610485 | 1.38E−73 |
| ENSG00000062716 | VMP1 | 2.04034146 | 2.713888 | 4.76E−73 |
| ENSG00000168615 | ADAM9 | 1.09665132 | 2.66972765 | 7.30E−71 |
| ENSG00000206503 | HLA-A | 6.85047792 | 2.64688475 | 2.91E−70 |
| ENSG00000135404 | CD63 | 8.52225721 | 2.64335333 | 3.81E−70 |
| ENSG00000011465 | DCN | 1.02033749 | 2.67132502 | 6.26E−70 |
| ENSG00000234745 | HLA-B | 5.63476246 | 2.61600871 | 1.26E−68 |
| ENSG00000105835 | NAMPT | 1.69172944 | 2.60619255 | 1.41E−67 |
| ENSG00000112715 | VEGFA | 4.77830112 | 2.5993549 | 3.89E−67 |
| ENSG00000105894 | PTN | 7.42108831 | 2.57308207 | 1.03E−66 |
| ENSG00000158710 | TAGLN2 | 1.20792338 | 2.55843037 | 3.45E−65 |
| ENSG00000249992 | TMEM158 | 1.07457883 | 2.52534622 | 8.88E−64 |
| ENSG00000019582 | CD74 | 3.11288889 | 2.48616331 | 2.79E−62 |
| ENSG00000125148 | MT2A | 44.2981341 | 2.47099387 | 1.27E−61 |
| ENSG00000150093 | ITGB1 | 1.58952287 | 2.46958778 | 2.84E−61 |
| ENSG00000122786 | CALD1 | 1.52651924 | 2.42166646 | 4.16E−59 |
| ENSG00000108679 | LGALS3BP | 1.34399668 | 2.3881849 | 1.67E−57 |
| ENSG00000144136 | SLC20A1 | 1.8275905 | 2.38033065 | 2.91E−57 |
| ENSG00000177697 | CD151 | 1.03252587 | 2.38222765 | 8.25E−57 |
| ENSG00000142089 | IFITM3 | 3.19726768 | 2.34126085 | 1.12E−55 |
| ENSG00000166710 | B2M | 10.7930561 | 2.33248744 | 1.86E−55 |
| ENSG00000113594 | LIFR | 1.33101999 | 2.31802452 | 1.75E−54 |
| ENSG00000118705 | RPN2 | 1.42798131 | 2.3153264 | 2.15E−54 |
| ENSG00000145824 | CXCL14 | 3.19726768 | 2.29573873 | 1.24E−53 |
| ENSG00000002586 | CD99 | 2.16449925 | 2.28033431 | 6.36E−53 |
| ENSG00000139289 | PHLDA1 | 1.10626256 | 2.28238255 | 1.17E−52 |
| ENSG00000136158 | SPRY2 | 1.38016768 | 2.26381838 | 4.45E−52 |
| ENSG00000111341 | MGP | 2.84113645 | 2.27064135 | 9.78E−52 |
| ENSG00000148926 | ADM | 1.66798938 | 2.26777313 | 2.18E−51 |
| ENSG00000115461 | IGFBP5 | 3.12914007 | 2.24386561 | 3.44E−51 |
| ENSG00000135046 | ANXA1 | 1.84511509 | 2.25296807 | 3.65E−51 |
| ENSG00000138434 | ITPRID2 | 1.12742547 | 2.24158614 | 4.33E−51 |
| ENSG00000106236 | NPTX2 | 1.15868474 | 2.2153727 | 7.05E−50 |
| ENSG00000185222 | TCEAL9 | 1.24227522 | 2.20407075 | 1.87E−49 |
| ENSG00000103187 | COTL1 | 1.08397783 | 2.18330218 | 1.51E−48 |
| ENSG00000204525 | HLA-C | 2.59342726 | 2.17883952 | 1.53E−48 |
| ENSG00000102024 | PLS3 | 1.18203064 | 2.17979207 | 2.07E−48 |
| ENSG00000143870 | PDIA6 | 1.40375614 | 2.17324256 | 3.50E−48 |
| ENSG00000100644 | HIF1A | 1.42194776 | 2.14266248 | 5.76E−47 |
| ENSG00000185624 | P4HB | 2.12908623 | 2.10785913 | 1.53E−45 |
| ENSG00000204580 | DDR1 | 1.81079357 | 2.08621227 | 1.36E−44 |
| ENSG00000166340 | TPP1 | 1.85684868 | 2.07020271 | 4.68E−44 |
| ENSG00000124225 | PMEPA1 | 1.02403645 | 2.05406382 | 3.00E−43 |
| ENSG00000183255 | PTTG1IP | 1.42852706 | 2.01531059 | 9.05E−42 |
| ENSG00000089157 | RPLP0 | 5.56678645 | 2.00470346 | 1.41E−41 |

In addition, the whole transcriptome sequencing library was selectively enriched for targeted sequencing using hybrid capture pulldown of a panel of human neuroscience-related transcripts. Targeted sequencing and analysis showed that genes were differentially expressed, for example, the genes shown in Table 16.

TABLE 16
top overexpressed genes in glioblastoma relative to normal, targeted sequencing
results.

| | | | Glioblastoma | |
|---|---|---|---|---|
| | | Glioblastoma | Log2 Fold | Glioblastoma |
| FeatureID | FeatureName | Average | Change | P-Value |
| ENSG00000132688 | NES | 3.43009676 | 4.6310742 | 4.84E−205 |
| ENSG00000187498 | COL4A1 | 1.28661071 | 4.22722676 | 3.65E−173 |
| ENSG00000172037 | LAMB2 | 1.72165588 | 3.78208577 | 7.19E−148 |
| ENSG00000113140 | SPARC | 9.16340877 | 3.6887985 | 1.17E−142 |
| ENSG00000115414 | FN1 | 2.13504587 | 3.59808635 | 3.61E−134 |
| ENSG00000067182 | TNFRSF1A | 1.0292648 | 3.32218081 | 1.52E−118 |
| ENSG00000204287 | HLA-DRA | 1.43134179 | 3.25290006 | 1.07E−114 |
| ENSG00000144908 | ALDH1L1 | 1.31319923 | 2.96682169 | 4.31E−98 |
| ENSG00000026508 | CD44 | 1.93373 | 2.89811978 | 5.48E−94 |
| ENSG00000196924 | FLNA | 1.75464387 | 2.71813036 | 4.13E−84 |
| ENSG00000105835 | NAMPT | 1.37749113 | 2.71648475 | 7.64E−84 |
| ENSG00000112715 | VEGFA | 3.3302016 | 2.68422099 | 5.11E−82 |
| ENSG00000125730 | C3 | 1.13187427 | 2.65710698 | 1.03E−80 |
| ENSG00000206503 | HLA-A | 5.27657205 | 2.57580858 | 6.24E−77 |
| ENSG00000030582 | GRN | 1.03818047 | 2.56578626 | 6.34E−76 |
| ENSG00000234745 | HLA-B | 3.65550476 | 2.52262648 | 4.97E−74 |
| ENSG00000118785 | SPP1 | 8.5402033 | 2.50180907 | 6.74E−73 |
| ENSG00000166340 | TPP1 | 2.24066687 | 2.4417344 | 8.04E−70 |
| ENSG00000115457 | IGFBP2 | 3.52339431 | 2.37417656 | 4.57E−66 |
| ENSG00000148926 | ADM | 1.65249008 | 2.36926752 | 3.29E−65 |
| ENSG00000100644 | HIF1A | 1.3881305 | 2.24350791 | 8.27E−60 |
| ENSG00000106366 | SERPINE1 | 1.45915869 | 2.10910808 | 7.13E−53 |
| ENSG00000170558 | CDH2 | 1.07200058 | 2.07406104 | 7.09E−52 |
| ENSG00000125398 | SOX9 | 1.08987155 | 1.82351282 | 5.27E−41 |
| ENSG00000119655 | NPC2 | 1.10399794 | 1.8151279 | 1.20E−40 |
| ENSG00000181449 | SOX2 | 1.6308349 | 1.7687618 | 6.86E−39 |
| ENSG00000205336 | ADGRG1 | 1.43494768 | 1.74713688 | 4.81E−38 |
| ENSG00000131981 | LGALS3 | 2.89394787 | 1.70038942 | 4.88E−36 |
| ENSG00000079215 | SLC1A3 | 4.93541881 | 1.53182532 | 5.49E−30 |
| ENSG00000108518 | PFN1 | 2.23670434 | 1.52433325 | 1.16E−29 |
| ENSG00000114353 | GNAI2 | 1.5285028 | 1.51892911 | 1.96E−29 |
| ENSG00000130203 | APOE | 13.9945538 | 1.47722043 | 4.82E−28 |
| ENSG00000160307 | S100B | 5.17523057 | 1.46571086 | 1.21E−27 |
| ENSG00000161011 | SQSTM1 | 1.8469706 | 1.40154042 | 1.96E−25 |
| ENSG00000112096 | SOD2 | 1.85770904 | 1.34069361 | 3.61E−23 |
| ENSG00000131095 | GFAP | 12.2349766 | 1.33087211 | 3.62E−23 |
| ENSG00000117984 | CTSD | 2.96440149 | 1.30991243 | 1.72E−22 |
| ENSG00000148180 | GSN | 1.74864065 | 1.24216165 | 2.16E−20 |
| ENSG00000113712 | CSNK1A1 | 1.07023726 | 1.09957174 | 2.48E−16 |
| ENSG00000102144 | PGK1 | 2.18243762 | 1.09112437 | 3.78E−16 |
| ENSG00000067560 | RHOA | 1.80364044 | 1.07206323 | 1.19E−15 |
| ENSG00000137710 | RDX | 1.12412755 | 1.06725974 | 1.77E−15 |
| ENSG00000184009 | ACTG1 | 7.72741102 | 1.03244178 | 1.15E−14 |
| ENSG00000122566 | HNRNPA2B1 | 2.36516927 | 1.03025827 | 1.33E−14 |
| ENSG00000123384 | LRP1 | 1.41273775 | 0.9924383 | 1.24E−13 |
| ENSG00000084207 | GSTP1 | 1.21027275 | 0.9897912 | 1.48E−13 |
| ENSG00000106211 | HSPB1 | 1.84550447 | 0.95859759 | 8.04E−13 |
| ENSG00000120885 | CLU | 15.3904707 | 0.94628642 | 1.40E−12 |
| ENSG00000133048 | CHI3L1 | 3.4428957 | 0.94483501 | 2.95E−12 |
| ENSG00000185896 | LAMP1 | 1.06946457 | 0.93909228 | 2.32E−12 |
| ENSG00000177700 | POLR2L | 1.23698014 | 0.83317238 | 4.86E−10 |
| ENSG00000168036 | CTNNB1 | 1.31092078 | 0.8153585 | 1.13E−09 |
| ENSG00000128272 | ATF4 | 1.18742882 | 0.76698972 | 1.07E−08 |
| ENSG00000152661 | GJA1 | 1.55762733 | 0.623434 | 3.24E−06 |
| ENSG00000136156 | ITM2B | 4.66743352 | 0.52479654 | 8.90E−05 |
| ENSG00000112531 | QKI | 1.93178836 | 0.4993827 | 0.00019628 |
| ENSG00000156508 | EEF1A1 | 2.69023467 | 0.37810806 | 0.00493817 |
| ENSG00000189403 | HMGB1 | 2.33101234 | 0.28006584 | 0.03811429 |
| ENSG00000189058 | APOD | 1.3076517 | 0.24009539 | 0.07689109 |
| ENSG00000240972 | MIF | 3.70820628 | 0.11748718 | 0.39079095 |
| ENSG00000018625 | ATP1A2 | 1.48800584 | 0.09612068 | 0.48612996 |
| ENSG00000136238 | RAC1 | 1.37061616 | 0.05141084 | 0.71150614 |
| ENSG00000115053 | NCL | 1.38064134 | 0.04106448 | 0.76710185 |

Several overexpressed genes appeared in the whole transcriptome and targeted sequencing results. See Table 17 below.

TABLE 17
overexpressed genes in glioblastoma relative to normal tissue, as shown
in both whole transcriptome and targeted sequencing approaches.
Overlapping Genes
ADM
CD44
FN1
HLA-A
HLA-B
HLA-DRA
LAMB2
NAMPT
NES
SPARC
SPP1
VEGFA

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of determining abundance of two or more analytes in a subject having Alzheimer's Disease, comprising
determining the abundance of the two or more analytes selected from the group consisting of pro-melanin concentrating hormone (PMCH), aldo-keto reductase family 1, member E1 (Akr1e1), autophagy related 4C cysteine peptidase (Atg4c), Gm14296, hypocretin neuropeptide precursor (Hcrt), transthyretin (Ttr), adenosine A2a receptor (Adora2a), thyrotropin releasing hormone preproprotein (Trh), prion protein (Prnp), protein kinase C theta (Prkck), complement C1q B chain (C1qb), glutamate-ammonia ligase (Glul), pituitary tumor-transforming gene 1 (Pttg1), aurora kinase A interacting protein 1 (Aurkaip1), cocaine- and amphetamine-regulated transcript protein (Cartpt), complement component 4B (C4b), mitotic spindle organizing protein 1 (Mzt1), tyrosine-protein phosphatase non-receptor type 3 (Ptpn3), phytanoyl-CoA 2-hydroxylase interacting protein (Phyhip), small nucleolar RNA host gene 11 (Snhg11), RAD23 homolog B (Rad23b), netrin G1 (Ntng1), serine/arginine-rich splicing factor 5 (Srsf5), tyrosine-protein phosphatase non-receptor type 4 (Ptpn4), 5′-nucleotidase domain containing 3 (Nt5dc3), insulin induced gene 1 (Insig1), oxytocin (Oxt), delta-aminolevulinate dehydratase (Alad), nudix hydrolase 19 (Nudt19), Gm10076 ribosomal protein L41 pseudogene (Gm10076), cyclase associated actin cytoskeleton regulatory protein 1 (Cap1), regulator of cell cycle (Rgcc), ubiquitin A-52 residue ribosomal protein fusion product 1 (Uba52), protein phosphatase 1 regulatory inhibitor subunit 1B (Ppp1r1b), phosphodiesterase 10A (Pde10a), Ubiquitin Conjugating Enzyme E2 M (Ube2m), hemoglobin alpha, adult chain 1 (Hba-a1), glutathione S-transferase pi gene (Gstp1), Mesencephalic Astrocyte Derived Neurotrophic Factor (Manf), G Protein-Coupled Receptor 88 (Gpr88), Sin3A-associated protein (Sap301), alkB homolog 6 (Alkbh6), Small nucleolar RNA host gene 6 (Snhg6), Arginine Vasopressin (Avp), Profilin-1 (Pfn1), tachykinin, precursor 1 (Tac1), byproducts, precursors and degradation products thereof, in a biological sample obtained from the subject.

2. The method of claim 1, wherein the biological sample from the subject comprises more than one biological sample from the subject from a plurality of time points and determining the abundance of the two or more analytes in the two or more biological samples from the plurality of time points from the subject.

3. A method of treating Alzheimer's Disease in a subject, wherein the method comprises:
(a) determining an abundance of one or more analytes selected from PMCH, Akr1e1, Atg4c, Gm14296, Hcrt, Ttr, Adora2a, and Trh, and byproducts, precursors and degradation products thereof, in the biological sample;
(b) identifying the subject having at least one of:
(1) an elevated abundance of the one or more analytes PMCH, Akr1e1, Atg4c, Gm14296, Hcrt, and Ttr, and byproducts, precursors and degradation products thereof, of step (a), and
(2) about the same or a decreased abundance of the one or more analytes Adora2a, Trh, byproducts, precursors and degradation products thereof, of step (a);
as having Alzheimer's disease; and
(c) administering a therapeutic treatment for Alzheimer's Disease to the subject.

4. The method of claim 3, wherein the one or more analytes are selected from PMCH, Akr1e1, Atg4c, Gm14296, Hcrt, Ttr, Adora2a, Trh, Prnp, Prkck, C1qb, Glul, Pttg1, Aurkaip1, Cartpt, C4b, Mzt1, Ptpn3, Phyhip, Snhg11, Rad23b, Ntng1, Srsf5, Ptpn4, Nt5dc3, Insig1, Oxt, Alad, Nudt19, Gm10076, Cap1, Rgcc, Uba52, Ppp1r1b, Pde10a, Ube2m, Hba-a1, Gstp1, Manf, Gpr88, Sap301, Alkbh6, Snhg6, Avp, Pfn1, Tac1, byproducts, precursors and degradation products thereof; and
wherein the method further comprises:
(a) identifying the subject having at least one of:
(1) an elevated abundance of the one or more analytes selected from PMCH, Akr1e1, Atg4c, Gm14296, Hcrt, Ttr, Prnp, Prkck, C1qb, Glul, Pttg1, Aurkaip1, Cartpt, C4b, Mzt1, Ptpn3, Phyhip, Snhg11, Rad23b, Ntng1, Srsf5, Ptpn4, Nt5dc3, Insig1, and byproducts, precursors and degradation products thereof, or
(2) about the same or a decreased abundance of the one or more analytes selected from Adora2a, Trh, Oxt, Alad, Nudt19, Gm10076, Cap1, Rgcc, Uba52, Ppp1r1b, Pde10a, Ube2m, Hba-a1, Gstp1, Manf, Gpr88, Sap301, Alkbh6, Snhg6, Avp, Pfn1, Tac1, byproducts, precursors and degradation products thereof,
as having Alzheimer's disease.

5-13. (canceled)

14. The method of claim 3, wherein the one or more analytes are pro-melanin concentrating hormone (PMCH), hypocretin neuropeptide precursor (Hcrt), oxytocin (Oxt), and phytanoyl-CoA 2-hydroxylase interacting protein (Phyhip), and byproducts, precursors and degradation products thereof.

15. (canceled)

16. The method of claim 1, further comprising administering a therapeutic treatment of Alzheimer's disease to the subject, adjusting a dosage of a therapeutic treatment of Alzheimer's disease for the subject, or adjusting a therapeutic treatment of Alzheimer's disease for the subject.

17. The method of claim 3, wherein the therapeutic treatment comprises:
(a) an antagonist of one or more of the analytes selected from the group consisting of PMCH, Akr1e1, Atg4c, Gm14296, Hcrt, Ttr, Prnp, Prkck, C1qb, Glul, Pttg1, Aurkaip1, Cartpt, C4b, Mzt1, Ptpn3, Phyhip, Snhg11, Rad23b, Ntng1, Srsf5, Ptpn4, Nt5dc3, and Insig1, and byproducts, precursors and degradation products thereof; or
(b) an agonist of one or more of the analytes selected from the group consisting of Adora2a, Trh, Oxt, Alad, Nudt19, Gm10076, Cap1, Rgcc, Uba52, Ppp1r1b, Pde10a, Ube2m, Hba-a1, Gstp1, Manf, Gpr88, Sap301, Alkbh6, Snhg6, Avp, Pfn1, Tac1, and byproducts, precursors and degradation products thereof.

18. The method of claim 3, wherein the therapeutic treatment comprises administering one or more agents selected from a cholinesterase inhibitor, an N-methyl-D-aspartate (NMDA) inhibitor, an antipsychotic, a tricyclic antidepressant, a benzodiazepine, insulin, and tacrine hydrochloride.

19. The method of claim 18, wherein the cholinesterase inhibitor is galantamine, rivastigmine, or donepezil; the NMDA inhibitor is memantine; the antipsychotic agent is aripiprazole, risperidone, olanzapine, quetiapine, or haloperidol; the benzodiazepine is lorazepam, oxazepam or temazepam; and the tricyclic antidepressant is nortriptyline.

20-51. (canceled)

52. The method of claim 1, wherein the two or more analytes is an mRNA molecule.

53. The method of claim 52, wherein the determining step comprises determining the abundance and location of the two or more analytes, the method comprising:
(a) contacting the biological sample with a substrate comprising a plurality of attached capture probes, wherein a capture probe of the plurality comprises (i) a spatial barcode and (ii) a capture domain that binds to a sequence present in the analyte;
(b) hybridizing the two or more analytes to the capture domain;
(c) extending a 3′ end of the capture probe using the analyte that is bound to the capture domain as a template to generate an extended capture probe;
(d) amplifying the extended capture probe; and
(e) determining (i) all or a portion of the sequence of the spatial barcode or the complement thereof, and (ii) all or a portion of the sequence of the analyte from the biological sample; and using the determined sequences of (i) and (ii) to identify the location of the analyte in the biological sample, thereby determining the abundance and location of the two or more analytes.

54. The method of claim 1, wherein the two or more analytes is a protein.

55. The method of claim 54, wherein the determining step comprises determining the abundance and location of the two or more analytes, the method comprising:
(a) attaching the biological sample with a plurality of analyte capture agents, wherein an analyte capture agent of the plurality of analyte capture agents comprises:
(i) an analyte binding moiety that binds to the two or more analytes;
(ii) an analyte binding moiety barcode that uniquely identifies an interaction between the two or more analytes and the analyte binding moiety; and
(iii) an analyte capture sequence, wherein the analyte capture sequence binds to a capture domain;
(b) contacting the biological sample with a substrate, wherein the substrate comprises a plurality of capture probes, wherein a capture probe of the plurality of capture probes comprises (i) the capture domain and (ii) a spatial barcode;
(c) hybridizing the two or more analytes to the capture probe; and
(d) determining (i) all or a part of a sequence corresponding to the analyte binding moiety barcode, and (ii) all or a part of a sequence corresponding to the spatial barcode, or a complement thereof, and using the determined sequence of (i) and (ii) to identify the abundance and spatial location of the two or more analytes in the biological sample.

56. The method of claim 1, wherein the biological sample comprises at least one of cerebrospinal fluid, whole blood, plasma, and serum.

57. The method of claim 3, wherein the one or more analytes is an mRNA molecule.

58. The method of claim 57 wherein the determining step comprises determining the abundance and location of the one or more analytes, the method comprising:
(a) contacting the biological sample with a substrate comprising a plurality of attached capture probes, wherein a capture probe of the plurality comprises (i) a spatial barcode and (ii) a capture domain that binds to a sequence present in the analyte;
(b) hybridizing the one or more analytes to the capture domain;
(c) extending a 3′ end of the capture probe using the analyte that is bound to the capture domain as a template to generate an extended capture probe;
(d) amplifying the extended capture probe; and
(e) determining (i) all or a portion of the sequence of the spatial barcode or the complement thereof, and (ii) all or a portion of the sequence of the analyte from the biological sample; and using the determined sequences of (i) and (ii) to identify the location of the analyte in the biological sample, thereby determining the abundance and location of the one or more analytes.

59. The method of claim 3, wherein the one or more analytes is a protein.

60. The method of claim 59, wherein the determining step comprises determining the abundance and location of the one or more analytes, the method comprising:
(a) attaching the biological sample with a plurality of analyte capture agents, wherein an analyte capture agent of the plurality of analyte capture agents comprises:
(i) an analyte binding moiety that binds to the one or more analytes;
(ii) an analyte binding moiety barcode that uniquely identifies an interaction between the one or more analytes and the analyte binding moiety; and
(iii) an analyte capture sequence, wherein the analyte capture sequence binds to a capture domain;
(b) contacting the biological sample with a substrate, wherein the substrate comprises a plurality of capture probes, wherein a capture probe of the plurality of capture probes comprises (i) the capture domain and (ii) a spatial barcode;
(c) hybridizing the one or more analytes to the capture probe; and
(d) determining (i) all or a part of a sequence corresponding to the analyte binding moiety barcode, and (ii) all or a part of a sequence corresponding to the spatial barcode, or a complement thereof, and using the determined sequence of (i) and (ii) to identify the abundance and spatial location of the one or more analytes in the biological sample.

61. The method of claim 3, wherein the biological sample comprises at least one of cerebrospinal fluid, whole blood, plasma, and serum.

62. A kit comprising:
(a) an antibody that binds specifically to two or more analytes selected from the group consisting of PMCH, AKR1E1, ATG4C, GM14296, HCR, TTR, ADORA2A, TRH, PRNP, PRKCK, C1QB, GLUL, PTTG1, AURKAIP1, CARTPT, C4B, MZT1, PTPN3, PHYHIP, SNHG11, RAD23B, NTNG1, SRSF5, PTPN4, NT5DC3, INSIG1, OXT, ALAD, NUDT19, GM10076, CAP1, RGCC, UBA52, PPP1R1B, PDE10A, UBE2M, HBA-A1, GSTP1, MANF, GPR88, SAP30L, ALKBH6, SNHG6, AVP, PFN1, TAC1, byproducts, precursors and degradation products thereof; and
(b) instructions for performing the method of claim 53.
